(19) United States
(12) Patent Application Publication
(10) Pub. No.: US 2023/0416307 A1
(43) Pub. Date: Dec. 28, 2023

(54) ENGINEERED CYCLIC PEPTIDES FOR ENHANCING VIRAL PRODUCTION

(71) Applicant: Synvivia, Inc., Berkeley, CA (US)

(72) Inventors: Gabriel Lopez, Oakland, CA (US); Charlie Huang, Oakland, CA (US)

(73) Assignee: Synvivia, Inc., Berkeley, CA (US)

(21) Appl. No.: 18/339,962

(22) Filed: Jun. 22, 2023

Related U.S. Application Data
(60) Provisional application No. 63/354,637, filed on Jun. 22, 2022.

(51) Int. Cl. C07K 7/64; C12N 15/86; C07K 14/00

(52) U.S. Cl. CPC C07K 7/64; C12N 15/86; C07K 14/001; C12N 2740/15043; C12N 2740/15052; C12N 2750/14143; C12N 2750/14152

(57) ABSTRACT

The present disclosure provides methods and compositions for production of recombinant viral vectors, such as adeno-associated virus (AAV) vectors or lentiviral vectors, in host cells using engineered cyclic peptides that increase viral titer and transduction efficiency of viral vector compositions. The present disclosure also provides methods for selecting genetically encoded, endogenously expressed cyclic peptides that enhance viral vector manufacturability.

### CROSS-REFERENCE TO RELATED APPLICATIONS

This application claims priority to U.S. Provisional Patent Application No. 63/354,637 filed Jun. 22, 2022, entitled “METHODS OF SELECTION AND USE OF ENGINEERED CYCLIC PEPTIDES FOR ENHANCING AAV PRODUCTION,” which is herein incorporated by reference in its entirety for all purposes.

### REFERENCE TO AN ELECTRONIC SEQUENCE LISTING

The contents of the electronic sequence listing (SYNV01US_SEQLIST.xml; Size: 279,155 bytes; and Date of Creation: Jun. 22, 2023) is herein incorporated by reference in its entirety.

### TECHNICAL FIELD

The present disclosure generally relates to biotechnology and, in particular, to methods and compositions for production of recombinant viral vectors, such as AAV vectors, in host cells. Recombinant viral vectors can be used as gene delivery vehicles for treatment of human diseases.

### INTRODUCTION

Production of highly infectious viral vector compositions has significant challenges. For example, recombinant adeno-associated virus (AAV) is a leading gene delivery platform for treatment of human diseases with many advantages, including good safety profile, long persistence of AAV-delivered genetic payloads in target cells, as well as strong and diverse tropism (ability to target specific tissues). However, AAV production at an industrial scale has been accomplished only to a limited degree. There are several major problems with the manufacturing and use of AAV vectors as gene therapy. When using helper virus strategies, the resulting AAVs are highly contaminated with pathogenic viruses, which can be challenging to remove. For this reason, helper virus free approaches are typically preferred. However, different challenges arise. First, up to 90% of viral capsids produced during manufacturing can be empty. Although it is possible to purify full capsids away from empty capsids, this procedure is costly, difficult to scale, and reduces yield. Second, among the properly packaged recombinant viral capsids, only a small fraction of them is able to deliver their payload into target cells (often 0.1% or less). To compensate for this during treatment, the dose needs to be increased significantly; however, this increases the cost of treatment and reduces the safety profile.

A cause of these problems is that the intracellular environment of packaging cells is not optimized for producing viral vectors. For example, it is known that during the AAV natural replication cycle, the associated adenovirus significantly perturbs and optimizes the cellular environment as part of its own lifecycle, which also provides a cellular milieu that is highly optimized for AAV replication. Since the use of “helper” adenoviruses to assist AAV production poses a significant safety risk, current AAV manufacturing platforms avoid the use of helper viruses instead relying on cloned helper genes. Expression of cloned helper genes poorly mimics the adenovirus induced changes to the intracellular environment and may have undesirable effects on the host cell used to package AAV vectors. The multitude of mechanisms that determine AAV vector quality are not well understood. Post-translational modifications, such as glycosylation, acetylation, phosphorylation of AAV capsid, as well as variable DNA methylation of the AAV genome, are all thought to play important roles.

There is a need for improved viral production methods that will satisfy the current demand of viral vector material for clinical trials and market supply. The current invention addresses this need by providing methods of production of highly infectious viral vector compositions.

These and other embodiments of the invention will be apparent upon reference to the following detailed description. To this end, various references are set forth herein which describe in more detail certain background information, procedures, compounds and/or compositions, and are each hereby incorporated by reference in their entireties.

### SUMMARY

The summary is not intended to be used to limit the scope of the claimed subject matter. Other features, details, utilities, and advantages of the claimed subject matter will be apparent from the detailed description including those embodiments disclosed in the accompanying drawings and in the appended claims.

The present teachings include methods that allow the discovery of endogenously expressed cyclic peptides that can positively modulate/enhance viral packaging and manufacturability. The proposed methods also improve infectiousness of the produced viral vector composition.

The present teachings include a method of obtaining an engineered cyclic peptide (CP) capable of increasing viral titer and/or transduction efficiency of a viral vector composition, the method comprising:
(a) culturing a first plurality of host cells permissive for replication of a virus under conditions suitable for recombinant viral production, wherein each host cell of the first plurality of host cells comprises:
(i) at least one viral replication gene essential for the replication of the virus;
(ii) at least one viral structural gene essential for formation of viral capsids;
(iii) at least one additional viral gene necessary for the production of the virus in the host cells; and
(iv) an engineered cyclic peptide produced from a polypeptide encoded by a first nucleotide sequence, wherein (v) the first nucleotide sequence is operably linked to one or more one or more viral-specific packaging sequences necessary for encapsulation of the first nucleotide sequence within the viral capsids or (vi) the first nucleotide sequence is associated with a second nucleotide sequence comprising a barcode that comprises identifying information regarding the engineered cyclic peptide produced in the host cell, and the second nucleotide sequence is operably linked to the one or more one or more viral-specific packaging sequences necessary for encapsulation of the second nucleotide sequence within the viral capsids, thereby obtaining a first plurality of viral vectors comprising the first nucleotide sequence and/or the second nucleotide sequence from the first plurality of host cells;
(b) optionally, repeating the following steps one or more times in cycles:
(b1) allowing a plurality of viral vectors of the previous cycle to infect a plurality of host cells of the present cycle permissive for replication of the virus; and
(b2) culturing the plurality of host cells of the present cycle under conditions suitable for recombinant viral production, wherein each host cell of the plurality of host cells of the present cycle comprises the elements (i)-(iii) of the first plurality of host cells, and further comprises the first nucleotide sequence operably linked to the one or more viral-specific packaging sequences producing the engineered cyclic peptide, thereby obtaining a plurality of viral vectors of the present cycle comprising the first nucleotide sequence;
(c) allowing the first plurality of viral vectors or the plurality of viral vectors of the present cycle to infect a final plurality of host cells; and (d) determining one or more engineered cyclic peptides capable of increasing viral titer and/or transduction efficiency of the viral vector composition by analyzing nucleotide sequences operably linked to the one or more viral-specific packaging sequences from (i) the final plurality of host cells and/or (ii) a final plurality of viral vectors produced in the final plurality of host cells.

The present teachings also include a plurality of host cells permissive for replication of a virus, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary for the production of the virus in the host cells; and
(iv) a nucleotide sequence operably linked to one or more viral-specific packaging sequences necessary for encapsulation of the nucleotide sequence within the viral capsids, wherein the nucleotide sequence encodes a payload; wherein the engineered cyclic peptide increases a characteristic of viral vectors produced by the plurality of host cells by at least 2-fold compared to a corresponding characteristic of viral vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of viral vectors is selected from the group consisting of viral titer and transduction efficiency.

The present teachings also include a method of producing a viral vector composition of increased viral titer and/or transduction efficiency, the method comprising:
(a) culturing a plurality of host cells permissive for replication of a virus under conditions suitable for recombinant viral production, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary to produce the virus in the host cells; and
(iv) a nucleotide sequence operably linked to one or more viral-specific packaging sequences necessary for encapsulation of the nucleotide sequence within the viral capsids, wherein the nucleotide sequence encodes a payload; and
(b) producing the viral vector composition of increased viral titer and/or transduction efficiency from the plurality of host cells, wherein the viral vector composition has an increased viral titer and/or transduction efficiency which is at least a 20%, 40%, 60%, 80%, 100%, 200%, or 500% greater than a viral titer and/or transduction efficiency of a reference viral vector composition produced in a plurality of reference host cells under essentially identical conditions, wherein each reference host cell of the plurality of reference host cells comprises the elements (i)-(iv) of the plurality of host cells and does not comprise the engineered cyclic peptide.

The present teachings also include a method of obtaining an engineered cyclic peptide capable of increasing viral titer and/or transduction efficiency of adeno-associated virus (AAV) vector composition, the method comprising:
(a) culturing a first plurality of host cells permissive for AAV replication under conditions suitable for recombinant AAV production, wherein each host cell of the first plurality of host cells comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) an engineered cyclic peptide produced from a polypeptide encoded by a first nucleotide sequence, wherein (v) the first nucleotide sequence is operably linked to at least two functional AAV inverted terminal repeats (ITRs) or (vi) the first nucleotide sequence is associated with a second nucleotide sequence comprising a barcode that comprises identifying information regarding the engineered cyclic peptide produced in the host cell, and the second nucleotide sequence is operably linked to at least two functional AAV ITRs, thereby obtaining a first plurality of AAV vectors comprising the first nucleotide sequence and/or the second nucleotide sequence from the first plurality of host cells;
(b) optionally, repeating the following steps one or more times in cycles:
(b1) allowing a plurality of AAV vectors of the previous cycle to infect a plurality of host cells of the present cycle permissive for AAV replication; and
(b2) culturing the plurality of host cells of the present cycle under conditions suitable for recombinant AAV production, wherein each host cell of the plurality of host cells of the present cycle comprises the elements (i)-(iii) of the first plurality of host cells, and further comprises the first nucleotide sequence operably linked to the at least two functional AAV ITRs producing the engineered cyclic peptide, thereby obtaining a plurality of AAV vectors of the present cycle comprising the first nucleotide sequence; (c) allowing the first plurality of AAV vectors or the plurality of AAV vectors of the present cycle to infect a final plurality of host cells; and (d) determining one or more engineered cyclic peptides capable of increasing viral titer and/or transduction efficiency of AAV vector composition by analyzing nucleotide sequences operably linked to the at least two functional AAV inverted terminal repeats (ITRs) from (i) the final plurality of host cells and/or (ii) a final plurality of AAV vectors produced in the final plurality of host cells.

The present teachings also include a method of producing an adeno-associated virus (AAV) vector composition, the method comprising:
(a) culturing a first plurality of host cells permissive for AAV replication under conditions suitable for recombinant AAV production, wherein each host cell of the first plurality of host cells comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) an engineered cyclic peptide produced from a polypeptide encoded by a first nucleotide sequence, wherein (v) the first nucleotide sequence is operably linked to at least two functional AAV inverted terminal repeats (ITRs) or (vi) the first nucleotide sequence is associated with a second nucleotide sequence comprising a barcode that comprises identifying information regarding the engineered cyclic peptide produced in the host cell, and the second nucleotide sequence is operably linked to at least two functional AAV inverted terminal repeats (ITRs), thereby obtaining a first plurality of AAV vectors comprising the first nucleotide sequence and/or the second nucleotide sequence from the first plurality of host cells;
(b) optionally, repeating the following steps one or more times in cycles:
(b1) allowing a plurality of AAV vectors of the previous cycle to infect a plurality of host cells of the present cycle permissive for AAV replication; and
(b2) culturing the plurality of host cells of the present cycle under conditions suitable for recombinant AAV production, wherein each host cell of the plurality of host cells of the present cycle comprises the elements (i)-(iii) of the first plurality of host cells, and further comprises the first nucleotide sequence operably linked to the at least two functional AAV ITRs producing the engineered cyclic peptide, thereby obtaining a plurality of AAV vectors of the present cycle comprising the first nucleotide sequence;
(c) allowing the first plurality of AAV vectors or the plurality of AAV vectors of the present cycle to infect a final plurality of host cells;
(d) determining one or more engineered cyclic peptides capable of increasing viral titer and/or transduction efficiency of AAV vector composition by analyzing nucleotide sequences operably linked to the at least two functional AAV inverted terminal repeats (ITRs) from (i) the final plurality of host cells and/or (ii) a final plurality of AAV vectors produced in the final plurality of host cells; and (e) obtaining new AAV vectors in the presence of an engineered cyclic peptide determined in step (d), thereby producing the AAV vector composition (e.g., an adeno-associated virus (AAV) vector composition of increased viral titer and/or transduction efficiency).

The present teachings also include a plurality of host cells permissive for AAV replication, wherein each host cell of the plurality of host cells comprises an engineered cyclic peptide and further comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) a nucleotide sequence operably linked to at least two functional AAV internal terminal repeats (ITRs), wherein the nucleotide sequence encodes a payload (e.g. therapeutic gene); wherein the engineered cyclic peptide increases a characteristic of AAV vectors produced by the plurality of host cells by at least 2-fold compared to a corresponding characteristic of AAV vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of AAV vectors is selected from the group consisting of physical titer, biological titer and transduction efficiency.

The present teachings also include a method of producing an adeno-associated virus (AAV) vector composition of increased viral titer and/or transduction efficiency, the method comprising: culturing a plurality of host cells permissive for AAV replication under conditions suitable for recombinant AAV production, wherein each host cell of the plurality of host cells comprises an engineered cyclic peptide and further comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) a nucleotide sequence operably linked between two functional AAV internal terminal repeats (ITRs), wherein the nucleotide sequence encodes a payload; and
(b) producing the AAV vector composition of increased viral titer and/or transduction efficiency from the plurality of host cells, wherein the AAV vector composition has an increased viral titer and/or transduction efficiency which is at least a 20%, 40%, 60%, 80%, 100%, 200%, or 500% greater than an viral titer and/or transduction efficiency of a reference AAV vector composition produced from a plurality of reference host cells under essentially identical conditions, wherein each reference host cell of the plurality of reference host cells comprises the elements (i)-(iv) of the plurality of host cells and does not comprise the engineered cyclic peptide.

The present teachings also include an AAV vector composition of increased viral titer and/or transduction efficiency produced by the disclosed methods.

In some embodiments, TU:VG ratio of the AAV vector composition of increased viral titer and/or transduction efficiency is from 1:100 to 1:50, from 1:50 to 1:20, from 1:20 to 1:10, from 1:10 to 1:5, from 1:5 to 1:2, or from 1:2 to 1:1.

In preferred embodiments, each host cell is a mammalian cell or an insect cell.

In some embodiments, the AAV vector composition of increased viral titer and/or transduction efficiency has a viral genome titer which is at least a 20%, 40%, 60%, 80%, 100%, 200%, or 500% greater than a viral genome titer of a reference AAV vector composition produced without engineered cyclic peptides.

The present teachings also include a method of obtaining an engineered cyclic peptide capable of increasing viral titer and/or transduction efficiency of lentivirus vector composition, the method comprising:
(a) culturing a first plurality of host cells permissive for lentivirus replication under conditions suitable for recombinant lentivirus production, wherein each host cell of the first plurality of host cells comprises:
(i) at least one lentiviral gag gene;
(ii) at least one lentiviral pol gene;
(iii) at least one lentiviral rev gene;
(iv) at least one env gene; and
(v) an engineered cyclic peptide produced from a polypeptide encoded by a first nucleotide sequence, wherein (vi) the first nucleotide sequence is operably linked to at a Psi sequence or (vii) the first nucleotide sequence is associated with a second nucleotide sequence comprising a barcode that comprises identifying information regarding the engineered cyclic peptide produced in the host cell, and the second nucleotide sequence is operably linked to a Psi sequence, thereby obtaining a first plurality of lentivirus vectors comprising the first nucleotide sequence and/or the second nucleotide sequence from the first plurality of host cells;
(b) optionally, repeating the following steps one or more times in cycles:
(b1) allowing a plurality of lentivirus vectors of the previous cycle to infect a plurality of host cells of the present cycle permissive for lentivirus replication; and
(b2) culturing the plurality of host cells of the present cycle under conditions suitable for recombinant lentivirus production, wherein each host cell of the plurality of host cells of the present cycle comprises the elements (i)-(iv) of the first plurality of host cells, and further comprises the first nucleotide sequence operably linked to a Psi sequence producing the engineered cyclic peptide, thereby obtaining a plurality of lentivirus vectors of the present cycle comprising the first nucleotide sequence;
(c) allowing the first plurality of lentivirus vectors or the plurality of lentivirus vectors of the present cycle to infect a final plurality of host cells; and (d) determining one or more engineered cyclic peptides capable of increasing viral titer and/or transduction efficiency of lentivirus vector composition by analyzing nucleotide sequences operably linked to a Psi sequence from (i) the final plurality of host cells and/or (ii) a final plurality of lentivirus vectors produced in the final plurality of host cells.

The present teachings also include a plurality of host cells permissive for lentivirus replication, wherein each host cell of the plurality of host cells comprises an engineered cyclic peptide and further comprises:
(i) at least one lentiviral gag gene;
(ii) at least one lentiviral pol gene;
(iii) at least one lentiviral rev gene;
(iv) at least one env gene; and
(v) a nucleotide sequence operably linked to a Psi sequence, wherein the nucleotide sequence encodes a payload; wherein the engineered cyclic peptide increases a characteristic of lentivirus vectors produced by the plurality of host cells by at least 2-fold compared to a corresponding characteristic of lentivirus vectors produced by a plurality of reference host cells under essentially identical conditions, wherein each reference host cell of the plurality of reference host cells comprises the elements (i)-(iv) of the plurality of host cells and does not comprise the engineered cyclic peptide, and wherein the characteristic of lentivirus vectors is selected from the group consisting of viral titer and transduction efficiency.

The present teachings also include a method of producing a lentivirus vector composition of increased viral titer and/or transduction efficiency, the method comprising:
(a) culturing a plurality of host cells permissive for lentiviral replication under conditions suitable for recombinant lentiviral production, wherein each host cell of the plurality of host cells comprises an engineered cyclic peptide and further comprises:
(i) at least one lentiviral gag gene;
(ii) at least one lentiviral pol gene;
(iii) at least one lentiviral rev gene;
(iv) at least one env gene; and
(v) a nucleotide sequence operably linked to a Psi sequence, wherein the nucleotide sequence encodes a payload (e.g. therapeutic gene); and (b) producing the lentivirus vector composition of increased viral titer and/or transduction efficiency from the plurality of host cells, wherein the lentivirus vector composition has an increased viral titer and/or transduction efficiency which is at least a 20%, 40%, 60%, 80%, 100%, 200%, or 500% greater than a viral titer and/or transduction efficiency of a reference lentivirus vector composition produced in a plurality of reference host cells under essentially identical conditions, wherein each reference host cell of the plurality of reference host cells comprises the elements (i)-(v) of the plurality of host cells and does not comprise the engineered cyclic peptide.

The present teachings also include a lentivirus vector composition of increased viral titer and/or transduction efficiency produced by the disclosed methods.

These and other features, aspects and advantages of the present teachings will become better understood with reference to the following description, examples and appended claims.

### BRIEF DESCRIPTION OF THE DRAWINGS

Those with skill in the art will understand that the drawings, described below, are for illustrative purposes only. The drawings are not intended to limit the scope of the present teachings in any way.

FIG. 1. Exemplary discovery of cyclic peptides that enhance viral production with a chemo-genetic selection and screening.
Part A. Packaging cells are transfected with a library of genes configured to produce cyclic peptides and flanked by two ITRs. The library may also include additional elements: fluorescent proteins (such as green fluorescent protein or GFP), reporter enzymes (luciferase) or a barcode. The packaging cells contain DNA encoding Rep, Cap, and helper genes (e.g. from triple transfection, stable integration, etc. . . . ). The cyclic peptide plasmid library (pCP) is depicted as circular plasmids 1, 2, . . . , and N.
Part B. The engineered cyclic peptide library and genes required for AAV production are expressed in host cells (e.g. through triple transfection, stable genome integration, helper virus, etc. . . . ). Engineered cyclic peptides that reduce AAV production reduce their own coding DNA from being packaged. Engineered cyclic peptides that enhance AAV production will enhance the packaging of their own coding DNA into AAV virions. A cartoon of petri dish with packaging cells is depicted.
Part C. The library of AAV virions were harvested from the AAV packaging cells. The AAV library harbors DNA encoding engineered cyclic peptides or DNA barcodes that can be used to identify the cyclic peptide present inside of the cell that the AAV was produced in. The library population is enriched for engineered cyclic peptide variants (or corresponding DNA barcodes) that enhance AAV titer (black AAV capsids). The library population is depleted in engineered cyclic peptide variants (or corresponding DNA barcodes) that reduce AAV titer. A portion of the AAV library is non-infectious (gray capsids).
Part D. The packaged cyclic peptide library was transduced into fresh packaging cells that have all necessary components for viral production (e.g. transfected with packaging plasmids, stable genome integration of packaging plasmids, or helper virus). This transduction step selects for DNA sequences that encode cyclic peptides whose presence in the previous viral packaging step improved the production of viral particles harboring their own cyclic peptide DNA coding sequence. Thus, cyclic peptide DNA coding sequences that express cyclic peptides that improve viral packaging gain a selective advantage that allows for increased nucleic acid transduction of such cyclic peptide coding sequences into fresh packaging cells. This couples the physiological impact of each cyclic peptide on viral packaging to the cyclic peptide coding sequence's ability to propagate, establishing a strong selective pressure for cyclic peptides that enhance viral titer or function/infectivity.
Part E. Packaging cells (containing necessary elements for viral production) that are successfully transduced by a cyclic peptide containing viral particle will produce a subsequent generation of viral particles harboring cyclic peptide coding sequences.
Part F. The resulting library of cyclic peptide DNA harboring viral particles were enriched for coding sequences for cyclic peptides that either increase physical titer or infectiousness (e.g., black particles) and depleted for coding sequences of cyclic peptides that interfere with viral packaging. At this point, there are more black, functional particles compared to gray, non-functional particles because of the selective enrichment for functional particles harboring cyclic peptide coding sequences that enhance viral production.
Part G. Subsequent rounds of enrichment are possible in a cyclic fashion by transducing the previous generation of AAV cyclic peptide library into fresh packaging cells. As in D-F, the coding sequences of cyclic peptides that mediate production of AAVs with increased infectivity and/or increased titer are selected for. More infectious AAVs are able to more efficiently deliver their DNA encoding the engineered cyclic peptide into packaging cells, which results in a greater number of more infectious AAVs being produced. Less infectious AAVs deliver their DNA encoding the engineered cyclic peptide into packaging cells less efficiently and are outcompeted. The coding sequences of cyclic peptides that mediate higher viral production titer and/or function will have a similar selective advantage. This enrichment loop is repeated as many times as desired. 2-4 rounds of enrichment were observed to be optimal.
Part H. After a desired number of rounds of selection, viral DNA is purified. DNA was purified at each round for NGS as described in FIG. 2 and FIG. 3. The ITRs are shown on either side of the gene as rectangular loops.
Part I. The cyclic peptide DNA library was moved from a viral transfer vector (e.g. that would be packaged into a viral particle as a result of ITRs) to a standard expression plasmid with no packaging sequences (i.e. so that it won't be packaged into a viral particle). The resulting library is cloned using standard molecular biology techniques (e.g. transform into E. coli, grow on petri dishes, pick individual colonies into liquid media, purify DNA). The cyclic peptide library is now separated into many individual plasmid isolates, providing a convenient form for genotypic and phenotypic analysis. The circles are the plasmids. DNA sequencing is used to validate proper plasmid assembly and determine the cyclic peptide identity.
Part J. The impact of individual cyclic peptides (and/or defined combinations) was evaluated by adding the cyclic peptide generator plasmids (or exogenous addition of synthetic cyclic peptides) to a fresh viral packaging process.
Part K. The resulting viral material produced by different cyclic peptide compositions was characterized (e.g. physical titer, biological titer, TU:VG ratio). Different cyclic peptide plasmids result in different viral titers (physical and biological) as well as infectivity metrics (TU:VG ratio) as depicted by different numbers and ratios of black, gray, and total viral particles. A cartoon of a bar chart represents data resulting from whatever form of analysis was performed.

FIG. 2. Exemplary discovery of cyclic peptides that enhance viral production by sequence analysis of viral genomes.
Part A. A plasmid library of cyclic peptide generators flanked by two ITRs. The library may also include additional elements: fluorescent proteins (GFP), reporter enzymes (luciferase) or a barcode.
Part B. Packaging cells were transfected with a library of genes configured to produce cyclic peptides from A. The engineered cyclic peptide library and genes required for AAV production are expressed in host cells. Engineered cyclic peptides that interfere with AAV production also interfere with their own coding DNA being packaged into AAV. Engineered cyclic peptides that enhance AAV production will enhance the packaging of their own coding DNA into AAV particles.
Part C. The cyclic peptide AAV library was harvested from the packaging cells. The AAV library harbors DNA encoding engineered cyclic peptides or DNA barcodes that can be used to identify the cyclic peptide present inside of the cell that the AAV was produced in. The library population was enriched for engineered cyclic peptide variants (or corresponding DNA barcodes) that enhanced AAV titer (black AAV capsids). The library population is depleted in engineered cyclic peptide variants (or corresponding DNA barcodes) that reduced AAV titer. A portion of the AAV library is non-infectious (gray capsids).
Part D. Cells (optionally configured for viral production) were transduced with the viral composition from Part C. Some of these viral particles will transduce either the cyclic peptide or a barcode that can be used to identify the cyclic peptide from A. The cells can optionally be in a living animal (e.g. mouse, primate), providing a means of evaluating the in vivo performance of viral material generated in the presence of cyclic peptides. Cyclic peptides that, during packaging, increased either the viral titer or infectivity of the viral particles harboring their own coding DNA will have a selective advantage. This couples the impact of cyclic peptides on viral packaging to the cyclic peptide coding DNA's ability to transduce its payload, creating a facile means of quantitatively measuring (F) the impact of cyclic peptides on viral titer and infectivity by sequencing the cyclic peptide library DNA at different points.
Part E. Viral genome DNA was harvested from the transduced cells in D (or from viral particles if cells in D were configured to produce a new generation of virus). The ITRs are shown on either side of the gene as rectangular loops.
Part F. The impact of each cyclic peptide library member on viral production and performance was assayed by NGS. Naive cyclic peptide library DNA and DNA from AAVs that have been packaged and/or transduced are sequenced by NGS. AAVs harboring DNA that encodes cyclic peptides that alter AAV production (e.g. viral titer, infectiousness) were easily identified by comparing changes in relative population frequency (e.g. naïve cyclic peptide library, packaged cyclic peptide library, re-packaged cyclic peptide library, transduced cyclic peptide library, etc. . . . ).
Part G. This sequence-based approach for evaluating the impact of chemicals on viral replication by using a functional virology system provided a novel, low cost, high throughput, facile, unbiased, and quantitative readout of the impact of a particular cyclic peptide on AAV production. Many cyclic peptides with desirable properties were rapidly identified. Desired cyclic peptides identified were subsequently evaluated for their impact on viral production in isolation and in different experimental contexts (e.g. in vivo, different media, different cell lines, etc. . . . ).
Part H. Candidate cyclic peptides were added (e.g., exogenously or via introduction of cyclic peptide generator DNA) to cells configured to produce viral material (e.g. all necessary rep, cap, helper, transgenes).
Part I. Viral material produced in the presence of cyclic peptides with high enrichment scores (e.g., increases in population from naive library to packaged library, from packaged library to transduced DNA sequence, from round to round, etc. . . . ) was characterized by measuring physical titer, biological titer, and/or potency (i.e. TU:VG ratio) in order to validate the ability of cyclic peptides to increase viral production.

FIG. 3. In vivo discovery of cyclic peptides that enhance viral production. This figure shows exemplary in vivo discovery of cyclic peptides that enhance viral production by sequencing transduced viral genomes encoding or associated with cyclic peptides.
Part A. Wildtype (WT) mammalian cells are transfected with DNA encoding Rep, Cap, helper genes and a library of genes configured to produce cyclic peptides. The cyclic peptide encoding DNA sequence, or a DNA barcode sequence allowing identification of the cyclic peptide variant, is flanked by two ITRs. The library may also include additional elements: fluorescent proteins (GFP), reporter enzymes (luciferase).
Part B. The engineered cyclic peptide library and genes required for AAV production are expressed in host cells to produce a library of AAVs comprising DNA encoding a cyclic peptide, or a DNA barcode sequence allowing identification of the cyclic peptide variant.
Part C. The AAV library is harvested. (The AAV library may optionally be enriched as described in FIG. 1G or FIG. 3J.)
Part D. The AAV library is used to transduce an animal. Consideration should be given to whether expression of the cyclic peptide in the mouse is desirable. In some contexts, this may be desirable (e.g., for drug discovery). However, in many contexts, expression is not desirable and either the use of barcodes or inducible expression systems that repress expression of the cyclic peptide in the animal model are preferred.
Part E. Cells are harvested from the animal that was transduced by the AAV library.
Part F. DNA is harvested from the cells of the animal that were transduced by the AAV library and prepared for NGS.
Part G. DNA encoding engineered cyclic peptides or barcodes identifying said engineered cyclic peptides are sequenced by NGS. DNA from the naive library (Part A), the previous enrichment round, or the AAV-packaged library (Part C), as well as the in vivo transduction, are sequenced.
Part H. The relative impact of an engineered cyclic peptide on AAV properties in vivo is determined by comparing the DNA sequence counts of cyclic peptide DNA from the in vivo selection to DNA from either the naive library (shown in A), previous enrichment rounds, or the AAV-packaged library (shown in C). AAVs that are more infectious in vivo will more efficiently deliver their engineered cyclic peptide coding sequences or associated barcodes into cells/tissues/organs of the animal. Poorly functional or non-functional AAVs are cleared by the animal immune system. The cells/tissues/organs of the animal will be enriched in DNA that encodes engineered cyclic peptide coding sequences (or associated barcode) conferring a selective/Darwinian advantage in infectivity, durability or other properties.

FIG. 4. Exemplary various pathways for discovering cyclic peptides that enhance viral production encompassing functional enrichment and sequence-guided characterization of packaged or transduced viral genomes.
Part A. Packaging cells were transfected with a library of genes configured to produce cyclic peptides and flanked by two ITRs. The library may also include additional elements: fluorescent proteins (GFP), reporter enzymes (luciferase) or a barcode. The packaging cells contain DNA encoding Rep, Cap, and helper genes (e.g., from triple transfection, stable integration, etc.).
Part B. The engineered cyclic peptide library and genes required for AAV production are expressed in host cells. Engineered cyclic peptides that reduce AAV production reduce their own coding DNA from being packaged. Engineered cyclic peptides that enhance AAV production enhanced the packaging of their own coding DNA into AAV virions.
Part C. The library of AAV virions is harvested from the AAV packaging cells. The AAV library harbors DNA encoding engineered cyclic peptides or DNA barcodes that can be used to identify the cyclic peptide present inside of the cell that the AAV was produced in. The library population is enriched for engineered cyclic peptide variants (or corresponding DNA barcodes) that enhance AAV production (black AAV capsids). The library population is depleted in engineered cyclic peptide variants (or corresponding DNA barcodes) that reduce AAV production (gray capsids).
Part D. Multiple rounds of enrichment are possible in a cyclic fashion by transducing the previous generation of AAV cyclic peptide library into fresh packaging cells. Cyclic peptides that mediate production of AAVs with increased infectivity and/or increased titer are selected for.
Part E. Cells were transduced with the viral composition from C or D. These viral particles will transduce either the cyclic peptide (e.g. from D) or a barcode that can be used to identify the cyclic peptide from A (most suitable for in vivo experiments). The cells can optionally comprise a living animal (e.g. a mouse), providing a means of evaluating the in vivo impact of cyclic peptide on viral production. (See FIG. 3D for additional considerations.)
Part F. Viral DNA that encodes cyclic peptides (or allows for their identification) was sequenced. Viral DNA from different rounds was sequenced to provide insight into the enrichment dynamics of cyclic peptides.
Part G. Sequence information from different rounds was used to determine the impact that various cyclic peptides have on viral packaging. For example, an increase in representation of a cyclic peptide in round 1 (e.g. naive plasmid library to first packaged library) is likely to indicate phenotypes that yield higher physical titer because there has been no selective pressure for increased infectivity. In contrast, cyclic peptides that showed negligible population increase in round 1, but display a noticeable jump in round 2 are more likely to mediate improvements in the production of infectious viral particles.
Part H. The sequence-based analysis makes it easy to identify cyclic peptides that are useful for optimizing viral manufacturing.

Part I. These cyclic peptide compounds can be used and characterized in a variety of ways (e.g. plasmid expressed cyclic peptides, genome expressed stable cell lines, helper virus borne, exogenous addition, etc. . . . ).

Part J. Viral production titer and/or potency was substantially increased when a cyclic peptide was present in the packaging cell.

FIG. 5. Exemplary molecular genetic flow of events that take place during cyclic peptide (CP)-modulated viral packaging, and a DNA sequence encoding a CP is packaged into a viral particle.
Part A. WT Mammalian cells are transfected with DNA encoding Rep, Cap, and helper genes; as well as a gene configured to produce cyclic peptides and flanked by two ITRs. The cyclic peptide encoding DNA may also include additional DNA payload elements: fluorescent proteins (GFP), reporter enzymes (luciferase) or a barcode.
Part B. A close-up of a cell produced in Part A.
Part C. The engineered cyclic peptide generator is expressed in the host cell while AAV biosynthesis and assembly occur (see Part F).
Part D. The transcribed and translated cyclic peptide generator undergoes an enzymatic reaction to produce cyclic peptide molecules.
Part E. The chemical structure of a random, 11 residue cyclic peptide.
Part F. Engineered cyclic peptides perturb various aspects of host cell physiology, viral biology, or both. The exact target or mechanism is not required. These perturbations can either enhance (arrow) or inhibit (T-bar) biological processes involved in viral production.
Part G. DNA encoding engineered cyclic peptides is configured to be packaged by AAV via flanking ITRs. In this way, cyclic peptides that enhance AAV production will enhance the packaging of their own coding DNA into AAV virions. Engineered cyclic peptides that reduce cell viability or AAV assembly reduce their own coding DNA from being packaged. In this way, a cyclic peptide's ability to enhance a packaging cell's ability to produce AAV particles can be connected to its ability to replicate, providing for a powerful genetic selection, which allows for the rapid identification of cyclic peptides that enhance viral titer.

FIG. 6. Exemplary molecular genetic flow of events that take place during CP-modulated viral packaging, where CP is provided on a plasmid, a DNA barcode sequence is packaged into a viral particle, but the CP is NOT packaged into a viral particle.
Part A. WT Mammalian cells are transfected with DNA encoding Rep, Cap, and helper genes; a gene configured to produce cyclic peptides that in such a way that it will not be packaged by AAV; as well as DNA barcode to identify the cyclic peptide that is flanked by two ITRs. The cyclic peptide encoding DNA may also include additional DNA payload elements: fluorescent proteins (GFP), reporter enzymes (luciferase).
Part B. A close-up of a cell produced in Part A.
Part C. The engineered cyclic peptide generator is expressed in the host cell while AAV biosynthesis and assembly occur (see Part F).
Part D. The transcribed and translated cyclic peptide generator undergoes an enzymatic reaction to produce cyclic peptide molecules.
Part E. The chemical structure of a random, 11-residue cyclic peptide.
Part F. Engineered cyclic peptides perturb various aspects of host cell physiology, viral biology, or both. The exact target or mechanism is not required. These perturbations can either enhance (arrow) or inhibit (T-bar) biological processes involved in viral production.

Part G. DNA encoding engineered cyclic peptides is operably linked to a DNA barcode flanked by two ITRs and is configured to be packaged by AAV via flanking ITRs. In this way, cyclic peptides that enhance AAV production will enhance the packaging of a DNA barcode used to identify the cyclic peptide into AAV virions. Engineered cyclic peptides that reduce cell viability or AAV assembly reduce their identifying DNA barcode from being packaged. In this way, a cyclic peptide's ability to enhance a packaging cell's ability to produce AAV particles can be connected to its ability to be identified, providing for a powerful genetic selection, which allows for the rapid identification of viral production-enhancing cyclic peptides.

FIG. 7. Exemplary molecular genetic flow of events that take place during CP-modulated viral packaging, where CP is supplied exogenously and the pAAV payload is packaged into a viral particle.
Part A. Wild-type mammalian cells are transfected with DNA encoding Rep, Cap, and helper genes, and a DNA payload flanked by two ITRs that is to be packaged into the AAV virion. The cells are also supplied with an exogenous cyclic peptide.
Part B. A close-up of a cell produced by step A.
Part C. Exogenously supplied cyclic peptides may exert their effect on the cell surface or cross the cell membrane to exert their effects intracellularly.
Part D. Exogenously supplied engineered cyclic peptides perturb various aspects of host cell physiology, viral biology, or both. The exact target or mechanism is not required. These perturbations can either enhance (arrow) or inhibit (T-bar) biological processes involved in viral production.
Part E. The ITR-flanked DNA payload is packaged into an AAV virion. Exogenously supplied engineered cyclic peptides that reduce cell viability or AAV assembly reduce the DNA payload from being packaged. In contrast, exogenously supplied engineered cyclic peptides that improve AAV production, increase DNA payload packaging. This approach is particularly suitable for high throughput screening of cyclic peptides that improve AAV production.

FIG. 8. Exemplary molecular genetic flow of events that take place during CP-modulated viral packaging, using a quadruple transfection in which CP is supplied on plasmid and not packaged, while the pAAV payload is packaged into a viral particle
Part A. WT Mammalian cells are transfected with DNA encoding Rep, Cap, and helper genes; as well as a gene configured to produce cyclic peptides; as well as a DNA payload flanked by two ITRs that is to be packaged into the AAV virion.
Part B. A close-up of a cell produced in Part A.
Part C. The engineered cyclic peptide generator is expressed in the host cell while AAV biosynthesis and assembly occur (see part E).
Part D. The transcribed and translated cyclic peptide generator undergoes an enzymatic reaction to produce cyclic peptide molecules.
Part E. Engineered cyclic peptides perturb various aspects of host cell physiology, viral biology, or both. The exact target or mechanism is not required. Perturbations that enhance (arrow) biological processes involved in viral production increase packaging of the ITR-flanked DNA payload into AAV virions.
Part F. The ITR-flanked DNA payload is packaged into an AAV virion. In contrast, the cyclic peptide is not packaged into the virion. This approach was used to characterize the effects of individual cyclic peptides on AAV production (e.g. increased physical titer, biological titer, transduction efficiency, etc. . . . ).

FIG. 9. Exemplary molecular genetic flow of events that take place during CP-modulated viral packaging, where CP is supplied by expression from a stable genome integration and a pAAV payload is packaged into a viral particle
Part A. WT Mammalian cells are transfected with DNA encoding Rep, Cap, and helper genes; as well as a DNA payload flanked by two ITRs that is to be packaged into the AAV virion. A cyclic peptide expression cassette is genomically integrated into the host cell.
Part B. A close-up of a cell produced in Part A.
Part C. The engineered cyclic peptide generator is expressed in the host cell while AAV biosynthesis and assembly occur (see part E).
Part D. The transcribed and translated cyclic peptide generator undergoes an enzymatic reaction to produce cyclic peptide molecules.
Part E. Engineered cyclic peptides perturb various aspects of host cell physiology, viral biology, or both. The exact target or mechanism is not required. Perturbations that enhance (arrow) biological processes involved in viral production increase packaging of the ITR-flanked DNA payload into AAV virions.
Part F. The ITR-flanked DNA payload is packaged into an AAV virion. In contrast, the cyclic peptide is not packaged into the virion because it is located on the host cell's chromosome.

FIG. 10. Exemplary basis for enrichment of cyclic peptides that enhance viral packaging (left panel) and depletion of cyclic peptides that disrupt viral packaging (right panel).
Part A. A cell engineered to produce AAV with a cyclic peptide that improves AAV production.
Part B. An ITR-flanked cyclic peptide expression cassette is transcribed and translated. The cyclic peptide generator performs a cyclization reaction to release the circularized cyclic peptide encoded by the DNA of the generator.
Part C. Engineered cyclic peptides perturb various aspects of host cell and/or viral physiology. The exact target or mechanism is not required.
Part D. Because this engineered cyclic peptide enhances AAV production, packaging of cyclic peptide encoding DNA into AAV virions is increased.
Part E. A cell engineered to produce AAV with a cyclic peptide that reduces AAV production.
Part F. An ITR-flanked cyclic peptide expression cassette is transcribed and translated. The cyclic peptide generator performs a cyclization reaction to release the circularized cyclic peptide encoded by the DNA of the generator.
Part G. Engineered cyclic peptides perturb various aspects of host cell and/or viral physiology. The exact target or mechanism is not required.
Part H. Because this engineered cyclic peptide is toxic or otherwise interferes with processes involved in AAV production, packaging of cyclic peptide encoding DNA into AAV virions is reduced.

FIG. 11. Exemplary basis for enrichment of cyclic peptides that increase viral titer and/or transduction efficiency of infectious AAV particles.
Part A. WT Mammalian cells are transfected with DNA encoding Rep, Cap, and helper genes. The same WT mammalian cells are also transduced by AAV harboring a DNA sequence configured to produce a cyclic peptide. The input AAV may, for example, be the result of a previous round of cyclic peptide enrichment.
Part B. A close-up of a cell produced in Part A.
Part C. Successful AAV transduction of the cyclic peptide DNA requires the AAV particle to bind the host cell, enter the cell, navigate cell trafficking, escape the endosome, avoid proteasomal destruction, enter the nucleus, uncoat, release DNA, process DNA, and avoid innate antiviral immune response. Successful transduction results in the AAV DNA payload containing the engineered cyclic peptide generator release and expression in the host cell.
Part D. Successfully delivered cyclic peptide generators are transcribed and translated by the host cell machinery. The cyclic peptide generator performs a cyclization reaction and releases the cyclized cyclic peptide encoded by the cyclic peptide generator DNA.
Part E. Engineered cyclic peptide modulates host cell and/or viral physiology during AAV production to generate a subsequent generation of AAV particles with increased infectiousness. The exact target or mechanism is not required (see part C. for examples).
Part F. Cyclic peptide encoding DNA sequences that more efficiently transduce cells and express engineered cyclic peptides that improve the transduced cell's ability to produce more infectious AAV will enhance the packaging of their own coding DNA into more infectious AAV virions. This confers a replicative advantage to DNAs encoding cyclic peptides the optimize host cells for AAV production.

FIG. 12. Exemplary basis for selective enrichment of cyclic peptide coding sequences that improve viral production and depletion of cyclic peptide coding sequences that reduce viral production. This Figure provides an example of the dynamics at play during selection of cyclic peptides that enhance viral packaging. Cyclic peptides that generate more infectious AAVs can more efficiently get their DNA encoding the engineered cyclic peptide into packaging cells, which results in a greater number of more infectious AAVs being produced (possibly akin to a “K-selected” reproductive strategy). Similarly, cyclic peptides that generate greater numbers of infectious AAVs have many chances to get their cyclic peptide DNA into packaging cells, resulting in ever greater numbers of AAVs harboring these cyclic peptides being produced (possibly akin to a “R-selected” reproductive strategy).
Part A. Cells with DNA encoding Rep, Cap, helper genes; as well as pCP, an ITR flanked gene configured to produce cyclic peptides that increase AAV production.
Part B. AAV virions produced in cells expressing cyclic peptides that improved AAV viral titer and/or transduction efficiency harbor the cyclic peptide coding sequences that improved those viral production attributes. These AAV particles infect other cells configured to produce AAVs (e.g., neighboring cells in the same round of enrichment or fresh cells in a subsequent round of enrichment).
Part C. AAV borne cyclic peptide generators that enhance viral production are amplified through multiple transductions and AAV replication cycles and can rapidly outcompete cyclic peptide generators conferring modest to negative effects on viral packaging (Parts D-E). Thus, cyclic peptide coding sequences that improve viral production are rapidly enriched.
Part D. Cells with DNA encoding Rep, Cap, helper genes; as well as pCP, an ITR flanked gene configured to produce cyclic peptides that interfere with AAV production.
Part E. Cells expressing cyclic peptides that interfere with AAV production do not generate AAV particles harboring these cyclic peptide constructs at sufficient quantities or levels of infectiousness to effectively compete against cyclic peptides that improve viral production. Thus, cyclic peptide coding sequences that reduce viral production are rapidly depleted.

Part F. Viral genomes can be collected and analyzed through NGS in order to characterize the effect of specific cyclic peptides on the production of AAV particles.

FIG. 13. Exemplary Basis for analysis of cyclic peptide (CP) coding sequences that improve or decrease viral production performance in cells not configured to produce additional AAV particles.
Part A. Cells with DNA encoding Rep, Cap, helper genes; as well as pCP, an ITR flanked gene configured to produce cyclic peptides that increase (top) or decrease (bottom) AAV production.
Part B. Cells infected by AAV particles that were produced in a cell in the presence of a cyclic peptide that increased AAV production are able to deliver their DNA payload (a cyclic peptide generator or associated barcode) more efficiently or in greater numbers compared to AAV particles produced in the presence of cyclic peptides that reduce AAV production.
Part C. Viral genomes can be collected and analyzed through NGS in order to characterize the effect of specific cyclic peptides on the production of AAV particles.

FIG. 14. Molecular genetic flow of events that take place during CP-modulated packaging lentiviral vectors.
Part A. A plasmid borne cyclic peptide generator (e.g. SICLOPPS) can be used to generate cyclic peptides during lentiviral packaging. This plasmid is co-transfected with the rest of the packaging plasmids (B and C).
Part B. The Lentiviral packaging DNA provides the necessary components for viral production. There are a variety of different systems; this figure illustrates a typical “3rd generation” system. This system splits the viral genes across multiple plasmids to reduce the risk of generating replication competent viral particles. Typically, a third-generation lentiviral packaging system consists of four plasmids, the gag-pol, env, rev, and transfer plasmid (C). Env, the envelope plasmid, expresses the viral envelope glycoprotein. Gag-pol expresses structural proteins (Gag) and enzymes (Pol). The Gag protein includes matrix (MA), capsid (CA), and nucleocapsid proteins (NC). Pol includes reverse transcriptase, integrase, and protease (pro). Rev expresses the Rev protein which facilitates nuclear export of the unspliced or partially spliced viral RNAs.
Part C. pTrans, (the transfer plasmid) contains the payload transgene of interest (e.g. a therapeutic payload) along with the long terminal repeats (LTRs), the Psi packaging signal, and typically the Rev response element (RRE). Some systems also include a central polypurine tract (cPPT) to enhance nuclear import of the pre-integration complex.
Part D. A close up of a cell produce when all 5 components (the CP generator and the packaging plasmids/transfer vector) are transfected into packaging cells (e.g. HEK293 cells). These cells begin to produce lentiviral particles under the influence of cyclic peptides.
Part E. The cyclic peptide generator is transcribed, translated, and undergoes splicing to produce a cyclic peptide.
Part F. The lentiviral packaging DNA is expressed, allowing the cell to generate viral particles.
Part G. The cyclic peptide modulates cellular environment in order to enhance viral production.
Part H. The transcribed RNA transgene is packaged into a lentivirus.
Part I. The transgene is packaged into a lentivirus, but the CP sequence is not.

FIG. 14 shows that cyclic peptides can also be used to enhance lentiviral vector production in the same way how they enhance AAV production. For actual implementation see Example 9 and FIG. 25 below.

FIG. 15. Exemplary Split-intein circular ligation of peptides and proteins (SICLOPPS) cyclic peptide generator (adopted from Ong Y S, Gao L, Kalesh K A, Yu Z, Wang J, Liu C, Li Y, Sun H, Lee S S. Recent Advances in Synthesis and Identification of Cyclic Peptides for Bioapplications. Curr Top Med Chem. 2017; 17(20):2302-2318). There are many ways to produce cyclic peptides. SICLOPPS is the preferred embodiment given the ease of manipulation and methods known in the art. There are a variety of intein splicing mechanisms that are known in the art; an exemplary mechanism is provided below.
Part A. SICLOPPS fusion protein with intein C-terminus on the construct's N-terminus, the intein N-terminus on the construct's C-terminus, and the peptide sequence to be circularized in the center, denoted by the degenerate amino acids XXXXX.
Part B. The two intein domains associate in order to create an active split intein.
Part C. An N—O or N—S(N—Z) shift when the side chain of the first residue (a serine, threoine, or cystine) of the intein portion of the precursor cyclic peptide nucleophilically attacks the peptide bond of the residue immediately upstream to form an ester or thioester.
Part D. A transesterification produces a lariat structure.
Part E. A side chain cyclization occurs.
Part F. An O—N or S—N(Z-to-N) acyl shift yields the cyclic peptide.

FIG. 16. Exemplary SICLOPPS cyclic peptide generator library architecture, expression, and DNA packaging.
Part A. A plasmid borne cyclic peptide generator transfer vector with an optional DNA payload. Arbitrary cyclic peptide AAV transfer vectors can be created by inserting the appropriate amino acid coding sequence into the SICLOPPS insertion site (C).
Part B. A random cyclic peptide library can be constructed by inserting DNA containing degenerate nucleotides. The preferred embodiment employs NNK codons that can encode any of the standard amino acids and one stop codon. The length and diversity of the cyclic peptide libraries is easily adjusted by inserting different DNA sequences into SICLOPPS cyclic peptide generator splicing sites.
Part C. The desired cyclic peptide or encoding DNA or a library is inserted into the engineered cyclic peptide generator DNA, which is encoded within a mammalian expression system that controls the expression of the engineered cyclic peptide generator in mammalian cells. The mammalian expression system typically consists of a promoter, regulatory sequences, enhancers and a polyA tail. The expression system, engineered cyclic peptide generator along with an optional supplementary payload, is flanked by and/or operably linked to ITRs. The optional supplementary payload can be DNA encoding a fluorescent reporter protein, an enzymatic reporter, a therapeutic payload, or a barcode DNA sequence, etc.
Part D. The ITR flanked engineered cyclic peptide generator and optional payload is encoded on a plasmid. The plasmid is transfected into mammalian cells for AAV packaging along with DNA encoding Rep, Cap, and helper genes.
Part E. Once transfected, the engineered cyclic peptide generator and optional supplementary payload are transcribed and translated by host cell machinery. The cyclic peptide generator undergoes a cyclization reaction to release the cyclized cyclic peptide. The remainder of the cyclic peptide generator is degraded and recycled by the host cell. Once released, the cyclic peptide interacts with various cellular targets to perturb host cell physiology.
Part F. During AAV biosynthesis and assembly, the ITR flanked DNA sequence is synthesized as ssDNA and packaged inside AAV virions. Engineered cyclic peptides that interfere with cell viability or AAV production reduce their own coding DNA from being packaged. Engineered cyclic peptides that enhance AAV production enhance the packaging of their own coding DNA into AAV virions.
Part G. An alternate architecture that illustrates a non-mobilizable SICLOPPS. A DNA barcode is placed into the mobilizable portion of the plasmid between the ITRs, while the cyclic peptide generator is on the vector backbone. In this arrangement, the cyclic peptide sequence will not be packaged into a cell, but the cyclic peptide's impact on viral activity can be determined by its associated, mobilizable DNA barcode. The cyclic peptide sequence is encoded by the degenerate codon oligo (NNK).

FIG. 17. Exemplary violin plots of image-based cytometry of HEK293T cells transduced with AAV2-GFP vectored cyclic peptide libraries enriched for cyclic peptide that improve AAV production.

An AAV-vectored cyclic peptide library with a GFP payload is produced in mammalian cells. In order to assess the overall transduction competence of the harvested AAV cyclic peptide library after enrichment, the AAV-vectored cyclic peptide library is transduced into fresh WT mammalian cells. The AAV payload encodes a supplemental GFP fluorescent reporter protein. When transduced, the GFP reporter protein is expressed and can be quantified by cytometry. Therefore, the GFP intensity directly correlates to functional properties of the AAV-vectored cyclic peptide library.

The violin plots depict the enrichment of 12 engineered cyclic peptide generator libraries. The Y-axis represents GFP fluorescence intensity of a cell in log scale. The X-axis represents the proportion of the cell population of a given RFU. The overall shape of the violin plot depicts the population distribution of cell fluorescence intensity after being transduced by an enriched AAV library. Raw data is provided as an overlaid scatter plot with the y-axis corresponding to RFU and the x-axis a random normal distribution jitter for visualization.

For each of the 12 engineered cyclic peptide libraries, the gray plot depicts the fluorescence intensity distribution of mammalian cells after being transduced by the AAV cyclic peptide library (with GFP reporter) harvested after two rounds of enrichment. The black plot depicts the fluorescent intensity distribution of mammalian cells after being transduced by the same AAV cyclic peptide library (with GFP reporter) harvested after three rounds of enrichment. The upward shift of GFP intensity of the population distribution indicates more viral transduction of the GFP payload, which represents improvements in viral particle titer and/or transduction efficiency. Detail is provided for the violin plot of library number 5 at the bottom.

FIG. 18. Exemplary microscopy illustrating that AAV2-GFP with CP library from round 3 of selection (top panel) has substantially higher transduction efficiency compared to the same CP library from round 2 of selection (bottom panel).
Part A. The brightfield micrograph depicts the total mammalian cells in the field of view. These micrographs are used to determine the total number of cells in each field of view. Cells below RFU<2000 cutoff are marked with a dark circle apparent in brightfield. Cells registering RFU>2000 cutoff are marked with a white circle apparent in the fluorescence micrographs.
Part B. The fluorescence micrographs collected at medium exposure depict the GFP expressing cells. The greater number/brighter GFP expressing cells visible after being transduced by the AAV cyclic peptide library harvested after three rounds of enrichment relative to cells that were transduced by the same AAV cyclic peptide library harvested after two rounds of enrichment indicates more transduction of the GFP payload after the third round of enrichment.
Part C. The fluorescence micrographs collected at high exposure illustrate significantly greater number of bright GFP expressing cells after being transduced by the AAV cyclic peptide library harvested after three rounds of enrichment.

FIG. 19A-J. Exemplary plots illustrating the vast amounts of functional cyclic peptide chemical diversity that was found to improve viral production.

Cyclic peptide-encoding DNA sequences were subjected to enrichment and sequence-based analysis as described in FIGS. 1-4. DNA encoding the cyclic peptide expression constructs was sequenced using Illumina NGS at each round of enrichment for between 2-4 rounds, as well as the naive plasmid library DNA. The relative frequency of each cyclic peptide was determined. The change in relative frequency of each cyclic peptide from one round to the next was determined across multiple experimental replicates and averaged (though analysis using median, quartiles, and modes typically yield consistent results). The relationship between cyclic peptide chemical identity, chemical similarity with respect to other cyclic peptides, and the unexpected quantity of cyclic peptides that favorably impact viral production are illustrated in this figure.

FIG. 19A. A portion of a t-SNE scatter plot illustrating clusters of similar cyclic peptides and their performance (dark depleted, light enriched) in an AAV5 cyclic peptide selection. 2D t-SNE representation was calculated on cyclic peptide chemical embedding vectors of 512 dimensions generated using a pre-trained transformer molecular encoder model. Each dot is an individual cyclic peptide. Similar cyclic peptides (e.g., those with similar chemical, structural, and/or sequence motifs) cluster together, while dissimilar cyclic peptides are positioned further apart. Dark dots reduce AAV production as measured by NGS depletion. Lighter spots increase AAV production as measured by NGS enrichment.

FIG. 19B. A portion of the t-SNE that is provided in detail in FIG. 19C.

FIG. 19C. Details of the t-SNE plot marked in FIG. 19B.

FIG. 19D. A cluster of cyclic peptide space dominated by variants that were enriched (light dots), indicating that these cyclic peptides can be used to enhance AAV5 production. A cluster of cyclic peptides are those that are chemically similar to one another. Clusters typically include cyclic peptides that share the same or similar sequence motifs.

FIG. 19E. A cluster of cyclic peptide space dominated by variants that were rapidly depleted (dark dots), indicating that these cyclic peptides are not useful for AAV5 production.

FIG. 19F. Distance matrix illustrating pairwise Euclidean distance of cyclic peptide chemical fingerprints. Cyclic peptides were isolated by screening following 3 rounds of selection. Chemical fingerprints were generated using RDKit's chemical fingerprinting module. The distance matrix was additionally subjected to clustering in order to place similar cyclic peptides next to one another. Dark colors indicate higher chemical similarity, while lighter colors indicate lower chemical similarity. The diagonal line is dark because the chemical similarity is maximized when comparing an identical compound. Note the presence of 6 dark clusters in the upper right-hand corner. Other than the clusters in the upper right, most of these cyclic peptides appear somewhat dissimilar from one another.

FIG. 19G. Plot showing enrichment factors for a random selection of about 23,000 cyclic peptides identified (less than 1%) using NGS analysis of cyclic peptide enrichment performed in AAV5. Enrichment factor is provided on the log 10 scaled Y-axis and cyclic peptide rank is on the X-axis. As expected, the majority of cyclic peptides are depleted (e.g., their enrichment scores are less than one, indicating a reduction in the relative frequency of certain library member variants from one round of selection to the next).

FIG. 19H. A plot showing the relationship between cyclic peptide ring size and average enrichment factors (Y-axis, log 10 scale) across AAV serotypes 1, 2, 5, 6. A random subset of cyclic peptides with ring size of between 5-8 amino acids appeared to have the highest enrichment factors.

FIG. 19I. A bar chart illustrating the relationship between cyclic peptide ring size (between 5-8 in this case) and the maximum enrichment factors identified for a given ring size. This is in agreement with FIG. 19H. This illustrates an unexpected correlation between cyclic peptide size and function in increasing viral production. It should be noted that there are larger cyclic peptides that confer improvements in viral manufacturing, but they are more difficult to identify due to the exponential increase in sequence space with each additional amino acid added to the ring size. In other words, the ratio of functional sequence space to total sequence space appears to be unexpectedly higher with cyclic peptides of ring size 5-8.

FIG. 19J. Volcano plots in scatter plot format (left panel) and hexbin format (right panel) illustrating CP enrichment, depletion, confidence, number of experimental replicates, and dataset density.

In the volcano scatter plot (left panel), the X-axis is log 10 of CP enrichment score (i.e., the log “effect size” per conventions for volcano plots). Anything greater than zero on the X-axis (10=1) is enriched (i.e., improves AAV production/performance). Anything less than zero on the X-axis (e.g., less than fold difference of 1 before log 10 is applied) is depleted (i.e., reduces AAV production/performance). Y-axis is statistical confidence as measured by −log 10(P-value) per conventions for volcano plots. Higher Y-axis means more confidence. Horizontal dashed line represents P-value=0.05 (adjusted to be −log 10 to match y axis). Vertical dashed lines are log 10(10) (i.e. 1) on the X-axis, corresponding to a 10 fold enrichment (i.e. log 10(enrichment=10)=1). Marker size is the number of independent experimental replicates (e.g., same cyclic peptides (CPs) found in enrichments of different serotypes, NGS replicates, rounds, etc. . . . ). As expected, bigger scatter plot points have higher confidence intervals on the Y-axis.

Just like the scatter plot, the hexbin plot (right panel, same data and axes) also has the X-axis representing the log 10 of CP enrichment score and the Y-axis representing the −log 10(P-value) per conventions. However, instead of using individual markers for each data point, the hexbin plot (a type of bivariate histogram) divides the graph into hexagonal bins and represents the number of data points in each hexagon through color intensity. This representation is useful when dealing with a large number of data points that may overlap, as it allows visualization of the data density in different regions of the graph.

Darker hexagons represent areas with lower data point density. Conversely, lighter hexagons (except white background) indicate regions with a higher density of data points, signifying a higher concentration of CPs with similar enrichment scores and confidence levels. Shading of bins is log-scaled, meaning that different shades of gray can represent orders of magnitude higher data density. This helps in quickly identifying areas with significant enrichment or depletion of CPs and also allows for a visualization of the quantity of CPs that increase viral titer or transduction efficiency (TU:VG ratio).

Vertical and horizontal dashed lines, as well as the significance of X and Y-axis values, are the same as described for the scatter plot. The hexbin plot does not represent the number of independent experimental replicates through marker size, as it primarily focuses on showing data density through hexagonal binning.

To provide additional insight into the unexpected scale and efficiency with respect to identifying CPs that improve viral yield, ˜2,000,000 unique cyclic peptides were observed to be enriched by at least 100 fold (X-axis>2) with a P-value of less than 0.05 (Y-axis˜>−1.3=−log 10(0.05)).

In addition to the exceptionally large number of CPs that improve viral production, a large number of CPs that disrupt viral packaging was also observed (left panels of both plots). These CPs may, for example, be toxic to human cells or possess antiviral properties. The ability to evaluate the impact of these compounds is useful in that a very large number of negative examples are crucial in developing robust computational models capable of efficiently navigating functional chemical space.

FIG. 20. Exemplary bar charts showing fold improvement of physical titer and/or biological titer for various CPs. Part A. The bar chart depicts the fold improvements in physical titer of AAVs packaged in cells expressing individual CPs (CKDYFS (SEQ ID NO: 34), CPKNP (SEQ ID NO: 53), CAAAS (SEQ ID NO: 48), CADTV (SEQ ID NO: 49), CYVTTD (SEQ ID NO: 62), CTCKK (SEQ ID NO: 59), CQSRS (SEQ ID NO: 54), CSESCP (SEQ ID NO: 57), CSDPSA (SEQ ID NO: 56), CYPLH (SEQ ID NO: 60), and CQVTRM (SEQ ID NO: 55)) relative to AAVs packaged in cells under essentially identical conditions but with no CP expression. The payload being packaged during AAV production is GFP only. The cyclic peptide generator is located on a separate DNA, is only expressed by the host cell, and is not able to be packaged. Physical titer is quantified by qPCR or ddPCR and expressed as viral genomes produced per mL (VG/ml) of AAV packaging cells and media. The fold improvement is calculated by dividing the physical titer of AAVs packaged in cells expressing a CP by the physical titer of AAVs packaged in cells that are not expressing a CP.
Part B. The bar chart depicts the fold improvements in biological titer of AAVs packaged in cells expressing individual CPs (CRRHYA (SEQ ID NO: 230), CSVPY (SEQ ID NO: 58), CARFR (SEQ ID NO: 51), CSESCP (SEQ ID NO: 57), CETDV (SEQ ID NO: 52), CYPLH (SEQ ID NO: 60), CYSPF (SEQ ID NO: 61), CYVTTD (SEQ ID NO: 62), CAAAS (SEQ ID NO: 48), CSDPSA (SEQ ID NO: 56), CANMPY (SEQ ID NO: 50), CQVTRM (SEQ ID NO: 55)) relative to AAVs packaged in cells under essentially identical conditions but with no CP expression. Biological titer is measured in vitro by transducing a standardized number of wild-type (WT) mammalian (HEK293) cells with diluted AAVs harvested after packaging with or without CP expression. The transductants are identified by GFP payload expression. The fold improvement is calculated by dividing the percentage of GFP expressing (transduced) cells after transduction with AAVs packaged in cells expressing a CP by the percentage of GFP expressing (transduced) cells after transduction with AAVs packaged in cells that are not expressing a CP.

FIG. 21. Exemplary bar chart showing a dose-response effect in fold change of AAV biological titer resulting from different nanogram dosages of CP plasmid DNA supplied during viral packaging. The nanogram (ng) amount of cyclic peptide plasmid generator DNA is an important parameter which can be adjusted and optimized. Each cyclic peptide generator plasmid (e.g., T2851-1) was tested at 3 different plasmid dosages (transfected at 500, 50, and 5 ng of plasmid DNA). AAVs were produced with 6 different cyclic peptide generator plasmids along with a “no CP” control. The biological titer and fold improvements were determined as described in FIG. 20. A clear dose-dependent effect on viral titer in response to the dosage of cyclic peptide plasmid DNA was observed. Order of magnitude steps in dosage are too large and the optimal dose window can be easy to miss. The dose dependent impact of cyclic peptide CYNHAR (SEQ ID NO: 35), which corresponds to T2851-8, is shown for illustrative purposes; this cyclic peptide was found to have an increased transduction efficiency (TU:VG ratio) of ˜2.4×(data not shown). While different cyclic peptides show different responses to different doses, the higher doses of cyclic peptide plasmids shown in this figure substantially reduced biological titer, exemplifying the potent biological effects these constructs can exert. (See also FIG. 23 for cyclic peptides that are less dose-responsive.)

FIG. 22. Exemplary bar chart showing fold change in AAV biological titer in media and cell pellet fractions for individual CPs, as well as how combining CPs leads to synergistic effects.

Cyclic peptides can perturb cellular physiology in different ways. It is predicted that cyclic peptides can synergize to perturb the cellular environment in an additive or multiplicative manner with respect to AAV biosynthesis and assembly.

The bar chart depicts synergistic and compensatory effects of CPQFGR (SEQ ID NO: 36) and CKDYFS (SEQ ID NO: 34). AAVs produced in mammalian cells expressing a transduction efficiency enhancing cyclic peptide cocktail (TU:VG Pool, left) have improved transduction efficiency (TU:VG ratio), but have lower physical and lower biological titers. However, an increase in the physical titer is desired to improve the economics of therapeutic manufacturing. AAVs produced in cells co-expressing CPQFGR (SEQ ID NO: 35), CKDYFS (SEQ ID NO: 34) and the transduction efficiency enhancing cyclic peptide cocktail (TU:VG Pool) have higher biological titer and higher transduction efficiency than AAVs produced in cells expressing any of the constituent cyclic peptide members, while the physical titer is closer to the average of its constituent members. This proves that CPs can act synergistically, and other cyclic peptide combinations can be explored.

FIG. 23. Exemplary bar chart illustrating 6 CPs that increase transduction efficiency of AAVs packaged in cells expressing cyclic peptides by reducing full, but non-functional viral capsids compared to the control. A greater percentage of the full AAV particles were capable of viral transduction when packaged in cells expressing a cyclic peptide relative to full the AAV particles packaged in cells expressing no cyclic peptide.

Full, but non-functional viral particles represent a major manufacturing challenge for AAV gene therapies. At present, there is no process capable of purifying the functional viral particles from the full, but non-functional fraction. This figure demonstrates the CP-mediated depletion of full, but non-functional virions by increasing the transduction efficiency of the viral composition. The fold increase in transduction efficiency is directly related to the proportion of full, but non-functional AAV particles.

The bar chart depicts the improvements in transduction efficiency of AAV2-GFP produced in the presence of 6 different CPs (CSSLT (SEQ ID NO: 37), CTHKVS (SEQ ID NO: 38), CCRPH (SEQ ID NO: 37), CKYEE (SEQ ID NO: 40), CRVSY (SEQ ID NO: 41), CQVFQL (SEQ ID NO: 42)) as illustrated in FIG. 8. Each cyclic peptide that enhanced AAV2-GFP packaging condition was tested at several dosages of cyclic peptide plasmid (without ITRs) DNA ranging from 0.3 ng to 100 ng, while the control AAV2-GFP was produced in the absence of any CPs (CTRL, far right). AAV2-GFP were harvested and assayed for biological and physical titer as described in FIG. 20

FIG. 24. Exemplary representative flow cytometry raw data illustrating one CP that reduces full, but non-functional viral capsids compared to the control. This figure depicts the raw flow cytometry data used to determine the biological titer of AAV samples. This is an example of the data that was used to determine the biological titers in FIG. 23. The X-axis of each graph represents the forward light scattering (FSC) from flow cytometry. The Y-axis represents the GFP intensity of each cell. A 1000-fold dilution of AAV2-GFP particles produced either in the presence (A) or absence (B) of cyclic peptide were used to transduce HEK293 cells. A 1000-fold dilution of the preparation was used (e.g., without physical titer normalization) to ensure a direct, batch-to-batch comparison of the impact of cyclic peptide on viral packaging performance.

Part A. Flow cytometry raw data (with minor forward and side scatter gating to remove irrelevant particles) of HEK293 transduced with 3.1E6 VG/ml of AAV2-GFP (a 1000-fold dilution of physical titer) produced in the presence of a cyclic peptide.
Part B. Flow cytometry raw data (with minor forward and side scatter gating to remove irrelevant particles) of HEK293 transduced with 3.8E7 VG/ml (more than 10-fold more than in Part A) of AAV2-GFP (a 1000-fold dilution of physical titer) produced without cyclic peptide.

Despite cells in Part A being transduced with ˜10×less AAV2-GFP compared to cells in Part B (3.1E6 VG/ml vs 3.8E7 VG/ml), the cells transduced with AAV2-GFP produced in the presence of a single cyclic peptide sequence show comparable flow cytometry profiles. Therefore, the AAV sample harvested from mammalian cells expressing a single CP contains a ˜10× reduction in full, but non-functional viral particles. This is unexpected and useful because the use of a CP allows for removal of full, but non-functional viral particles, which represent by far the dominant product-related impurity, typically accounting for more than 99% of viral material.

FIG. 25. Exemplary improvement of lentiviral production by cyclic peptides.

Cyclic peptides are used herein to improve viral titer and transduction efficiency during lentiviral production. Lentivirus was produced using Takara Bio lentivirus packaging kit either with (left panels) or without (right panels; bars labeled CTRL) a cyclic peptide generator plasmid. The cyclic peptides produced by different amounts (e.g., 0.5 ng, 5 ng and 50 ng) of corresponding generator plasmids were CSSLT (SEQ ID NO: 37), CGSTKS (SEQ ID NO: 43), CKYEE (SEQ ID NO: 40), CPYTD (SEQ ID NO: 44), CPLQVP (SEQ ID NO: 45). 5 ng plasmid experiment for CGSTKS (SEQ ID NO: 43) failed and is not shown. The virus was collected, and particle titers were enumerated using Takara Bio Lenti-X GoStix Plus quantitative lentiviral titer test. The biological titer was determined by transducing fresh HEK293 cells with the various lentiviral preparations (e.g., packaged with or without a cyclic peptide). The fold difference in particle titer and transduction unit titer is calculated by normalizing to the average of six control lentiviral transductions from packaging runs without cyclic peptide. The control (CTRL) conditions illustrate negligible noise. An unexpected degree of improvement in both physical titer and biological titer was demonstrated.

FIG. 26. Exemplary representative flow cytometry raw data illustrating that when cyclic peptide CGSTKS (SEQ ID NO: 43) is present during lentiviral production, the biological titer increases by over 8-fold compared to when the cyclic peptide is absent. This figure depicts the raw flow cytometry data used to determine the biological titer of lentiviral samples. This is an example of the data that was used to determine the lentiviral biological titers in FIG. 25. The X-axis of each graph represents the forward light scattering (FSC) from flow cytometry. The Y-axis represents the GFP intensity of each cell. A 100-fold dilution of Lentivirus with a GFP payload produced either in the presence (A) or absence (B) of cyclic peptide were used to transduce HEK293 cells. A 100-fold dilution of the preparation was used (e.g., without physical titer normalization) to ensure a direct, batch-to-batch comparison of the impact of the cyclic peptide on viral packaging performance.
Part A—Flow cytometry raw data (with minor forward and side scatter gating to remove irrelevant particles) of HEK293 transduced with a lentiviral preparation produced in the presence of cyclic peptide CGSTKS (SEQ ID NO: 43) generated from a plasmid constructed transduced into packaging cells at 0.5 ng.
Part B—Flow cytometry raw data (with minor forward and side scatter gating to remove irrelevant particles) of HEK293 transduced with a lentiviral preparation produced without cyclic peptide.

Lentiviral vectors produced in the presence of this cyclic peptide (and other selected CPs including CPs shown in FIG. 25) show substantially higher transduction unit titers compared to lentiviral vectors produced in the absence of cyclic peptides.

FIG. 27. Exemplary heatmap plot illustrating that cyclic peptides are predicted to promiscuously interact with multiple protein targets.

X-axis is a sub-selection of 800 human proteins for which crystal structures have been solved or for which high confidence predictions exist. Y-axis is 366 different CPs; each CP was chosen when it had the highest enrichment factor from the CP clusters identified in the t-SNE scatter plot in FIG. 19. The heatmap illustrates CP-protein binding interactions predicted by multimodal machine learning transformer models that were trained to predict multiple drug-target interaction metrics including Kd, Ki, IC50, EC50, and binding energy. Prediction accuracy was validated by multiple methods; predictions from multiple model architectures trained on different datasets were in agreement. Darker colors correspond to a stronger pairwise interaction between a given cyclic peptide and protein target. The heatmap was hierarchically clustered for improved visualization.

This heatmap indicates that cyclic peptides can interact with diverse protein targets. Additionally, CP-target interactions are not one-to-one, but may act on multiple targets simultaneously. This illustrates an unexpected outcome and a key strength. The unexpected number of CP-target interactions indicates the exceptionally high search space enabled. If it is conservatively assumed to be about 100 strong CP-protein interactions per CP, this increases the chance by 100-fold an interaction that improves viral production is identified.

FIG. 28A-F. Exemplary CP-protein interactions for 6 CPs and their likely targets.

CP-protein binding interactions were predicted by machine learning transformer models trained on multiple drug-target interaction features. CP-protein interaction targets predicted to have strong and specific interactions with particular protein targets were evaluated by molecular docking to validate the predicted interaction and to investigate structural and energetic aspects of the association. Molecular docking of the examples resulted in strong binding energies and consistent binding poses of the cyclic peptide to specific locations on the protein targets.

FIG. 28A. CP binding to an adapter protein involved in asymmetrical cell division and cell polarization processes. Cell polarity is frequently targeted by many diverse viruses to create a replication-permissive environment. It is noteworthy that the CP engages the target on a shallow surface, demonstrating that CPs do not need deep pockets like small molecules do.

FIG. 28B. CP binding to a repressor of translation initiation that is involved in 7-methylguanosine-containing mRNA cap recognition. This protein target has been shown to be manipulated by a variety of different viruses during viral infection. Interestingly, viral mechanisms targeting this protein impact not only viral protein synthesis, but also the viral replication cycle, including proliferation of infected cells or viral reactivation. In contrast to CP in FIG. 28A, this CP engages a pocket deep in the protein structure, illustrating the flexibility of molecular targeting afforded by various CPs. An antibody fragment would never be able to target the residues engaged by the CP.

FIG. 28C. CP binding a tRNA-splicing endonuclease. Human cells may target tRNA for destruction in response to viral infections. Little is known about this protein's role in viral replication, making it an interesting example of how the unbiased discovery approach can uncover new ways of optimizing viral production. This CP provides another example of binding a flat, pocket-less protein target.

FIG. 28D. CP binding a target involved in cell protection during the inflammatory response and regulation of apoptosis. Little is known about this protein's role in viral replication.

FIG. 28E. CP binding a target involved in RNA-binding protein that acts as a pre-mRNA splicing factor. It may be involved in several viral responses in human cells, but few studies exist to verify this.

FIG. 28F. CP binding a kelch repeat protein of unknown function.

Computational and structural biology analysis illustrate that CPs are able to target a wide variety of structurally and functionally distinct targets. Some targets are known to be involved in viral processes, while few or no reports exist for involvement of other targets in viral replication. A key point illustrated by this figure is that the cyclic peptide discovery approach, being an unbiased functional enrichment, is able to rapidly identify compounds targeting non-obvious targets. For example, the cyclic peptide interactions illustrated in FIG. 28D-F are targets that are poorly characterized. These targets might otherwise be ignored in a hypothesis-based engineering approach, where effort would be focused on targets based on evidence, which constrains the solution space. It is shown here that CPs can engage protein targets that have both deep pockets or flat binding pockets, illustrating flexibility of the approach for optimizing viral production. The CP-target binding modes also illustrate key advantages over mutational approaches in that CPs may interacting with residues that are proximal in 3D space, but that are far apart in DNA/amino acid sequence; it would be very challenging to identify or even construct generate similar phenotypes via mutagenesis because, as an example, 9 different residues from 6 different parts of the DNA/amino acid sequence would need to be simultaneously mutated.

FIG. 29A-F. A heatmap plot illustrating mutational tolerance of cyclic peptides identified from enrichment performed in AAV6.

Heatmap plot of comprehensive, single-site amino acid substitutions of 6 highly enriched cyclic peptides (CPs) for AAV6 is shown. This plot visually depicts the functional impact of mutations on a particular cyclic peptide as measured by enrichment score. The heatmap provides valuable insights into the mutational landscape of cyclic peptides, highlighting positions where certain amino acid substitutions are either not tolerated (e.g., resulting in depletion), are neutral (e.g., resulting in similar enrichment scores), or are favored (e.g., resulting in higher enrichment, indicating enhanced cyclic peptide variants).

The heatmaps may be used to identify mutational hotspots where specific amino acid substitutions are favored or disfavored. This aids in understanding the preferences and constraints of a given cyclic peptide's sequence, guiding efforts to optimize performance in a data-driven manner. The deep mutational scanning enrichment map provides a comprehensive overview of the cyclic peptide mutational landscape, offering valuable information for additional engineering, molecular design, and understanding the functional consequences of amino acid substitutions. A key benefit of these plots in the context of cyclic peptides is that they teach not only sequence space (as they are typically used in proteins), but also provide insights useful for molecular engineering beyond natural amino acid sequence space (e.g., via non natural amino acids or synthetic chemistry approaches).

The heatmap is structured with rows indicating the position of the original amino acid, while the columns represent the position of the mutated amino acid (for a given row). The X-axis corresponds to the 20 natural amino acid substitutions for a given cyclic peptide residue index. The Y axis represents the amino acids present in a specific cyclic peptide, starting from the top with the “scar” residue from the peptide cyclization splicing reaction of DNA expressed cyclic peptides. The scar residue is typically amino acids containing a nucleophilic side chain like cysteine, serine, or threonine. Each row is labeled by the native amino acid of a given CP with amino acid positions going from top to bottom (e.g. index 1 is at the top, followed by index 2 followed by, index 3, etc. . . . ). The final amino acid at the bottom cyclizes with the scar residue at top. Though they are cyclic and have no termini, in this figure the top can be considered the imaginary N terminus and the bottom as the imaginary C terminus (with an invisible peptide bond between the top and bottom residues.

The pixels within the heatmap correspond to the enrichment of a cyclic peptide when the amino acid residue, represented by a specific row (e.g., C of index 1), is mutated to the residue of a specific column. Enrichment is represented by shades of gray. Light colors represent strong enrichment (improve viral production). Dark colors represent depletion (reduce viral production). Black represents below limit of detection (assumed to be very high depletion). Enrichment factors are log-scaled. If a pixel corresponds to the original amino acid residue of the cyclic peptide (where the row and column feature the same amino acid), then that amino acid is indicated (e.g., a Q mutated to a Q would be labeled with a Q). These pixels also convey the enrichment score of the cyclic peptide under analysis. The scar amino acid (usually Cysteine) will typically be highly intolerant of substitutions due to its critical role in intein splicing.

Many thousands of such plots have generated illustrating mutational sensitivity of different cyclic peptides across different viral serotypes, but due to space constraints, only a representative selection is provided herein. Multiple cyclic peptides were validated in FIGS. 23 and 25 for AAV and lentivirus, respectively, in order to provide a comparison between enrichment factors and improvements in viral titer and/or transduction efficiency (e.g., VG/ml, TU/ml, TU:VG, etc. . . . ).

FIG. 29A. Cyclic Peptide CSSLT (SEQ ID NO: 37) was highly tolerant of amino acid substitutions. In addition, Ser2Pro (the substitution of serine in residue index 2 of the cyclic peptide row with proline column) and Leu4Tyr are predicted to yield improved cyclic peptides based on the substantially increased enrichment factors indicated by the brighter pixels at those positions. The performance of this cyclic peptide is visualized in FIG. 23 for AAV2 and FIG. 25 for lentivirus.

FIG. 29B. Cyclic Peptide CSARR (SEQ ID NO: 46) was only somewhat tolerant of amino acid substitutions, with the majority of substitutions being less enriched (darker shades of gray) compared to the original cyclic peptide amino acid residues.

FIG. 29C. Cyclic Peptide CPLRSP (SEQ ID NO: 47) was highly intolerant of cyclic peptide substitutions Q or W at any position and acidic amino acids D and E to a lesser degree. However, Pro2His and Ser5Val are predicted to yield improved cyclic peptide based on the substantially increased enrichment factor indicated by the brighter white pixel at that position.

FIG. 29D. Cyclic Peptide CPYTD (SEQ ID NO: 44) was somewhat tolerant of amino acid substitutions in its sequence, but the majority of substitutions are not as highly enriched compared to the original amino acids. However, Pro2 is highly sensitive to substitutions, indicating a possible important structural role in this cyclic peptide. The performance of this cyclic peptide is visualized in FIG. 23 for AAV2 and FIG. 25 for lentivirus.

FIG. 29E. Cyclic Peptide CQVFQL (SEQ ID NO: 42) was highly sensitive to amino acid substitutions due to the majority of substitutions being completely depleted (black), while in some cases, a small number of substitutions appear to substantially increase performance of the cyclic peptide. In this case, the cyclic peptide is completely intolerant of W, Y, N, and T. The performance of this cyclic peptide is visualized in FIG. 23 for AAV2.

FIG. 29F. Cyclic Peptide CPLQVP (SEQ ID NO: 45) has the cyclic peptide map illustrating total intolerance to N at any position as well as strong intolerance to W and Y. At the same time, bright white colors indicate mutations that were highly enriched; Leu3Pro, Gln4Arg, and Val5Ala in particular. The performance of this cyclic peptide is visualized in FIG. 25 for lentivirus.

In conclusion, this set of amino acid mutational scanning for 6 CPs illustrates an exceptional means of not only identifying cyclic peptides that are capable of improving viral production, but also provides the sequence-function relationships of these compositions, allowing for understanding and further improving of their performance.

FIG. 30. Exemplary representative gels showing assembly of various AAV CP library constructs.
Part A. Image of agarose gel electrophoresis of representative SICLOPPS CP library generators with 6 or 7 NNK codons to generate CP diversity.
Part B. Image of agarose gel electrophoresis of representative CP DNA encoding fragments (with 5-8 amino acid ring size) for various libraries that were enriched in AAV1, AAV2, AAV5, AAV6, and prepared for NGS.

FIG. 31. Exemplary representative gels showing assembly of various Lentivirus CP library constructs.

Part A. Image of agarose gel electrophoresis of EcoRI/BamHI digest of Lentivirus CP library enrichment constructs, showing a small CP dropout. CP libraries range in size from 4 to 7 NNK degenerate library codons. The constructs at far right are controls and should not have dropouts.
Part B. Image of agarose gel electrophoresis of undigested Lentivirus CP library enrichment constructs. CP libraries range in size from 4 to 7 NNK degenerate library codons. The constructs at far right are controls and should not have dropouts.

### DETAILED DESCRIPTION OF VARIOUS EMBODIMENTS

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.

The terms “level” or “levels” are used to refer to the presence and/or amount of a target, e.g., a substance or an organism that can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a normal control.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, the term “mammalian cell” includes host cells from any member of the order Mammalia, such as, for example, human cells, mouse cells, hamster cells, etc. Exemplary human cells include human embryonic kidney (HEK) cells, such as HEK 293, a HeLa cell, or a HT1080 cell. Mammalian cells include mammalian cell cultures which can be either adherent cultures or suspension cultures. Adherent cultures refer to cells that are grown on a solid support surface, for example, on a plastic plate, or other suitable cell culture growth platform. Suspension cultures refer to cells that can be maintained in, for example, culture flasks or other vessels without attachment to a surface, which offers a large surface area for gas and nutrient exchange. Exemplary host cells useful for methods and compositions of the present invention include HEK 293 cells, HEK 293 T cells, Expi293 cells, Chinese hamster ovary (CHO) cells, HeLa cells, HeLa S3 cells, PER.C6 cells, HKB11 cells, CAP cells, Baby Hamster Kidney fibroblasts (BHK cells) (e.g., BHK-21 cells), mouse myeloma cells (e.g., Sp2/0 cells, NSO cells), green African monkey kidney cells (e.g., COS cells and Vero cells), A549 cells, rhesus fetal lung cells (e.g., FRhL-2 cells), or a derivative of any thereof cells.

As used herein, the term “adeno-associated virus (AAV)” refers to a small, replicative-defective, nonenveloped virus which belongs to the genus Dependoparvovirus and the family Parvoviridae. Over 10 adeno-associated virus serotypes have been identified so far, including serotypes AAV1, AAV2, AAV3, AAV4, AAV5, AAV6, AAV7, AAV8, AAV9, AAV10, AAV 11, AAV12, and AAV13. In addition to these serotypes, AAV pseudotypes have been developed, where AAV pseudotype contains the capsid of a first serotype and the genome of a second serotype. In addition, there are many AAV vectors derived from the classical serotypes. In addition, there are many animal-derived AAV vectors, including bovine AAV, primate AAV, equine AAV, ovine AAV, canine AAV, mouse AAV, rate AAV, avian AAV, and others. In addition, there are synthetic serotypes that are the result of directed evolution or artificial intelligence design that do not fit into phylogenetic categories or have negligible homology to naturally occurring AAV serotypes. In addition, there are chimeric AAVs that may contain sequence identity of multiple serotypes. An example is an AAV that has AAV2 capsid, but AAV8 ITRs. Another example is an AAV that has a chimeric capsid derived from AAV3, AAV4, and AAV5, while the ITRs are derived from AAV6. The term “AAV vector” as used herein refers to an active, infectious form of the AAV (i.e., viral particle or virion), which is used for delivery the DNA sequence operably linked to one or two functional AAV inverted terminal repeats (ITRs) into infecting cell. Among helper viruses that help AAV to replicate in host cells are adenoviruses, herpesviruses, or papillomaviruses.

The canonical AAV genome is composed of a linear single-stranded DNA molecule which contains approximately 4681 bases. The genome includes inverted terminal repeats (ITRs) at each end which function in cis as origins of DNA replication and as packaging signals for the virus. The ITRs are approximately 145 bp in length. Inverted terminal repeats flank the unique coding nucleotide sequences for the non-structural replication (Rep) proteins and the structural (VP) proteins. The VP proteins (VP1, −2 and −3) form the capsid. The terminal 145 nucleotides are self-complementary and are organized so that an energetically stable intramolecular duplex forming a T-shaped hairpin may be formed. These hairpin structures function as an origin for viral DNA replication, serving as primers for the cellular DNA polymerase complex as well as genome packaging, integration, rescue.

The term “recombinant” as applied to an AAV or AAV vectors, refers to the virus or vector that is the product of various non-natural, human-made manipulations, such as genetic alterations (such as encapsulation of a heterologous nucleotide sequence of interest), propagation in non-natural environment, and other procedures that result in a virus or vector that is distinct from a virus or vector found in nature. In preferred embodiments, AAV vectors used herein are recombinant AAV vectors, referring to an AAV vector comprising a polynucleotide sequence not of AAV origin (i.e., a polynucleotide heterologous to AAV), typically a sequence of interest for the genetic transformation of a cell (payload). In general, the heterologous polynucleotide is operably linked to two AAV inverted terminal repeat sequences (ITRs). The other components needed for production of recombinant AAV vectors are provided in trans, for example from plasmids, helper viruses, or packaging cell genome.

As used herein, the term “AAV replication gene” refers to a gene that is involved in the replication and regulation of the adeno-associated virus (AAV) genome, ensuring the efficient replication, packaging, and maintenance of the viral genome. In some embodiments, the AAV replication gene is selected from the group consisting of Rep78, Rep68, Rep52, and Rep40. Rep78 and Rep68 encode large multifunctional proteins with overlapping functions, including DNA binding, helicase activity, and ATPase activity. They are primarily responsible for initiating viral DNA replication and regulating various stages of the AAV life cycle. Rep52 and Rep40 encode smaller isoforms, each enhancing efficiency of DNA replication, playing role in packaging of viral DNA into capsids. By selecting the appropriate AAV replication gene, one can manipulate and control AAV replication dynamics. The choice of AAV replication gene influences factors such as replication efficiency, viral genome copy number, and the ability to establish persistent infection.

As used herein, the term “AAV capsid encoding gene” refers to a gene that encodes a structural protein (essential component) of the AAV capsid, which is responsible for encapsulating the viral genome and/or facilitating viral entry into target cells. In some embodiments, the AAV capsid encoding gene encodes a protein that is selected from the group consisting of VP1, VP2, and VP3. VP1, VP2, and VP3 are the major capsid proteins of AAV. These proteins self-assemble to form the icosahedral capsid structure, providing stability and protection to the viral genome during transmission and infection. In some embodiments, the AAV capsid encoding gene also encodes an accessory protein, such as the Assembly-Activating Protein (AAP) or MAAP, which play a role in the capsid-assembly process and influence the final capsid structure. By selecting the appropriate AAV capsid encoding gene, one can customize and engineer the AAV capsid, influencing vector tropism, immunogenicity, and other characteristics. The choice of AAV capsid encoding gene impacts the specific properties and behavior of the resulting AAV vector in terms of target cell specificity and transduction efficiency.

As used herein, the term “AAV helper gene” refers to a gene that is required for the replication, transcription, or packaging of an AAV viral vector in addition to proteins encoded by AAV replication gene and AAV capsid encoding gene. AAV viral helper genes can be classified into two categories: essential helper genes and non-essential helper genes. Essential helper genes are indispensable for replication, transcription, or packaging of the viral vector, while non-essential helper genes enhance the efficiency of vector production without being mandatory for these processes. As used herein, “AAV helper gene” refers to an essential AAV helper gene. Adenoviruses are a common source of essential AAV helper genes. Examples of essential adenoviral AAV helper genes include, but are not limited to, Adenovirus E1A, Adenovirus E1B55K, Adenovirus E2A, Adenovirus E4 or f6, and Adenovirus VA, which play vital roles in facilitating replication, transcription, and packaging of adenoviruses and AAV. AAV helper genes can be derived from various viruses, including but not limited to herpes simplex viruses (Adeno-associated virus DNA replication complexes in herpes simplex virus or adenovirus-infected cells; 1979), Human Papillomavirus (Productive Replication of Adeno-Associated Virus Can Occur in Human Papillomavirus Type 16 (HPV-16) Episome-Containing Keratinocytes and Is Augmented by the HPV-16 E2 Protein; 2000), Vaccinia virus (Vaccinia virus, herpes simplex virus, and carcinogens induce DNA amplification in a human cell line and support replication of a helper virus dependent parvovirus; 1986), hepatitis B virus (Hepatitis B virus infection enhances susceptibility toward adeno-associated viral vector transduction in vitro and in vivo; 2014), Human Bocavirus (Human Bocavirus 1 Is a Novel Helper for Adeno-associated Virus Replication; 2017), recombinant baculoviruses engineered to express helper genes from the previous viruses (A Recombinant Baculovirus Efficiently Generates Recombinant Adeno-Associated Virus Vectors in Cultured Insect Cells and Larvae; 2018). For, example, HSV-derived AAV helper genes include genes encoding HSV helicase-primase complex (UL5, UL8, UL52) and the major DNA-binding protein (UL29), which have been shown to provide sufficient helper gene function for AAV replication (A subset of herpes simplex virus replication genes provides helper functions for productive adeno-associated virus replication; 1991). In another example, human papillomavirus-derived AAV helper genes include the gene encoding HPV E1 protein, or HPV E1, E2, and E6 genes. In yet another example, Herpes Simplex Virus (HSV)-derived AAV helper genes include genes such as UL5 (Helicase-Primase Complex), UL8 (Helicase-Primase Complex), ICP8 (Single-strand DNA-binding protein), and ICP27 (Transcriptional regulator). In yet another example, Herpes Simplex Virus (HSV)-derived AAV helper genes include genes such as p80 (Late expression factor), p143 (DNA replication factor), p40 (Nucleocapsid assembly factor), and p32 (Single-strand DNA-binding protein). AAV helper genes derived from other viruses, or potentially obtained through artificial intelligence, can also be utilized in the methods disclosed herein.

As used herein, the term “lentiviral gag gene” refers to a gene that participates in a lentivirus assembly in host cells and typically encodes a structural protein. As used herein, the term “lentiviral pol gene” refers to a gene that encodes an enzyme required for reverse transcription and/or integration of lentivirus into the host cell genome. As used herein, the term “lentiviral rev gene” refers to a gene that facilitates nuclear export of unspliced or partially spliced viral RNAs in host cells. As used herein, the term “env gene” refers to an envelope gene that participates in a lentivirus assembly in host cells and encoding a glycoprotein from an enveloped virus.

The term “engineered” as used in reference to a cyclic peptide molecule, e.g., an engineered cyclic peptide, to a protein, or to a nucleic acid sequence, implies that such molecules are created by human intervention and/or they are non-naturally occurring. An engineered nucleic acid sequence can include any type of modification that can be made to a nucleic acid (e.g., introduction, substitution, deletion, replacement, rearrangement, epigenetic modification, etc.). In some embodiments, an engineered cyclic peptide may be selected or determined by the methods disclosed herein and then may be further modified to obtain a further engineered cyclic peptide. In some embodiments, a further engineered cyclic peptide has one or more improved characteristics compared to the starting engineered cyclic peptide, for example, increased membrane permeability or increased stability in host cells. Sequence of a further engineered cyclic peptide can contain 1, 2, 3, 4, 5, 6, 7, 8, 9, 10, or more amino acid differences (e.g., substitutions and/or additions) compared to the sequence of starting engineered cyclic peptide. A further engineered cyclic peptide generally exhibits at least 70%, 80%, 85%, 86%, 87%, 88%, 89%, 90%, 91%, 92%, 93%, 94%, 95%, 96%, 97%, 98%, 99% or more sequence identity to a corresponding starting engineered cyclic peptide. Non-naturally occurring amino acids as well as naturally occurring amino acids are included within the scope of permissible substitutions or additions. In some embodiments, a further engineered cyclic peptide has a different cyclization pattern compared to the starting engineered cyclic peptide. The term “engineered” in the context of engineered cyclic peptide is not to be construed as imposing any condition for any particular starting composition or method by which the engineered cyclic peptide is created. Thus, engineered cyclic peptide denotes a composition and not necessarily a product produced by any given process

In some embodiments, variants of a cyclic peptide (such as a further engineered cyclic peptide described above) displaying only non-substantial or negligible differences in structure can be generated by making conservative amino acid substitutions in the engineered cyclic peptide. By doing this, engineered cyclic peptide variants that comprise a sequence having at least 90% (90%, 91%, 92%, 93%, 94%, 95%, 96%, 97%, 98%, and 99%) sequence identity with the engineered cyclic peptide sequences can be generated, retaining at least one functional activity of the engineered cyclic peptide, e.g., ability to increase viral titer and/or transduction efficiency of a viral vector composition. Examples of conservative amino acid changes are known in the art. Examples of non-conservative amino acid changes that are likely to cause major changes in peptide structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e.g., glycine. Methods of making targeted amino acid substitutions, deletions, truncations, and insertions in peptides are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “sequence identity” is a measure of identity between peptides at the amino acid level, and a measure of identity between nucleic acids at nucleotide level. The peptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website. Another program that can be used to calculate sequence identity is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty:1.00; Gap Penalty: 1.00; Gap Size Penalty: 0.33; and Joining Penalty: 30.0.

The terms “corresponding to position(s)” or “position(s) . . . with reference to position(s)” of or within a peptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the peptide upon alignment with the disclosed sequence using a standard alignment algorithm, such as the BLAST algorithm (NCBI). One skilled in the art can identify any given amino acid residue in a given peptide at a position corresponding to a particular position of a reference sequence, such as set forth in the Sequence Listing, by performing alignment of the peptide sequence with the reference sequence (for example, by using BLASTP publicly available through the NCBI website), matching the corresponding position of the reference sequence with the position in peptide sequence and thus identifying the amino acid residue within the peptide.

The term “host cell” refers to a mammalian or insect cell. The term “host cell permissive for AAV replication” refers to a cell, such as a mammalian or insect cell, in which AAV can replicate and generate AAV vectors when certain elements necessary for intracellular AAV replication are present or introduced into such a cell. Elements necessary for intracellular AAV replication, packaging and/or vector generation include AAV replication gene(s), AAV capsid encoding gene(s), and viral helper gene(s). Since AAV is replication-defective, specific viral helper gene(s) that originated from adenoviruses, herpesviruses (HSV), bocaviruses or papillomaviruses need(s) to be inserted into a host cell to make it permissive for AAV replication. In exemplary embodiments, viral helper gene is an adenovirus helper gene. In other embodiments, viral helper gene is HSV helper gene, bocavirus helper gene, or a papillomavirus helper gene. As referred to herein, the term “adenovirus helper gene” refers to a gene that is composed of one or more nucleic acid sequences derived from one or more adenovirus subtypes or serotypes that contributes to AAV replication, packaging and/or generation of AAV vectors.

In some embodiments, AAV vectors produced in host cells by the methods disclosed herein are used as therapies themselves. In some embodiments, produced AAV vectors are used in the research, production, and/or manufacturing processes that can generate therapies. For example, AAV vectors can be used in many ways that include but are not limited to vaccines, cancer therapies (e.g., oncolytic therapies), and/or gene therapies (e.g., in vivo gene and/or genomic editing). Methods of the present disclosure can be used to generate host cells with beneficial characteristics for expression of an AAV vector. Methods of the present disclosure can be used to generate helper viruses with beneficial characteristics for production of an AAV vector. Methods of the present disclosure can be used to generate packaging plasmid sets with improved/beneficial characteristics for production of an AAV vector.

In preferred embodiments, a host cell provided herein includes one or more one or more AAV replication genes encoding non-structural replication (Rep) proteins (such as, for example and without limitation, Rep 78, Rep 68, Rep 52 and Rep 40); one or more AAV capsid encoding genes that encode structural (VP) proteins (such as, without limitation, VP1, −2 and −3) forming the AAV capsid; and one or more viral helper genes (such as, without limitation, Adenovirus E1A, Adenovirus E1B55K, Adenovirus E2A, Adenovirus E4 or f6, and Adenovirus VA). Viral helper genes may include various adenoviral virus genes, HSV genes, bocavirus genes and papillomavirus genes. These genes (e.g., AAV replication genes, AAV capsid encoding genes, and/or viral helper genes) are inserted into a host cell operable linked to (under control of) other transcriptional regulatory sequences, including promoters (e.g., regulatable promoters). Exemplary description of regulatory sequences including suitable promoters for use in the disclosed methods can be found in US 20200199627 A1 and U.S. Pat. No. 6,924,128 B2, incorporated herein. In some embodiments, two or more AAV replication genes, AAV capsid encoding genes, and/or viral helper genes may be utilized simultaneously in the disclosed methods to produce AAV vectors.

In some embodiments, one or more nucleic acid sequences essential for production of AAV vectors in host cells comprise a heterologous promoter sequence that is or comprises an SV40 promoter, an elongation factor (EF)-1 promotor, a cytomegalovirus (CMV) promoter, a phosphoglycerate kinase (PGK)1 promoter, a ubiquitin (Ubc) promoter, a human beta actin promoter, a tetracycline response element (TRE) promoter, a spleen focus-forming virus (SFFV) promoter, a murine stem cell virus (MSCV) promoter, a supercore promoter (SCP), a CAG promoter, or a derivative thereof. In some embodiments, one or more nucleic acid sequences essential for production of AAV vectors in host cells comprise a heterologous enhancer sequence that is or comprises a CMV early enhancer, a cAMP response-element (CRE) enhancer, or a derivative thereof. In some embodiments, one or more nucleic acid sequences essential for production of AAV vectors in host cells can be integrated into a mammalian cell genome and under the control of an inducible transcriptional control element (e.g., inducible promoter and/or inducible enhancer). In some embodiments, one or more nucleic acid sequences essential for production of AAV vectors in host cells can be present episomally in a mammalian cell and under the control of an inducible transcriptional control element (e.g., inducible promoter and/or inducible enhancer).

In some embodiments, the elements necessary for intracellular AAV replication, packaging and/or vector generation in a host cell are contained within the host cell in separate nucleic acid molecules, for example separate chromosomes, plasmids, or vectors. In other embodiments, the nucleic acid molecules encoding the various elements necessary for AAV replication, packaging and/or vector generation are included on the same chromosome, plasmid, or vector. In further embodiments, certain of the elements are contained on the same nucleic acid molecule (e.g., AAV capsid encoding genes and AAV replication genes), while other genes are contained on separate nucleic acid molecules (e.g., helper genes). In yet other embodiments, certain of the elements are integrated into genome of the host cell.

The term “manufacturability” refers to the degree to which a product (e.g., AAV vector for gene therapy) can be effectively manufactured given its design, cost, purity, yield, safety, and efficacy requirements. Manufacturability is centered on a) overall feasibility, e.g. rAAV that works when produced at lab scale fails to work when produced at larger scale for any one or combination of reasons including, but not limited to, higher toxicity, lower safety, lower viral titer, lower potency/transduction efficiency, tropism, higher contamination, higher impurities (product and process-related), immunogenicity, higher purification requirements, stability, downstream processing requirements, batch failure rate; and b) excess cost, e.g. the intrinsic inefficiency of rAAV production can result in products that cost more to manufacture than they can be sold for.

In the context of AAV manufacturing, the most common manufacturability challenges include: viral titer (as measured by VG/ml or VG/cell); full:empty capsid ratio (commonly assessed by comparing the genome copy number, or physical titer, to the total viral particle counts based on capsid protein); infectious unit titer (as measured by IU/ml or IU/cell), also referred to as transducing unit titer (as measured by TU/ml or TU/cell); a related feature is transduction efficiency, or the TU:VG ratio, which indicates how many functional AAV vectors are contained out of the total number of full, genome-containing AAV vectors. The term “Infectious unit titer” as used herein is a measurement of the number of viral particles that can transduce cells (e.g. per cell or per ml; provided as IU/ml or IU/cell). Infectious unit titers are typically quantified with cell transduction assays (e.g. FACS or fluorometric microscopy on transduced cells, TCID50).

The terms “reference host cell”, “reference plurality of host cells” as used herein refer to a host cell or a plurality of host cells, respectively, not comprising an engineered cyclic peptide, according to various embodiments of the present invention. Similarly, “reference AAV vector composition” as used herein refers to a AAV vector composition produced in host cell in the absence of an engineered cyclic peptide (reference AAV vector composition have the same serotype or pseudotype as the AAV vector composition to which reference AAV vector composition is compared). These terms are used to designate standard or control host cells (or AAV vector composition), which are not modified by an engineered cyclic peptide. Reference AAV vector composition is produced from host cells under identical, or nearly identical conditions, as the AAV vector composition to which reference AAV vector composition is compared, except from absence of engineered cyclic peptides in the host cells during production (reference AAV vector composition is produced from the same host cells, but without engineered cyclic peptides). In some embodiments, reference or control host cells (or AAV vector composition) are tested substantially simultaneously with the testing host cells of interest (e.g., host cells comprising an engineered cyclic peptide, which is either produced endogenously or supplied exogenously). Typically, as would be understood by those skilled in the art, reference or control host cells (or AAV vector composition) are characterized under comparable conditions or circumstances to those under assessment. Those skilled in the art will appreciate when sufficient similarities are present to justify reliance on and/or comparison to a particular possible reference or control.

As used herein, the term “infectivity” refers to the ability of the viral vector to enter and successfully transduce the target cells. Optimizing viral production in cells, such as by expressing a cyclic peptide (CP), may improve infectivity by generating viral particles with enhanced capsid structures that facilitate cellular entry and successful delivery of the genetic payload. An optimized cell environment for viral production may influence various intracellular processes that contribute to the overall quality of the viral particles. These processes may include post-translational modifications like glycosylation or sumoylation, methylation of viral payload DNA, the activity of various proteases in capsid maturation, and vesicular pH, which has been shown to play a crucial role in capsid processing. By optimizing these cell behaviors and pathways, the quality of the viral particles can be improved, resulting in enhanced infectivity and better clinical outcomes.

As used herein, the term “transduction efficiency” refers to the effectiveness (efficiency) of viral particles in delivering their genetic payload to the target cells. This efficiency may be represented by the transducing units to vector genomes ratio (TU:VG). Viral formulations with enhanced transduction efficiency can achieve greater numbers of DNA delivery events for the same number of DNA-containing viral particles. For example, if formulation A requires 10 viral particles to achieve one transduction event (transduction efficiency of 0.1) and formulation B requires 5 viral particles to achieve on transduction event (transduction efficiency of 0.2), then formulation B would have a 2 fold greater transduction efficiency compared to formulation A (0.2/0.1=2). Enhanced transduction efficiency may be achieved by optimizing the cellular environment for viral production, leading to viral particles with improved capsid structure, viral particle biochemistry, or viral particle assembly. Increasing transduction efficiency refers to improvements in the TU:VG ratio, which encompasses both infectivity and biological titer. Higher transduction efficiency implies a higher proportion of infectious particles to total vector genomes, which can result from a higher biological titer and/or lower VG levels.

As used herein, the term “viral titer” refers to the concentration or quantity of viral particles present in a given sample. By measuring the viral titer of a given sample, one can gain valuable insight into the manufacturability and clinical utility of viral compositions. Viral titer measurements typically fall into two broad categories: physical titer or biological titer (each defined separately). Viral titer is reported using different metrics depending on what type of virus is being evaluated and what aspect of the viral material is being measured. In the context of AAV, physical titer is often reported in viral genomes per milliliter (vg/ml) or genome copies per milliliter (ge/ml) and can be measured using techniques like qPCR or ddPCR; however, biological titer is becoming an increasingly important metric as the clinical use of AAV matures. In the context of lentivirus, biological titers are more commonly reported with transducing units per milliliter (TU/ml) or infectious units per milliliter (IU/ml) being typically used metrics; however, lentiviral physical titers are also commonly reported in viral particles per milliliter (vp/ml). This titer is often determined by measuring the amount of p24 antigen, a viral protein, in the sample via ELISA or by quantifying the amount of viral RNA present using qRT-PCR.

As used herein, the term “physical titer” refers to the total count of viral particles in a sample, irrespective of their infectivity. This is generally quantified by assessing a component of the viral particle, such as viral RNA, DNA, or protein(s). Physical titer may be reported in a variety of ways depending both on the conventions for a given virus and the methods by which physical titer is determined. The most commonly reported physical titer metrics are viral genomes per milliliter (vg/ml), genome copies per milliliter (gc/ml), or viral particles per milliliter (vp/ml). In the context of AAV, qPCR or ddPCR are commonly used to determine and report vg/ml or gc/ml. In the context of lentivirus, vp/ml is commonly reported and is often determined by measuring p24 antigen by ELISA or quantifying viral RNA by qRT-PCR. For herpes simplex virus (HSV), the physical titer is typically reported in gc/ml; adenovirus in vp/ml; and baculovirus in occlusion bodies per milliliter (OBs/ml) or vp/ml for occluded and non-occluded baculoviruses respectively.

As used herein, the term “biological titer” refers to the count of biologically functional viral particles in a sample (i.e., viral particles that are capable of infecting target cells or transducing genes (i.e., payload) into target cells, which may or may not lead to gene expression). Optimizing viral production in cells, such as by expressing a cyclic peptide (CP) as disclosed herein, may enhance the biological titer by increasing the proportion of functional viral particles. Higher biological titers may result from optimized cellular environments that support the generation of viral particles with improved capsid structures, tropism, and immune evasion properties. Depending on the virus being measured and specific method used to quantify the viral material, biological titer is typically reported in infectious units per milliliter (IU/ml or IFU/ml), transducing units per milliliter (TU/ml), or plaque-forming units per milliliter (PFU/ml).

TU/ml is determined by quantifying the number of target cells that express the transgene after being exposed to a known volume of the viral vector preparation. This measurement provides a more functional assessment of the viral vector, as it considers the vector's ability to deliver and express the transgene, e.g., green fluorescent protein (GFP), in target cells. IU/ml represents the ability of the viral vector to infect target cells, regardless of whether it leads to transgene expression or not. It is determined by quantifying the number of target cells that are infected (i.e., contain viral genomes) after being exposed to a known volume of the viral vector preparation. PFU/ml is measured by the ability of a virus to form plaques on a cell monolayer. By counting the number of plaques formed by a given dilution of viral material, the number of PFU/ml can be determined.

Different viruses and their applications dictate a specific metric to be used for biological titers. In the context of gene therapy, where a virus acts as a vector, the titer may be reported in TU/ml or IU/ml to reflect the number of cells successfully transduced. For example, adeno-associated virus (AAV) biological titers are often reported TU/ml, determined by assays that measure either the ability of the AAV to transduce cells and express a particular gene like green fluorescent protein (GFP). Lentiviral biological titers are also commonly reported as TU/ml or IU/ml, again reflecting either the transducing capability or the general infectivity of the virus. Herpes simplex virus (HSV) or adenovirus, biological titers are often reported as IU/ml, TU/ml, or PFU/ml, based on whether the assay measures general infectivity, gene transduction, or plaque formation.

As used herein, the term “payload” refers to any entity of interest for delivery by an AAV vector produced by methods of the present disclosure. For example, such a payload may be desired to be introduced into a cell, organ, organism, and/or cells. In some embodiments, a payload sequence is or comprises a heterologous nucleic acid sequence for delivery by an AAV vector. In some embodiments, a payload sequence comprises an encoding region and one or more of a gene regulatory element and a transcription terminator. Non-limiting examples of gene regulatory elements include promoters, transcriptional activators, enhancers, and polyadenylation signals. In some embodiments, a payload sequence comprises an encoding region, a gene regulatory element, and a transcription terminator, positioned relative to each other such that the encoding region is between the gene regulatory element and the transcription terminator. In some embodiments, a coding sequence encodes a gene product. In some embodiments, the gene product is an RNA molecule. In some embodiments, an encoding region encodes a polypeptide. In some embodiments, the payload may incorporate multiple functional units (e.g., a promoter region, an intron, a Kozak sequence, an enhancer, a polyadenylation sequence, and/or a cleavage sites or sequence that encode a protein). Some payloads may be nucleic acid-based and not encode a protein, such as miRNA, siRNA, or aptamers. For example, AAV vectors may contain as a payload the viral genome, either in whole or in part (e.g., only essential components), of any naturally occurring and/or recombinant AAV serotype nucleotide sequence or variant. The payload may be single-stranded (and containing 2 ITRs) or self-complementary (and containing 3 ITRs), and can be produced or modified using various methods known in the art.

The term “peptide” as used herein refers to a molecule comprising a chain of three or more amino acids joined by peptide bonds. In some embodiments, a peptide comprises 4 to 80 amino acid residues. The term “cyclic peptide” or “CP” indicated that the peptide chain contains a circular sequence of peptide bonds. In preferred embodiments, a cyclic peptide comprises 5 to 20 amino acid residues. In preferred embodiments, a cyclic peptide has N-to-C(or head-to-tail) cyclization, which is amide bond formation between amino and carboxyl termini. In other embodiments, a cyclic peptide may contain a different cyclization pattern, e.g., amide bond formation between two non-neighboring amino acid residues, other than between amino and carboxyl termini. In various embodiments of the disclosed methods and compositions, cyclization of cyclic peptides may be obtained by any one of a variety of methods known in the art, including, without limitation, methods based on protein tags (e.g., using inteins or engineered protein domains for isopeptide bond formation), chemical methods (e.g., using native chemical ligation, direct backbone cyclisation, disulfide formation, aldehyde-based ligations), and enzymatic methods (e.g., using non-ribosomal peptide synthetases, subtiligase variants, transglutaminases). The amino acids of cyclic peptides are typically alpha L-amino acids, but may also be D-amino acids, modified amino acids, amino acid analogs, amino acid mimetics, beta amino acids, gamma amino acids, delta amino acids or any combination thereof. The term “cyclic peptide” also encompasses an amino acid polymer that has been modified naturally or by intervention; for example, disulfide bond formation, glycosylation, lipidation, acetylation, phosphorylation, or any other manipulation or modification. Chain cyclization of cyclic peptides (CPs) may occur outside cells (in vitro), or inside cells (for example, post-translationally, such as self-cyclization induced by inteins). In some embodiments, a cyclic peptide comprising only natural amino acid residues may be selected by using methods described herein; then, a modified version of the cyclic peptide (comprising one or more modified or non-standard amino acid residues) may be prepared and used to enhance viral production. Various modifications are known in the art to enhance cellular permeability, stability or other properties of the selected CPs.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or He), Lysine (K or Lys), Leucine (L or Leu), Methionine (M or Met), Asparagine (N or Asn), Proline (P or Pro), Glutamine (Q or Gin), Arginine (R or Arg), Serine (S or Ser), Threonine (T or Thr), Valine (V or Val), Tryptophan (W or Tip), and Tyrosine (Y or Tyr). An amino acid may be an L-amino acid or a D-amino acid. Non-standard amino acids may be modified amino acids, amino acid analogs, amino acid mimetics, nonstandard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, gamma amino acids, delta amino acids, Homo-amino acids, Proline and Pyruvic acid derivatives, 3-substituted alanine derivatives, glycine derivatives, ring-substituted phenylalanine and tyrosine derivatives, N-methyl amino acids.

As used herein, each amino acid residue used in the disclosed CPs may be categorized into the five categories based on the properties of their side chains/R-groups: HYD, ARO, POL, POS and NEG. As used herein, 20 standard amino acid residues are categorized as follows:
1) HYD group: amino acid residues with hydrophobic/aliphatic/non-polar/non-aromatic R-groups, which consists of residues selected from the following group: ‘G’, ‘A’, ‘V’, ‘I’, ‘L’, ‘M’, ‘P’;
2) ARO group: amino acid residues with hydrophobic aromatics R-groups, which consists of residues selected from the following group: ‘F’, ‘W’, ‘Y’;
3) POL group: amino acid residues with polar uncharged R-groups, which consists of residues selected from the following group: ‘S’, ‘T’, ‘Q’, ‘N’, ‘C’;
4) POS group: amino acid residues with positively charged (Basic) R-groups, which consists of residues selected from the following group: ‘K’, ‘R’, ‘H’; and
5) NEG group: amino acid residues with negatively charged (Acidic) R-groups, which consists of residues selected from the following group: ‘D’, ‘E’.

In some embodiments where non-natural and/or modified amino acid residues are used in a CP, such residues may also be categorized into one or more of the same five categories based on the chemical properties of their side chains/R-groups. There are cases where non-natural and/or modified amino acid residues have chemical properties (between two or more categories); in such cases, such residues can be assigned to more than one category (e.g., assigned to both HYD and POL, or to both ARO and POL), see some specific examples below.

As used herein, the term “post-translational modification” refers to modifications that occur on a peptide after its translation by ribosomes is complete. A post-translational modification may be a covalent modification or enzymatic modification.

As used herein, the term “linker” refers to one or more of a nucleotide, a nucleotide analog, an amino acid, a peptide, a peptide, a polymer, or a non-nucleotide chemical moiety that is used to join two molecules. A linker may be used to join a binding agent with a coding tag, a recording tag with a peptide, a peptide with a support, a recording tag with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site (e.g., the site to which the binding agent binds).

As used herein, the term “barcode” refers to a nucleic acid molecule, such as DNA molecule, of about 3 to about 100 bases that provides a unique identifier tag (identifying information) for a cyclic peptide produced in a host cell. A barcode can be an artificial sequence or a naturally occurring sequence. In certain embodiments, each barcode within a population of barcodes is different. In other embodiments, a portion of barcodes in a population of barcodes is different, e.g., at least about 10%, 50%, 90% of the barcodes in a population of barcodes is different.

The term “provirus” as used herein refers to the genetic material of a virus that has been integrated into the genome of a host cell. In this integrated state, the virus is not actively replicating and does not produce virions. Instead, the viral genome is passively replicated along with the host genome as part of the normal host replication cycle. For example, AAVs can enter a provirus state where the AAV genome is integrated into the host cell genome. Many viruses are known to have provirus stages of their replication cycle. Proviruses can also be created via genetic engineering, for example via transposon integrations. This is useful for creating proviruses in situations where a virus may not naturally integrate into the host genome.

In certain conditions, proviruses can become mobilized and rescued to produce viral particles. In some cases, the provirus remains dormant or latent within the host cell. It can be activated by various stimuli such as environmental stress, exposure to certain chemicals, ultraviolet radiation, changes in the host's health, viral infection (e.g. of helper viruses). Activation initiates the transcription of the proviral DNA.

Sometimes, proviruses may be defective and unable to produce new virus particles on their own. They can be “rescued” if the host cell is infected with a similar virus. In this case, the proteins produced by the new infection can package the genetic material of the provirus, leading to the production of viral particles with the proviral genome.

In the context of AAV, in the absence of a helper virus (like adenovirus or herpesvirus), the AAV can remain latent and not produce any viral particles. However, when the cell is subsequently infected with a helper virus, the AAV provirus can be activated or “rescued.” The helper virus provides necessary factors that initiate the replication of AAV. The AAV genome is then transcribed and translated to produce viral proteins.

The term “helper virus” as used herein refers to a virus that provides one or more helper functions encoded by one or more helper genes encoded on the helper virus genome. A helper virus allows an otherwise deficient coinfecting virus to replicate. Helper viruses are also commonly used to replicate and spread viral vectors for gene therapy. A helper provides essential functions or factors enabling a replication defective or replication dependent virus to complete its replication cycle within a host cell. The helper virus contributes elements that the defective virus lacks, which are necessary for the synthesis, assembly, and sometimes the release of new viral particles.

In the case of Adeno-Associated Virus (AAV), it is known as a dependoparvovirus because it requires the help of a helper virus to replicate efficiently. AAV, in its natural state, relies on the presence of helper viruses such as Adenoviruses or Herpes Simplex Viruses. When cells are coinfected with AAV and one of these helper viruses, the helper virus provides essential replication factors that AAV lacks, allowing AAV to undergo a productive infection. Helper viruses can be natural or recombinant, with the exemplary recombinant helper virus being baculovirus expressing Adenoviral helper genes necessary for the production of AAV.

The term “chimera” or “chimeric virus” as used herein refers to a virus comprising genetic material originating from two or more distinct viruses. For example, lentivirus chimeras are commonly made by substituting the wildtype HIV-1 derived envelope glycoprotein gene with a variety of other glycoprotein gene derived from enveloped viruses.

The term “transfer vector” or “transfer plasmid” as used herein refers to a plasmid that contains a DNA payload sequence intended to be packaged in a viral vector and a packaging sequence. For example, an AAV transfer vector will encode a desired DNA payload flanked by two ITR sequences. An exemplary lentiviral transfer vector will contain a desired nucleotide sequence payload operably linked to a Psi packaging sequence. Transfer vectors, at a minimum, require a packaging nucleotide sequence (e.g. AAV ITRs, lentiviral Psi sequences) to ensure the nucleic acid is packaged into viral. Depending on the desired behavior of the viral vector, transfer vectors may comprise additional components, including, but not limited to promoters, terminators, regulatory elements, replication genes, capsid encoding genes, helper genes, integration elements, replication sequences (e.g. ITRs, LTRs).

The term “nested virus” or “nested viral vector” as used herein refers to a viral composition containing at minimum the transfer vector of a “guest virus” that is operably linked to a “host virus” as the payload of the host virus and in which the host virus and guest virus genomes are packaged into the host virus viral particle. For example, the transfer vector of guest virus A, may be inserted as a payload into the transfer vector of host virus B and the nested viral genome may be packaged into viral particles of host virus B.

### Disclosure of Exemplary Methods and Compositions

In some embodiments, the present teachings disclose methods for selecting genetically encoded, endogenously expressed cyclic peptides that enhance AAV vector manufacturability (packaging and infectivity of AAV capsids). AAV capsid quality is a complex matter. A single AAV viral preparation in host cells contains a heterogeneous mix of viral particles with diverse post translational modification profiles, capsid protein stoichiometries (estimated to be 1891), alternatively spliced capsid protein arrangements, packaged DNA, and payload DNA methylation states. Within this mix, it is observed that some viral particles are capable of more effectively delivering the payload DNA and having the DNA express robustly (high quality/performance rAAV particles). In contrast, an rAAV that is empty or fails to deliver its DNA payload (may be due to suboptimal post translational modifications), or results in low gene expression, would collectively be regarded as low quality/performance rAAVs. The ratio of functional to non-functional particles can determine clinical safety and potency. The infectious titer can be determined by quantifying the number of rAAV virions that successfully deliver the DNA payload. This number is often much lower than the total viral titer, which is typically quantified by counting the number of viral genomes (e.g. with qPCR or ddPCR). A skilled person in the art will recognize that different rAAV designs may yield dramatically different viral titers and infectious titers, even when the manufacturing strategy is held constant.

In some embodiments, the present teachings provide methods of identifying an engineered cyclic peptide (CP) capable of improving manufacturability of AAV particles in host cells and increase quantity and/or quality (infectiousness) of produced AAV vectors.

Genetic engineering seems to be an obvious approach to improve properties of cell lines for viral production. However, genetic modification linkage between cell genome and viral phenotype is lost as soon as the virus leaves the cell; therefore, it is hard to link the phenotype of a virus to the cell that produced it. This means that if a cell mutant were to be improved and generate more/better viruses, the viruses would provide no indication of which cell mutant from the high diversity cell pool with improved production properties they originated from. Some efforts have found solutions to this by using the virus to deliver and store genetic modifications, such as CRISPRi or siRNA. However, these approaches are limited in that they only target a small number of genes and they only modulate expression. Other efforts have attempted to use viruses to deliver mutations, such as by encoding repair fragments and guide RNAs to packaging cells. However, this is challenging and suffers from only targeting a single gene at a time.

The methods and compositions provided herein have several advantages over existing approaches. In contrast to a single siRNA or sgRNA, or any other programmable gene expression modulator that will be able to modulate only one single target (determined by its base-pairing complementarity), a single cyclic peptide (CP) has the potential to interact with and modulate multiple (or even majority of) biomolecules in a cell to varying degrees, including simultaneously targeting of multiple targets. Each CP is likely to have a various degree of affinity to multiple protein targets, nucleic acids and/or small molecules. Because CPs exploit a post-translational approach, they can interact with a much greater number of biomolecules inside of a cell, and the accumulated effects of these interactions and perturbations collapse into a single cellular phenotype.

In addition, whereas genetic regulation only provides for an increase or decrease in the amount of expression of a specific gene, it cannot change the function of the underlying gene beyond what is reachable through alternative splicing, post translational modification, etc. In contrast, CPs can engage their targets in ways that can fundamentally alter the target's behavior. For example, CPs have been successfully used, without limitation, to disrupt DNA binding activity of multiple transcription factors, to alter ribo-switching properties of a riboswitch, to rescue lethal mutants, or to alter enzymatic activity of metabolic genes. In all these cases, the new biological activity effected by the CP was not encoded by the original organism. No amount of genetic regulation would have allowed the emergence of the phenotype that the CP was able to generate.

Based on available research, a single CP is expected to strongly interact with 10-100 proteins and protein assemblies, have weaker affinity for an additional population of protein targets, and potentially interact with several nucleic acids (Pal S, et al., RNA-Binding Macrocyclic Peptides. Front Mol Biosci. 2022 Apr. 19; 9:883060), small molecules (Haberhauer G, et al., A very stable complex of a modified marine cyclopeptide with chloroform. Nat Commun. 2013; 4:2945), and ions (Kubik S. Synthetic Receptors Based on Abiotic Cyclo(pseudo)peptides. Molecules. 2022 Apr. 28; 27(9):2821). While this is undesirable in the context of therapeutic development, as an engineering tool for bioproduction, this increased breadth of perturbation is highly desirable. For each of these interactions, in addition to simple increases or decreases in activity, allosteric modulation can increase the number of biological behaviors, for example altering specificity of binding proteins, stabilizing rare protein conformations to generate new enzymatic reactions, or increasing promiscuity of proteolysis. In such cases, there is not a simple increase or decrease in protein's canonical behavior; instead, the collection of all possible protein behaviors, which might be described as a protein's latent functional promiscuity, can be rapidly and reversibly unlocked by such post-translational perturbations from an introduced CP. The accumulation of multiple potential changes in activity across multiple targets greatly surpasses the breadth in functional perturbation that can be achieved by targeted methods of genetic regulation.

Summarizing the above, CPs combine some advantages of small molecules and targeted macromolecules (e.g., antibodies) when used for regulating intracellular environment. In some embodiments, the methods described herein utilize concept of selfish elements (e.g., CP gene sequences) that can improve the efficiency of their replication in a competitive environment (e.g., a DNA library encoding CPs). By performing one or more rounds of selection with a library encoding CPs, cells with high viral manufacturing capacity can be generated. Typically, more rounds of selection would enrich cell (or viral) population with sequences that encode CPs that provide selective advantages for AAV vectors incorporating these CPs. Some of the described methods provide way for CPs to competitively self-replicate using a viral vector; accordingly, tools of directed evolution and selective/competitive enrichment are used for the discovery of CPs. In some embodiments, the virus is used to harbor a selfish element that directly alters cell behavior and is easily identified. In some embodiments, the virus is used to hold the element that optimizes the cell rapidly and directly with the element also being readily identifiable using standard techniques like molecular cloning, and sequencing/NGS. Further, CP gene sequence libraries can be pooled and the performance of all hits easily validated using NGS. This contrasts with drug screening approaches in which pooling an entire library of millions of compounds would make it impossible to effectively identify hits, which leads to the requirement of microwell plate and automated high throughput screening approaches. Because CPs are genetically encodable and can be expressed ribosomally, the DNA that encodes these highly efficient chemical perturbation modules can be packaged into the viral vector, providing an unexpected way to exploit genetic information storage and transmission for the identification of CP-based viral replication enhancers.

CPs provide many advantages over other types of molecules, including linear peptides, during the described selection process. CPs are conformationally constrained which both improves target selectivity and potency making them an ideal tool for general purpose, post-translational perturbation of cellular proteome. CPs have enhanced metabolic stability and resistance to proteolytic degradation because they have fewer exposed termini and less conformational flexibility compared to linear peptides. In general, cyclic peptides are more permeable than linear peptides, meaning that more cells will still be exposed to CP effects. In general, cyclic peptides tend to have lower aggregation tendencies due to their conformational rigidity and stable structure, which can contribute to their solubility. Linear peptides have a more flexible and dynamic structure, which can lead to the exposure of hydrophobic residues that can promote aggregation through intermolecular interactions. CPs can have lower toxicity and good biocompatibility, because they are derived from natural amino acids, compared to small molecule modulators. The entire surface/entity of a CP is a functional element that involves in interactions with molecular targets, in contrast to, for example, nanobodies, where only CDRs are functional, and the rest of the protein provides the structure that allows the CDRs to exist. CPs can be synthesized by chemical or biological methods and conjugated with various functional groups; methods of synthesis and conjugation are well-known to skilled in the art. Due to their size, CPs have better potential to modulate protein-protein interactions compared to small molecule modulators. Cyclic peptides exhibit diverse structures and can interact with target proteins through various binding modes. Cyclic peptides can target “undruggable” molecules due to their ability to interact with large, flat, and shallow binding surfaces that are difficult for small molecules to engage. Small molecules typically require well-defined pockets to bind effectively, whereas cyclic peptides can exploit their structural diversity and conformational flexibility to interact with challenging targets. Cyclic peptides can interact with non-protein molecules, such as nucleic acids, lipids, and carbohydrates. Their structural diversity and adaptable binding modes enable them to recognize and bind to various types of molecules, providing a versatile approach for cellular perturbation. Cyclic peptides can exhibit higher binding specificity and affinity for their target proteins compared to small molecules. CPs can be designed or optimized to interact with multiple targets simultaneously or sequentially. CPs can interact with target proteins at allosteric sites, which are distinct from the active site. This mode of interaction can modulate protein function in a more nuanced and potentially reversible manner, offering an alternative approach to direct inhibition or activation of the target protein.

Cyclic peptides can target alternatively spliced transcripts as well as overlapping protein products (as in the case of AAV Rep (replication) protein). This is particularly useful in the case of AAV, where the Rep protein encodes multiple overlapping proteins. This overlapping sequence makes it very difficult to successfully genetically engineer the rep gene sequence to selectively engineer a single protein because each desired gene mutation may have undesired results for the multiple overlapping and alternate reading frame protein products. In contrast, a cyclic peptide, because it modulates its targets post translationally, can potentially selectively target a single protein species from such overlapping, alternative reading frame, or alternatively spliced genetic constructs. This provides an unusual advantage over more traditional, pure genetic engineering-based approaches.

The present teachings include a method of obtaining an engineered cyclic peptide capable of increasing viral titer and/or transduction efficiency of a viral vector composition, the method comprising:
(a) culturing a first plurality of host cells permissive for replication of a virus under conditions suitable for recombinant viral production, wherein each host cell of the first plurality of host cells comprises:
(i) at least one viral replication gene essential for the replication of the virus;
(ii) at least one viral structural gene essential for formation of viral capsids;
(iii) at least one additional viral gene necessary for the production of the virus in the host cells; and
(iv) an engineered cyclic peptide produced from a polypeptide encoded by a first nucleotide sequence, wherein (v) the first nucleotide sequence is operably linked to one or more viral-specific packaging sequences necessary for encapsulation of the first nucleotide sequence within the viral capsids or (vi) the first nucleotide sequence is associated with a second nucleotide sequence comprising a barcode that comprises identifying information regarding the engineered cyclic peptide produced in the host cell, and the second nucleotide sequence is operably linked to the one or more viral-specific packaging sequences necessary for encapsulation of the second nucleotide sequence within the viral capsids, thereby obtaining a first plurality of viral vectors comprising the first nucleotide sequence and/or the second nucleotide sequence from the first plurality of host cells;
(b) optionally, repeating the following steps one or more times in cycles:
(b1) allowing a plurality of viral vectors of the previous cycle to infect a plurality of host cells of the present cycle permissive for replication of the virus; and
(b2) culturing the plurality of host cells of the present cycle under conditions suitable for recombinant viral production, wherein each host cell of the plurality of host cells of the present cycle comprises the elements (i)-(iii) of the first plurality of host cells, and further comprises the first nucleotide sequence operably linked to the one or more viral-specific packaging sequences producing the engineered cyclic peptide, thereby obtaining a plurality of viral vectors of the present cycle comprising the first nucleotide sequence;
(c) allowing the first plurality of viral vectors or the plurality of viral vectors of the present cycle to infect a final plurality of host cells; and (d) determining one or more engineered cyclic peptides capable of increasing viral titer and/or transduction efficiency of the viral vector composition by analyzing nucleotide sequences operably linked to the one or more viral-specific packaging sequences from (i) the final plurality of host cells and/or (ii) a final plurality of viral vectors produced in the final plurality of host cells.

In some embodiments of the disclosed method, in step (a), the first nucleotide sequence is operably linked to the one or more viral-specific packaging sequences, and the method comprises culturing the final plurality of host cells under conditions suitable for recombinant viral production, wherein each host cell of the final plurality of host cells comprises the elements (i)-(iii) of the first plurality of host cells, and further comprises the first nucleotide sequence operably linked to one or more viral-specific packaging sequences and producing the engineered cyclic peptide encoded by the first nucleotide sequence, thereby producing the final plurality of viral vectors from the final plurality of host cells and determining the engineered cyclic peptide by analyzing nucleotide sequences operably linked to the one or more viral-specific packaging sequences from the final plurality of viral vectors.

In some preferred embodiments of the disclosed method, the viral vector composition is an adeno-associated virus (AAV) vector composition; the at least one viral replication gene comprises at least one AAV replication gene; the at least one viral structural gene comprises at least one AAV capsid encoding gene; the at least one additional viral gene comprises at least one AAV helper gene; and the one or more viral-specific packaging sequences comprise at least two functional AAV inverted terminal repeats (ITRs). In other preferred embodiments of the disclosed method, the viral vector composition is a lentivirus vector composition; the at least one viral replication gene comprises at least one lentiviral pol gene; the at least one viral structural gene comprises at least one lentiviral gag gene and at least one env gene; the at least one additional viral gene comprises at least one lentiviral rev gene; and the one or more viral-specific packaging sequences comprise a Psi sequence.

In some embodiments of the disclosed method, the first nucleotide sequence operably linked to the one or more viral-specific packaging sequences further encodes a reporter, a therapeutic payload or a selectable marker. In some embodiments, the disclosed method further comprises step (e): generating new viral vectors in the presence of an engineered cyclic peptide obtained in step (d), thereby producing the viral vector composition of increased viral titer and/or transduction efficiency. In some embodiments, the disclosed method produces the viral vector composition having a characteristic, which is at least 2-fold higher than a corresponding characteristic of a reference viral vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from the group consisting of viral titer and transduction efficiency.

The present teachings also include a plurality of host cells permissive for replication of a virus, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary for the production of the virus in the host cells; and
(iv) a nucleotide sequence operably linked to one or more viral-specific packaging sequences necessary for encapsulation of the nucleotide sequence within the viral capsids, wherein the nucleotide sequence encodes a payload; wherein the engineered cyclic peptide increases a characteristic of viral vectors produced by the plurality of host cells by at least 2-fold compared to a corresponding characteristic of viral vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of viral vectors is selected from the group consisting of viral titer and transduction efficiency.

In some preferred embodiments of the disclosed method, the virus is an adeno-associated virus (AAV); the at least one viral replication gene comprises at least one AAV replication gene; the at least one viral structural gene comprises at least one AAV capsid encoding gene; the at least one additional viral gene comprises at least one AAV helper gene; and the one or more viral-specific packaging sequences comprise at least two functional AAV inverted terminal repeats (ITRs). In other preferred embodiments of the disclosed method, the virus is a lentivirus; the at least one viral replication gene comprises at least one lentiviral pol gene; the at least one viral structural gene comprises at least one lentiviral gag gene and at least one env gene; the at least one additional viral gene comprises at least one lentiviral rev gene; and the one or more viral-specific packaging sequences comprise a Psi sequence.

In some embodiments, the plurality of host cells comprises at least 10,000 host cells.

The present teachings also include a method of producing a viral vector composition of increased viral titer and/or transduction efficiency, the method comprising:
(a) culturing a plurality of host cells permissive for replication of a virus under conditions suitable for recombinant viral production, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary to produce the virus in the host cells; and (iv) a nucleotide sequence operably linked to one or more viral-specific packaging sequences necessary for encapsulation of the nucleotide sequence within the viral capsids, wherein the nucleotide sequence encodes a payload; and
(b) producing the viral vector composition of increased viral titer and/or transduction efficiency from the plurality of host cells, wherein the viral vector composition has an increased viral titer and/or transduction efficiency which is at least a 20%, 40%, 60%, 80%, 100%, 200%, or 500% greater than a viral titer and/or transduction efficiency of a reference viral vector composition produced in a plurality of reference host cells under essentially identical conditions, wherein each reference host cell of the plurality of reference host cells comprises the elements (i)-(iv) of the plurality of host cells and does not comprise the engineered cyclic peptide.

In some preferred embodiments of the disclosed method, the viral vector composition is an adeno-associated virus (AAV) vector composition; the at least one viral replication gene comprises at least one AAV replication gene; the at least one viral structural gene comprises at least one AAV capsid encoding gene; the at least one additional viral gene comprises at least one AAV helper gene; and the one or more viral-specific packaging sequences comprise at least two functional AAV inverted terminal repeats (ITRs). In other preferred embodiments of the disclosed method, the viral vector composition is a lentivirus vector composition; the at least one viral replication gene comprises at least one lentiviral pol gene; the at least one viral structural gene comprises at least one lentiviral gag gene and at least one env gene; the at least one additional viral gene comprises at least one lentiviral rev gene; and the one or more viral-specific packaging sequences comprise a Psi sequence.

Disclosed herein is also a method of obtaining an engineered cyclic peptide capable of increasing viral titer and/or transduction efficiency of adeno-associated virus (AAV) vector composition, the method comprising:
(a) culturing a first plurality of host cells permissive for AAV replication under conditions suitable for recombinant AAV production, wherein each host cell of the first plurality of host cells comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) an engineered cyclic peptide produced from a polypeptide encoded by a first nucleotide sequence, wherein (v) the first nucleotide sequence is operably linked to at least two functional AAV inverted terminal repeats (ITRs) or (vi) the first nucleotide sequence is associated with a second nucleotide sequence comprising a barcode that comprises identifying information regarding the engineered cyclic peptide produced in the host cell, and the second nucleotide sequence is operably linked to at least two functional AAV ITRs, thereby obtaining a first plurality of AAV vectors comprising the first nucleotide sequence and/or the second nucleotide sequence from the first plurality of host cells;
(b) optionally, repeating the following steps one or more times in cycles:
(b1) allowing a plurality of AAV vectors of the previous cycle to infect a plurality of host cells of the present cycle permissive for AAV replication; and
(b2) culturing the plurality of host cells of the present cycle under conditions suitable for recombinant AAV production, wherein each host cell of the plurality of host cells of the present cycle comprises the elements (i)-(iii) of the first plurality of host cells, and further comprises the first nucleotide sequence operably linked to the at least two functional AAV ITRs producing the engineered cyclic peptide, thereby obtaining a plurality of AAV vectors of the present cycle comprising the first nucleotide sequence;
(c) allowing the first plurality of AAV vectors or the plurality of AAV vectors of the present cycle to infect a final plurality of host cells; and (d) determining one or more engineered cyclic peptides capable of increasing viral titer and/or transduction efficiency of AAV vector composition by analyzing nucleotide sequences operably linked to the at least two functional AAV inverted terminal repeats (ITRs) from (i) the final plurality of host cells and/or (ii) a final plurality of AAV vectors produced in the final plurality of host cells.

Disclosed herein is also a method of producing an adeno-associated virus (AAV) vector composition, the method comprising:
(a) culturing a first plurality of host cells permissive for AAV replication under conditions suitable for recombinant AAV production, wherein each host cell of the first plurality of host cells comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) an engineered cyclic peptide produced from a polypeptide encoded by a first nucleotide sequence, wherein (v) the first nucleotide sequence is operably linked to at least two functional AAV inverted terminal repeats (ITRs) or (vi) the first nucleotide sequence is associated with a second nucleotide sequence comprising a barcode that comprises identifying information regarding the engineered cyclic peptide produced in the host cell, and the second nucleotide sequence is operably linked to at least two functional AAV inverted terminal repeats (ITRs), thereby obtaining a first plurality of AAV vectors comprising the first nucleotide sequence and/or the second nucleotide sequence from the first plurality of host cells;
(b) optionally, repeating the following steps one or more times in cycles:
(b1) allowing a plurality of AAV vectors of the previous cycle to infect a plurality of host cells of the present cycle permissive for AAV replication; and
(b2) culturing the plurality of host cells of the present cycle under conditions suitable for recombinant AAV production, wherein each host cell of the plurality of host cells of the present cycle comprises the elements (i)-(iii) of the first plurality of host cells, and further comprises the first nucleotide sequence operably linked to the at least two functional AAV ITRs producing the engineered cyclic peptide, thereby obtaining a plurality of AAV vectors of the present cycle comprising the first nucleotide sequence;
(c) allowing the first plurality of AAV vectors or the plurality of AAV vectors of the present cycle to infect a final plurality of host cells;
(d) determining one or more engineered cyclic peptides capable of increasing viral titer and/or transduction efficiency of AAV vector composition by analyzing nucleotide sequences operably linked to the at least two functional AAV inverted terminal repeats (ITRs) from (i) the final plurality of host cells and/or (ii) a final plurality of AAV vectors produced in the final plurality of host cells; and (e) obtaining new AAV vectors in the presence of an engineered cyclic peptide determined in step (d), thereby producing the AAV vector composition. In some preferred embodiments, the disclosed AAV vector composition is of increased viral titer and/or transduction efficiency compared to a reference AAV vector composition produced in a plurality of reference host cells under essentially identical conditions, wherein each reference host cell of the plurality of reference host cells comprises the elements (i)-(iii) of the plurality of host cells and does not comprise the engineered cyclic peptide. In some preferred embodiments of this method, it produces the AAV vector composition having a characteristic, which is at least 2-fold higher than a corresponding characteristic of a reference AAV vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from viral titer and transduction efficiency. In some embodiments of this method, the engineered cyclic peptide is not essentially present in the produced AAV vector composition (e.g., the engineered cyclic peptide is present in host cells, but is not transferred to the produced AAV vector composition).

Disclosed herein is also a plurality of host cells permissive for AAV replication, wherein each host cell of the plurality of host cells comprises an engineered cyclic peptide and further comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and
(iv) a nucleotide sequence operably linked to at least two functional AAV internal terminal repeats (ITRs), wherein the nucleotide sequence encodes a payload (e.g. therapeutic gene); wherein the engineered cyclic peptide increases a characteristic of AAV vectors produced by the plurality of host cells by at least 2-fold compared to a corresponding characteristic of AAV vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of AAV vectors is selected from viral titer and transduction efficiency.

Disclosed herein is also a method of producing an adeno-associated virus (AAV) vector composition of increased viral titer and/or transduction efficiency, the method comprising: culturing a plurality of host cells permissive for AAV replication under conditions suitable for recombinant AAV production, wherein each host cell of the plurality of host cells comprises an engineered cyclic peptide and further comprises:
(i) at least one AAV replication protein produced from at least one AAV replication gene;
(ii) at least one AAV capsid encoding protein produced from at least one AAV capsid encoding gene;
(iii) at least one AAV helper protein produced from at least one AAV helper gene; and (iv) a nucleotide sequence operably linked between two functional AAV internal terminal repeats (ITRs), wherein the nucleotide sequence encodes a payload; and (b) producing the AAV vector composition of increased viral titer and/or transduction efficiency from the plurality of host cells, wherein the AAV vector composition has an increased viral titer and/or transduction efficiency which is at least a 20%, 40%, 60%, 80%, 100%, 200%, or 500% greater than an viral titer and/or transduction efficiency of a reference AAV vector composition produced from a plurality of reference host cells under essentially identical conditions, wherein each reference host cell of the plurality of reference host cells comprises the elements (i)-(iv) of the plurality of host cells and does not comprise the engineered cyclic peptide.

The present teachings also include a method of obtaining an engineered cyclic peptide capable of increasing viral titer and/or transduction efficiency of lentivirus vector composition, the method comprising:
(a) culturing a first plurality of host cells permissive for lentivirus replication under conditions suitable for recombinant lentivirus production, wherein each host cell of the first plurality of host cells comprises:
(i) at least one lentiviral gag gene;
(ii) at least one lentiviral pol gene;
(iii) at least one lentiviral rev gene;
(iv) at least one env gene; and
(v) an engineered cyclic peptide produced from a polypeptide encoded by a first nucleotide sequence, wherein (vi) the first nucleotide sequence is operably linked to a Psi sequence or (vii) the first nucleotide sequence is associated with a second nucleotide sequence comprising a barcode that comprises identifying information regarding the engineered cyclic peptide produced in the host cell, and the second nucleotide sequence is operably linked to a Psi sequence, thereby obtaining a first plurality of lentivirus vectors comprising the first nucleotide sequence and/or the second nucleotide sequence from the first plurality of host cells;
(b) optionally, repeating the following steps one or more times in cycles:
(b1) allowing a plurality of lentivirus vectors of the previous cycle to infect a plurality of host cells of the present cycle permissive for lentivirus replication; and
(b2) culturing the plurality of host cells of the present cycle under conditions suitable for recombinant lentivirus production, wherein each host cell of the plurality of host cells of the present cycle comprises the elements (i)-(iv) of the first plurality of host cells, and further comprises the first nucleotide sequence operably linked to the Psi sequence producing the engineered cyclic peptide, thereby obtaining a plurality of lentivirus vectors of the present cycle comprising the first nucleotide sequence;
(c) allowing the first plurality of lentivirus vectors or the plurality of lentivirus vectors of the present cycle to infect a final plurality of host cells; and (d) determining one or more engineered cyclic peptides capable of increasing viral titer and/or transduction efficiency of lentivirus vector composition by analyzing nucleotide sequences operably linked to the Psi sequence from (i) the final plurality of host cells and/or (ii) a final plurality of lentivirus vectors produced in the final plurality of host cells.

In some embodiments of the disclosed method, in step (a), the first nucleotide sequence is operably linked to the Psi sequence, and the method comprises culturing the final plurality of host cells under conditions suitable for recombinant lentivirus production, wherein each host cell of the final plurality of host cells comprises the elements (i)-(iv) of the first plurality of host cells, and further comprises the first nucleotide sequence operably linked to the Psi sequence and producing the engineered cyclic peptide encoded by the first nucleotide sequence, thereby producing the final plurality of lentivirus vectors from the final plurality of host cells and determining the engineered cyclic peptide by analyzing nucleotide sequences operably linked to the Psi sequence from the final plurality of lentivirus vectors. In some embodiments of the disclosed method, the first nucleotide sequence operably linked to the Psi sequence further encodes a reporter, a therapeutic payload or a selectable marker.

In some embodiments, the disclosed method further comprises step (e): generating new lentivirus vectors in the presence of an engineered cyclic peptide obtained in step (d), thereby producing the lentivirus vector composition of increased viral titer and/or transduction efficiency.

In some embodiments, the disclosed method produces the lentivirus vector composition having a characteristic, which is at least 2-fold higher than a corresponding characteristic of a reference lentivirus vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from the group consisting of viral titer and transduction efficiency.

In some embodiments, the engineered cyclic peptide is not essentially present in the lentivirus vector composition of increased viral titer and/or transduction efficiency.

The present teachings also include a plurality of host cells permissive for lentivirus replication, wherein each host cell of the plurality of host cells comprises an engineered cyclic peptide and further comprises:
(i) at least one lentiviral gag gene;
(ii) at least one lentiviral pol gene;
(iii) at least one lentiviral rev gene;
(iv) at least one env gene; and
(v) a nucleotide sequence operably linked to a Psi sequence, wherein the nucleotide sequence encodes a payload; wherein the engineered cyclic peptide increases a characteristic of lentivirus vectors produced by the plurality of host cells by at least 2-fold compared to a corresponding characteristic of lentivirus vectors produced by a plurality of reference host cells under essentially identical conditions, wherein each reference host cell of the plurality of reference host cells comprises the elements (i)-(iv) of the plurality of host cells and does not comprise the engineered cyclic peptide, and wherein the characteristic of lentivirus vectors is selected from the group consisting of viral titer and transduction efficiency.

In some embodiments, the plurality of host cells comprises at least 10,000 host cells.

The present teachings also include a method of producing a lentivirus vector composition of increased viral titer and/or transduction efficiency, the method comprising: (a) culturing a plurality of host cells permissive for lentiviral replication under conditions suitable for recombinant lentiviral production, wherein each host cell of the plurality of host cells comprises an engineered cyclic peptide and further comprises:
(i) at least one lentiviral gag gene;
(ii) at least one lentiviral pol gene;
(iii) at least one lentiviral rev gene;
(iv) at least one env gene; and (v) a nucleotide sequence operably linked to a Psi sequence, wherein the nucleotide sequence encodes a payload (e.g. therapeutic gene); and (b) producing the lentivirus vector composition of increased viral titer and/or transduction efficiency from the plurality of host cells, wherein the lentivirus vector composition has an increased viral titer and/or transduction efficiency which is at least a 20%, 40%, 60%, 80%, 100%, 200%, or 500% greater than a viral titer and/or transduction efficiency of a reference lentivirus vector composition produced in a plurality of reference host cells under essentially identical conditions, wherein each reference host cell of the plurality of reference host cells comprises the elements (i)-(v) of the plurality of host cells and does not comprise the engineered cyclic peptide.

The present teachings also include a method of obtaining an engineered cyclic peptide capable of increasing viral titer and/or transduction efficiency of recombinant adenovirus (AdV) vector composition, the method comprising: (a) culturing a first plurality of host cells permissive for AdV replication under conditions suitable for recombinant AdV production, wherein each host cell of the first plurality of host cells comprises:
(i) at least one adenoviral capsid gene selected from the group consisting of hexon, penton base, and fiber;
(ii) at least one adenoviral core protein selected from the group consisting of protein VII and terminal protein produced from their respective genes; (iii) at least one adenoviral early gene product selected from the group consisting of E1A, E1B, E2A, and E2B produced from their respective genes; and
(iv) an engineered cyclic peptide produced from a polypeptide encoded by a first nucleotide sequence, wherein (v) the first nucleotide sequence is operably linked to at least two functional AdV inverted terminal repeats (ITRs) or (vi) the first nucleotide sequence is associated with a second nucleotide sequence comprising a barcode that comprises identifying information regarding the engineered cyclic peptide produced in the host cell, and the second nucleotide sequence is operably linked to at least two functional AdV ITRs, thereby obtaining a first plurality of AdV vectors comprising the first nucleotide sequence and/or the second nucleotide sequence from the first plurality of host cells;
(b) optionally, repeating the following steps one or more times in cycles:
(b1) allowing a plurality of AdV vectors of the previous cycle to infect a plurality of host cells of the present cycle permissive for AdV replication; and
(b2) culturing the plurality of host cells of the present cycle under conditions suitable for recombinant AdV production, wherein each host cell of the plurality of host cells of the present cycle comprises the elements (i)-(iii) of the first plurality of host cells, and further comprises the first nucleotide sequence operably linked to the at least two functional AdV ITRs producing the engineered cyclic peptide, thereby obtaining a plurality of AdV vectors of the present cycle comprising the first nucleotide sequence;
(c) allowing the first plurality of AdV vectors or the plurality of AdV vectors of the present cycle to infect a final plurality of host cells; and (d) determining one or more engineered cyclic peptides capable of increasing viral titer and/or transduction efficiency of AdV vector composition by analyzing nucleotide sequences operably linked to the at least two functional AdV inverted terminal repeats (ITRs) from (i) the final plurality of host cells and/or (ii) a final plurality of AdV vectors produced in the final plurality of host cells.

In some embodiments of the disclosed method, in step (a), the first nucleotide sequence is operably linked to the at least two functional AdV ITRs, and the method comprises culturing the final plurality of host cells under conditions suitable for recombinant AdV production, wherein each host cell of the final plurality of host cells comprises the elements (i)-(iii) of the first plurality of host cells, and further comprises the first nucleotide sequence operably linked to the at least two functional AdV ITRs and producing the engineered cyclic peptide encoded by the first nucleotide sequence, thereby producing the final plurality of AdV vectors from the final plurality of host cells and determining the engineered cyclic peptide by analyzing nucleotide sequences operably linked to the at least two functional AdV ITRs from the final plurality of AdV vectors.

The present teachings also include a plurality of host cells permissive for AdV replication, wherein each host cell of the plurality of host cells comprises an engineered cyclic peptide and further comprises:
(i) at least one adenoviral capsid gene selected from the group consisting of hexon, penton base, and fiber;
(ii) at least one adenoviral core protein selected from the group consisting of protein VII and terminal protein produced from their respective genes; (iii) at least one adenoviral early gene product selected from the group consisting of E1A, E1B, E2A, and E2B produced from their respective genes; and
(iv) a nucleotide sequence operably linked to at least two functional AdV inverted terminal repeats (ITRs), wherein the nucleotide sequence encodes a payload (e.g. therapeutic gene); wherein the engineered cyclic peptide increases a characteristic of AdV vectors produced by the plurality of host cells by at least 2-fold compared to a corresponding characteristic of AdV vectors produced by a plurality of reference host cells under essentially identical conditions, wherein each reference host cell of the plurality of reference host cells comprises the elements (i)-(iv) of the plurality of host cells and does not comprise the engineered cyclic peptide, and wherein the characteristic of AdV vectors is selected from the group consisting of viral titer and transduction efficiency.

The present teachings also include a method of producing a recombinant adenovirus (AdV) vector composition of increased viral titer and/or transduction efficiency, the method comprising:
(a) culturing a plurality of host cells permissive for AdV replication under conditions suitable for AdV production, wherein each host cell of the plurality of host cells comprises an engineered cyclic peptide and further comprises:
(i) at least one adenoviral capsid gene selected from the group consisting of hexon, penton base, and fiber;
(ii) at least one adenoviral core protein selected from the group consisting of protein VII and terminal protein produced from their respective genes;
(iii) at least one adenoviral early gene product selected from the group consisting of E1A, E1B, E2A, and E2B produced from their respective genes; and
(iv) a nucleotide sequence operably linked to at least two functional AdV inverted terminal repeats (ITRs), wherein the nucleotide sequence encodes a payload (e.g. therapeutic gene); and (b) producing the AdV vector composition of increased viral titer and/or transduction efficiency from the plurality of host cells, wherein the AdV vector composition has an increased viral titer and/or transduction efficiency which is at least a 20%, 40%, 60%, 80%, 100%, 200%, or 500% greater than a viral titer and/or transduction efficiency of a reference AdV vector composition produced in a plurality of reference host cells under essentially identical conditions, wherein each reference host cell of the plurality of reference host cells comprises the elements (i)-(iv) of the plurality of host cells and does not comprise the engineered cyclic peptide.

The present teachings also include a method of obtaining an engineered cyclic peptide capable of increasing viral titer and/or transduction efficiency of herpes simplex virus (HSV) vector composition, the method comprising:
(a) culturing a first plurality of host cells permissive for HSV replication under conditions suitable for recombinant HSV production, wherein each host cell of the first plurality of host cells comprises:
(i) at least one HSV capsid protein selected from the group consisting of VP5, VP19C, VP23, and VP21 produced from their respective genes;
(ii) at least one HSV tegument protein selected from the group consisting of VP16, VP19A, and VP22 produced from their respective genes;
(iii) at least one HSV regulatory protein selected from the group consisting of ICP0, ICP4, ICP22, and ICP27 produced from their respective genes; and (iv) an engineered cyclic peptide produced from a polypeptide encoded by a first nucleotide sequence, wherein (v) the first nucleotide sequence is operably linked to at least one of TRL, IRL, IRS, TRS, or Pac sequences for packaging, or (vi) the first nucleotide sequence is associated with a second nucleotide sequence comprising a barcode that comprises identifying information regarding the engineered cyclic peptide produced in the host cell, and the second nucleotide sequence is operably linked to at least one of TRL, IRL, IRS, TRS, or Pac sequences for packaging, thereby obtaining a first plurality of HSV vectors comprising the first nucleotide sequence and/or the second nucleotide sequence from the first plurality of host cells;
(b) optionally, repeating the following steps one or more times in cycles:
(b1) allowing a plurality of HSV vectors of the previous cycle to infect a plurality of host cells of the present cycle permissive for HSV replication; and
(b2) culturing the plurality of host cells of the present cycle under conditions suitable for recombinant HSV production, wherein each host cell of the plurality of host cells of the present cycle comprises the elements (i)-(iii) of the first plurality of host cells, and further comprises the first nucleotide sequence operably linked to at least one of TRL, IRL, IRS, TRS, or Pac sequences for packaging, producing the engineered cyclic peptide, thereby obtaining a plurality of HSV vectors of the present cycle comprising the first nucleotide sequence;
(c) allowing the first plurality of HSV vectors or the plurality of HSV vectors of the present cycle to infect a final plurality of host cells; and (d) determining one or more engineered cyclic peptides capable of increasing viral titer and/or transduction efficiency of HSV vector composition by analyzing nucleotide sequences operably linked to the at least one of TRL, IRL, IRS, TRS, or Pac sequences from (i) the final plurality of host cells and/or (ii) a final plurality of HSV vectors produced in the final plurality of host cells.

In some embodiments of the disclosed method, in step (a), the first nucleotide sequence is operably linked to the at least one of TRL, IRL, IRS, TRS, or Pac sequences, and the method comprises culturing the final plurality of host cells under conditions suitable for recombinant HSV production, wherein each host cell of the final plurality of host cells comprises the elements (i)-(iii) of the first plurality of host cells, and further comprises the first nucleotide sequence operably linked to the at least one of TRL, IRL, IRS, TRS, or Pac sequences, and producing the engineered cyclic peptide encoded by the first nucleotide sequence, thereby producing the final plurality of HSV vectors from the final plurality of host cells and determining the engineered cyclic peptide by analyzing nucleotide sequences operably linked to the at least one of TRL, IRL, IRS, TRS, or Pac sequences from the final plurality of HSV vectors.

The present teachings also include a plurality of host cells permissive for HSV replication, wherein each host cell of the plurality of host cells comprises an engineered cyclic peptide and further comprises:
(i) at least one HSV capsid protein selected from the group consisting of VP5, VP19C, VP23, and VP21 produced from their respective genes;
(ii) at least one HSV tegument protein selected from the group consisting of VP16, VP19A, and VP22 produced from their respective genes;
(iii) at least one HSV regulatory protein selected from the group consisting of ICP0, ICP4, ICP22, and ICP27 produced from their respective genes; and
(iv) a nucleotide sequence operably linked to at least one of TRL, IRL, IRS, TRS, or Pac sequences for packaging, wherein the nucleotide sequence encodes a payload (e.g. therapeutic gene); wherein the engineered cyclic peptide increases a characteristic of HSV vectors produced by the plurality of host cells by at least 2-fold compared to a corresponding characteristic of HSV vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of HSV vectors is selected from the group consisting of viral titer and transduction efficiency.

The present teachings also include a method of producing a herpes simplex virus (HSV) vector composition of increased viral titer and/or transduction efficiency, the method comprising:
(a) culturing a plurality of host cells permissive for HSV replication under conditions suitable for recombinant HSV production, wherein each host cell of the plurality of host cells comprises an engineered cyclic peptide and further comprises:
(i) at least one HSV capsid protein selected from the group consisting of VP5, VP19C, VP23, and VP21 produced from their respective genes;
(ii) at least one HSV tegument protein selected from the group consisting of VP16, VP19A, and VP22 produced from their respective genes;
(iii) at least one HSV regulatory protein selected from the group consisting of ICP0, ICP4, ICP22, and ICP27 produced from their respective genes; and
(iv) a nucleotide sequence operably linked to at least one of TRL, IRL, IRS, TRS, or Pac sequences for packaging, wherein the nucleotide sequence encodes a payload (e.g. therapeutic gene); and (b) producing the HSV vector composition of increased viral titer and/or transduction efficiency from the plurality of host cells, wherein the HSV vector composition has an increased viral titer and/or transduction efficiency which is at least a 20%, 40%, 60%, 80%, 100%, 200%, or 500% greater than a viral titer and/or transduction efficiency of a reference HSV vector composition produced in a plurality of reference host cells under essentially identical conditions, wherein each reference host cell of the plurality of reference host cells comprises the elements (i)-(iv) of the plurality of host cells and does not comprise the engineered cyclic peptide.

Various embodiments apply equally to the aspects provided herein but will for the sake of brevity be recited only once. Thus, various of the following embodiments apply equally to aspects recited above.

Exemplary embodiments of the disclosed methods are illustrated in FIGS. 1-14.

In some embodiments, disclosed herein are cyclic peptides, ranging from 4 to 12 amino acids in length, that can enhance viral vector production. The cyclic peptide may be a homodetic cyclic peptide, in which the ring is composed exclusively of normal peptide bonds between the alpha carboxyl of one residue to the alpha amine of another. Alternatively, the cyclic peptide can be a cyclic isopeptide that contains at least one non-alpha amide linkage, such as a linkage between the side chain of one residue to the alpha carboxyl group of another residue. Further still, the cyclic peptide can be a cyclic depsipeptide, having at least one lactone (ester) linkage in place of one of the amides, or it can be a bicyclic peptide containing a bridging group between two of the side chains.

In particular instances, the cyclic peptide may be cyclized through a disulfide bond between two cysteines. Additionally, the cyclic peptide may be biosynthesized in cells via a two-step process that includes the translation of a linear peptide chain and its subsequent cyclization into a cyclic structure through activities of a protease-like enzyme or other means.

The incorporation of the described cyclic peptide into the viral vector production process can offer a number of advantages. These may include increased vector yield, enhanced vector stability, and improved transduction efficiency into target cells, among others.

In certain embodiments, the cyclic peptide involved in enhancing viral vector production is comprised not only of conventional L-amino acids but can incorporate non-natural amino acids, modified peptide bonds, or non-peptide bonds. Such diversity in the peptide chain broadens the potential scope of the invention and adds versatility in its applications (e.g., enhanced proteolytic stability, altered membrane permeability profiles).

In some embodiments, the cyclic peptide may include peptoids, which are peptide mimics where the side chain is attached to the nitrogen atom of the peptide backbone rather than the alpha carbon. This structural difference results in molecules with similar size and charge distribution to peptides but with unique structural properties, potentially leading to enhanced biological stability and altered binding characteristics.

In other embodiments, the cyclic peptide may incorporate beta-amino acids, resulting in beta-peptides. The addition of an extra carbon in the peptide backbone can offer improved metabolic stability compared to conventional alpha-peptides. This structural variation can contribute to a higher resistance to proteolytic degradation, leading to increased peptide longevity and functional efficacy.

In yet another set of embodiments, the cyclic peptide may act as a peptide secondary structure mimic. These molecules mimic the secondary structure of peptides (e.g., alpha-helices, beta-sheets) without necessarily having the same primary structure. This property can enhance specific interactions with biological targets, thereby potentially improving the efficiency of vector production.

In some instances, the cyclic peptide may include non-natural amino acids. These can encompass D-amino acids, amino acids with modified side chains, or entirely synthetic amino acids. Such modifications can enhance peptide stability, improve target binding specificity, or introduce novel functionality into the peptide sequence.

Furthermore, in certain embodiments, the cyclic peptide may include non-amide bonds. Examples of alternative types of bonds that can be used to link amino acids or amino acid mimics include ester bonds, triazole bonds formed in click chemistry reactions, or disulfide bonds. These variations in bonding can enhance the chemical diversity of the peptide, potentially introducing unique physical or chemical properties that further improve vector production.

In some embodiments, cyclic peptides may be designed to self-assemble into higher order structures. Such self-assembling cyclic peptides can form unique geometries like nanotubes, nanofibers, or other three-dimensional structures. These self-assembled structures can provide an advanced scaffold for packaging, offering a more efficient spatial arrangement of components, enhancing viral assembly and potentially improving the yield and stability of vectors.

In other embodiments, cyclic peptides can be designed to interact specifically with components of the vector or target cells. These interactions can involve specific binding to proteins such as gag, pol, or env, leading to improved vector stability or enhanced packaging efficiency. Alternatively, the cyclic peptides can interact with specific cell surface proteins on target cells, aiding in the targeting and transduction of specific cell types, and potentially improving vector entry and the efficiency of gene delivery.

In further embodiments, cyclic peptides can be incorporated into a delivery system such as nanoparticles or liposomes. These peptides can be either conjugated to the surface or encapsulated within these delivery vehicles. In the case of surface conjugation, cyclic peptides can provide targeting capabilities, guiding the delivery vehicle to specific cell types, or modulating its interaction with cell membranes to facilitate uptake. If encapsulated, the cyclic peptide can potentially be protected from degradation, extending its half-life and improving its availability for enhancing packaging.

Additionally, in some embodiments, the delivery system can also include other components, such as imaging agents for tracking the delivery and distribution of the cyclic peptide-enhanced vectors, or therapeutic agents that can be co-delivered with the viral vectors for synergistic therapies.

In some embodiments of the disclosed methods and compositions, the engineered cyclic peptide is produced ribosomally in each host cell of the plurality of host cells.

In some embodiments of the disclosed methods and compositions, the engineered cyclic peptide is exogenously supplied to each host cell of the plurality of host cells.

In some embodiments of the disclosed methods and compositions, each host cell of the plurality of host cells is a mammalian host cell.

In some embodiments of the disclosed methods and compositions, each host cell of the plurality of host cells is an insect host cell.

In some embodiments of the disclosed methods and compositions, the payload comprises a therapeutic gene.

In some embodiments of the disclosed methods and compositions, the plurality of host cells comprises at least 10,000 host cells.

In some embodiments of the disclosed methods and compositions, the engineered cyclic peptide is not essentially present in the viral vector composition of increased viral titer and/or transduction efficiency.

In some embodiments of the disclosed methods and compositions, each host cell of a plurality of host cells comprises: (i) at least one viral replication protein essential for the replication of the virus produced from at least one corresponding viral replication gene; (ii) at least one viral structural protein essential for formation of viral capsids produced from at least one corresponding viral structural gene; and (iii) at least one additional viral protein necessary for the production of the virus in the host cells, produced from at least one corresponding viral gene.

In some embodiments of the disclosed methods and compositions, each host cell of a plurality of host cells comprises: (i) at least one AAV replication protein produced from at least one corresponding AAV replication gene; (ii) at least one AAV capsid encoding protein produced from at least one corresponding AAV capsid encoding gene; and (iii) at least one AAV helper protein produced from at least one corresponding AAV helper gene.

In some embodiments of the disclosed methods and compositions, each host cell of a plurality of host cells comprises: (i) at least one lentiviral gag protein produced from at least one corresponding lentiviral gag gene; (ii) at least one lentiviral pol protein produced from at least one corresponding lentiviral pol gene; (iii) at least one lentiviral rev protein produced from at least one corresponding lentiviral rev gene; and (iv) at least one lentiviral env protein produced from at least one corresponding lentiviral env gene. In some embodiments, the at least one env gene is an envelope gene encoding a glycoprotein from an enveloped virus. In one particular embodiment, the at least one env gene encodes the protein comprising amino acid sequence set forth in SEQ ID NO:27.

In some embodiments of the disclosed methods and compositions, each host cell of a plurality of host cells comprises: (i) at least one adenoviral capsid protein selected from the group consisting of hexon, penton base, and fiber produced from their respective genes; (ii) at least one adenoviral core protein selected from the group consisting of protein VII and terminal protein produced from their respective genes; and (iii) at least one adenoviral early gene product selected from the group consisting of E1A, E1B, E2A, and E2B produced from their respective genes.

In some embodiments of the disclosed methods and compositions, each host cell of a plurality of host cells comprises: (i) at least one HSV capsid protein selected from the group consisting of VP5, VP19C, VP23, and VP21 produced from their respective genes; (ii) at least one HSV tegument protein selected from the group consisting of VP16, VP19A, and VP22 produced from their respective genes; and (iii) at least one HSV regulatory protein selected from the group consisting of ICP0, ICP4, ICP22, and ICP27 produced from their respective genes.

In some embodiments, the engineered cyclic peptide comprises an amino acid sequence having at least 90% or more sequence identity to any one of the amino acid sequences selected from the group consisting of SEQ ID NO: 34—SEQ ID NO: 62, SEQ ID NO: 64—SEQ ID NO: 228, and SEQ ID NO: 230.

In some particular embodiments, the AAV replication gene present in each host cell of the first plurality of host cells encodes a protein that comprises an amino acid sequence having at least 90% or more sequence identity to any one of the amino acid sequences selected from the group consisting of SEQ ID NO: 1—SEQ ID NO: 4, SEQ ID NO: 10, SEQ ID NO: 12 and SEQ ID NO: 14.

In some particular embodiments, the AAV capsid encoding gene present in each host cell of the first plurality of host cells encodes a protein that comprises an amino acid sequence having at least 90% or more sequence identity to any one of the amino acid sequences selected from the group consisting of SEQ ID NO: 5—SEQ ID NO: 7, SEQ ID NO: 11, SEQ ID NO: 13 and SEQ ID NO: 15.

In some particular embodiments, the AAV helper gene present in each host cell of the first plurality of host cells encodes a protein that comprises an amino acid sequence having at least 90% or more sequence identity to any one of the amino acid sequences selected from the group consisting of SEQ ID NO: 16—SEQ ID NO: 23 and SEQ ID NO: 30—SEQ ID NO: 33.

In some embodiments, after culturing a plurality of host cells permissive for AAV replication under conditions suitable for recombinant AAV production, the AAV vector composition of increased viral titer and/or transduction efficiency is produced from the plurality of host cells by methods known in the art. In preferred embodiments, the plurality of host cells permissive for AAV replication is cultured in vitro in a liquid culture medium such that host cells of the plurality of host cells produce AAV viral particles, which then are harvested from the culture medium. In some embodiments, producing AAV vector composition of increased viral titer and/or transduction efficiency from the plurality of host cells comprises purifying the AAV viral particles from the culture medium. In some embodiments, harvested AAV viral particles comprise heterologous nucleic acid(s) encoding one or more heterologous gene products. In some embodiments, heterologous gene products comprise a polynucleotide or a polypeptide. In some embodiments, the harvested AAV viral particles are purified. In some embodiments, the AAV viral particles are purified to at least 80%, at least 90%, at least 95%, at least 98%, at least 99%, or greater than 99%, purity. Suitable liquid culture media include any culture medium that provides for growth and/or viability of a mammalian cell (if host cells are mammalian cells) or a insect cell (if host cells are insect cells) in in vitro culture.

In some embodiments, the AAV vector composition of increased viral titer and/or transduction efficiency has an infectious unit titer which is at least a 20%, 40%, 60%, 80%, 100%, 200%, or 500% greater than an infectious unit titer of a reference AAV vector composition produced without engineered cyclic peptides.

In some embodiments, the harvesting comprise harvesting the first, second, third, or higher order plurality of AAV capsids and infecting a murine or primate animal with the first, second, third, or higher order plurality of AAV capsids, wherein the most highly infectious AAV vectors of the AAV vector composition efficiently deliver the nucleotide sequence positioned between two ITRs to animals cells, while the least infectious AAV vectors of the AAV vector composition fail to deliver or inefficiently deliver the nucleotide sequence positioned between two ITRs to animals cells.

Exemplary host cells suitable for the methods and the compositions provided herein include, without limitation, the following cell lines. (1) HEK293 cells and derivatives or related strains. (2) PER.C6 cells; PER.C6 cells are a human retinal pigment epithelial cell line that are also commonly used for AAV production. They are easy to grow and maintain, and they have high transfection efficiency. (3) BTI-TN-5B1-4 cells (High Five cells). These cells are derived from Trichoplusia ni (cabbage looper) ovarian cells and can also be used in the baculovirus expression vector system. High Five cells can grow to high densities in suspension culture and are known to produce high yields of recombinant proteins. (4) Sf9 insect cells. (5) Viro9 cells. (6) CHO cells and derivatives or related strains. (7) BHK cells; Baby hamster kidney (BHK) cells have also been used for AAV production, particularly for some AAV serotypes that may not be efficiently produced in other cell lines. BHK cells can be transfected with plasmids encoding the AAV vector genome, rep and cap genes, and helper functions from adenoviruses.

In some embodiments, the viral vectors produced by the plurality of host cells are viral vectors of a non-enveloped virus. In other embodiments, the viral vectors produced by the plurality of host cells are viral vectors of a enveloped virus.

Culture of the viral vector composition-producing host cell can be performed under known culture conditions. For example, the host cell is cultured at a temperature of 30 to 370° C., a humidity of 95%, and a CO2 concentration of 5 to 10% (v/v). However, culture conditions of the viral vector composition-producing host cell are not limited to the above-mentioned culture conditions. The cell culture may be performed at a temperature, a humidity, and a CO2 concentration out of the above-mentioned ranges, as long as desired cell growth and production of the viral vector composition are accomplished. A culture period is not particularly limited, and for example, the cell culture is continued for 12 to 150 hours, preferably 48 to 120 hours.
In some embodiments, the plurality of host cells comprising elements necessary for production of the viral vector composition also comprise a nucleotide sequence operably linked to one or more viral-specific packaging sequences necessary for encapsulation of the nucleotide sequence within the viral capsids, wherein the nucleotide sequence encodes a payload. In some embodiments, the payload comprises a reporter, a therapeutic moiety (e.g., protein, RNA, DNA, enzyme, growth factor, cytokine, receptor, etc.) or a selectable marker. To control expression of the payload, a suitable promoter, enhancer, terminator, and/or other transcriptional regulatory elements may be inserted into the nucleic acid encoding the payload. In some particular embodiments, the payload comprises a therapeutic payload (e.g., therapeutic gene). Non limiting examples of FDA-approved therapeutic payloads include: 1) human retinoid isomerohydrolase RPE65 (Serotype: AAV2); human survival motor neuron protein (Serotype: AAV9); and hFIX-Padua (Coagulation factor) (Serotype: AAV5). Multiple examples of viral vector compositions exist in clinical trials (see, e.g., Clinical Trials Database).

In some embodiments, TU:VG ratio of the AAV vector composition of increased viral titer and/or transduction efficiency is from 1:100 to 1:50, from 1:50 to 1:20, from 1:20 to 1:10, from 1:10 to 1:5, from 1:5 to 1:2, or from 1:2 to 1:1.

In some embodiments, the AAV vector composition of increased viral titer and/or transduction efficiency has a viral genome titer which is at least a 20%, 40%, 60%, 80%, 100%, 200%, or 500% greater than a viral genome titer of a reference AAV vector composition produced without engineered cyclic peptides.

In some embodiments, the first nucleotide sequence encodes a gene product configured to undergo a cyclization (and optionally a post-translational modification) resulting in an engineered cyclic peptide (CP). In other embodiments, CP can be supplied exogenously or produced endogenously, and the first nucleotide sequence comprises a barcode that identifies the CP.

In some embodiments, the above-described method produces AAV composition having a titer of 20 IU/cell or more, wherein a reference titer of infectious units produced without the CP is 10 IU/cell (at least 2-fold increase in IU/cell).

It should be noted that different AAV serotypes have different viral titers. Additionally, different packaging strategies have different viral titers.

The method works by applying competitive genetic selection on the functional attributes of rAAV vectors (e.g. infectiousness). Said rAAV vectors were assembled/packaged in a cell comprising a genetically encoded, ribosomally synthesized, post-translationally modified cyclic peptide. Said rAAV harbors DNA encoding a cyclic peptide (or indicating via barcode) that is/was present during assembly/packaging of the rAAV. The peptide-induced modulation of the host cell's packaging environment alters the rAAV assembly in ways that dramatically alter yield, infectiousness and other rAAV attributes. The CP-mediated enhancements of rAAV packaging is easily coupled to the propagation of DNA molecules that encode the endogenously expressed cyclic peptide (or identifying DNA barcodes). After AAV packaging, a viral capsid contains DNA encoding/identifying a peptide library element. Different library members are expected to possess different functional attributes as a result of their having been assembled in the presence of the cyclic peptide encoded by the DNA that they harbor. These rAAVs are used to transfect naive cells. The transfer of DNAs encoding/identifying peptide library elements to naive cells is linked to the quality/infectivity of the AAV capsid, which may be positively, negatively, or neutrally impacted by the presence of the cyclic peptide during rAAV packaging. Cyclic peptides that reduce rAAV packaging, yield, infectivity, or other attributes that would facilitate their propagation in a competitive enrichment assay are rapidly depleted from the library. On the other hand, cyclic peptides that confer large improvements to rAAV packaging, yield, infectivity, or other attributes that would facilitate their propagation in a competitive enrichment assay are strongly selected for and rAAVs harboring DNAs that encode or identify these cyclic peptides rapidly increase their population. In this way, we can exploit the functional attributes of rAAVs to enrich for cyclic peptides that increase the manufacturability of rAAVs. The DNA molecules can be recovered after transfection for analysis and identification of the peptide library elements. The approach can be applied to combinations of cyclic peptides as well in order to identify combinations of cyclic peptides that synergize to further enhance the yield and quality of rAAV.

The value of this method is a large throughput advantage as an entire peptide library is selected for the ability of the library elements to positively modulate/enhance AAV manufacturability. In contrast, library elements that negatively modulate AAV manufacturability or cell viability are immediately depleted from the library and/or out-competed by performance-enhancing CPs. The method is intracellular; the cyclic peptide library is expressed endogenously. This increases the local concentration bioavailability of the peptide library elements, eliminates the need for membrane permeability of the cyclic peptides, and obviates the need for exogenous synthesis.

The present disclosure also provides platform technologies for optimized production of AAV vectors as well as methods for identifying said optimizations. The present disclosure provides methods that use a library-based approaches for identifying cyclic peptides, that when added to or produced by host cells during AAV production, increase manufacturability of AAVs. The present disclosure provides a novel discovery platform technology, where DNA that encodes cyclic peptides is expressed by host cells that are simultaneously producing AAVs, wherein DNA encoding said CP (or a related barcode) can be packaged into AAV capsids. The effect of the CP on AAV production results in the DNA encoding said CP (or a related barcode) being packaged more/less efficiently or into more/less infectious AAV particles. Viral vector libraries (containing CPs or barcodes) produced from mammalian cells can be analyzed as a pool by NGS in order to understand the functional properties conferred upon the AAV vector by the presence of the CP during the viral packaging in the mammalian cell. Desired characteristics include but are not limited to enhanced or improved viral vector production, infectiousness, empty:full capsid ratio, gene expression.

In some embodiments, an AAV vector is therapeutically active. However, in some embodiments, provided methods may yield non-functional AAV vectors that lack one or more functional characteristics, but retain other characteristics of interest. In some embodiments, an AAV vector is non-functional or has reduced function for a particular characteristic. For example, in some embodiments an AAV vector may have a reduced ability to transfer a payload or may not be able to transfer a payload. In some embodiments, an AAV vector may have reduced ability to kill cancer cells. In some embodiments, an AAV vector may be therapeutically inactive.

In some embodiments of the disclosed method, the first nucleotide sequence is operably linked to at least one functional AAV inverted terminal repeat (ITR) as disclosed in Samulski R J, Berns K I, Tan M, Muzyczka N. Cloning of adeno-associated virus into pBR322: rescue of intact virus from the recombinant plasmid in human cells. Proc Natl Acad Sci USA. 1982 March; 79(6):2077-81. In other embodiments of the disclosed method, the first nucleotide sequence is operably linked to two or three functional AAV inverted terminal repeats (ITRs). In some embodiments, two functional AAV ITR sequences comprise human AAV1 ITRs, human AAV2 ITRs, human AAV3b ITRs, human AAV4 ITRs, human AAV5 ITRs, human AAV6 ITRs, human AAV7 ITRs, human AAV8 ITRs, human AAV9 ITRs, human AAV 10 ITRs, human AAV 11 ITRs, human AAV 12 ITRs, or human AAV13 ITRs. In other embodiments, two functional AAV ITR sequences comprise bovine AAV (b-AAV) ITRs, canine AAV (CAAV) ITRs, mouse AAV1 ITRs, caprine AAV ITRs, rat AAV ITRs, or avian AAV (AAAV) ITRs

In some embodiments, the first nucleotide sequence may encode a cyclic peptide, a barcode that identifies the cyclic peptide, or both cyclic peptide and barcode.

In some embodiments of the disclosed method, the nucleotide sequence positioned between two inverted terminal repeats (ITRs) comprise a sequence that encodes a polypeptide configured to produce a cyclic peptide during rAAV production in the host cell. In other embodiments of the disclosed method, the nucleotide sequence positioned between two inverted terminal repeats (ITRs) comprise a barcode sequence that comprises identifying information regarding the cyclic peptide produced in the host cell, while a sequence that encodes a polypeptide configured to produce a cyclic peptide is present outside two ITRs. The latter embodiments still allow for selection of AAV-enhancing cyclic peptides, since after an infection round, enriched AAV vectors may be purified, and corresponding barcode sequences can be isolated and sequenced, thus decoding AAV-enhancing cyclic peptides.

In some embodiments, the cyclic peptide produced during production in the host cell contains more than 3 and less than 60 amino acid residues.

In some embodiments, the cyclic peptide contains a serine or a cysteine residue.

In some embodiments, the cyclic peptide contains disulfide bonds, creating bicyclic, tricyclic, tetracyclic, or higher order cyclic peptide conformations.

In some embodiments, the cyclic peptide is derived from a single transcriptional and translational element.

In some embodiments, the cyclic peptide is generated from a polypeptide by enzymatic cyclization that is catalyzed by a second enzyme.

In some embodiments, the cyclic peptide contains an isopeptide bond.

In some embodiments, the cyclic peptide contains a disulfide bond.

In some embodiments, the cyclic peptide contains a posttranslational modification.

In some embodiments, the cyclic peptide contains a covalent bond between two different amino acid side chains.

In some embodiments, the cyclic peptide was selected from a large genetically encoded library of polypeptides configured to undergo cyclization.

In some embodiments, the one or more AAV replication genes are those that encode proteins selected from the group consisting of: Rep78, Rep68, Rep52, and Rep40

In some embodiments, the one or more AAV capsid encoding genes are those that encode proteins selected from the group consisting of: VP1, VP2, and VP3.

In some embodiments, the one or more adenovirus helper genes are selected from the group consisting of: Adenovirus E1A, Adenovirus E1B55K, Adenovirus E2A, Adenovirus E4 or f6, and Adenovirus VA.

In some embodiments, the one or more HSV helper genes are selected from the group consisting of: UL5, UL8, UL52, ICP8. In other embodiments, the one or more HPV helper genes are selected from the group consisting of: E1, E2, E6. In yet other embodiments, the one or more HBoVI helper genes are selected from the group consisting of: NS2, NS4, NP1, BocaSR.

In some embodiments, each host cell of first, second, third or higher order plurality of host cells is a mammalian host cell. In other embodiments, each host cell of first, second, third or higher order plurality of host cells is an insect host cell.

In some embodiments, the nucleotide sequence positioned between two ITRs comprises both a sequence that encodes a polypeptide configured to produce a cyclic peptide during production in the host cell, and/or a barcode that comprises identifying information regarding the cyclic peptide produced in the host cell.

In some embodiments, the nucleotide sequence positioned between two ITRs further encodes a reporter protein (e.g., GFP, luciferase) and/or a selectable marker (such as an antibiotic resistance gene, transcription factor, or other enzyme, which may allow to select for CP-mediated increase of payload gene expression) and/or a toxic marker.

In some embodiments, the disclosed method produces the AAV vector composition of increased viral titer and/or transduction efficiency having a titer of infectious particles per cell, which is at least 20%, 40%, 60%, 80%, 100%, 200%, or 500% higher than a titer of a reference AAV vector composition, which is produced by the same procedure and the same pluralities of host cells, except for host cells used to produce the reference AAV vector composition do not comprise the nucleotide sequence that encodes the polypeptide configured to produce a cyclic peptide.

In some embodiments, the first plurality of host cells at step (b) comprises at least 10,000 host cells that produce different cyclic peptides (e.g. for a cyclic peptide library with diversity of about 10,000). In some embodiments, the first plurality of host cells at step (b) comprises at least a one hundred thousand (100,000) host cells that produce different cyclic peptides (e.g. for a cyclic peptide library with diversity of about 100,000). In other embodiments, the first plurality of host cells at step (b) comprises at least one million (1,000,000) host cells that produce different cyclic peptides (e.g. for a cyclic peptide library with diversity of about 1,000,000). In other embodiments, the first plurality of host cells at step (b) comprises at least ten million (10,000,000) host cells that produce different cyclic peptides (e.g. for a cyclic peptide library with diversity of about 10,000,000).

In some embodiments, the nucleotide sequence positioned between two ITRs encodes an amino acid sequence that comprises any one of the sequences set forth in SEQ ID NO: 10-14 (e.g., amino acid sequence of intein polypeptide).

In some embodiments, AAV genome may be split in a host cell, which means that one ITR is integrated into genome of the host cell and another ITR is on a plasmid and functionally connected with a reporter protein, a therapeutic payload, a selectable marker or a nucleotide sequence that encodes a cyclic peptide. By integrating the plasmid into the correct genomic locus, one can generate a functional nucleotide sequence that is flanked by two ITRs, which can be further utilized, for example, during AAV packaging and/or payload production.

In some embodiments, the AAV vector composition of increased viral titer and/or transduction efficiency produced by the methods disclosed herein, has one or more useful properties, including: enhanced infectiousness (greater number of rAAV particles are transduction competent); enhanced payload expression (average level of gene expression per transduction event is higher); more optimal full:empty ratio (more full capsids and fewer empty capsids); higher viral genome titer; and/or lower level of immunogenicity.

In some embodiments, the AAV vector composition of increased viral titer and/or transduction efficiency produced by the methods disclosed herein comprises one or more improved features, wherein one or more improved features comprise altered ability to transfer viral nucleic acid, altered AAV therapeutic activity, and/or decreased in percentage of the AAV population that are nonfunctional, and/or increase in the percentage of viral vector under a manufacturing practice that contain all and/or the essential nucleic acid sequences and/or other elements for their intended application.

In some embodiments, the first plurality of recombinant AAV at step (f) are used to infect an animal model such as a mouse or rat, so that AAVs harboring CPs or barcodes that confer enhanced AAV manufacturability are transduced into the cells of the animal and maintained at a higher level compared to CPs/barcodes that confer no in vivo transduction enhancements.

In some embodiments, the mechanism of action of the cyclic peptide can be inferred by various approaches including molecular docking (e.g. reverse docking of the cyclic peptide to host and viral proteins), single cell RNA seq (i.e. to observe transcriptional consequences), machine learning prediction, mutational analysis, biochemical analysis of the rAAV capsid (e.g. gc/ms to determine capsid protein stoichiometry or post translational modification states), biochemical analysis of the rAAV DNA payload (e.g. methylation sequencing), quantitative microscopy/image-based profiling (e.g. in order to determine altered biological processes).

In some embodiments, next generation sequencing (NGS) is used to observe enrichment of different CPs, allowing one skilled in the art to infer the relative increase in AAV packaging fitness conferred by a given CP.

In some embodiments, two or more CPs in a single cell can yield synergistic effects.

In some embodiments, the CP generator is targeted to a specific cell organelle (e.g. endoplasmic reticulum) or cell process (e.g. degradation of remaining CP generator).

In some embodiments, additional functional properties of rAAV capsids can be enriched, including enhanced stability (e.g. by performing multiple freeze thaw cycles, exposure to elevated temperatures, exposure to various pH levels, exposure to neutralizing antibodies) on the libraries before subsequent rounds of reinfection and enrichment.

In some embodiments, the cyclic peptide diversity is generated with an NNK codon, an NNS codon, an NNN codon, or other degenerate codons that allow control over the amino acid composition of a particular residue position in the cyclic peptide.

In some embodiments, rAAVs may be harvested from cells (e.g. centrifugation of cells and extracting rAAV from pellet), from the media (i.e. to enrich rAAVs that were secreted into media or released by lysis), from fractions of density gradient centrifugation or capillary electrophoresis (i.e. to enrich for rAAVs that have properly packaged genomes).

In some embodiments of the disclosed methods, provided host cells comprise at least one cyclic peptide (CP) generator (e.g. a library of CP precursors). In some embodiments, the CP generator comprises a RiPP (ribosomally synthesized and post-translationally modified peptide), SICLOPPS, Cyanobactins, Lanthipeptides, Lasso peptides, Microcins, Thiopeptides, Autoinducing Peptides, conotoxins, Cyclotide, Class IIc bacteriocins, Linaridins, Microviridins, Orbitides, Proteusins, Sactipeptides (or other genetically encoded, ribosomally synthesized and post-translationally modified peptides).

In some embodiments, provided herein are mammalian host cells and/or mammalian host cell populations that comprise a plurality of engineered sequences comprising at least one library variant (CP precursor) and at least one identifier (barcode), and wherein the at least one identifier is positioned between the two AAV ITR sequences, and where the at least one library variant is positioned outside the two AAV ITR sequences. In some embodiments, provided mammalian host cells and/or mammalian host cell populations comprise a plurality of engineered sequences comprising at least one library variant, at least one identifier (barcode), and at least one payload, where the at least one identifier and the at least one payload are positioned between the two AAV ITR sequences, and where the at least one library variant is positioned outside the two AAV ITR sequences. In some embodiments, provided library constructs comprise: at least one library variant, at least one identifier (barcode), and at least one payload, where the at least one library variant, the at least one identifier, and the at least one payload are positioned between the two AAV ITR sequences.

Provided library constructs can be introduced into host cells using any appropriate method known in the art. In some embodiments, a library construct is introduced into a host cell by transfection and/or transduction. In some embodiments, a library construct is introduced into a host cell by viral-mediated transduction.

In some embodiments of the disclosed methods, provided host cells produce AAV vectors that are more functional and/or enhanced in an application, relative to a reference population. In some embodiments, provided host cells produce AAV vectors that are more functional and/or enhanced at transferring nucleic acid to a cell, relative to a reference population. In some embodiments, provided host cells produce AAV vectors that are more functional and/or enhanced therapeutically, relative to a reference population. In some embodiments, provided host cells produce AAV vectors that are more functional and/or enhanced in their intended application, relative to a reference population. In some embodiments, provided host cells comprise at least one engineered sequence (e.g., encoding CP) that provides an increase in AAV vector production under a manufacturing practice relative to a reference cell population.

Disclosed herein is also a plurality of host cells permissive for AAV replication, wherein each host cell of the plurality of host cells comprises an engineered cyclic peptide and further comprises:
(i) one or more AAV replication genes;
(ii) one or more AAV capsid encoding genes;
(iii) one or more viral helper genes; and
(iv) a nucleotide sequence operably linked to at least one functional AAV inverted terminal repeat (ITR), wherein the nucleotide sequence encodes a payload (e.g., therapeutic gene);

wherein the engineered cyclic peptide increases infectiousness (infectious unit titer) of AAV vectors produced by the plurality of host cells by at least 20%, 40%, 60%, 80%, 100%, 200%, or 500% compared to infectiousness of AAV vectors produced by a reference plurality of host cells that do not comprise the engineered cyclic peptide.

In some embodiments, the presence of the engineered cyclic peptide in the plurality of host cells is associated with an increase in AAV infectivity relative to a reference plurality of host cells that lacks the engineered cyclic peptide. In some embodiments, the presence of the engineered cyclic peptide in the plurality of host cells generates at least a 10%, 20%, 30%, 40%, 50%, 75%, 100%, 200%, or 500% increase in AAV infectivity relative to a reference plurality of host cells that lacks the engineered cyclic peptide.

In preferred embodiments, each host cell of the plurality of host cells is a mammalian host cell population.

In some embodiments, the payload is a therapeutic protein. In other embodiments, the payload is an RNA molecule.

In some embodiments, each host cell of the plurality of host cells produces the engineered cyclic peptide.

In some embodiments, each host cell of the plurality of host cells comprises at least 10\hat{ }2-10\hat{ }6 AAV genomes per cell (10-10\hat{ }6 infectious particles per cell).

In some embodiments, each host cell of the plurality of host cells is configured to produce at least 10\hat{ }2-10\hat{ }6 AAV genomes per cell (10-10\hat{ }6 infectious particles per cell).

In some embodiments, AAV nucleic acids of AAV vectors described herein typically include the cis-acting 5′ and 3′ ITR sequences. In some embodiments, at least 80% of a typical ITR sequence (e.g., at least 85%, at least 90%, or at least 95%) is incorporated into constructs provided herein. Generally, ITRs are able to form a hairpin. The ability to form a hairpin can contribute to an ITR's ability to self-prime, allowing primase-independent synthesis of a second DNA strand. ITRs can also aid in efficient encapsulation of an AAV construct in an AAV vector. An AAV ITR sequence may be obtained from any known AAV, including mammalian AAV types. In some embodiments, an ITR includes one or more modifications, e.g., truncations, deletions, substitutions or insertions, of a naturally occurring ITR sequence. In some embodiments, an ITR comprises fewer than 145 nucleotides.

In some embodiments, a barcode and/or a payload sequence is flanked by 5′ and 3′ AAV ITR sequences. In some embodiments, an AAV nucleic acid comprises a barcode and a payload flanked by 5′ and 3′ AAV ITR sequences. The AAV ITR sequences may be obtained from any known AAV, including presently identified AAV types.

In some embodiments, the plurality of host cells configured to produce a AAV vector composition of increased viral titer and/or transduction efficiency having TU:VG ratio from 1:100 to 1:1. In some embodiments, TU:VG ratio of the AAV vector composition of increased viral titer and/or transduction efficiency is from 1:100 to 1:50, from 1:50 to 1:20, from 1:20 to 1:10, from 1:10 to 1:5, from 1:5 to 1:2, or from 1:2 to 1:1.

In some embodiments, the engineered cyclic peptide contains more than 3 and less than 60 amino acid residues, and wherein the engineered cyclic peptide is either produced endogenously or supplied exogenously.

In some embodiments, the one or more AAV replication genes are those that encode proteins selected from the group consisting of: Rep78, Rep68, Rep52, and Rep40.

In some embodiments, the one or more AAV capsid encoding genes are those that encode proteins selected from the group consisting of: VP1, VP2, and VP3.

In some embodiments, the one or more viral helper genes are selected from the group consisting of: Adenovirus E1A, Adenovirus E1B55K, Adenovirus E2A, Adenovirus E4 or f6, and Adenovirus VA.

In some embodiments, each host cell of the plurality of host cells comprises an exogenously supplied engineered cyclic peptide.

In some embodiments, the cyclic peptide is generated endogenously by the host cell.

In other embodiments a cyclic peptide is generated endogenously by one cell, exits the cell (e.g. diffusion, cell lysis, exosome/microvesicle secretion), and enters a second cell (e.g. through diffusion, endocytosis, transporter, microvesicle fusion, etc. . . . ) in order to enhance rAAV production.

In other embodiments, the cyclic peptide is generated synthetically (e.g. by chemical synthesis, cell free synthesis, etc. . . . ), is supplied exogenously to the cells (optionally mixed with a carrier/excipient/delivery vehicle e.g. liposomal delivery), and enters the host cell in order to enhance rAAV production.

Disclosed herein is also a method of producing a highly infectious adeno-associated virus (AAV) vector composition, the method comprising:
(a) providing a plurality of host cells permissive for AAV replication, wherein each host cell of the plurality of host cells comprises an engineered cyclic peptide and further comprises:
(i) one or more AAV replication genes;
(ii) one or more AAV capsid encoding genes;
(iii) one or more viral helper genes; and
(iv) a nucleotide sequence positioned between two inverted terminal repeats (ITRs), wherein the nucleotide sequence encodes a payload (e.g. therapeutic gene);

wherein the engineered cyclic peptide increases infectiousness (infectious unit titer) of AAV vectors produced by the plurality of host cells by at least 20%, 40%, 60%, 80%, 100%, 200%, or 500% compared to infectiousness (infectious unit titer) of AAV vectors produced by a reference plurality of host cells that do not comprise the engineered cyclic peptide;
(b) incubating the plurality of host cells in a cell culture medium under suitable conditions, and collecting AAV vectors, thereby producing the AAV vector composition of increased viral titer and/or transduction efficiency, wherein the AAV vector composition of increased viral titer and/or transduction efficiency has an infectious unit titer which is at least a 20%, 40%, 60%, 80%, 100%, 200%, or 500% greater than an infectious unit titer of a reference AAV vector composition produced without engineered cyclic peptides.

In some embodiments, TU:VG ratio of the AAV vector composition of increased viral titer and/or transduction efficiency produced by the methods disclosed herein is from 1:100 to 1:50, from 1:50 to 1:20, from 1:20 to 1:10, from 1:10 to 1:5, from 1:5 to 1:2, or from 1:2 to 1:1.

In some embodiments, the AAV vector composition of increased viral titer and/or transduction efficiency has a viral genome titer which is at least a 20%, 40%, 60%, 80%, 100%, 200%, or 500% greater than a viral genome titer of a reference AAV vector composition produced without engineered cyclic peptides.

In some embodiments, an AAV vector payload is less than 4 kb. In some embodiments, an AAV vector payload can include a sequence that is at least 500 bp, at least 1 kb, at least 2 kb, at least 3 kb, at least 4 kb. In some embodiments, an AAV vector payload can include a sequence that is at most 7.5 kb. In some embodiments, an AAV vector payload can include a sequence that is about 1 kb to about 2 kb, about 1 kb to about 3 kb, about 1 kb to about 4 kb, about 1 kb to about 5 kb. In some embodiments, an AAV vector can direct long-term expression of a payload. In other embodiments, an AAV vector can direct transient expression of a payload.

In some embodiments, the engineered cyclic peptide is produced endogenously in the plurality of host cells or supplied exogenously to the plurality of host cells, and contains more than 3 and less than 60 amino acid residues.

In some embodiments, the one or more AAV replication genes are those that encode proteins selected from the group consisting of: Rep78, Rep68, Rep52, and Rep40. In some embodiments, a combination of two or more AAV replication genes may be used.

In some embodiments, the one or more AAV capsid encoding genes are those that encode proteins selected from the group consisting of: VP1, VP2, and VP3. In some embodiments, a combination of two or more AAV capsid encoding genes may be used.

In some embodiments, the one or more viral helper genes are selected from the group consisting of. Adenovirus E1A, Adenovirus E1B55K, Adenovirus E2A, Adenovirus E4 or f6, and Adenovirus VA. In some embodiments, a combination of two or more viral helper genes may be used.

In some embodiments, the cyclic peptide is generated endogenously by the host cell through ribosomal synthesis and post-translational modification/cyclisation.

In some embodiments, the cyclic peptide is generated endogenously by one cell, exits the cell (e.g. diffusion, cell lysis, exosome/microvesicle secretion), and enters a second cell (e.g. through diffusion, endocytosis, transporter, microvesicle fusion, etc. . . . ) in order to enhance rAAV production.

In some embodiments, the cyclic peptide is generated synthetically (e.g. by chemical synthesis, cell free synthesis, etc. . . . ), is supplied exogenously to the cells (optionally mixed with a carrier/excipient/delivery vehicle e.g. liposomal delivery), and enters the host cell in order to create the cell comprising an engineered cyclic peptide with enhanced rAAV production.

In some embodiments, CP analogs may be synthesized (optionally, with minor chemical modifications), mixed with an acceptable excipient/carrier substance, and added to the host cells permissive for AAV replication, allowing the CPs to enter the AAV packaging cells, and increasing AAV vector infectivity.

In some embodiments, two host cell populations may be employed; the first host cell population contains all AAV production machinery, but no expression of the CP; and the second host cell population contains the CP-producing machinery. CPs produced by the second host cell population may be able to diffuse into neighboring cells of the first host cell population, where the CPs can enhance infectivity of AAV vectors.

In some embodiments, the engineered cyclic peptide identified using the discovery method disclosed herein, is derivatized (e.g. addition/elimination of a functional group of the engineered cyclic peptide) in order to alter the properties of the further engineered cyclic peptide (e.g. reactivity, solubility, boiling point, melting point, membrane permeability, proteolytic resistance, target specificity, toxicity, etc. . . . ).

In some embodiments, the engineered cyclic peptide or further engineered cyclic peptide (e.g. derivatized engineered cyclic peptide) is mixed with a plurality of host cells permissive for AAV replication (e.g. containing Rep, Cap, and helper genes) in order to create host cells that further comprise an exogenously supplemented, synthetically derivatized cyclic peptide.

In some embodiments, derivatization of the engineered cyclic peptide includes (but is not limited to) methylation, ethylation, alkylation, glycosylation, phosphorylation, palmitoylation, halogenation (e.g. fluoronation, chlorination, bromination, iodonation), amination, amidation, click chemistry modifications

In some embodiments, derivatization of the engineered cyclic peptide increases membrane permeability (e.g. via backbone N-Methylation, alpha-carbon modifications, etc. . . . ).

In some embodiments, the cyclic peptide may contain one or more peptidomimetic elements (e.g. D-amino acids, beta-amino acids, non-alpha amino acids, N-substituted glycines, etc. . . . ).

In some embodiments, the cyclic peptide contains a lactam bridge between glutamic/aspartic acid and lysine residues.

In some embodiments, the cyclic peptide contains a lactone or thiolactone bridge between amino acids containing carboxyl, hydroxyl or mercapto functional groups

In some embodiments, the cyclic peptide contains thioether or ether bridges between the amino acids containing hydroxyl or mercapto functional groups

In some embodiments, the cyclic peptide is the result of alternative cyclization architectures, including head-to-tail, side-chain-to-side-chain, head-to-side-chain, and side-chain-to-tail. Head-to-tail cycles are typically formed by amide bond formation. Side-chain-to-side-chain cycles are most commonly formed by Cys-Cys disulfide bridges, amide bond formation, hydrocarbon-stapling (e.g. via olefin metathesis), click chemistry (e.g. alkyne-modified amino acid followed by click reaction with azido acid)

In some embodiments, the engineered cyclic peptide is present throughout the entire rAAV production process (e.g. in the case of endogenously produced, engineered cyclic peptides)

In some embodiments, the engineered cyclic peptide is added to cells before or during the rAAV production process in order to create cells comprising an engineered cyclic peptide (e.g. in the case of chemically synthesized, exogenously supplied engineered cyclic peptides)

In some embodiments, cells comprising engineered cyclic peptides further comprise both endogenously produced engineered cyclic peptides and exogenously supplied engineered cyclic peptides.

In some embodiments, cells comprising engineered cyclic peptides further comprise combinations of 2, 3, 4, 5, or more different engineered cyclic peptides that synergize in order to further increase yield and quality of rAAV produced by the host cell.

In some embodiments, cells comprising combinations of 2 or more cyclic peptides my further comprise any combination of cyclic peptides of the same amino acid ring sizes, different amino acid ring sizes, endogenously produced (e.g. ribosomally synthesized, post translationally cyclized), or exogenously supplied (e.g. via cross feeding from other endogenously produced engineered cyclic peptides or supplementation of chemically synthesized engineered cyclic peptides) engineered cyclic peptides.

In some embodiments, the first nucleotide sequence operably linked between two functional AAV inverted terminal repeats (ITRs) comprises two or more sequences, wherein each of the two or more sequences encodes a unique cyclic peptide.

In some embodiments, the cyclic peptide does not impact efficiency of AAV production, but only impacts the stability of the host cell line that stably expresses the elements required for AAV production.

In some embodiments, the viral helper genes critical for the replication, transcription, or packaging of a viral vector are derived from adenoviruses. These essential adenoviral helper genes, which may include but are not limited to Adenovirus E1A, Adenovirus E1B55K, Adenovirus E2A, Adenovirus E4 or f6, and Adenovirus VA, facilitate the processes essential for vector production. In particular embodiments, these helper genes can be supplied as part of the whole helper virus or as a subset of the genes.

In other embodiments, helper genes are obtained from herpesviruses. These herpes simplex virus (HSV) genes, including UL5, UL8, UL52, and the major DNA-binding protein UL29, have been found to provide helper gene function for AAV replication. In certain instances, the HSV-1 DNA polymerase complex, composed of UL30/UL42, is instrumental for AAV DNA replication. Other herpesvirus genera that support AAV replication, such as varicella-zoster virus (VZV), human cytomegalovirus (HCMV), Epstein-Barr virus (EBV), and human herpesvirus 6 (HHV-6), may be employed in specific embodiments.

In some embodiments, non-essential helper genes are sourced from Human Papillomavirus (HPV). The HPV E1 protein provides helper function analogous to AAV Rep78 but without Rep78's endonuclease/covalent attachment activity. HPV genes E1, E2, and E6 can be employed to boost rAAV and wt AAV replication and yield, particularly when used in combination with adenovirus helper genes.

In certain embodiments, non-essential helper genes are derived from Human Bocavirus (HBoV). Essential for AAV2 duplex DNA genome replication and progeny virion production are the HBoVI NP1 and NS4 proteins, and a newly identified viral long noncoding RNA, BocaSR. Specifically, HBoVI NS2, NP1, and BocaSR are required for productive infection of HEK293 and HeLa cells by AAV2.

In various embodiments, alternative helper genes are utilized from different viruses such as Herpes Simplex Virus (HSV) or Baculovirus to enhance vector production. For instance, in certain instances, HSV helper genes including UL5, UL8, ICP8, and ICP27 are employed. In other scenarios, Baculovirus helper genes like p80, p143, p40, and p32 might be used. These alternative helper genes provide the flexibility to enhance replication and packaging processes.

Collectively, these embodiments reflect diverse sources of viral helper genes vital for the replication, transcription, or packaging of a viral vector, offering flexibility and adaptability in the production process.

In certain embodiments, derivatives of the Chinese Hamster Ovary (CHO) cell line are utilized. Known for their extensive application in biopharmaceutical production, these CHO cells provide a mammalian cell platform that can potentially be optimized for both AAV and lentiviral vector production. In some such embodiments, CHO cells are employed to produce advanced recombinant proteins, which necessitate proper protein folding and post-translational modifications. These CHO cell lines may further be engineered to enhance bioproduction efficiency and improve product quality.

In specific embodiments, the cell lines utilized for viral production, including adeno-associated virus (AAV) and lentivirus, originate from Human Embryonic Kidney 293 (HEK293) cells. These cells, along with their derivative or related strains, are routinely employed in biotechnology due to their notable transfection efficiency and ability to proliferate to high densities, thus facilitating efficient viral production. In particular embodiments, the HEK293 cell line is utilized as an expression host for proteins requiring human-specific post-translational modifications. Furthermore, these HEK293 cells may be used for the production of recombinant AAV and lentiviral particles, owing to the expression of the essential helper factors E1A and E1B.

In some embodiments, the 293T cell line, an established lineage from the parental HEK293 line, may express the temperature sensitive allele of the large T antigen of Simian virus 40, contributing to improved recombinant protein production and therapeutic protein production. This cell line can potentially be used for both AAV and lentiviral production.

In some embodiments, the 293E cell line, another lineage derived from the parental HEK293 cell line, may express the Epstein-Barr virus nuclear antigen EBNA1, assisting in optimized recombinant protein production and the production of therapeutic proteins. These cells might also be suitable for AAV and lentivirus production.

In some embodiments, the 293-F and 293-H cell lines, industrially relevant suspension cell lines, are adapted for high-density suspension growth in serum-free medium, enabling large-scale cultivation and bioproduction in bioreactors with fast growth and high transfectivity. They can potentially be used for AAV and lentivirus production.

In some embodiments, the Freestyle 293-F cell line, a derivative of the HEK293 cell line, is adapted for high-density suspension growth in serum-free medium, facilitating large-scale production of therapeutic proteins in bioreactors due to its ability to increase volumetric cell density without cell clump formation. It can be employed for both AAV and lentivirus production.

In other embodiments, PER.C6 cells, a human retinal pigment epithelial cell line, are utilized for AAV and potentially lentivirus production. These cells, known for their easy maintenance and high transfection efficiency, offer a robust platform for the production of viral vectors.

In certain embodiments, BTI-TN-5B1-4 cells, commonly known as High Five cells, are employed. These cells, derived from Trichoplusia ni, the cabbage looper, ovarian cells, can be used in the baculovirus expression vector system for AAV and potentially lentivirus production. Due to their capacity to grow to high densities in suspension culture and their reputation for high yield recombinant protein production, High Five cells serve as a powerful tool for viral production.

In some embodiments, Sf9 insect cells are used for viral production. As an established cell line for the baculovirus expression vector system, Sf9 cells offer a robust platform for recombinant protein production and AAV and potentially lentiviral vector generation.

In other embodiments, potential cell lines for viral production can include viro9 cells. While the utility of these cells for AAV and lentivirus production would require further investigation, their potential use contributes to the broad applicability of this approach.

In various embodiments, Baby Hamster Kidney (BHK) cells are employed for AAV and potentially lentivirus production. BHK cells can be transfected with plasmids encoding the viral vector genome, rep and cap genes, and helper functions from adenoviruses, making them particularly valuable for the production of certain viral serotypes.

Collectively, these embodiments reflect the diversity of cell lines that can be utilized for viral vector production, offering a range of options to accommodate specific requirements for AAV and lentivirus production.

In some embodiments, endogenously produced engineered cyclic peptides may be operably linked to an inducible promoter (e.g. tet-ON) so that engineered cyclic peptide expression and timing can be tuned in order to further optimize rAAV production.

Different AAV serotypes have different capsid protein sequences, replication protein sequences, ITR sequences, and other genes. These differences have large impacts on each serotype's ability to infect different cell types; this is the primary motivation for use of different AAV serotypes. However, the same differences that provide enhanced targeting to one cell type over another also have large impacts on the manufacturability (e.g. viral genome titer, full:empty ratio, infectivity/infectious titer, etc. . . . ). These benefits and challenges become even more pronounced when considering the use of chimeric, pseudotypes (e.g. AAV5 capsid with AAV2 genome elements), or machine-designed AAV vectors, which often do not fit neatly into any given serotype. This presents a problem for the clinical translational of AAV vectors to gene therapy products. For example, AAV serotypes AAV1-AAV9 produced in Hek293 cells or HeLa cells showed dramatically different infectious titers both within cell lines (e.g. across serotypes) and across cell lines (e.g. within serotypes). Considering the case of HEK293 cells (a preferred host cell line), AAV2 is produced at ˜1E9 IU/ml (infectious units per ml), while AAV9 is produced at ˜1E6 IU/ml—a 1000 fold difference.

When considering the productivity at the cell level, a nearly 3 order of magnitude range between different serotypes has been reported, spanning from ˜1000 viral genomes per cell (AAV4 produced in HEK293) to over ˜100,000 viral genomes per cell (AAV3 produced in Sf9 cells).

Thus, for one skilled in the art, it is readily apparent that there is substantial variation in the expected viral titer, infectious titer, and other properties of AAV products depending principally on the serotype (e.g. natural, chimeric, totally artificial), production process (e.g. cell line, triple transfection, helper virus), and potentially other aspects (e.g. the identity of the DNA payload to be deliver). The problem to be solved is to maximize viral yield (e.g. viral titer) and quality (e.g. infectiousness, full:empty, etc. . . . ) for a variety of different therapeutic products that may have different starting points.

Thus, one skilled in the art will recognize that relative increases in viral titer, infectiousness, and other desirable properties are to be expected (e.g. because different AAV products will have dramatically different levels of manufacturing optimization required). One skilled in the art will reasonably conclude that a 2-fold increase in viral titer or infectiousness as compared to a reference viral composition, is both definite and provides substantial utility. One skilled in the art will further recognize that multiple 2-fold increases in viral titer or infectiousness (e.g. from a cell comprising multiple engineered cyclic peptides), can exploit synergies that exist between different engineered cyclic peptides.

In some embodiments, a cyclic peptide capable of increasing viral titer and/or transduction efficiency of a viral vector composition is disclosed herein, the cyclic peptide having between 4 to 14 amino acids and having the following chemical attributes selected from the group consisting of: the molecular weight is between 250 and 2000 daltons; the number of valence electrons is between 100 and 850; the heavy atom count is between 16 and 160; the number of rotatable bonds is less than 50; the number of rings is less than 25; there are 4 or more hydrogen bond donors; and there are 4 or more hydrogen bond acceptors.

In some embodiments, a cyclic peptide capable of increasing viral titer and/or transduction efficiency of a viral vector composition is disclosed herein, the cyclic peptide having between 4 to 12 amino acids and having the following chemical attributes selected from the group consisting of: the molecular weight is between 250 and 1400 daltons; the number of valence electrons is between 100 and 550; the heavy atom count is between 16 and 100; the number of rotatable bonds is less than 40; the number of rings is less than 13; the number hydrogen bond donors is between 4 and 30; and the number hydrogen bond acceptors is between 4 and 25.

In some embodiments, a cyclic peptide capable of increasing viral titer and/or transduction efficiency of a viral vector composition disclosed herein has one or more of the chemical attributes selected from the group consisting of:
1) contains at least one residue with a side chain that contains at least one Oxygen atom;
2) contains at least one residue with a side chain that contains at least one Nitrogen atom;
3) contains at least one residue with a side chain that contains at least one Halogen atom;
4) contains at least one residue with a side chain that contains at least one Sulfur atom;
5) contains at least one residue with a side chain that contains at least one Phosphorous atom;
6) contains at least one residue with a side chain that contains at least one Boron atom;
7) contains at least one residue with a side chain that contains at least one Selenium atom;
8) Contains at least one or more N-methylations of the amides in the peptide backbone; and
9) Contains a serine, cysteine, or threonine which form two peptide bonds to any other amino acids upstream or downstream of themselves.

In some embodiments, a cyclic peptide capable of increasing viral titer and/or transduction efficiency of a viral vector composition is disclosed herein, the cyclic peptide having between 4 and 20 amino acids and containing at least one of the following peptide motifs selected from the group consisting of: one or more of [‘G’, ‘A’, ‘V’, ‘I’, ‘L’, ‘M’, ‘P’] followed by one or more of [‘S’, ‘T’, ‘Q’, ‘N’, ‘C’]; one or more of [‘K’, ‘R’, ‘H’] followed by one or more of [‘G’, ‘A’, ‘V’, ‘I’, ‘L’, ‘M’, ‘P’]; one or more of [‘S’, ‘T’, ‘Q’, ‘N’, ‘C’] followed by one or more of [‘K’, ‘R’, ‘H’]; one or more of [‘S’, ‘T’, ‘Q’, ‘N’, ‘C’] followed by one or more of [‘F’, ‘W’, ‘Y’]; one or more of [‘G’, ‘A’, ‘V’, ‘I’, ‘L’, ‘M’, ‘P’] followed by one or more of [‘K’, ‘R’, ‘H’]; one or more of [‘G’, ‘A’, ‘V’, ‘I’, ‘L’, ‘M’, ‘P’] followed by one or more of [‘D’, ‘E’]; one or more of [‘F’, ‘W’, ‘Y’] followed by one or more of [‘F’, ‘W’, ‘Y’]; one or more of [‘K’, ‘R’, ‘H’] followed by one or more of [‘F’, ‘W’, ‘Y’]; one or more of [‘K’, ‘R’, ‘H’] followed by one or more of [‘K’, ‘R’, ‘H’]; one or more of [‘D’, ‘E’] followed by one or more of [‘D’, ‘E’]; one or more of [‘D’, ‘E’] followed by one or more of [‘F’, ‘W’, ‘Y’]; one or more of [‘F’, ‘W’, ‘Y’] followed by one or more of [‘K’, ‘R’, ‘H’]; one or more of [‘D’, ‘E’] followed by one or more of [‘S’, ‘T’, ‘Q’, ‘N’, ‘C’]; one or more of [‘F’, ‘W’, ‘Y’] followed by one or more of [‘S’, ‘T’, ‘Q’, ‘N’, ‘C’]; one or more of [‘K’, ‘R’, ‘H’] followed by one or more of [‘S’, ‘T’, ‘Q’, ‘N’, ‘C’]; one or more of [‘F’, ‘W’, ‘Y’] followed by one or more of [‘G’, ‘A’, ‘V’, ‘I’, ‘L’, ‘M’, ‘P’]; one or more of [‘D’, ‘E’] followed by one or more of [‘G’, ‘A’, ‘V’, ‘I’, ‘L’, ‘M’, ‘P’]; and one or more of [‘S’, ‘T’, ‘Q’, ‘N’, ‘C’] followed by one or more of [‘S’, ‘T’, ‘Q’, ‘N’, ‘C’].

In some embodiments, a cyclic peptide capable of increasing viral titer and/or transduction efficiency of a viral vector composition is disclosed herein, the cyclic peptide having between 4 and 20 amino acids and containing at least one of the following peptide motifs selected from the group consisting of: one or more hydrophobic non-aromatic amino acids next to one or more polar uncharged or essentially neutral at pH 7.4 amino acids; one or more basic (positive) amino acids next to one or more hydrophobic non-aromatic amino acids; one or more basic (positive) amino acids next to one or more polar uncharged or essentially neutral at pH 7.4 amino acids; one or more aromatic amino acids next to one or more polar uncharged or essentially neutral at pH 7.4 amino acids; one or more acidic (negative) amino acids next to one or more polar uncharged or essentially neutral at pH 7.4 amino acids; one or more acidic (negative) amino acids next to one or more hydrophobic non-aromatic amino acids; two or more consecutive amino polar uncharged or essentially neutral at pH 7.4 amino acids; two or more consecutive acidic (negative) amino acids; two or more consecutive basic (positive) amino acids; two or more consecutive hydrophobic non-aromatic amino acids; two or more consecutive aromatic amino acids; hydrophobic non-aromatic includes the natural amino acids Glycine (G), Alanine (A), Valine (V), Isoleucine (I), Leucine (L), Methionine (M), and Proline (P), as well as non-natural amino acids that have hydrophobic non-aromatic side chains; aromatic includes the natural amino acids Phenylalanine (F), Tryptophan (W), and Tyrosine (Y), as well as non-natural amino acids that have aromatic side chains; negatively charged (proton donor, acidic) includes the natural amino acids Aspartic Acid (D) and Glutamic Acid (E), as well as non-natural amino acids that behave as proton donors under physiological conditions (pH 7.4), thus possessing acidic properties; positively charged (proton acceptor, basic) includes the natural amino acids Lysine (K), Arginine (R), and Histidine (H), as well as non-natural amino acids that behave as proton acceptors under physiological conditions (pH 7.4), thus possessing basic properties; and polar, uncharged or essentially neutral at pH 7.4 includes the natural amino acids Serine (S), Threonine (T), Glutamine (Q), Asparagine (N), and Cysteine (C), as well as non-natural amino acids that have polar side chains and are uncharged or essentially neutral at physiological conditions (pH 7.4).

In some embodiments, a cyclic peptide capable of increasing viral titer and/or transduction efficiency of a viral vector composition is disclosed herein, the cyclic peptide comprises various peptide motifs. These peptide motifs are defined as triplets of amino acid. Each amino acid is defined as one of five types based on the properties of their side chains/R-groups. As used herein, the five types of amino acid residues used in the disclosed CPs are defined based on the following historical conventional categories:
1) HYD: amino acid residues with hydrophobic/aliphatic/non-polar/non-aromatic R-groups, which consists of residues selected from the following group: ‘G’, ‘A’, ‘V’, ‘I’, ‘L’, ‘M’, ‘P’.
2) ARO: amino acid residues with hydrophobic aromatics R-groups, which consists of residues selected from the following group: ‘F’, ‘W’, ‘Y’.
3) POL: amino acid residues with polar uncharged R-groups, which consists of residues selected from the following group: ‘S’, ‘T’, ‘Q’, ‘N’, ‘C’.
4) POS: amino acid residues with positively charged (Basic) R-groups, which consists of residues selected from the following group: ‘K’, ‘R’, ‘H’.
5) NEG: amino acid residues with negatively charged (Acidic) R-groups, which consists of residues selected from the following group: ‘D’, ‘E’.

Based on these categories, one can determine whether a particular CP has a specific motif.

An example of a motif might be ARO_HYD_POL. This ARO_HYD_POL-motif would contain [‘F’, ‘W’, ‘Y’] followed by [‘G’, ‘A’, ‘V’, ‘I’, ‘L’, ‘M’, ‘P’] followed by [‘S’, ‘T’, ‘Q’, ‘N’, ‘C’].

The following 28 motifs were detected to have particularly potent effects in increasing viral titer and/or transduction efficiency of adeno-associated virus (AAV) vector composition when present in cyclic peptides:
ARO_HYD_POL-motif: [‘F’, ‘W’, ‘Y’] followed by [‘G’, ‘A’, ‘V’, ‘I’, ‘L’, ‘M’, ‘P’] followed by [‘S’, ‘T’, ‘Q’, ‘N’, ‘C’]; ARO_POL_ARO-motif: [‘F’, ‘W’, ‘Y’] followed by [‘S’, ‘T’, ‘Q’, ‘N’, ‘C’] followed by [‘F’, ‘W’, ‘Y’]; ARO_POL_POS-motif: [‘F’, ‘W’, ‘Y’] followed by [‘S’, ‘T’, ‘Q’, ‘N’, ‘C’] followed by [‘K’, ‘R’, ‘H’]; HYD_ARO_POL-motif: [‘G’, ‘A’, ‘V’, ‘I’, ‘L’, ‘M’, ‘P’] followed by [‘F’, ‘W’, ‘Y’] followed by [‘S’, ‘T’, ‘Q’, ‘N’, ‘C’]; HYD_HYD_POL-motif: [‘G’, ‘A’, ‘V’, ‘I’, ‘L’, ‘M’, ‘P’] followed by [‘G’, ‘A’, ‘V’, ‘I’, ‘L’, ‘M’, ‘P’] followed by [‘S’, ‘T’, ‘Q’, ‘N’, ‘C’]; HYD_NEG_POL-motif: [‘G’, ‘A’, ‘V’, ‘I’, ‘L’, ‘M’, ‘P’] followed by [‘D’, ‘E’] followed by [‘S’, ‘T’, ‘Q’, ‘N’, ‘C’]; HYD_POS_POL-motif: [‘G’, ‘A’, ‘V’, ‘I’, ‘L’, ‘M’, ‘P’] followed by [‘K’, ‘R’, ‘H’] followed by [‘S’, ‘T’, ‘Q’, ‘N’, ‘C’]; NEG_HYD_POL-motif: [‘D’, ‘E’] followed by [‘G’, ‘A’, ‘V’, ‘I’, ‘L’, ‘M’, ‘P’] followed by [‘S’, ‘T’, ‘Q’, ‘N’, ‘C’]; NEG_POL_ARO-motif: [‘D’, ‘E’] followed by [‘S’, ‘T’, ‘Q’, ‘N’, ‘C’] followed by [‘F’, ‘W’, ‘Y’]; NEG_POL_NEG-motif: [‘D’, ‘E’] followed by [‘S’, ‘T’, ‘Q’, ‘N’, ‘C’] followed by [‘D’, ‘E’]; NEG_POL_POS-motif: [‘D’, ‘E’] followed by [‘S’, ‘T’, ‘Q’, ‘N’, ‘C’] followed by [‘K’, ‘R’, ‘H’]; POL_HYD_ARO-motif: [‘S’, ‘T’, ‘Q’, ‘N’, ‘C’] followed by [‘G’, ‘A’, ‘V’, ‘I’, ‘L’, ‘M’, ‘P’] followed by [‘F’, ‘W’, ‘Y’]; POL_HYD_HYD-motif: [‘S’, ‘T’, ‘Q’, ‘N’, ‘C’] followed by [‘G’, ‘A’, ‘V’, ‘I’, ‘L’, ‘M’, ‘P’] followed by [‘G’, ‘A’, ‘V’, ‘I’, ‘L’, ‘M’, ‘P’]; POL_HYD_NEG-motif: [‘S’, ‘T’, ‘Q’, ‘N’, ‘C’] followed by [‘G’, ‘A’, ‘V’, ‘I’, ‘L’, ‘M’, ‘P’] followed by [‘D’, ‘E’]; POL_HYD_POL-motif: [‘S’, ‘T’, ‘Q’, ‘N’, ‘C’] followed by [‘G’, ‘A’, ‘V’, ‘I’, ‘L’, ‘M’, ‘P’] followed by [‘S’, ‘T’, ‘Q’, ‘N’, ‘C’]; POL_HYD_POS-motif: [‘S’, ‘T’, ‘Q’, ‘N’, ‘C’] followed by [‘G’, ‘A’, ‘V’, ‘I’, ‘L’, ‘M’, ‘P’] followed by [‘K’, ‘R’, ‘H’]; POL_NEG_ARO-motif: [‘S’, ‘T’, ‘Q’, ‘N’, ‘C’] followed by [‘D’, ‘E’] followed by [‘F’, ‘W’, ‘Y’]; POL_NEG_HYD-motif: [‘S’, ‘T’, ‘Q’, ‘N’, ‘C’] followed by [‘D’, ‘E’] followed by [‘G’, ‘A’, ‘V’, ‘I’, ‘L’, ‘M’, ‘P’]; POL_NEG_NEG-motif: [‘S’, ‘T’, ‘Q’, ‘N’, ‘C’] followed by [‘D’, ‘E’] followed by [‘D’, ‘E’]; POL_NEG_POS-motif: [‘S’, ‘T’, ‘Q’, ‘N’, ‘C’] followed by [‘D’, ‘E’] followed by [‘K’, ‘R’, ‘H’]; POL_POL_ARO-motif: [‘S’, ‘T’, ‘Q’, ‘N’, ‘C’] followed by [‘S’, ‘T’, ‘Q’, ‘N’, ‘C’] followed by [‘F’, ‘W’, ‘Y’]; POL_POL_HYD-motif: [‘S’, ‘T’, ‘Q’, ‘N’, ‘C’] followed by [‘S’, ‘T’, ‘Q’, ‘N’, ‘C’] followed by [‘G’, ‘A’, ‘V’, ‘I’, ‘L’, ‘M’, ‘P’]; POL_POL_NEG-motif: [‘S’, ‘T’, ‘Q’, ‘N’, ‘C’] followed by [‘S’, ‘T’, ‘Q’, ‘N’, ‘C’] followed by [‘D’, ‘E’]; POL_POL_POL-motif: [‘S’, ‘T’, ‘Q’, ‘N’, ‘C’] followed by [‘S’, ‘T’, ‘Q’, ‘N’, ‘C’] followed by [‘S’, ‘T’, ‘Q’, ‘N’, ‘C’]; POL_POL_POS-motif: [‘S’, ‘T’, ‘Q’, ‘N’, ‘C’] followed by [‘S’, ‘T’, ‘Q’, ‘N’, ‘C’] followed by [‘K’, ‘R’, ‘H’]; POS_HYD_POL-motif: [‘K’, ‘R’, ‘H’] followed by [‘G’, ‘A’, ‘V’, ‘I’, ‘L’, ‘M’, ‘P’] followed by [‘S’, ‘T’, ‘Q’, ‘N’, ‘C’]; POS_POL_ARO-motif: [‘K’, ‘R’, ‘H’] followed by [‘S’, ‘T’, ‘Q’, ‘N’, ‘C’] followed by [‘F’, ‘W’, ‘Y’]; and POS_POL_POS-motif: [‘K’, ‘R’, ‘H’] followed by [‘S’, ‘T’, ‘Q’, ‘N’, ‘C’] followed by [‘K’, ‘R’, ‘H’].

These 28 motifs were found in substantially different frequencies in the enriched cyclic peptide libraries compared to sets of random cyclic peptides. In particular, these 28 motifs were detected in substantially different frequencies in the enriched cyclic peptide libraries compared to sets of random cyclic peptides with an average P-value of 1.7E-8 over 1000 replicates of analysis.

In some embodiments, the amino acid residues in CPs disclosed herein are non-natural and/or modified. In some embodiments, the amino acid backbone atoms of amino acid residues in CPs are modified (as previously discussed). In other embodiments, the side chains/R-groups of amino acid residues in CPs are modified, a derivative of a naturally occurring amino acid, or entirely synthetic. In some embodiments, CPs comprise any one of 210 non-natural amino acids and amino acid derivatives described in the SwissSidechain database, published in Nucleic Acids Research: Gfeller D, Michielin O, Zoete V, Nucleic Acids Research, 41, D327-D332 (2013). In some embodiments, CPs comprise any one of 593 non-natural amino acids and amino acid derivatives described in (Liang G, et al., An index for characterization of natural and non-natural amino acids for peptidomimetics. PLoS One. 2013 Jul. 23; 8(7):e67844).

The chemical properties of the amino acid side chain determine which group the amino acid belongs to. The evaluation of amino acid side chain chemical properties is known in the art, thus one can assign non-naturally occurring amino acid residues to at least one of five categories as shown above. While the natural amino acids have historically agreed upon categories despite individual amino acids often possessing hybrid properties, non-natural amino acids do not have universally agreed upon categorization. There are cases where non-natural amino acids sit between multiple categories and in such cases, they may be assigned to more than one category (e.g., assigned to both HYD and POL, or to both ARO and POL).

A few non-limiting examples of assignments for non-natural side chains are shown below. 7-hydroxy-1-tryptophan is a derivative of tryptophan with an additional hydroxyl group. Tryptophan is typically classified as hydrophobic aromatic due to its indole ring. The hydroxyl group makes it more polar, but it retains aromatic character. It would therefore be included in the ARO category. However, in specific contexts where the polar interactions are being prioritized, it may also be considered for the POL category. Phosphotyrosine is a derivative of tyrosine with a phosphate group attached to the phenol ring. The phosphate group is highly polar and charged, and the benzene ring retains its aromaticity. It would therefore be included in the NEG category. Pentafluoro-phenylalanine is a phenylalanine derivative, with the benzene ring substituted with five fluorine atoms. Fluorine atoms are highly electronegative, which leads to the generation of partial charges, making the molecule polar. At the same time, it is known that fluorinated compounds can often be hydrophobic due to the mitigation of polar interactions by the C-F bonds. It may therefore be categorized as either ARO (due to aromatic phenyl ring and hydrophobic fluorines) or POL (due to highly electronegative fluorines). 4-Hydroxy-tryptophan is a derivative of tryptophan with an additional hydroxyl group. Tryptophan is hydrophobic aromatic due to its indole ring. The hydroxyl group makes it more polar, but it retains aromatic character. The molecule has hybrid properties: the aromatic indole ring suggests hydrophobic aromatics, whereas the hydroxyl group suggests polar character. Considering its structural similarity to tryptophan and the retention of the aromatic indole ring, it would therefore be more closely aligned with the ARO category. However, in specific contexts where the polar interactions are being prioritized, it may also be considered for the POL category. 3,3-Dimethyl-methionine sulfoxide is a derivative of methionine, with an additional sulfoxide group and two methyl groups. Methionine is traditionally classified as a hydrophobic amino acid due to its thioether side chain. The sulfoxide group in 3,3-dimethyl-methionine sulfoxide adds polarity to the molecule, while the two additional methyl groups enhance the hydrophobic character. Additionally, the molecule does not have any charged groups at physiological pH. Therefore, this amino acid would be included in the HYD category. Canaline is an amino acid derivative that contains a side chain with a hydroxyurea functionality. The hydroxyurea group introduces polarity to the molecule due to the presence of both hydroxyl and amine functional groups, which are known for forming hydrogen bonds. While the amine group can get protonated at lower pH, at physiological pH the molecule would likely have a zwitterionic form similar to standard amino acids. Therefore, Canaline may be categorized as POL.

In some embodiments, a cyclic peptide capable of increasing viral titer and/or transduction efficiency of a viral vector composition is disclosed herein, the cyclic peptide having between 4 and 20 amino acids and comprising at least one of the following peptide motifs selected from the group consisting of. [‘G’, ‘A’, ‘V’, ‘I’, ‘L’, ‘M’, ‘P’] followed by [‘K’, ‘R’, ‘H’], followed by [‘S’, ‘T’, ‘Q’, ‘N’, ‘C’]; [‘G’, ‘A’, ‘V’, ‘I’, ‘L’, ‘M’, ‘P’] followed by [‘G’, ‘A’, ‘V’, ‘I’, ‘L’, ‘M’, ‘P’], followed by [‘G’, ‘A’, ‘V’, ‘I’, ‘L’, ‘M’, ‘P’]; [‘G’, ‘A’, ‘V’, ‘I’, ‘L’, ‘M’, ‘P’] followed by [‘S’, ‘T’, ‘Q’, ‘N’, ‘C’], followed by [‘G’, ‘A’, ‘V’, ‘I’, ‘L’, ‘M’, ‘P’]; [‘S’, ‘T’, ‘Q’, ‘N’, ‘C’] followed by [‘D’, ‘E’], followed by [‘S’, ‘T’, ‘Q’, ‘N’, ‘C’]; [‘S’, ‘T’, ‘Q’, ‘N’, ‘C’] followed by [‘G’, ‘A’, ‘V’, ‘I’, ‘L’, ‘M’, ‘P’], followed by [‘S’, ‘T’, ‘Q’, ‘N’, ‘C’]; [‘G’, ‘A’, ‘V’, ‘I’, ‘L’, ‘M’, ‘P’] followed by [‘S’, ‘T’, ‘Q’, ‘N’, ‘C’], followed by [‘F’, ‘W’, ‘Y’]; [‘G’, ‘A’, ‘V’, ‘I’, ‘L’, ‘M’, ‘P’] followed by [‘G’, ‘A’, ‘V’, ‘I’, ‘L’, ‘M’, ‘P’], followed by [‘F’, ‘W’, ‘Y’]; [‘K’, ‘R’, ‘H’] followed by [‘G’, ‘A’, ‘V’, ‘I’, ‘L’, ‘M’, ‘P’], followed by [‘S’, ‘T’, ‘Q’, ‘N’, ‘C’]; [‘S’, ‘T’, ‘Q’, ‘N’, ‘C’] followed by [‘F’, ‘W’, ‘Y’], followed by [‘S’, ‘T’, ‘Q’, ‘N’, ‘C’]; [‘K’, ‘R’, ‘H’] followed by [‘S’, ‘T’, ‘Q’, ‘N’, ‘C’], followed by [‘S’, ‘T’, ‘Q’, ‘N’, ‘C’]; [‘S’, ‘T’, ‘Q’, ‘N’, ‘C’] followed by [‘D’, ‘E’], followed by [‘K’, ‘R’, ‘H’]; [‘D’, ‘E’] followed by [‘K’, ‘R’, ‘H’], followed by [‘S’, ‘T’, ‘Q’, ‘N’, ‘C’]; [‘S’, ‘T’, ‘Q’, ‘N’, ‘C’] followed by [‘K’, ‘R’, ‘H’], followed by [‘S’, ‘T’, ‘Q’, ‘N’, ‘C’]; [‘F’, ‘W’, ‘Y’] followed by [‘K’, ‘R’, ‘H’], followed by [‘G’, ‘A’, ‘V’, ‘I’, ‘L’, ‘M’, ‘P’]; [‘S’, ‘T’, ‘Q’, ‘N’, ‘C’] followed by [‘S’, ‘T’, ‘Q’, ‘N’, ‘C’], followed by [‘D’, ‘E’]; [‘D’, ‘E’] followed by [‘G’, ‘A’, ‘V’, ‘I’, ‘L’, ‘M’, ‘P’], followed by [‘G’, ‘A’, ‘V’, ‘I’, ‘L’, ‘M’, ‘P’]; [‘D’, ‘E’] followed by [‘S’, ‘T’, ‘Q’, ‘N’, ‘C’], followed by [‘S’, ‘T’, ‘Q’, ‘N’, ‘C’]; [‘S’, ‘T’, ‘Q’, ‘N’, ‘C’] followed by [‘K’, ‘R’, ‘H’], followed by [‘D’, ‘E’]; [‘D’, ‘E’] followed by [‘S’, ‘T’, ‘Q’, ‘N’, ‘C’], followed by [‘G’, ‘A’, ‘V’, ‘I’, ‘L’, ‘M’, ‘P’]; [‘G’, ‘A’, ‘V’, ‘I’, ‘L’, ‘M’, ‘P’] followed by [‘G’, ‘A’, ‘V’, ‘I’, ‘L’, ‘M’, ‘P’], followed by [‘S’, ‘T’, ‘Q’, ‘N’, ‘C’]; [‘D’, ‘E’] followed by [‘G’, ‘A’, ‘V’, ‘I’, ‘L’, ‘M’, ‘P’], followed by [‘F’, ‘W’, ‘Y’]; [‘D’, ‘E’] followed by [‘G’, ‘A’, ‘V’, ‘I’, ‘L’, ‘M’, ‘P’], followed by [‘S’, ‘T’, ‘Q’, ‘N’, ‘C’]; [‘G’, ‘A’, ‘V’, ‘I’, ‘L’, ‘M’, ‘P’] followed by [‘F’, ‘W’, ‘Y’], followed by [‘G’, ‘A’, ‘V’, ‘I’, ‘L’, ‘M’, ‘P’]; [‘K’, ‘R’, ‘H’] followed by [‘S’, ‘T’, ‘Q’, ‘N’, ‘C’], followed by [‘G’, ‘A’, ‘V’, ‘I’, ‘L’, ‘M’, ‘P’]; [‘G’, ‘A’, ‘V’, ‘I’, ‘L’, ‘M’, ‘P’] followed by [‘F’, ‘W’, ‘Y’], followed by [‘F’, ‘W’, ‘Y’]; [‘F’, ‘W’, ‘Y’]followed by [‘S’, ‘T’, ‘Q’, ‘N’, ‘C’], followed by [‘D’, ‘E’]; [‘G’, ‘A’, ‘V’, ‘I’, ‘L’, ‘M’, ‘P’] followed by [‘G’, ‘A’, ‘V’, ‘I’, ‘L’, ‘M’, ‘P’], followed by [‘D’, ‘E’]; [‘F’, ‘W’, ‘Y’] followed by [‘F’, ‘W’, ‘Y’], followed by [‘G’, ‘A’, ‘V’, ‘I’, ‘L’, ‘M’, ‘P’]; [‘K’, ‘R’, ‘H’] followed by [‘F’, ‘W’, ‘Y’], followed by [‘G’, ‘A’, ‘V’, ‘I’, ‘L’, ‘M’, ‘P’]; [‘F’, ‘W’, ‘Y’] followed by [‘S’, ‘T’, ‘Q’, ‘N’, ‘C’], followed by [‘K’, ‘R’, ‘H’]; [‘F’, ‘W’, ‘Y’] followed by [‘D’, ‘E’], followed by [‘S’, ‘T’, ‘Q’, ‘N’, ‘C’]; [‘K’, ‘R’, ‘H’] followed by [‘S’, ‘T’, ‘Q’, ‘N’, ‘C’], followed by [‘K’, ‘R’, ‘H’]; [‘F’, ‘W’, ‘Y’] followed by [‘S’, ‘T’, ‘Q’, ‘N’, ‘C’], followed by [‘S’, ‘T’, ‘Q’, ‘N’, ‘C’]; [‘F’, ‘W’, ‘Y’] followed by [‘G’, ‘A’, ‘V’, ‘I’, ‘L’, ‘M’, ‘P’], followed by [‘K’, ‘R’, ‘H’]; [‘S’, ‘T’, ‘Q’, ‘N’, ‘C’] followed by [‘G’, ‘A’, ‘V’, ‘I’, ‘L’, ‘M’, ‘P’], followed by [‘F’, ‘W’, ‘Y’]; [‘G’, ‘A’, ‘V’, ‘I’, ‘L’, ‘M’, ‘P’] followed by [‘S’, ‘T’, ‘Q’, ‘N’, ‘C’], followed by [‘D’, ‘E’]; [‘K’, ‘R’, ‘H’] followed by [‘D’, ‘E’], followed by [‘G’, ‘A’, ‘V’, ‘I’, ‘L’, ‘M’, ‘P’]; [‘D’, ‘E’] followed by [‘D’, ‘E’], followed by [‘S’, ‘T’, ‘Q’, ‘N’, ‘C’]; [‘K’, ‘R’, ‘H’] followed by [‘F’, ‘W’, ‘Y’], followed by [‘F’, ‘W’, ‘Y’]; [‘K’, ‘R’, ‘H’] followed by [‘K’, ‘R’, ‘H’], followed by [‘G’, ‘A’, ‘V’, ‘I’, ‘L’, ‘M’, ‘P’]; [‘K’, ‘R’, ‘H’] followed by [‘G’, ‘A’, ‘V’, ‘I’, ‘L’, ‘M’, ‘P’], followed by [‘D’, ‘E’]; [‘D’, ‘E’] followed by [‘S’, ‘T’, ‘Q’, ‘N’, ‘C’], followed by [‘D’, ‘E’]; [‘S’, ‘T’, ‘Q’, ‘N’, ‘C’] followed by [‘D’, ‘E’], followed by [‘G’, ‘A’, ‘V’, ‘I’, ‘L’, ‘M’, ‘P’]; [‘F’, ‘W’, ‘Y’] followed by [‘G’, ‘A’, ‘V’, ‘I’, ‘L’, ‘M’, ‘P’], followed by [‘F’, ‘W’, ‘Y’]; [‘D’, ‘E’] followed by [‘G’, ‘A’, ‘V’, ‘I’, ‘L’, ‘M’, ‘P’], followed by [‘K’, ‘R’, ‘H’]; [‘G’, ‘A’, ‘V’, ‘I’, ‘L’, ‘M’, ‘P’] followed by [‘F’, ‘W’, ‘Y’], followed by [‘K’, ‘R’, ‘H’]; [‘S’, ‘T’, ‘Q’, ‘N’, ‘C’] followed by [‘G’, ‘A’, ‘V’, ‘I’, ‘L’, ‘M’, ‘P’], followed by [‘K’, ‘R’, ‘H’]; [‘G’, ‘A’, ‘V’, ‘I’, ‘L’, ‘M’, ‘P’] followed by [‘F’, ‘W’, ‘Y’], followed by [‘S’, ‘T’, ‘Q’, ‘N’, ‘C’]; [‘S’, ‘T’, ‘Q’, ‘N’, ‘C’] followed by [‘K’, ‘R’, ‘H’], followed by [‘K’, ‘R’, ‘H’]; [‘G’, ‘A’, ‘V’, ‘I’, ‘L’, ‘M’, ‘P’] followed by [‘D’, ‘E’], followed by [‘D’, ‘E’]; [‘D’, ‘E’] followed by [‘K’, ‘R’, ‘H’], followed by [‘F’, ‘W’, ‘Y’]; [‘G’, ‘A’, ‘V’, ‘I’, ‘L’, ‘M’, ‘P’] followed by [‘G’, ‘A’, ‘V’, ‘I’, ‘L’, ‘M’, ‘P’], followed by [‘K’, ‘R’, ‘H’]; [‘S’, ‘T’, ‘Q’, ‘N’, ‘C’] followed by [‘F’, ‘W’, ‘Y’], followed by [‘G’, ‘A’, ‘V’, ‘I’, ‘L’, ‘M’, ‘P’]; [‘S’, ‘T’, ‘Q’, ‘N’, ‘C’] followed by [‘D’, ‘E’], followed by [‘D’, ‘E’]; [‘S’, ‘T’, ‘Q’, ‘N’, ‘C’] followed by [‘F’, ‘W’, ‘Y’], followed by [‘D’, ‘E’]; [‘F’, ‘W’, ‘Y’] followed by [‘F’, ‘W’, ‘Y’], followed by [‘D’, ‘E’]; [‘F’, ‘W’, ‘Y’] followed by [‘K’, ‘R’, ‘H’], followed by [‘F’, ‘W’, ‘Y’]; [‘D’, ‘E’] followed by [‘F’, ‘W’, ‘Y’], followed by [‘D’, ‘E’]; [‘G’, ‘A’, ‘V’, ‘I’, ‘L’, ‘M’, ‘P’] followed by [‘K’, ‘R’, ‘H’], followed by [‘F’, ‘W’, ‘Y’]; [‘G’, ‘A’, ‘V’, ‘I’, ‘L’, ‘M’, ‘P’] followed by [‘S’, ‘T’, ‘Q’, ‘N’, ‘C’], followed by [‘K’, ‘R’, ‘H’]; [‘K’, ‘R’, ‘H’] followed by [‘D’, ‘E’], followed by [‘F’, ‘W’, ‘Y’]; [‘F’, ‘W’, ‘Y’] followed by [‘D’, ‘E’], followed by [‘G’, ‘A’, ‘V’, ‘I’, ‘L’, ‘M’, ‘P’]; [‘F’, ‘W’, ‘Y’] followed by [‘S’, ‘T’, ‘Q’, ‘N’, ‘C’], followed by [‘F’, ‘W’, ‘Y’]; [‘F’, ‘W’, ‘Y’]followed by [‘F’, ‘W’, ‘Y’], followed by [‘F’, ‘W’, ‘Y’]; [‘S’, ‘T’, ‘Q’, ‘N’, ‘C’] followed by [‘G’, ‘A’, ‘V’, ‘I’, ‘L’, ‘M’, ‘P’], followed by [‘D’, ‘E’]; [‘G’, ‘A’, ‘V’, ‘I’, ‘L’, ‘M’, ‘P’] followed by [‘K’, ‘R’, ‘H’], followed by [‘K’, ‘R’, ‘H’]; [‘K’, ‘R’, ‘H’] followed by [‘D’, ‘E’], followed by [‘K’, ‘R’, ‘H’]; [‘S’, ‘T’, ‘Q’, ‘N’, ‘C’] followed by [‘S’, ‘T’, ‘Q’, ‘N’, ‘C’], followed by [‘K’, ‘R’, ‘H’]; [‘G’, ‘A’, ‘V’, ‘I’, ‘L’, ‘M’, ‘P’] followed by [‘K’, ‘R’, ‘H’], followed by [‘G’, ‘A’, ‘V’, ‘I’, ‘L’, ‘M’, ‘P’]; [‘S’, ‘T’, ‘Q’, ‘N’, ‘C’] followed by [‘K’, ‘R’, ‘H’], followed by [‘G’, ‘A’, ‘V’, ‘I’, ‘L’, ‘M’, ‘P’]; [‘F’, ‘W’, ‘Y’] followed by [‘D’, ‘E’], followed by [‘K’, ‘R’, ‘H’]; [‘D’, ‘E’] followed by [‘F’, ‘W’, ‘Y’], followed by [‘K’, ‘R’, ‘H’]; [‘S’, ‘T’, ‘Q’, ‘N’, ‘C’] followed by [‘F’, ‘W’, ‘Y’], followed by [‘F’, ‘W’, ‘Y’]; [‘K’, ‘R’, ‘H’] followed by [‘K’, ‘R’, ‘H’], followed by [‘S’, ‘T’, ‘Q’, ‘N’, ‘C’]; [‘K’, ‘R’, ‘H’] followed by [‘F’, ‘W’, ‘Y’], followed by [‘K’, ‘R’, ‘H’]; [‘S’, ‘T’, ‘Q’, ‘N’, ‘C’] followed by [‘S’, ‘T’, ‘Q’, ‘N’, ‘C’], followed by [‘S’, ‘T’, ‘Q’, ‘N’, ‘C’]; [‘F’, ‘W’, ‘Y’] followed by [‘D’, ‘E’], followed by [‘D’, ‘E’]; [‘D’, ‘E’] followed by [‘F’, ‘W’, ‘Y’], followed by [‘S’, ‘T’, ‘Q’, ‘N’, ‘C’]; [‘S’, ‘T’, ‘Q’, ‘N’, ‘C’] followed by [‘G’, ‘A’, ‘V’, ‘I’, ‘L’, ‘M’, ‘P’], followed by [‘G’, ‘A’, ‘V’, ‘I’, ‘L’, ‘M’, ‘P’]; [‘D’, ‘E’] followed by [‘D’, ‘E’], followed by [‘K’, ‘R’, ‘H’]; [‘S’, ‘T’, ‘Q’, ‘N’, ‘C’] followed by [‘K’, ‘R’, ‘H’], followed by [‘F’, ‘W’, ‘Y’]; [‘K’, ‘R’, ‘H’] followed by [‘S’, ‘T’, ‘Q’, ‘N’, ‘C’], followed by [‘F’, ‘W’, ‘Y’]; [‘G’, ‘A’, ‘V’, ‘I’, ‘L’, ‘M’, ‘P’] followed by [‘D’, ‘E’], followed by [‘G’, ‘A’, ‘V’, ‘I’, ‘L’, ‘M’, ‘P’]; [‘D’, ‘E’] followed by [‘F’, ‘W’, ‘Y’], followed by [‘F’, ‘W’, ‘Y’]; [‘D’, ‘E’] followed by [‘K’, ‘R’, ‘H’], followed by [‘K’, ‘R’, ‘H’]; [‘K’, ‘R’, ‘H’] followed by [‘G’, ‘A’, ‘V’, ‘I’, ‘L’, ‘M’, ‘P’], followed by [‘F’, ‘W’, ‘Y’]; [‘S’, ‘T’, ‘Q’, ‘N’, ‘C’] followed by [‘D’, ‘E’], followed by [‘F’, ‘W’, ‘Y’]; [‘K’, ‘R’, ‘H’] followed by [‘K’, ‘R’, ‘H’], followed by [‘K’, ‘R’, ‘H’]; [‘F’, ‘W’, ‘Y’] followed by [‘K’, ‘R’, ‘H’], followed by [‘D’, ‘E’]; [‘S’, ‘T’, ‘Q’, ‘N’, ‘C’] followed by [‘S’, ‘T’, ‘Q’, ‘N’, ‘C’], followed by [‘F’, ‘W’, ‘Y’]; [‘F’, ‘W’, ‘Y’] followed by [‘G’, ‘A’, ‘V’, ‘I’, ‘L’, ‘M’, ‘P’], followed by [‘S’, ‘T’, ‘Q’, ‘N’, ‘C’]; [‘S’, ‘T’, ‘Q’, ‘N’, ‘C’] followed by [‘S’, ‘T’, ‘Q’, ‘N’, ‘C’], followed by [‘G’, ‘A’, ‘V’, ‘I’, ‘L’, ‘M’, ‘P’]; [‘K’, ‘R’, ‘H’] followed by [‘D’, ‘E’], followed by [‘S’, ‘T’, ‘Q’, ‘N’, ‘C’]; [‘G’, ‘A’, ‘V’, ‘I’, ‘L’, ‘M’, ‘P’] followed by [‘K’, ‘R’, ‘H’], followed by [‘D’, ‘E’]; [‘D’, ‘E’] followed by [‘S’, ‘T’, ‘Q’, ‘N’, ‘C’], followed by [‘K’, ‘R’, ‘H’]; [‘F’, ‘W’, ‘Y’] followed by [‘F’, ‘W’, ‘Y’], followed by [‘K’, ‘R’, ‘H’]; [‘K’, ‘R’, ‘H’] followed by [‘F’, ‘W’, ‘Y’], followed by [‘S’, ‘T’, ‘Q’, ‘N’, ‘C’]; [‘K’, ‘R’, ‘H’] followed by [‘S’, ‘T’, ‘Q’, ‘N’, ‘C’], followed by [‘D’, ‘E’]; [‘K’, ‘R’, ‘H’] followed by [‘G’, ‘A’, ‘V’, ‘I’, ‘L’, ‘M’, ‘P’], followed by [‘G’, ‘A’, ‘V’, ‘I’, ‘L’, ‘M’, ‘P’]; [‘F’, ‘W’, ‘Y’] followed by [‘F’, ‘W’, ‘Y’], followed by [‘S’, ‘T’, ‘Q’, ‘N’, ‘C’]; [‘G’, ‘A’, ‘V’, ‘I’, ‘L’, ‘M’, ‘P’] followed by [‘D’, ‘E’], followed by [‘K’, ‘R’, ‘H’]; [‘K’, ‘R’, ‘H’] followed by [‘F’, ‘W’, ‘Y’], followed by [‘D’, ‘E’]; [‘G’, ‘A’, ‘V’, ‘I’, ‘L’, ‘M’, ‘P’] followed by [‘S’, ‘T’, ‘Q’, ‘N’, ‘C’], followed by [‘S’, ‘T’, ‘Q’, ‘N’, ‘C’]; [‘D’, ‘E’] followed by [‘D’, ‘E’], followed by [‘F’, ‘W’, ‘Y’]; [‘F’, ‘W’, ‘Y’] followed by [‘S’, ‘T’, ‘Q’, ‘N’, ‘C’], followed by [‘G’, ‘A’, ‘V’, ‘I’, ‘L’, ‘M’, ‘P’] followed by [‘D’, ‘E’], followed by [‘F’, ‘W’, ‘Y’]; [‘S’, ‘T’, ‘Q’, ‘N’, ‘C’] followed by [‘F’, ‘W’, ‘Y’], followed by [‘K’, ‘R’, ‘H’]; [‘F’, ‘W’, ‘Y’] followed by [‘G’, ‘A’, ‘V’, ‘I’, ‘L’, ‘M’, ‘P’], followed by [‘G’, ‘A’, ‘V’, ‘I’, ‘L’, ‘M’, ‘P’]; [‘D’, ‘E’] followed by [‘K’, ‘R’, ‘H’], followed by [‘G’, ‘A’, ‘V’, ‘I’, ‘L’, ‘M’, ‘P’]; [‘K’, ‘R’, ‘H’] followed by [‘D’, ‘E’], followed by [‘D’, ‘E’]; [‘K’, ‘R’, ‘H’] followed by [‘G’, ‘A’, ‘V’, ‘I’, ‘L’, ‘M’, ‘P’], followed by [‘K’, ‘R’, ‘H’]; [‘D’, ‘E’] followed by [‘D’, ‘E’], followed by [‘G’, ‘A’, ‘V’, ‘I’, ‘L’, ‘M’, ‘P’]; [‘D’, ‘E’] followed by [‘S’, ‘T’, ‘Q’, ‘N’, ‘C’], followed by [‘F’, ‘W’, ‘Y’]; and [‘G’, ‘A’, ‘V’, ‘I’, ‘L’, ‘M’, ‘P’] followed by [‘D’, ‘E’], followed by [‘S’, ‘T’, ‘Q’, ‘N’, ‘C’].

These motifs were found in different frequencies in the enriched cyclic peptide libraries compared to sets of random cyclic peptides.

In some embodiments, cyclic peptides can improve viral production of both lentivirus and AAV vector composition (e.g., CSSLT (SEQ ID NO: 37 as shown in FIG. 23 and FIG. 25).

In some embodiments, cyclic peptides can improve production of both lentivirus and AAV vector composition in the same host cell.

In some embodiments, the packaging system is a natural, replication-competent virus. In other embodiments, the packaging system is 2nd, 3rd, or 4^{th} lentiviral packaging system.

In some embodiments, cyclic peptide encoding sequences are part of a genetic circuit controlled by regulatory elements.

In some embodiments, the cyclic peptide improves packaging of a toxic payload gene.

In some embodiments, a cyclic peptide encoding sequence is incorporated onto the lentivirus genome and integrated into the host cell genome.

In some embodiments, a payload of the lentivirus is a cyclic peptide encoding sequence that is operably linked to AAV ITRs.

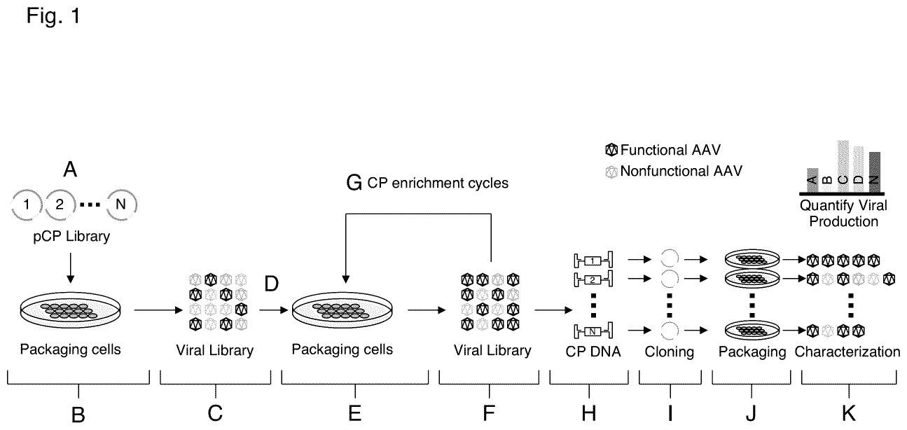

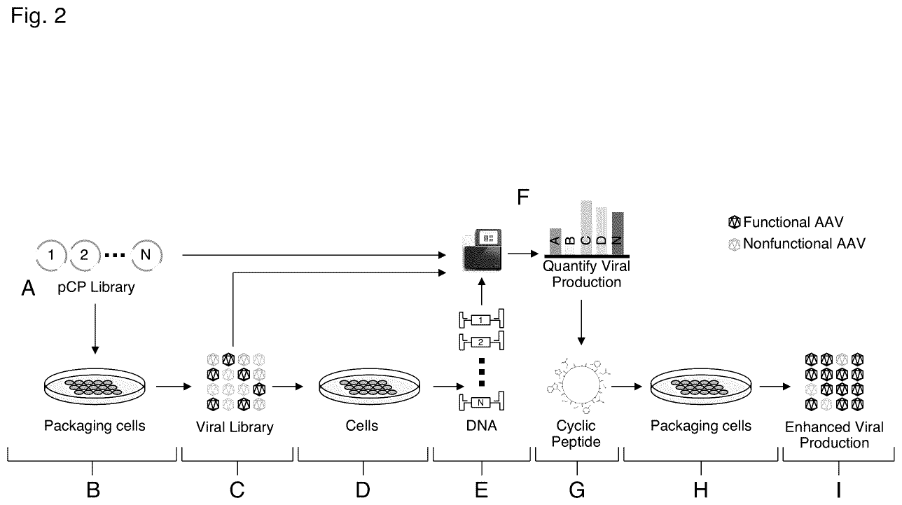

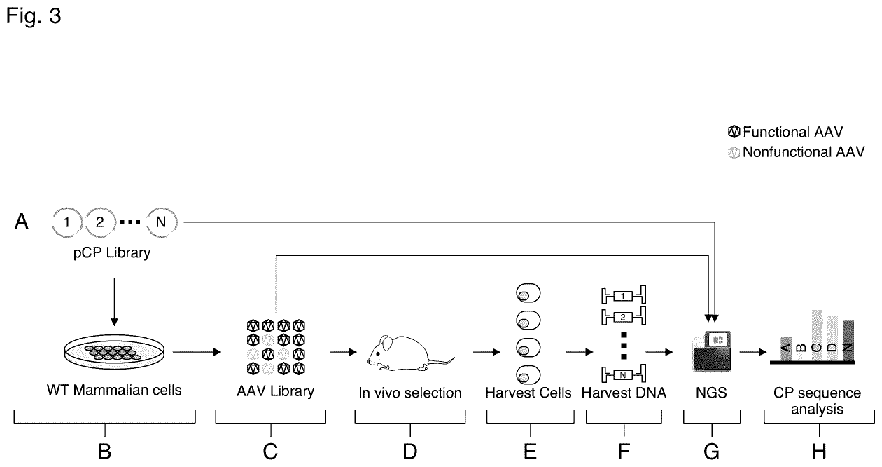

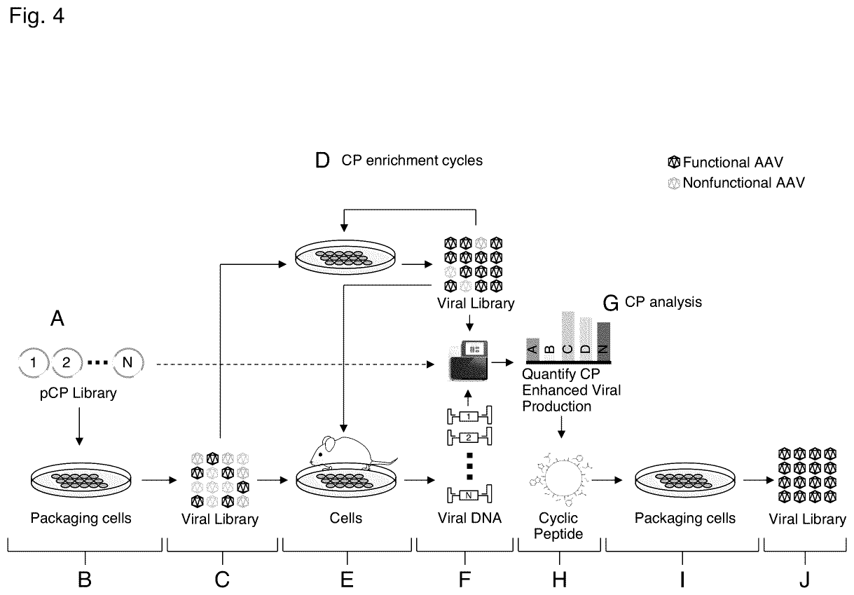

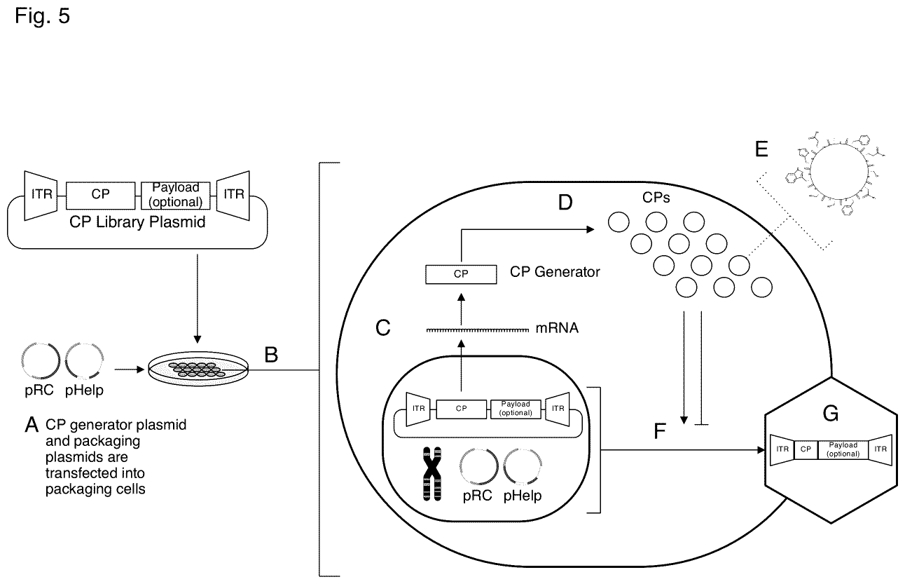

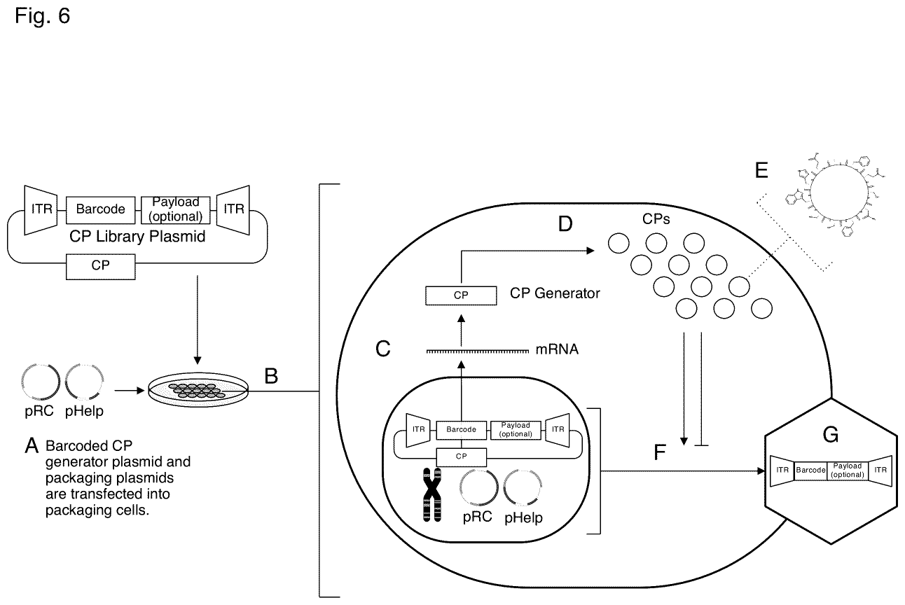

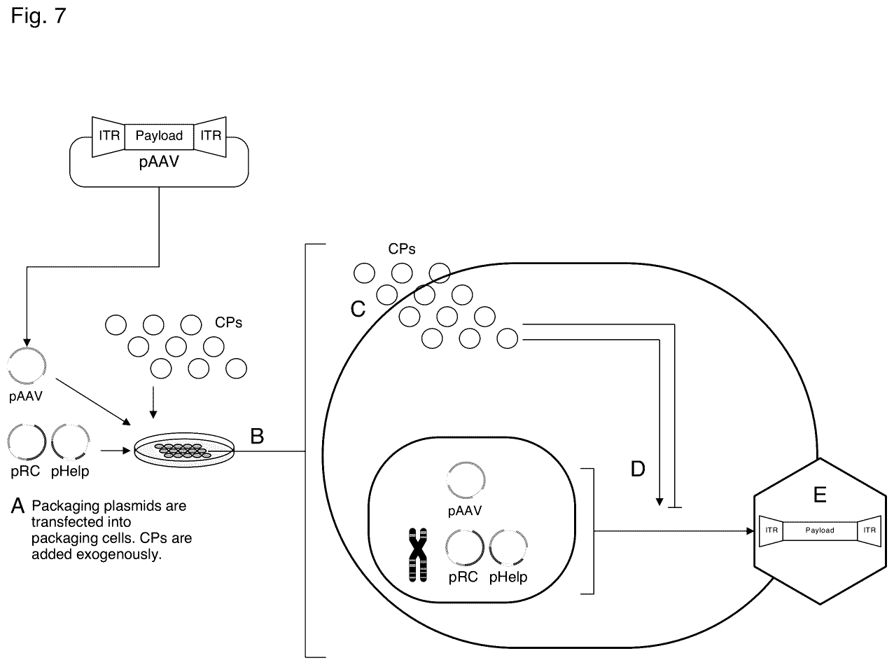

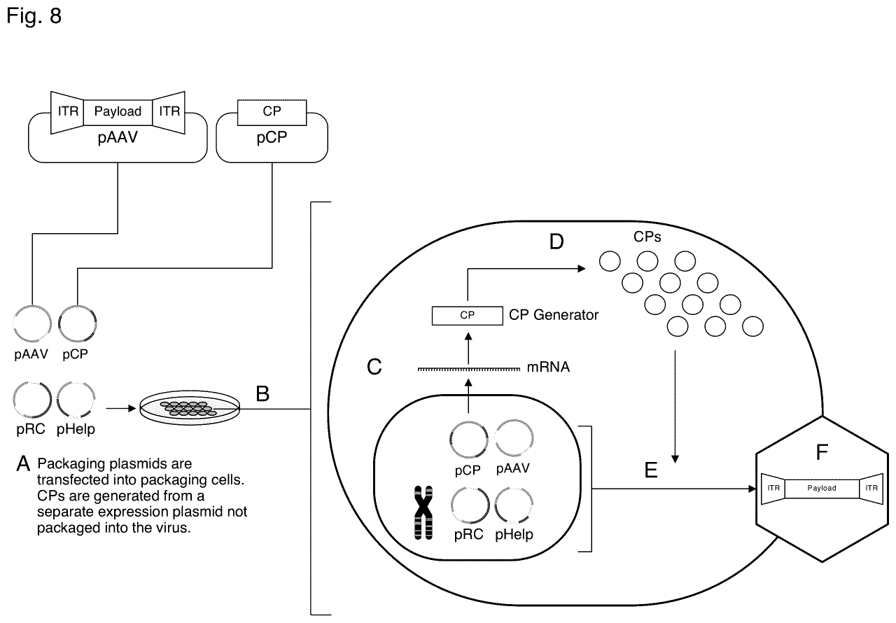

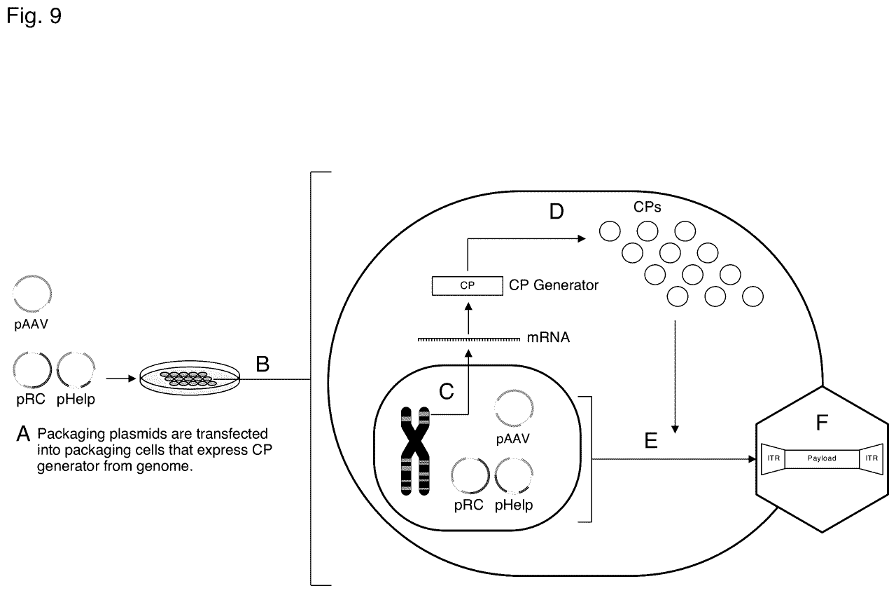

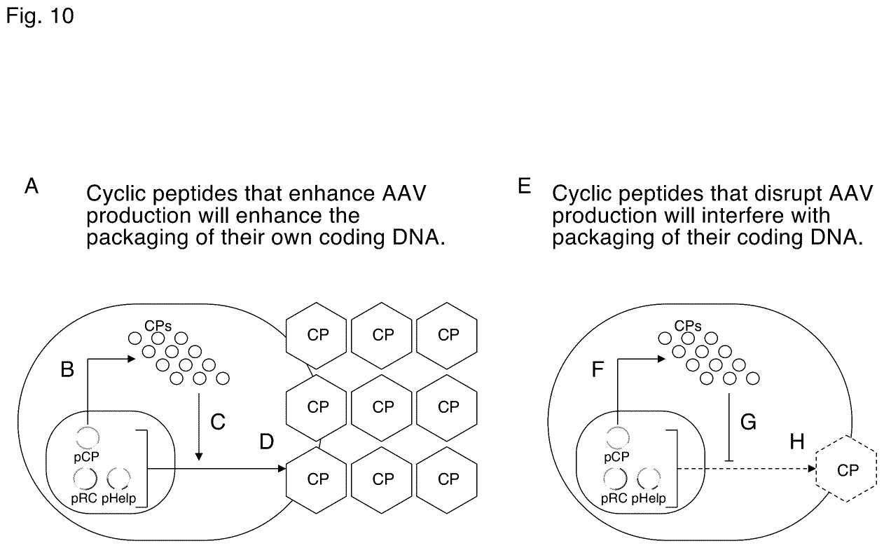

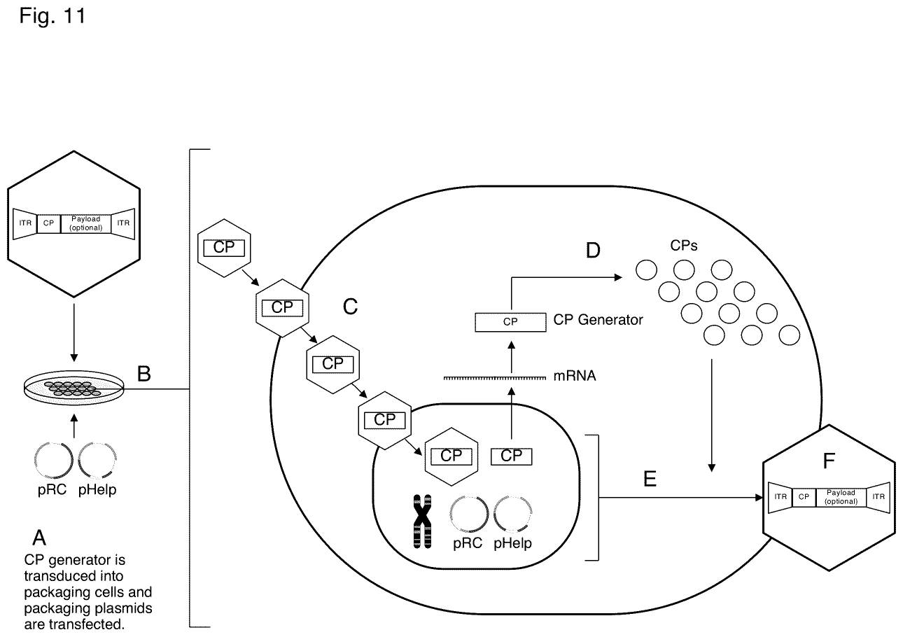

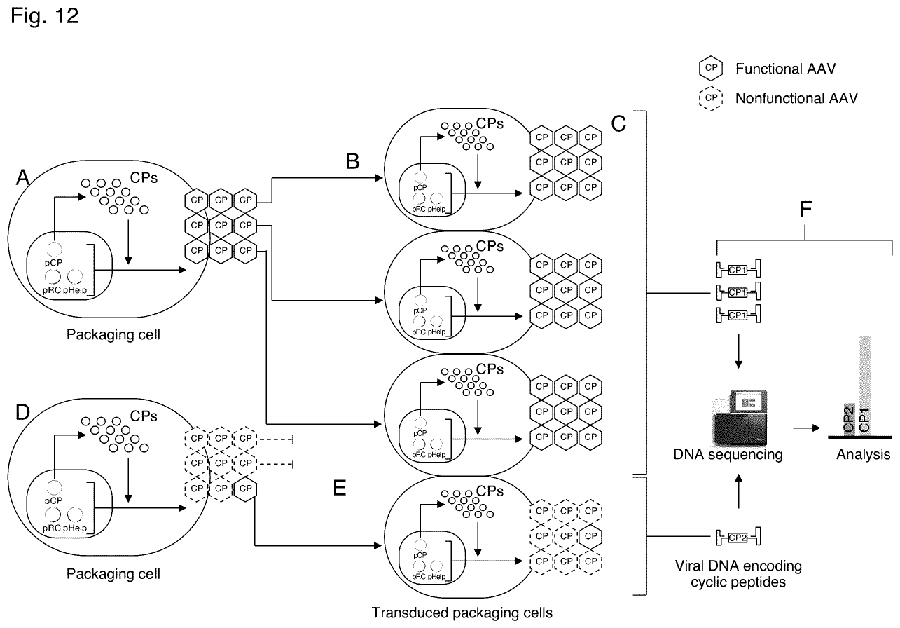

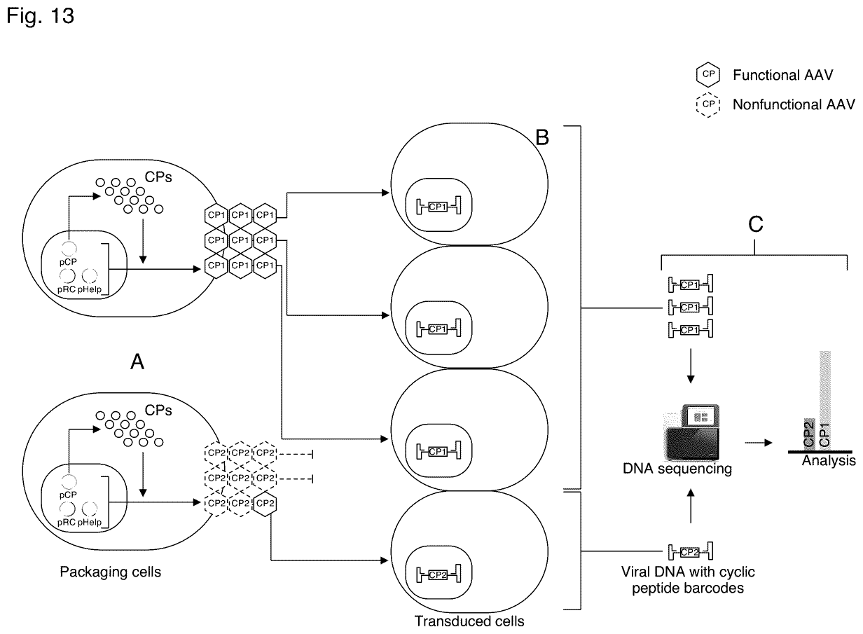

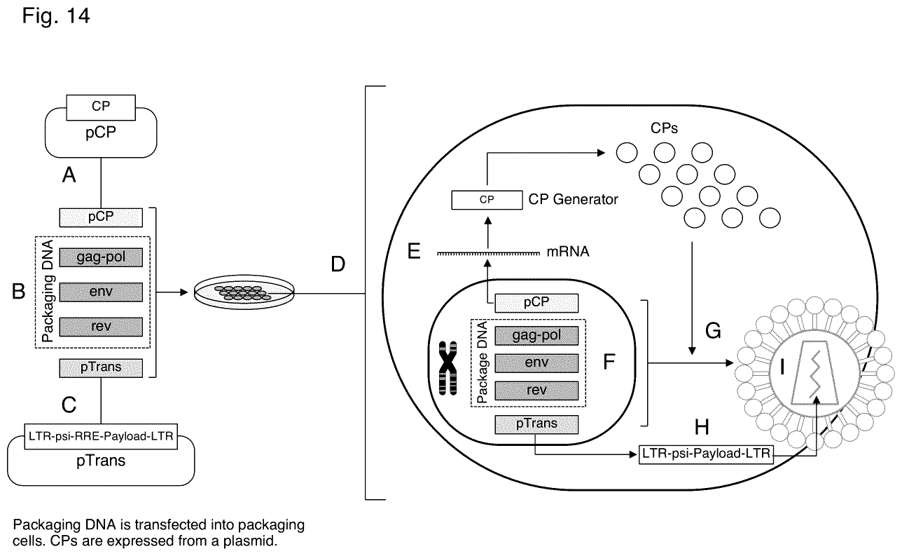

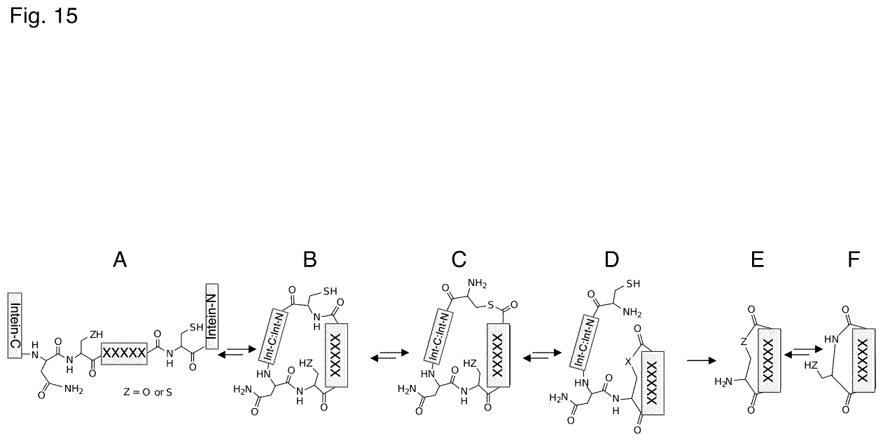

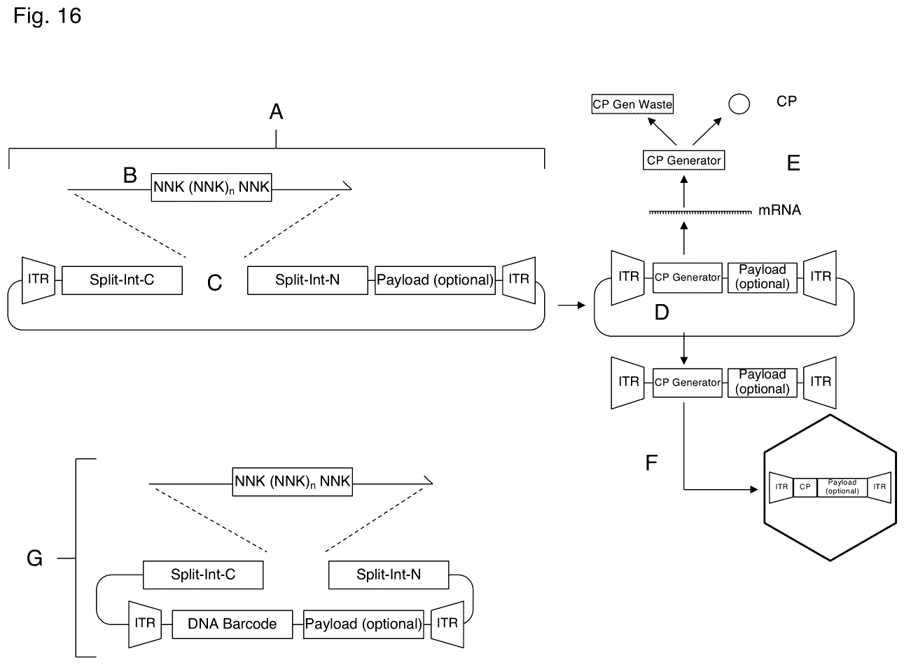

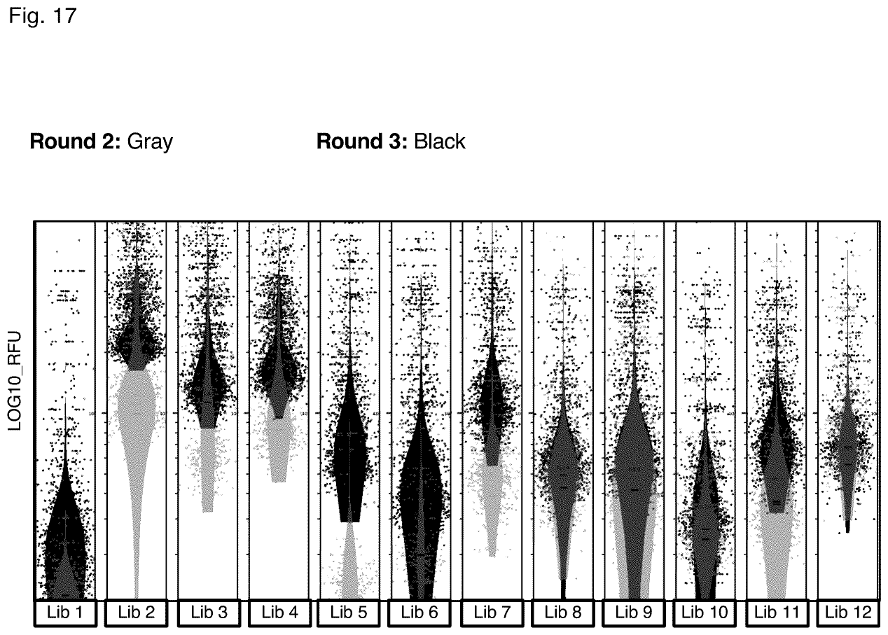

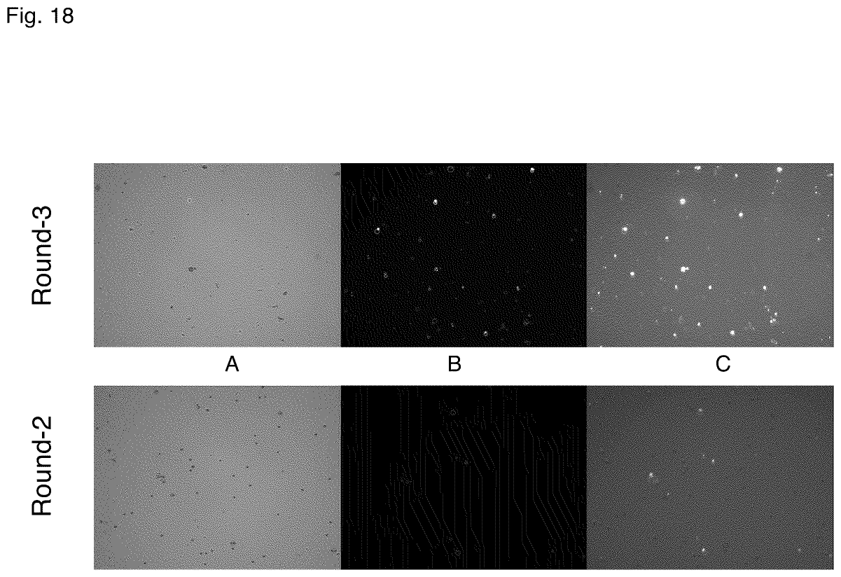

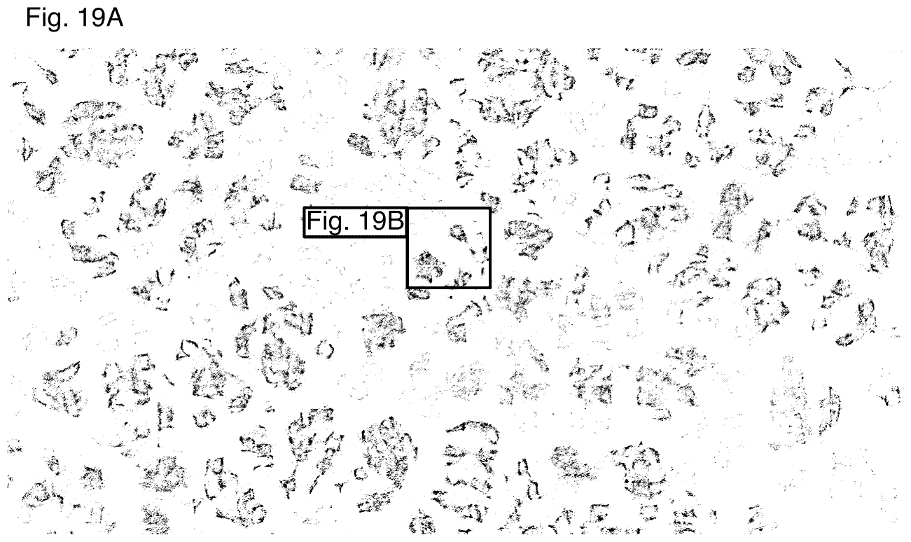

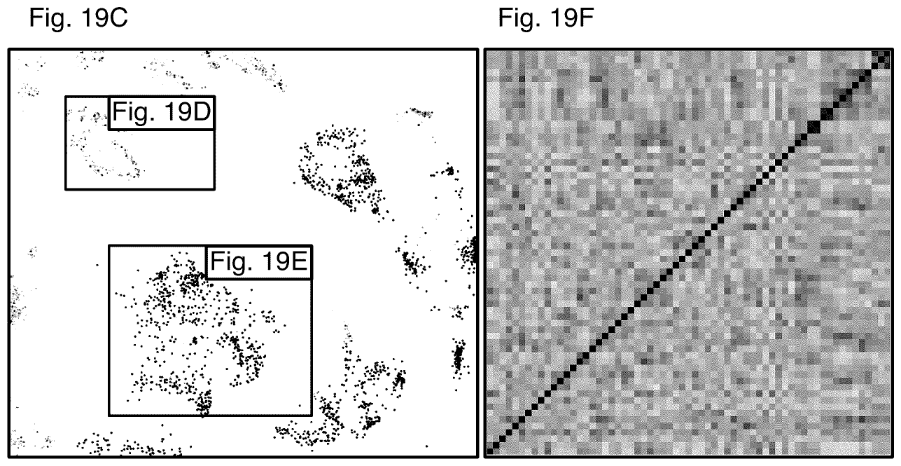

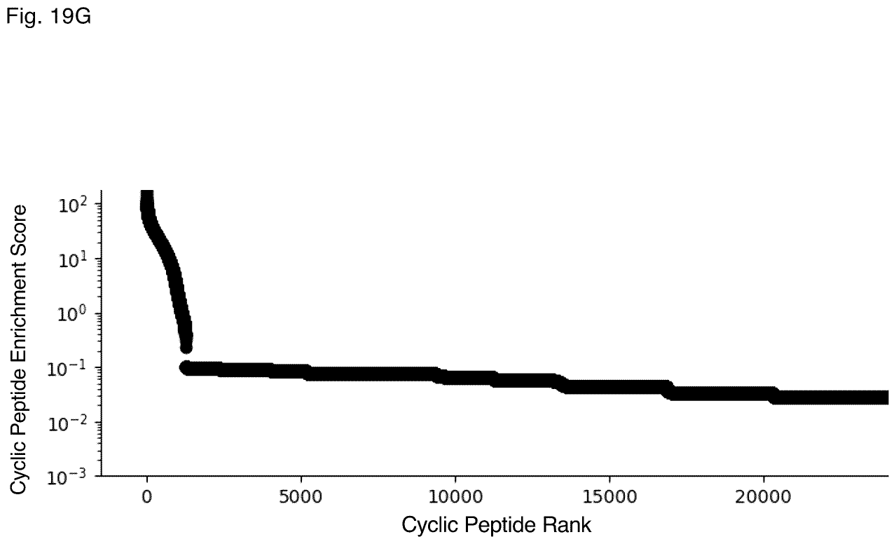

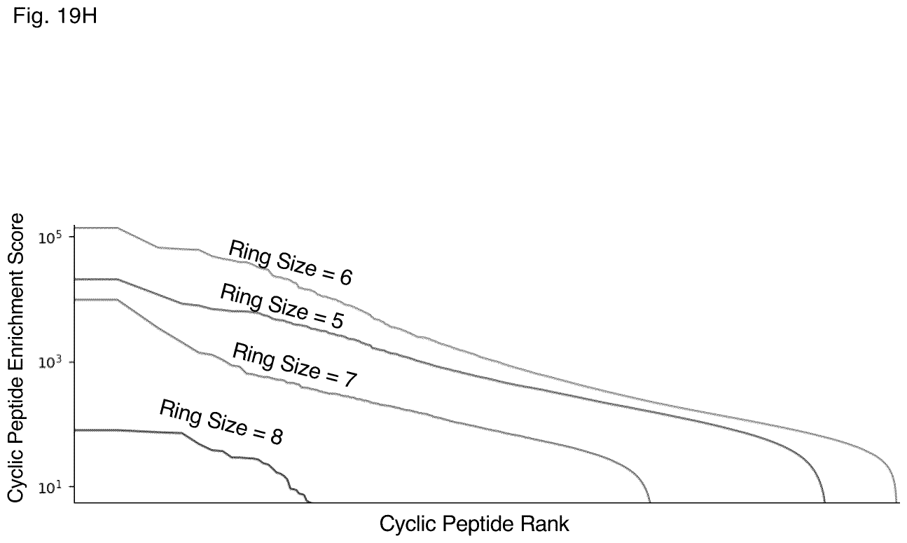

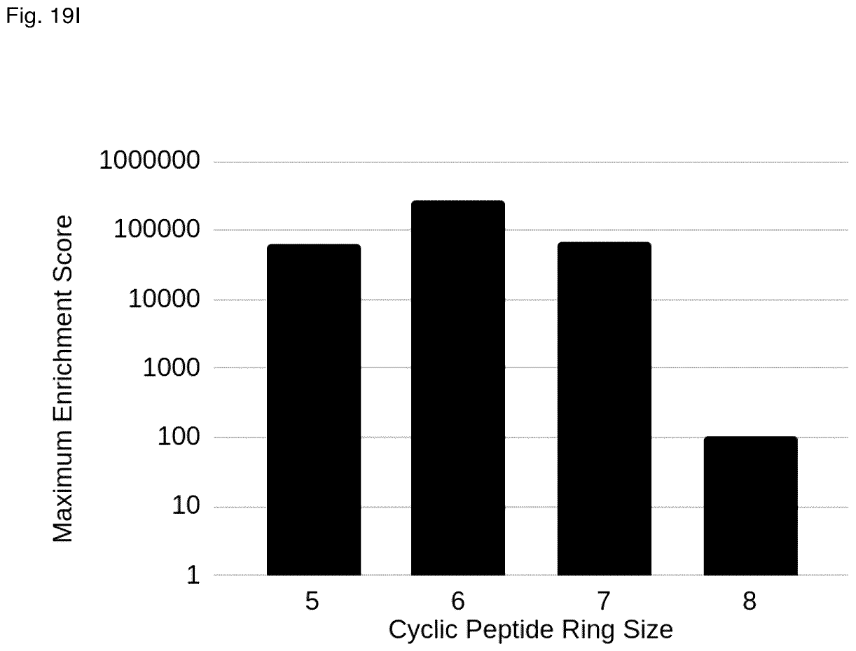

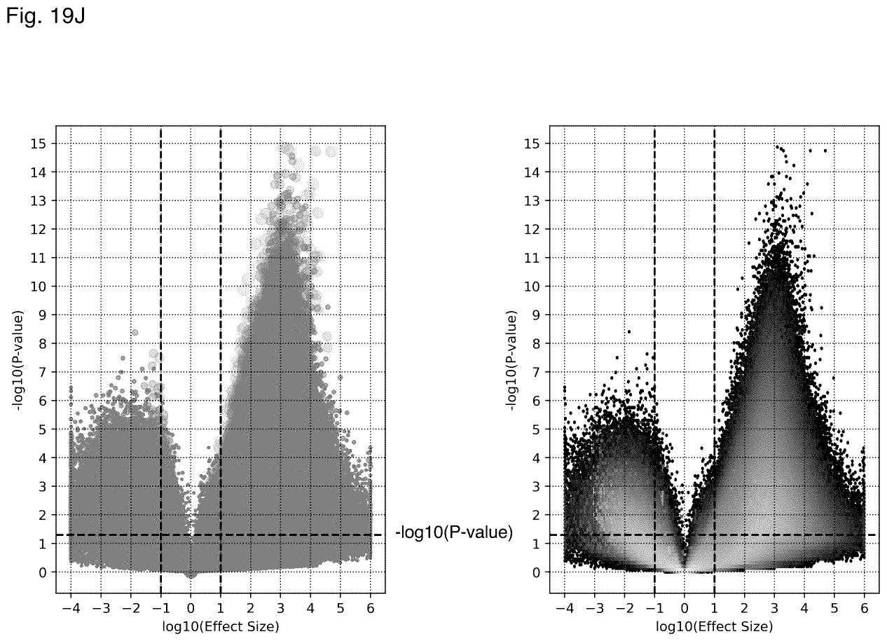

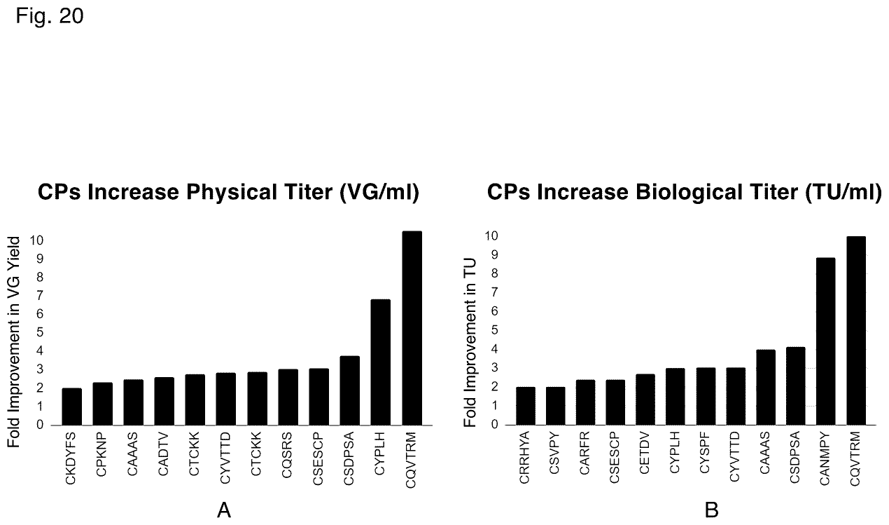

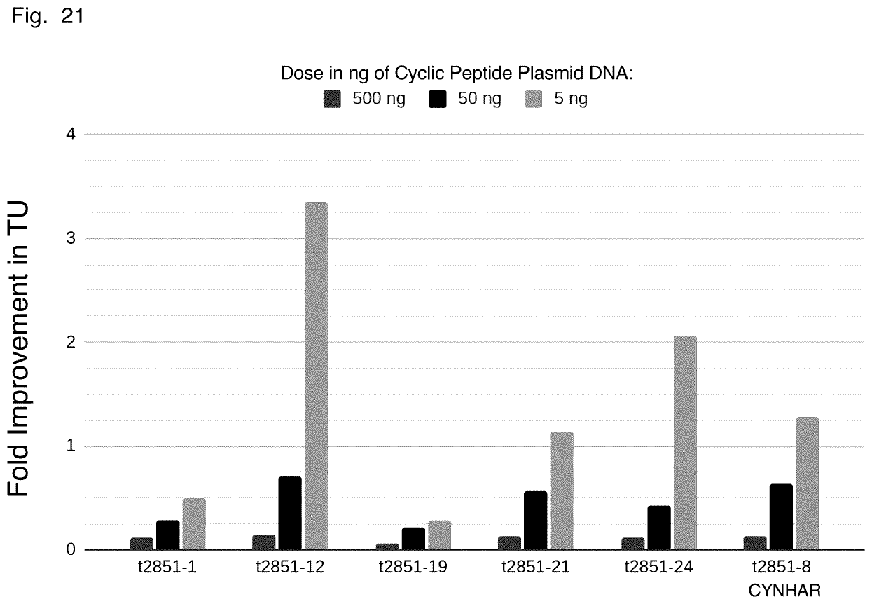

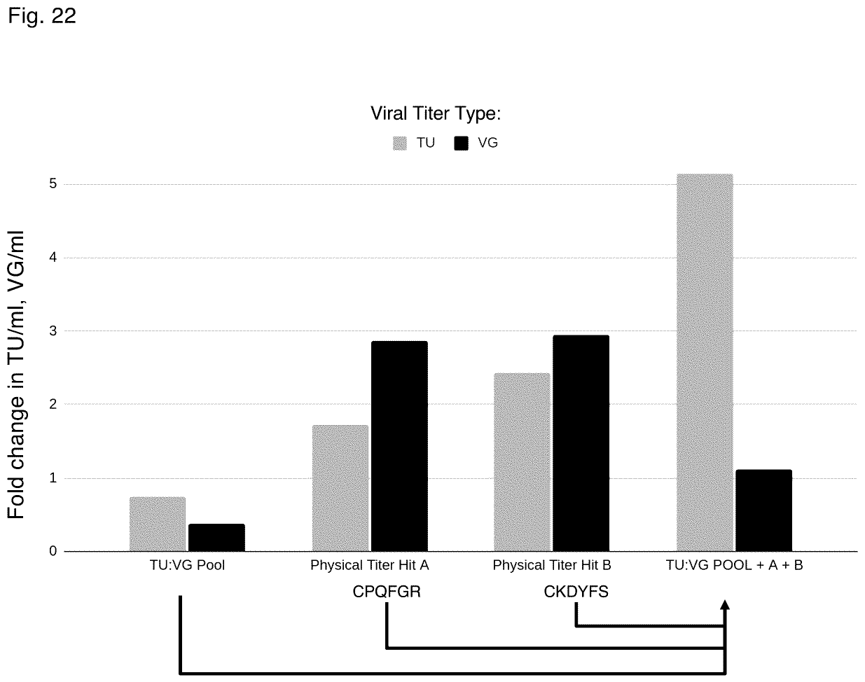

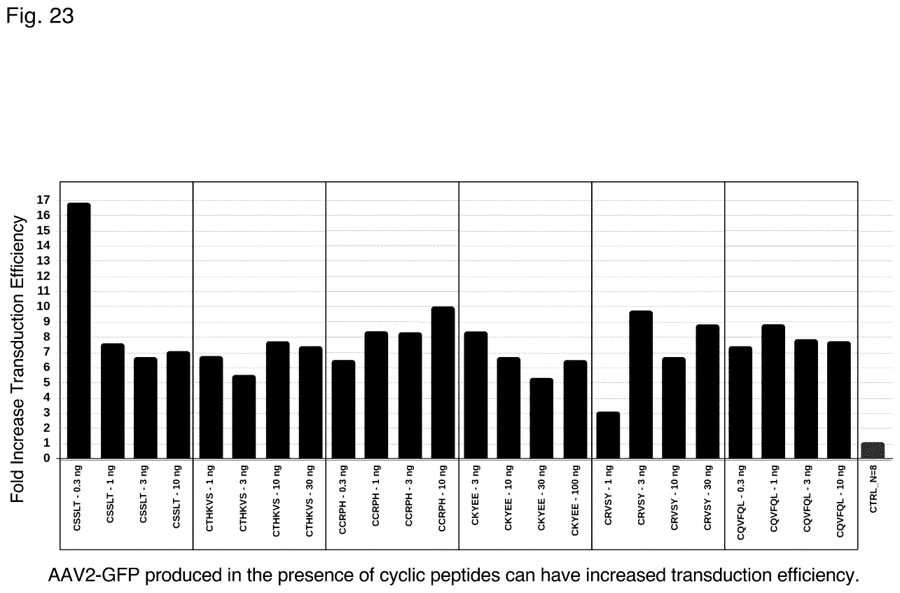

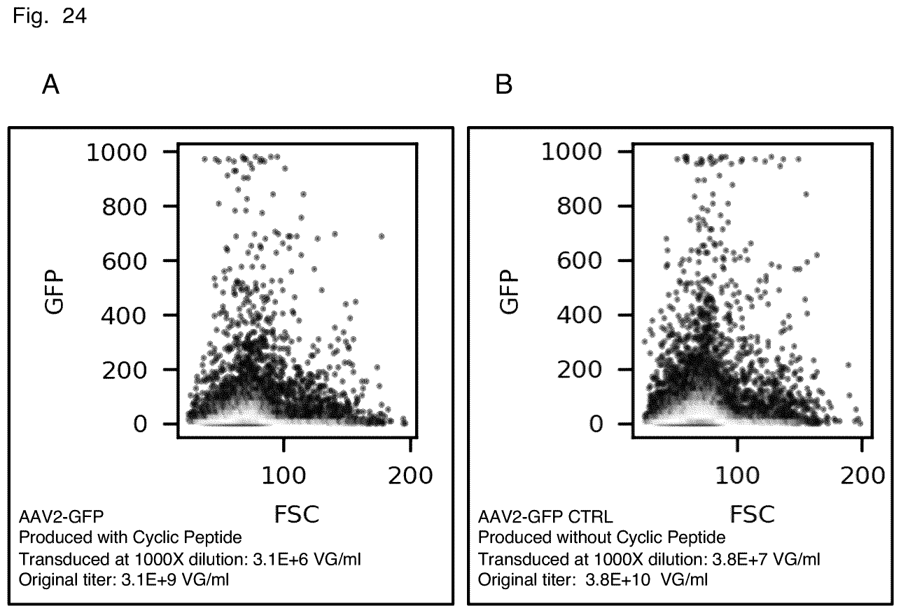

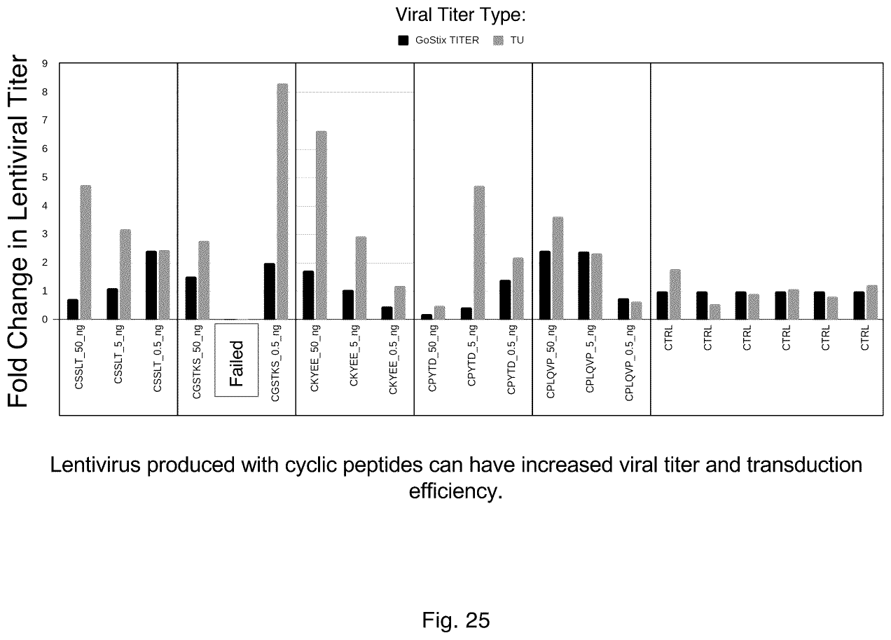

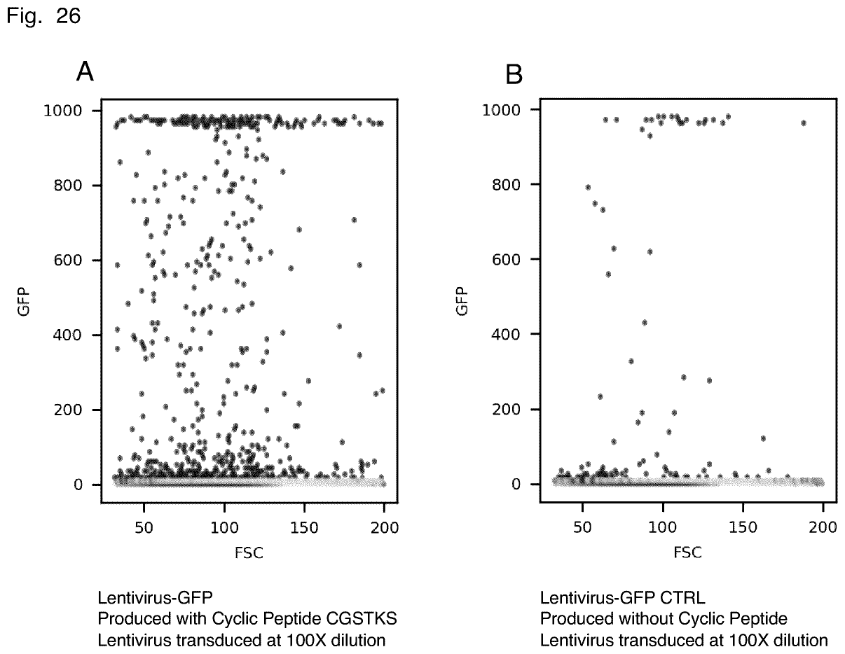

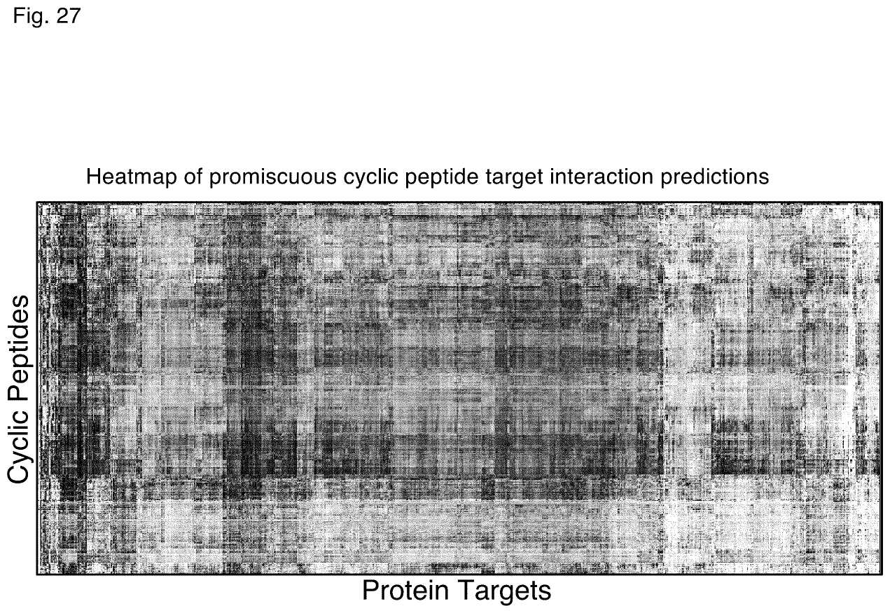

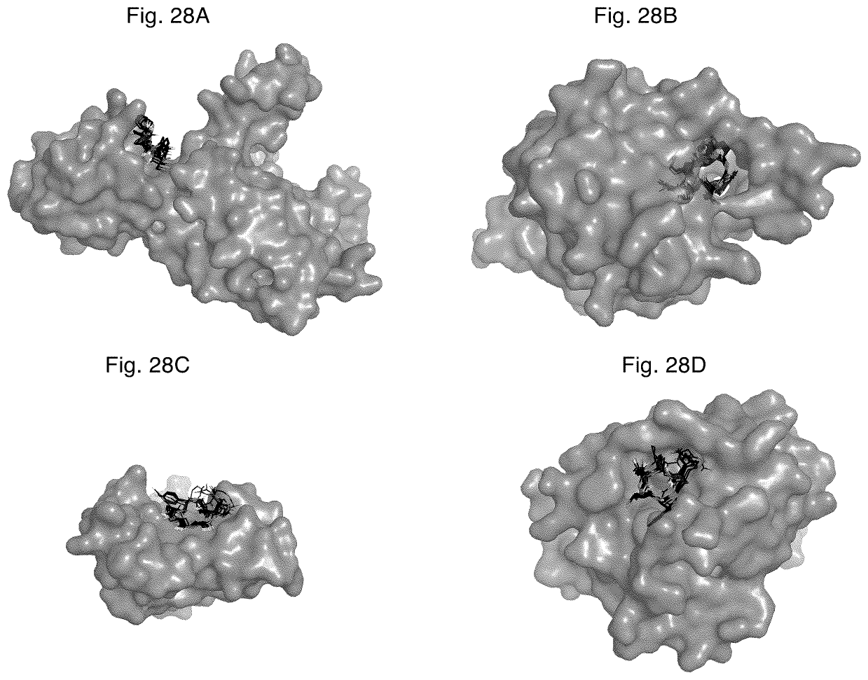

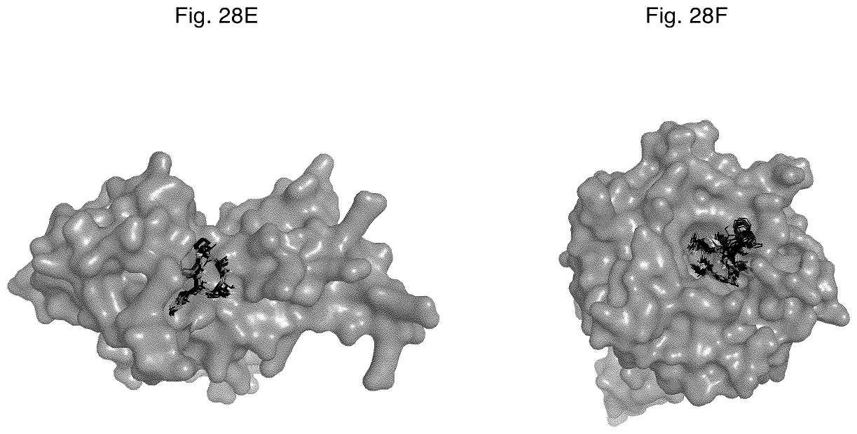

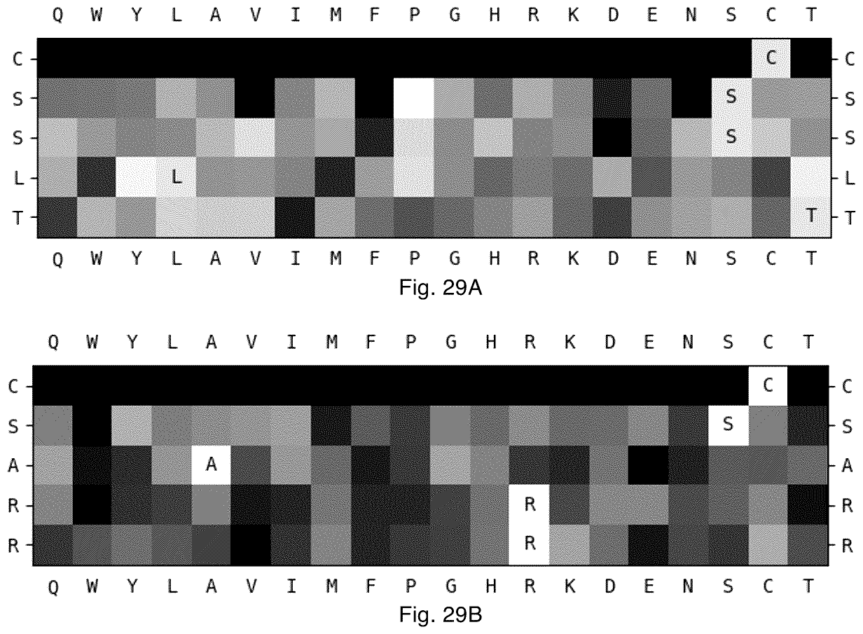

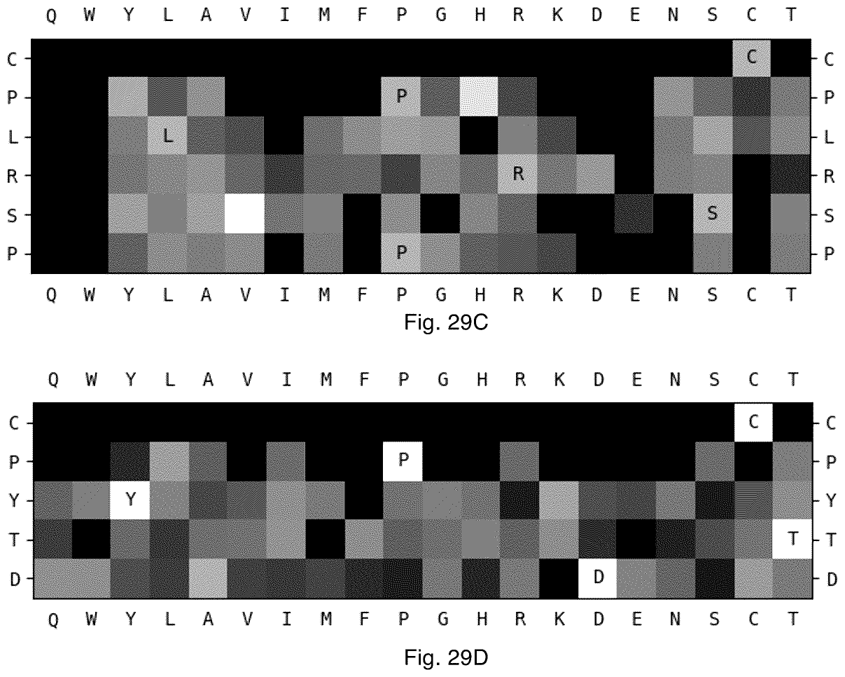

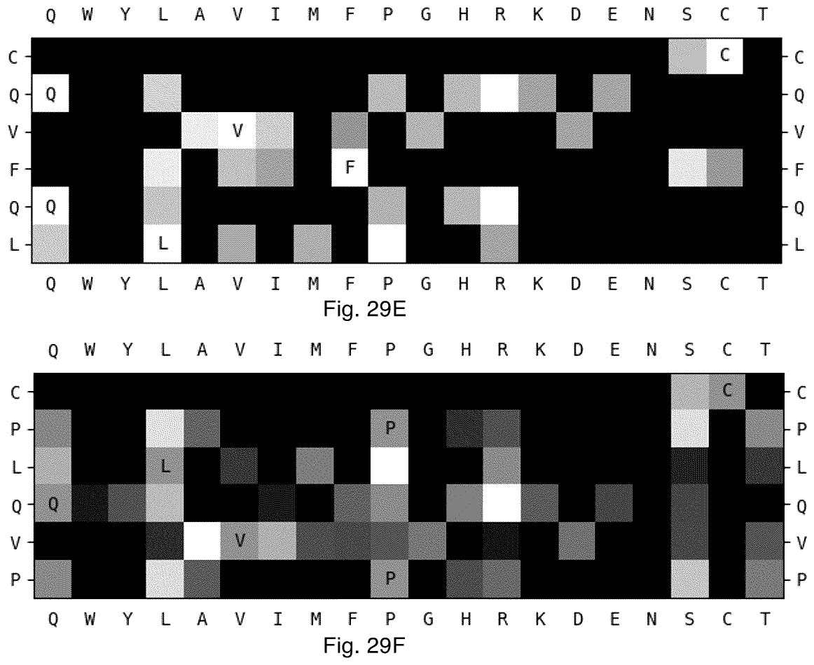

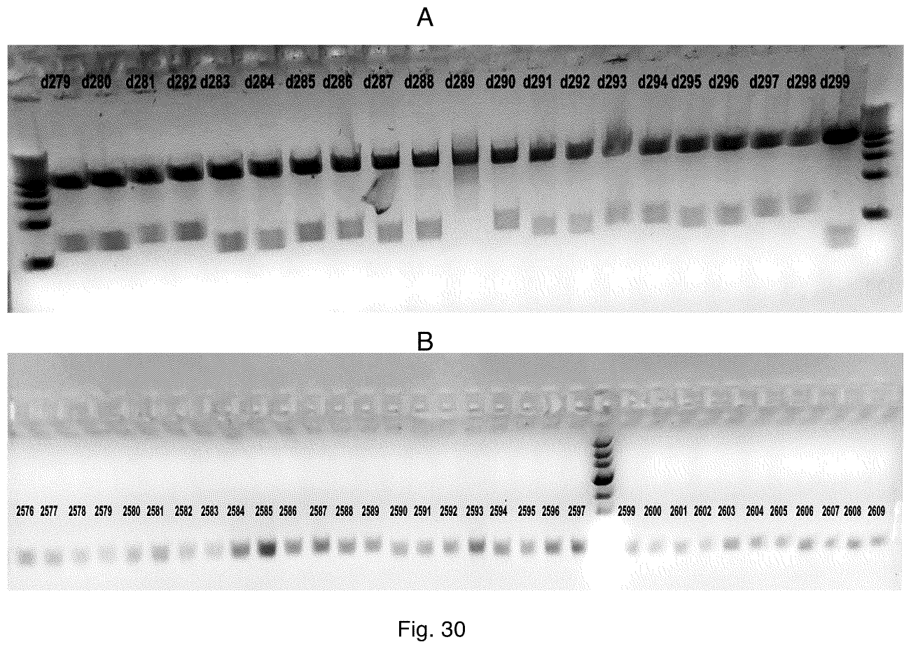

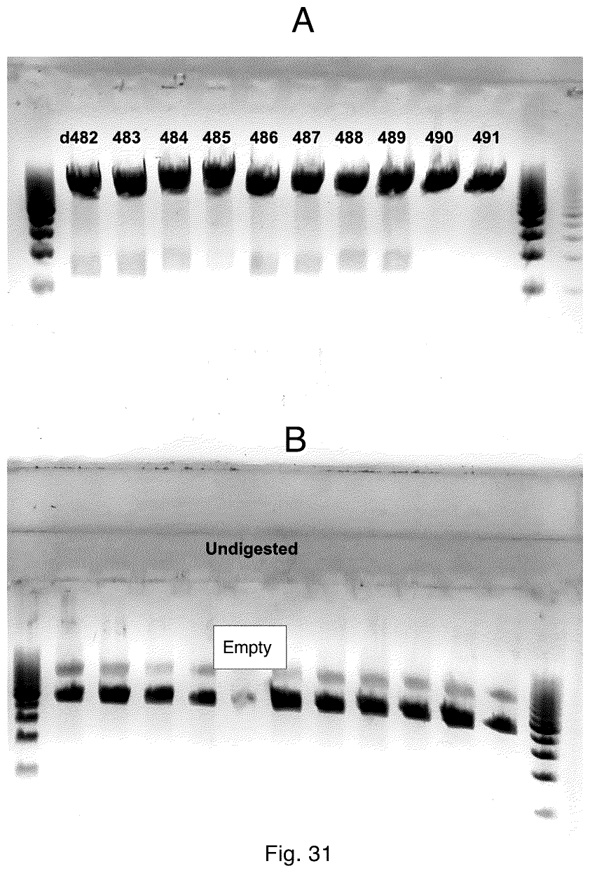

In some embodiments, the cyclic peptides are discovered in a lentiviral enrichment scheme (e.g., FIG. 1—FIG. 13 can be adapted to what is shown in FIG. 14).

In some embodiments, the method is scaled up for large-scale industrial production of lentiviral vectors. This expansion might involve the use of industrial-scale bioreactors or other cell culture systems capable of handling large volumes, to enhance the production capacity of lentiviral vectors for widespread commercial or clinical use.

In some embodiments, the cyclic peptides can be altered or engineered to improve their stability or activity during the packaging process. Such engineering can include modifications to the amino acid sequence (e.g., FIG. 29) or structure of the peptides (e.g., non-canonical side chains or N-methylations), which can increase their efficiency in promoting lentiviral vector packaging.

In some embodiments, the cells transduced by lentivirus packaged in the presence of cyclic peptide are human white blood cells.

In some embodiments, the lentivirus packaged in the presence of cyclic peptide harbors one or more of a Chimeric Antigen Receptor (CAR), T-Cell Receptor (TCR), cytokine, gene editing payload (e.g. cas9), interfering RNA, transcription factor.

In some embodiments, the technique can be employed for packaging replication-competent lentiviral vectors. This can enable the production of lentiviral vectors capable of multiple rounds of infection, which may be advantageous in certain research or therapeutic applications.

In some embodiments, the method can be used to enhance the production of lentiviral vectors for gene therapy applications. These can include treatments for genetic diseases, cancers, or other conditions that can benefit from gene-based therapies.

In some embodiments, the cyclic peptides can be designed to interact with specific viral or cellular proteins to boost packaging efficiency. This targeted interaction can enable a more efficient and effective packaging process.

In some embodiments, the cyclic peptides might be modified to enhance their resistance to degradation during the packaging process. This can include chemical modifications that increase the stability of the peptides, leading to enhanced longevity and function during lentiviral vector production.

In specific embodiments, the cyclic peptides can be incorporated into a packaging cell line to create a stable producer cell line for continuous production of lentiviral vectors. This can simplify the production process by eliminating the need for repeated transfections of the cyclic peptide generator.

In some embodiments, the cyclic peptides can be utilized to enhance the production of lentiviral vectors for research applications, such as gene functional studies or disease modeling. This can significantly improve the capability and utility of lentiviral vectors as tools for biological research.

In some embodiments, the cyclic peptides are part of a lentiviral packaging kit. In some embodiments, the cyclic peptides are part of a lentiviral packaging kit.

In any of the embodiments herein, a kit disclosed herein may further comprise one or more additional components necessary for carrying out a method described herein, such as sample preparation reagents, buffers, labels, and the like. As such, the kits may include one or more containers such as vials or bottles, with each container containing one or more separate components of the kit, and reagents for carrying out one or more steps of a method described herein. The kits may also include a denaturation reagent, buffers such as binding buffers and hybridization buffers, wash mediums, enzyme substrates, reagents for generating a labeled molecule, negative and positive controls and written instructions for using the kit components for carrying out a method, for example, for analyzing a polypeptide as described herein. The instructions may be printed on a substrate, such as paper or plastic, etc. As such, the instructions may be present in the kits as a package insert, in the labeling of the container of the kit or components thereof (e.g., associated with the packaging or sub-packaging) etc. Any one or more of the kit components and instructions may be packaged, stored, and/or shipped separately from other kit components and instructions, or together with any one or more other kit components and instructions.

In some embodiments, pseudotyped lentiviral vectors, which use envelope proteins derived from other viruses to expand tropism, can be supplemented with cyclic peptides to further boost their transduction efficiency and target cell specificity.

In some embodiments, in specific scenarios, chimeric lentiviral vectors, which combine elements from different viruses, may be augmented with cyclic peptides to enhance their specific characteristics, such as nuclear localization from Adeno-Associated Virus elements.

In various embodiments, self-inactivating (SIN) lentiviral vectors, known for their safety through modifications that render them self-inactivating, might utilize cyclic peptides to increase their packaging efficiency, maintaining their safety profile while improving lentiviral vector yield.

In some embodiments, inducible lentiviral vectors, which allow temporal control of transgene expression through inducible promoter elements, can benefit from the addition of cyclic peptides to further increase their overall efficiency and performance.

In some embodiments, the cyclic peptide enhances packaging of a chimeric or pseudotyped virus. In further embodiments, the chimeric or pseudotyped virus may be related.

In some embodiments, the cyclic peptide enhances packaging of a nested virus. In further embodiments the nested virus is an AAV virus nested inside of an Adenovirus. In further embodiments the nested virus is an AAV virus nested inside of an HSV. In further embodiments the nested virus is an AAV virus nested inside of an HPV. In further embodiments the nested virus is an AAV virus nested inside of an Lentivirus. In further embodiments the nested virus is an AAV virus nested inside of an Retrovirus. In further embodiments the nested virus is an AAV nested inside of a baculovirus. In further embodiments the nested virus is a lentivirus nested inside of an HSV. In further embodiments the nested virus is a retrovirus nested inside of an HSV. In further embodiments the nested virus is an Adenovirus nested inside of an HSV. In further embodiments the nested virus construct is able to support viral packaging of both viruses. In further embodiments the nested virus construct is integrated into the host cell genome. In further embodiments the nested virus construct is self-limiting. In further embodiments the nested virus construct is self-limiting through use of a chemical dependency. In further embodiments, the nested virus construct is self-limiting in certain cell lines, but not other cell lines. In further embodiments, the nested virus construct is self-limiting in certain cell lines, but not other cell lines based on the presence of cyclic peptides. In further embodiments, the nested virus construct is self-limiting in certain cell lines, but not other cell lines.

In some embodiments cyclic peptides that increase production of one virus serotype (e.g., AAV1) may also increase production of a second, related virus serotype (e.g., AAV2).

In some embodiments cyclic peptides that increase production of one virus (e.g., AAV) may also increase production of a second, unrelated virus (e.g., lentivirus).

In some embodiments, the cyclic peptide enrichment/discovery process may alternate between different viruses (e.g., AAV, Lentivirus, HSV, Adenovirus, Baculovirus) as well as different serotypes of different viruses (e.g., AAV1, AAV5, AAV8, AAV9, Adenovirus 1-5, HIV, FIV, BIV, HSV1, HSV2). In some embodiments, the cyclic peptide enrichment/discovery process may include enrichment for cyclic peptides that are indirectly related viral production. In some embodiments, cyclic peptides may be enriched for those that are non-toxic to host cells.

In some embodiments, the cyclic peptide may enhance production of a provirus that is integrated onto the host cell genome. In further embodiments, the provirus is a lentivirus. In further embodiments, the provirus is a retrovirus. In further embodiments the provirus is an AAV. In further embodiments, the provirus is a nested virus. In further embodiments, the provirus is a non-integrating virus nested inside of a provirus.

In another exemplary implementation of the present invention, a combinatorial cyclic peptide (CP) library is generated employing a solid phase synthesis methodology. This CP library is synthesized on a substrate, which in a particular aspect, includes beads. Each bead, advantageously, accommodates one or a limited number of CP sequences (preferably not exceeding 10 sequences, most preferably 1 sequence per bead). Moreover, the bead carrying CPs is also equipped with a unique DNA barcode specific to the CP sequence. This unique DNA barcode facilitates the identification of the CPs associated with a particular bead and can be used in identifying the functional impact of the CP on viral production.

In a subsequent phase, the CP library, synthesized through the solid phase method, is mixed with a plurality of cells. These cells are configured to produce viral vectors. In preferred embodiments, the CP bead library and cells are mixed using microfluidic droplet encapsulation so that one bead is mixed with one packaging cell. CPs are then liberated from the beads within the droplets (preferably by cleavage of a photo-cleavable linker), allowing them to interact with, and influence, the viral packaging cells that are producing viral vectors.

After the viral packaging step has occurred, a processing step is employed to release of nucleic acids from the cells and viruses within each droplet (heat treatment is the preferred embodiment for simplicity). This results in the RNA and DNA from both the cells and viruses to mix with the CP-identifying DNA barcodes that are linked to the solid phase synthesis bead. This enables a standard single-cell sequencing protocol to be followed. The DNA barcodes are linked to the viral and cell nucleic acids (e.g. homology to poly A tails for RNA, or viral-specific sequences) allowing analysis of viral amplification as well as the cell state of the packaging cell by measurement of the transcriptional profile, which will contain information about both the cell and viral gene expression.

Lastly, an evaluation is conducted to assess the impact of the solid phase synthesis on viral packaging, which is achieved by analyzing DNA sequencing. In particular, the ratio of viral nucleic acids to packaging cell nucleic acids can be exploited as an indication of the single-cell viral packaging efficiency under the influence of a particular cyclic peptide.

In some embodiments, nucleic acid dyes are incorporated into the droplets. This incorporation facilitates the categorization of each micro-packaging experiment based on nucleic acid population. In other embodiments, protein dyes are added to the droplets allowing evaluation of total protein content and calculation of protein:nucleic acid ratios. In further embodiments, the droplets are microfluidically sorted using the dyes after viral packaging, but before the single cell sequencing.

In some embodiments, the CP library is generated using a solid phase synthesis approach. In further embodiments, the CP library is synthesized on beads, with one or less than 10 CPs per bead. In further embodiments, the CP bead additionally comprises a DNA barcode to identify the 1 or more CPs.

In further embodiments, the solid phase synthesized CP library is mixed with cells configured to produce viral vectors and are microfluidically encapsulated (i.e. droplet encapsulation).

In further embodiments, the solid phase synthesized CPs are released from the bead so that they may exert their influence on the packaging cells.

In further embodiments, nucleic acids from the cell and viral material in each droplet are released by heat-activatable enzymatic treatment, so that the RNA and DNA from the cell and virus are mixed with the CP DNA barcode (preferably linked to and coating the solid phase CP synthesis bead).

In further embodiments, nucleic acid dyes are added to the droplets to sort each micro-packaging experiment by nucleic acid outputs.

In further embodiments, sorted droplets (both positive and negative) are sequenced after a DNA barcode ligation reaction tags all nucleic acids in a droplet

In further embodiments, the effect of solid phase synthesis on viral packaging is evaluated by analyzing DNA synthesis.

Disclosed herein is also a viral packaging kit comprising at least one nucleotide sequence containing:
(i) at least one AAV replication gene;
(ii) at least one AAV capsid encoding gene;
(iii) at least one AAV helper gene; and
(iv) at least one gene that expresses a polypeptide configured to cyclize, wherein (i)-(iv) are provided on one or more expression plasmids and, when transfected into packaging cells, produces a viral vector composition having a characteristic, which is at least 2-fold higher than a corresponding characteristic of a reference viral vector produced under essentially identical conditions, wherein each reference host cell of the plurality of reference host cells comprises the elements (i)-(iii) of the first plurality of host cells and does not comprise (iv).

Disclosed herein is also a viral packaging kit comprising at least one nucleotide sequence containing:
(i) at least one AAV replication gene;
(ii) at least one AAV capsid encoding gene;
(iii) at least one AAV helper gene; and
(iv) at least one cyclic peptide reagent, wherein (i)-(iii) are provided on one or more expression plasmids and, when transfected into packaging cells with (iv), produces a viral vector composition having a characteristic, which is at least 2-fold higher than a corresponding characteristic of a reference viral vector produced under essentially identical conditions, wherein each reference host cell of the plurality of reference host cells comprises the elements (i)-(iii) of the first plurality of host cells and does not comprise (iv).

Disclosed herein is also a viral packaging cell line kit comprising the following:
(i) a mammalian cell line;
(ii) at least one AAV replication gene, genomically integrated;
(iii) at least one AAV capsid encoding gene, genomically integrated;
(iv) at least one AAV helper gene, genomically integrated; and
(v) at least one genomically integrated gene that expresses a polypeptide configured to cyclize, wherein, at least one of (i)-(v) is under inducible expression control and upon induction, the cell line produces a viral vector composition having a characteristic, which is at least 2-fold higher than a corresponding characteristic of a reference viral vector produced under essentially identical conditions, wherein each reference host cell of the plurality of reference host cells comprises the elements (i)-(iv) of the first plurality of host cells and does not comprise (v).

Disclosed herein is also a viral packaging kit comprising at least one nucleotide sequence containing:
(i) at least one lentiviral pol gene;
(ii) at least one lentiviral gag gene and at least one env gene;
(iii) at least lentiviral rev gene; and
(iv) at least one gene that expresses a polypeptide configured to cyclize, wherein (i)-(iv) are provided on one or more expression plasmids and, when transfected into packaging cells, produces a viral vector composition having a characteristic, which is at least 2-fold higher than a corresponding characteristic of a reference viral vector produced under essentially identical conditions, wherein each reference host cell of the plurality of reference host cells comprises the elements (i)-(iii) of the first plurality of host cells and does not comprise (iv).

Disclosed herein is also a viral packaging kit comprising at least one nucleotide sequence containing:
(i) at least one lentiviral pol gene;
(ii) at least one lentiviral gag gene and at least one env gene;
(iii) at least lentiviral rev gene; and
(iv) at least one cyclic peptide reagent, wherein (i)-(iii) are provided on one or more expression plasmids and, when transfected into packaging cells with (iv), produces a viral vector composition having a characteristic, which is at least 2-fold higher than a corresponding characteristic of a reference viral vector produced under essentially identical conditions, wherein each reference host cell of the plurality of reference host cells comprises the elements (i)-(iii) of the first plurality of host cells and does not comprise (iv).

Disclosed herein is also a viral packaging cell line kit comprising the following:
(i) a mammalian cell line;
(ii) at least one AAV replication gene, genomically integrated;
(iii) at least one AAV capsid encoding gene, genomically integrated;
(iv) at least one AAV helper gene, genomically integrated; and
(v) at least one genomically integrated gene that expresses a polypeptide configured to cyclize, wherein, at least one of (i)-(v) is under inducible expression control and upon induction, the cell line produces a viral vector composition having a characteristic, which is at least 2-fold higher than a corresponding characteristic of a reference viral vector produced under essentially identical conditions, wherein each reference host cell of the plurality of reference host cells comprises the elements (i)-(iv) of the first plurality of host cells and does not comprise (v).

Disclosed herein is also a viral packaging cell line kit comprising the following:
(i) a mammalian cell line;
(ii) at least one lentiviral pol gene, genomically integrated;
(iii) at least one lentiviral gag gene and at least one env gene, genomically integrated;
(iv) at least one lentiviral rev gene, genomically integrated; and
(v) at least one genomically integrated gene that expresses a polypeptide configured to cyclize, wherein, at least one of (i)-(v) is under inducible expression control and upon induction, the cell line produces a viral vector composition having a characteristic, which is at least 2-fold higher than a corresponding characteristic of a reference viral vector produced under essentially identical conditions, wherein each reference host cell of the plurality of reference host cells comprises the elements (i)-(iv) of the first plurality of host cells and does not comprise (v).

Disclosed herein is also a kit that comprises at least one engineered cyclic peptide capable of increasing viral titer and/or transduction efficiency of a viral vector composition. In some embodiments, the kit disclosed herein is for obtaining a viral vector composition of increased viral titer and/or transduction efficiency. In some particular embodiments, the kit disclosed herein is for obtaining an AAV vector composition of increased viral titer and/or transduction efficiency. In other particular embodiments, the kit disclosed herein is for obtaining a lentivirus vector composition of increased viral titer and/or transduction efficiency. In some particular embodiments, the kit disclosed herein comprises at least one engineered cyclic peptide capable of increasing viral titer and/or transduction efficiency of a viral vector composition, the at least one engineered cyclic peptide having between 4 and 20 amino acid residues and comprising one or more of the following peptide motifs selected from the group consisting of: ARO_HYD_POL-motif: [‘F’, ‘W’, ‘Y’] followed by [‘G’, ‘A’, ‘V’, ‘I’, ‘L’, ‘M’, ‘P’] followed by [‘S’, ‘T’, ‘Q’, ‘N’, ‘C’]; ARO_POL_ARO-motif: [‘F’, ‘W’, ‘Y’] followed by [‘S’, ‘T’, ‘Q’, ‘N’, ‘C’] followed by [‘F’, ‘W’, ‘Y’]; ARO_POL_POS-motif: [‘F’, ‘W’, ‘Y’] followed by [‘S’, ‘T’, ‘Q’, ‘N’, ‘C’] followed by [‘K’, ‘R’, ‘H’]; HYD_ARO_POL-motif: [‘G’, ‘A’, ‘V’, ‘I’, ‘L’, ‘M’, ‘P’] followed by [‘F’, ‘W’, ‘Y’] followed by [‘S’, ‘T’, ‘Q’, ‘N’, ‘C’]; HYD_HYD_POL-motif: [‘G’, ‘A’, ‘V’, ‘I’, ‘L’, ‘M’, ‘P’] followed by [‘G’, ‘A’, ‘V’, ‘I’, ‘L’, ‘M’, ‘P’] followed by [‘S’, ‘T’, ‘Q’, ‘N’, ‘C’]; HYD_NEG_POL-motif: [‘G’, ‘A’, ‘V’, ‘I’, ‘L’, ‘M’, ‘P’] followed by [‘D’, ‘E’] followed by [‘S’, ‘T’, ‘Q’, ‘N’, ‘C’]; HYD_POS_POL-motif: [‘G’, ‘A’, ‘V’, ‘I’, ‘L’, ‘M’, ‘P’] followed by [‘K’, ‘R’, ‘H’] followed by [‘S’, ‘T’, ‘Q’, ‘N’, ‘C’]; NEG_HYD_POL-motif: [‘D’, ‘E’] followed by [‘G’, ‘A’, ‘V’, ‘I’, ‘L’, ‘M’, ‘P’] followed by [‘S’, ‘T’, ‘Q’, ‘N’, ‘C’]; NEG_POL_ARO-motif: [‘D’, ‘E’] followed by [‘S’, ‘T’, ‘Q’, ‘N’, ‘C’] followed by [‘F’, ‘W’, ‘Y’]; NEG_POL_NEG-motif: [‘D’, ‘E’] followed by [‘S’, ‘T’, ‘Q’, ‘N’, ‘C’] followed by [‘D’, ‘E’]; NEG_POL_POS-motif: [‘D’, ‘E’] followed by [‘S’, ‘T’, ‘Q’, ‘N’, ‘C’] followed by [‘K’, ‘R’, ‘H’]; POL_HYD_ARO-motif: [‘S’, ‘T’, ‘Q’, ‘N’, ‘C’] followed by [‘G’, ‘A’, ‘V’, ‘I’, ‘L’, ‘N’, ‘P’] followed by [‘F’, ‘W’, ‘Y’]; POL_HYD_HYD-motif: [‘S’, ‘T’, ‘Q’, ‘N’, ‘C’] followed by [‘G’, ‘A’, ‘V’, ‘I’, ‘L’, ‘M’, ‘P’] followed by [‘G’, ‘A’, ‘V’, ‘I’, ‘L’, ‘M’, ‘P’]; POL_HYD_NEG-motif: [‘S’, ‘T’, ‘Q’, ‘N’, ‘C’] followed by [‘G’, ‘A’, ‘V’, ‘I’, ‘L’, ‘M’, ‘P’] followed by [‘D’, ‘E’]; POL_HYD_POL-motif: [‘S’, ‘T’, ‘Q’, ‘N’, ‘C’] followed by [‘G’, ‘A’, ‘V’, ‘I’, ‘L’, ‘M’, ‘P’] followed by [‘S’, ‘T’, ‘Q’, ‘N’, ‘C’]; POL_HYD_POS-motif: [‘S’, ‘T’, ‘Q’, ‘N’, ‘C’] followed by [‘G’, ‘A’, ‘V’, ‘I’, ‘L’, ‘M’, ‘P’] followed by [‘K’, ‘R’, ‘H’]; POL_NEG_ARO-motif: [‘S’, ‘T’, ‘Q’, ‘N’, ‘C’] followed by [‘D’, ‘E’] followed by [‘F’, ‘W’, ‘Y’]; POL_NEG_HYD-motif: [‘S’, ‘T’, ‘Q’, ‘N’, ‘C’] followed by [‘D’, ‘E’] followed by [‘G’, ‘A’, ‘V’, ‘I’, ‘L’, ‘M’, ‘P’]; POL_NEG_NEG-motif: [‘S’, ‘T’, ‘Q’, ‘N’, ‘C’] followed by [‘D’, ‘E’] followed by [‘D’, ‘E’]; POL_NEG_POS-motif: [‘S’, ‘T’, ‘Q’, ‘N’, ‘C’] followed by [‘D’, ‘E’] followed by [‘K’, ‘R’, ‘H’]; POL_POL_ARO-motif: [‘S’, ‘T’, ‘Q’, ‘N’, ‘C’] followed by [‘S’, ‘T’, ‘Q’, ‘N’, ‘C’] followed by [‘F’, ‘W’, ‘Y’]; POL_POL_HYD-motif: [‘S’, ‘T’, ‘Q’, ‘N’, ‘C’] followed by [‘S’, ‘T’, ‘Q’, ‘N’, ‘C’] followed by [‘G’, ‘A’, ‘V’, ‘I’, ‘L’, ‘M’, ‘P’]; POL_POL_NEG-motif: [‘S’, ‘T’, ‘Q’, ‘N’, ‘C’] followed by [‘S’, ‘T’, ‘Q’, ‘N’, ‘C’] followed by [‘D’, ‘E’]; POL_POL_POL-motif: [‘S’, ‘T’, ‘Q’, ‘N’, ‘C’] followed by [‘S’, ‘T’, ‘Q’, ‘N’, ‘C’] followed by [‘S’, ‘T’, ‘Q’, ‘N’, ‘C’]; POL_POL_POS-motif: [‘S’, ‘T’, ‘Q’, ‘N’, ‘C’] followed by [‘S’, ‘T’, ‘Q’, ‘N’, ‘C’] followed by [‘K’, ‘R’, ‘H’]; POS_HYD_POL-motif: [‘K’, ‘R’, ‘H’]followed by [‘G’, ‘A’, ‘V’, ‘I’, ‘L’, ‘M’, ‘P’] followed by [‘S’, ‘T’, ‘Q’, ‘N’, ‘C’]; POS_POL_ARO-motif: [‘K’, ‘R’, ‘H’] followed by [‘S’, ‘T’, ‘Q’, ‘N’, ‘C’] followed by [‘F’, ‘W’, ‘Y’]; and POS_POL_POS-motif: [‘K’, ‘R’, ‘H’] followed by [‘S’, ‘T’, ‘Q’, ‘N’, ‘C’] followed by [‘K’, ‘R’, ‘H’].

In some embodiments, kits disclosed herein comprise at least one engineered cyclic peptide and also comprise at least one non-peptide macromolecule, which is used together with the at least one engineered cyclic peptide to obtain a viral vector composition of increased viral titer and/or transduction efficiency. In some embodiments, the non-peptide macromolecule of the kit is selected from the group consisting of one or more cationic polymers, one or more cationic lipids, and one or more dendrimers. In some embodiments, the components of the kit disclosed herein are used to together in a transfection reagent to transfect a plurality of host cells to increase a characteristic of viral vectors produced by the plurality of host cells, wherein the characteristic of viral vectors is selected from the group consisting of: viral titer and transduction efficiency. In some embodiments, the non-peptide macromolecule of the kit comprises at least one of the moieties selected from the group consisting of: a linear or branched polyethyleneimine (PEI), PEI dendrimer, a polypropyleneimine (PPI), Poly(amidoamine) (PAA) and dendrimers (PAMAM), cationic cyclodextrin, polyalkylamine, a polyhydroxyalkylamine, poly(butyleneimine) (PBI), spermine, a N-substituted polyallylamine, N-substituted chitosan, a N-substituted polyornithine, a N-substituted polylysine (PLL), a N-substituted polyvinylamine, poly(β-amino ester), hyperbranched poly(amino ester) (h-PAE), networked poly(amino ester) (n-PAE), poly(4-hydroxy-1-proline ester) (PHP-ester), a poly-β-aminoacid, 1,2-dioleoyl-3-trimethylammonium-propane (DOTAP), 1,2-dimyristoyl-3-trimethylammonium-propane (DMTAP), 3β-[N-(N′,N′-dimethylaminoethane)-carbamoyl] cholesterol (DC-Chol), N-[1-(2,3-dioleyloxy)propyl]-N,N,N-trimethylammonium chloride (DOTMA), 1,2-dipalmitoyl-3-trimethylammonium-propane (DPTAP), cationic liposome, and dendrimer. In some embodiments, the components of the kit (e.g., the cyclic peptide and at least one of the moieties listed above) are conjugated. In some embodiments, the components of the kit (e.g., the cyclic peptide and the at least one of the moieties listed above) are non-covalently associated with each other (e.g., through hydrogen bonding and/or electrostatic interactions, and so on). Non-limiting examples of dendrimers include poly(amidoamine) (PAMAM) dendrimers, poly(propylene imine) (PPI) dendrimers, polyether-copolyester (PEPE) dendrimers, PEGylated dendrimers and peptide dendrimers. In some embodiments, the components of the kit are used to together in a transfection reagent which is used to deliver viral packaging plasmids into mammalian cells, and where the transfection results in at least a 2-fold higher yield of viral vector composition compared to a reference transfection reagent that that does not contain the CP.

The following enumerated embodiments are representative of the invention:
1. A method of obtaining an engineered cyclic peptide capable of increasing viral titer and/or transduction efficiency of a viral vector composition, the method comprising:
(a) culturing a first plurality of host cells permissive for replication of a virus under conditions suitable for recombinant viral production, wherein each host cell of the first plurality of host cells comprises:
(i) at least one viral replication gene essential for the replication of the virus;
(ii) at least one viral structural gene essential for formation of viral capsids;
(iii) at least one additional viral gene necessary to produce the virus in the host cells; and
(iv) an engineered cyclic peptide produced from a polypeptide encoded by a first nucleotide sequence, wherein (v) the first nucleotide sequence is operably linked to one or more viral-specific packaging sequences necessary for encapsulation of the first nucleotide sequence within the viral capsids or (vi) the first nucleotide sequence is associated with a second nucleotide sequence comprising a barcode that comprises identifying information regarding the engineered cyclic peptide produced in the host cell, and the second nucleotide sequence is operably linked to the one or more viral-specific packaging sequences necessary for encapsulation of the second nucleotide sequence within the viral capsids, thereby obtaining a first plurality of viral vectors comprising the first nucleotide sequence and/or the second nucleotide sequence from the first plurality of host cells;
(b) optionally, repeating the following steps one or more times in cycles:
(b1) allowing a plurality of viral vectors of the previous cycle to infect a plurality of host cells of the present cycle permissive for replication of the virus; and
(b2) culturing the plurality of host cells of the present cycle under conditions suitable for recombinant viral production, wherein each host cell of the plurality of host cells of the present cycle comprises the elements (i)-(iii) of the first plurality of host cells, and further comprises the first nucleotide sequence operably linked to the one or more viral-specific packaging sequences producing the engineered cyclic peptide, thereby obtaining a plurality of viral vectors of the present cycle comprising the first nucleotide sequence;
(c) allowing the first plurality of viral vectors or the plurality of viral vectors of the present cycle to infect a final plurality of host cells; and
(d) determining one or more engineered cyclic peptides capable of increasing viral titer and/or transduction efficiency of the viral vector composition by analyzing nucleotide sequences operably linked to the one or more viral-specific packaging sequences from (i) the final plurality of host cells and/or (ii) a final plurality of viral vectors produced in the final plurality of host cells.
2. The method of embodiment 1, wherein in step (a), the first nucleotide sequence is operably linked to the one or more viral-specific packaging sequences, and the method comprises culturing the final plurality of host cells under conditions suitable for recombinant viral production, wherein each host cell of the final plurality of host cells comprises the elements (i)-(iii) of the first plurality of host cells, and further comprises the first nucleotide sequence operably linked to one or more viral-specific packaging sequences and producing the engineered cyclic peptide encoded by the first nucleotide sequence, thereby producing the final plurality of viral vectors from the final plurality of host cells and determining the engineered cyclic peptide by analyzing nucleotide sequences operably linked to the one or more viral-specific packaging sequences from the final plurality of viral vectors.
3. The method of embodiment 1 or embodiment 2, wherein
(i) the viral vector composition is an adeno-associated virus (AAV) vector composition;
(ii) the at least one viral replication gene comprises at least one AAV replication gene;
(iii) the at least one viral structural gene comprises at least one AAV capsid encoding gene;
(iv) the at least one additional viral gene comprises at least one AAV helper gene; and
(v) the one or more viral-specific packaging sequences comprise at least two functional AAV inverted terminal repeats (ITRs).
4. The method of embodiment 1 or embodiment 2, wherein
(i) the viral vector composition is a lentivirus vector composition;
(ii) the at least one viral replication gene comprises at least one lentiviral pol gene;
(iii) the at least one viral structural gene comprises at least one lentiviral gag gene and at least one env gene;
(iv) the at least one additional viral gene comprises at least one lentiviral rev gene; and
(v) the one or more viral-specific packaging sequences comprise a Psi sequence.
5. The method of any one of embodiments 1-4, wherein each host cell of first plurality of host cells and each host cell of final plurality of host cells are mammalian host cells.
6. The method of any one of embodiments 1-5, wherein the first nucleotide sequence operably linked to the one or more viral-specific packaging sequences further encodes a reporter, a therapeutic payload or a selectable marker.
7. The method of any one of embodiments 1-6, wherein the first plurality of host cells at step (a) comprises at least 1,000 host cells each producing a unique engineered cyclic peptide.
8. The method of any one of embodiments 1-7, further comprising (e): generating new viral vectors in the presence of an engineered cyclic peptide obtained in step
(d), thereby producing the viral vector composition of increased viral titer and/or transduction efficiency.
9. The method of embodiment 8, which produces the viral vector composition having a characteristic, which is at least 2-fold higher than a corresponding characteristic of a reference viral vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from the group consisting of viral titer and transduction efficiency.
10. The method of embodiment 8 or embodiment 9, wherein the engineered cyclic peptide is not essentially present in the viral vector composition of increased viral titer and/or transduction efficiency.
11. A plurality of host cells permissive for replication of a virus, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary to produce the virus in the host cells; and
(iv) a nucleotide sequence operably linked to one or more viral-specific packaging sequences necessary for encapsulation of the nucleotide sequence within the viral capsids, wherein the nucleotide sequence encodes a payload; wherein the engineered cyclic peptide increases a characteristic of viral vectors produced by the plurality of host cells by at least 2-fold compared to a corresponding characteristic of viral vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of viral vectors is selected from the group consisting of viral titer and transduction efficiency.
12. The plurality of host cells of embodiment 11, wherein
(i) the virus is an adeno-associated virus (AAV);
(ii) the at least one viral replication gene comprises at least one AAV replication gene;
(iii) the at least one viral structural gene comprises at least one AAV capsid encoding gene;
(iv) the at least one additional viral gene comprises at least one AAV helper gene; and
(v) the one or more viral-specific packaging sequences comprise at least two functional AAV inverted terminal repeats (ITRs).
13. The plurality of host cells of embodiment 11, wherein
(i) the virus is a lentivirus;
(ii) the at least one viral replication gene comprises at least one lentiviral pol gene;
(iii) the at least one viral structural gene comprises at least one lentiviral gag gene and at least one env gene;
(iv) the at least one additional viral gene comprises at least one lentiviral rev gene; and
(v) the one or more viral-specific packaging sequences comprise a Psi sequence.
14. The plurality of host cells of any one of embodiments 11-13, wherein the engineered cyclic peptide is produced ribosomally in each host cell of the plurality of host cells.
15. The plurality of host cells of any one of embodiments 11-14, wherein the engineered cyclic peptide is exogenously supplied to each host cell of the plurality of host cells.
16. The plurality of host cells of any one of embodiments 11-15, wherein each host cell of the plurality of host cells is a mammalian host cell.
17. The plurality of host cells of any one of embodiments 11-16, wherein each host cell of the plurality of host cells is an insect host cell.
18. The plurality of host cells of any one of embodiments 11-17, wherein the payload comprises a therapeutic gene.
19. The plurality of host cells of any one of embodiments 11-18, wherein the plurality of host cells comprises at least 10,000 host cells.
20. A method of producing a viral vector composition of increased viral titer and/or transduction efficiency, the method comprising:
(a) culturing a plurality of host cells permissive for replication of a virus under conditions suitable for recombinant viral production, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary to produce the virus in the host cells; and
(iv) a nucleotide sequence operably linked to one or more viral-specific packaging sequences necessary for encapsulation of the nucleotide sequence within the viral capsids, wherein the nucleotide sequence encodes a payload; and
(b) producing the viral vector composition of increased viral titer and/or transduction efficiency from the plurality of host cells, wherein the viral vector composition has an increased viral titer and/or transduction efficiency which is at least a 20%, 40%, 60%, 80%, 100%, 200%, or 500% greater than a viral titer and/or transduction efficiency of a reference viral vector composition produced in a plurality of reference host cells under essentially identical conditions, wherein each reference host cell of the plurality of reference host cells comprises the elements (i)-(iv) of the plurality of host cells and does not comprise the engineered cyclic peptide.
21. The method of embodiment 20, wherein
(i) the viral vector composition is an adeno-associated virus (AAV) vector composition;
(ii) the at least one viral replication gene comprises at least one AAV replication gene;
(iii) the at least one viral structural gene comprises at least one AAV capsid encoding gene;
(iv) the at least one additional viral gene comprises at least one AAV helper gene; and
(v) the one or more viral-specific packaging sequences comprise at least two functional AAV inverted terminal repeats (ITRs).
22. The method of embodiment 20, wherein
(i) the viral vector composition is a lentivirus vector composition;
(ii) the at least one viral replication gene comprises at least one lentiviral pol gene;
(iii) the at least one viral structural gene comprises at least one lentiviral gag gene and at least one env gene;
(iv) the at least one additional viral gene comprises at least one lentiviral rev gene; and
(v) the one or more viral-specific packaging sequences comprise a Psi sequence.
23. The method of any one of embodiments 20-22, wherein the engineered cyclic peptide is produced ribosomally in each host cell of the plurality of host cells.
24. The method of any one of embodiments 20-23, wherein the engineered cyclic peptide is exogenously supplied to each host cell of the plurality of host cells.
25. The method of any one of embodiments 20-24, wherein each host cell of the plurality of host cells is a mammalian host cell.
26. The method of any one of embodiments 20-25, wherein each host cell of the plurality of host cells is an insect host cell.
27. The method of any one of embodiments 20-26, wherein the plurality of host cells comprises at least 10,000 host cells.
28. The method of any one of embodiments 20-27, wherein the engineered cyclic peptide is not essentially present in the viral vector composition of increased viral titer and/or transduction efficiency.
29. A method of obtaining an engineered cyclic peptide capable of increasing viral titer and/or transduction efficiency of adeno-associated virus (AAV) vector composition, the method comprising:
(a) culturing a first plurality of host cells permissive for AAV replication under conditions suitable for recombinant AAV production, wherein each host cell of the first plurality of host cells comprises:
(i) at least one AAV replication gene;
(ii) at least one AAV capsid encoding gene;
(iii) at least one AAV helper gene; and
(iv) an engineered cyclic peptide produced from a polypeptide encoded by a first nucleotide sequence, wherein (v) the first nucleotide sequence is operably linked to at least two functional AAV inverted terminal repeats (ITRs) or (vi) the first nucleotide sequence is associated with a second nucleotide sequence comprising a barcode that comprises identifying information regarding the engineered cyclic peptide produced in the host cell, and the second nucleotide sequence is operably linked to at least two functional AAV ITRs, thereby generating a first plurality of AAV vectors comprising the first nucleotide sequence and/or the second nucleotide sequence from the first plurality of host cells;
(b) optionally, repeating the following steps one or more times in cycles:
(b1) allowing a plurality of AAV vectors of the previous cycle to infect a plurality of host cells of the present cycle permissive for AAV replication; and
(b2) culturing the plurality of host cells of the present cycle under conditions suitable for recombinant AAV production, wherein each host cell of the plurality of host cells of the present cycle comprises the elements (i)-(iii) of the first plurality of host cells, and further comprises the first nucleotide sequence operably linked to the at least two functional AAV ITRs producing the engineered cyclic peptide, thereby generating a plurality of AAV vectors of the present cycle comprising the first nucleotide sequence;
(c) allowing the first plurality of AAV vectors or the plurality of AAV vectors of the present cycle to infect a final plurality of host cells; and
(d) obtaining one or more engineered cyclic peptides capable of increasing viral titer and/or transduction efficiency of AAV vector composition by analyzing nucleotide sequences operably linked to the at least two functional AAV inverted terminal repeats (ITRs) from (i) the final plurality of host cells and/or (ii) a final plurality of AAV vectors produced in the final plurality of host cells.
30. The method of embodiment 29, wherein in step (a), the first nucleotide sequence is operably linked to the at least two functional AAV ITRs, and the method comprises culturing the final plurality of host cells under conditions suitable for recombinant AAV production, wherein each host cell of the final plurality of host cells comprises the elements (i)-(iii) of the first plurality of host cells, and further comprises the first nucleotide sequence operably linked to the at least two functional AAV ITRs and producing the engineered cyclic peptide encoded by the first nucleotide sequence, thereby producing the final plurality of AAV vectors from the final plurality of host cells and obtaining the engineered cyclic peptide by analyzing nucleotide sequences operably linked to the at least two functional AAV ITRs from the final plurality of AAV vectors.
31. The method of embodiment 29 or embodiment 30, wherein the engineered cyclic peptide produced during AAV production of the polypeptide contains more than 3 and less than 80 amino acid residues.
32. The method of any one of embodiments 29-31, wherein each host cell of first plurality of host cells and each host cell of final plurality of host cells are mammalian host cells.
33. The method of any one of embodiments 29-32, wherein the first nucleotide sequence operably linked to the at least two functional AAV ITRs further encodes a reporter, a therapeutic payload or a selectable marker.
34. The method of any one of embodiments 29-33, wherein the first plurality of host cells at step (a) comprises at least 1,000 host cells each producing a unique engineered cyclic peptide.
35. The method of any one of embodiments 29-34, further comprising (e): generating new AAV vectors in the presence of an engineered cyclic peptide obtained in step (d), thereby producing the AAV vector composition of increased viral titer and/or transduction efficiency.
36. The method of embodiment 35, which produces the AAV vector composition having a characteristic, which is at least 2-fold higher than a corresponding characteristic of a reference AAV vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from the group consisting of viral titer and transduction efficiency.
37. The method of embodiment 35, wherein the engineered cyclic peptide is not essentially present in the AAV vector composition of increased viral titer and/or transduction efficiency.
38. The method of any one of embodiments 29-37, wherein the engineered cyclic peptide comprises an amino acid sequence having at least 90% or more sequence identity to any one of the amino acid sequences selected from the group consisting of SEQ ID NO: 34—SEQ ID NO: 62, SEQ ID NO: 64—SEQ ID NO: 228, and SEQ ID NO: 230.
39. A plurality of host cells permissive for AAV replication, wherein each host cell of the plurality of host cells comprises an engineered cyclic peptide and further comprises:
(i) at least one AAV replication gene;
(ii) at least one AAV capsid encoding gene;
(iii) at least one AAV helper gene; and
(iv) a nucleotide sequence operably linked to at least two functional AAV internal terminal repeats (ITRs), wherein the nucleotide sequence encodes a payload; wherein the engineered cyclic peptide increases a characteristic of AAV vectors produced by the plurality of host cells by at least 2-fold compared to a corresponding characteristic of AAV vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of AAV vectors is selected from the group consisting of viral titer and transduction efficiency.
40. The plurality of host cells of embodiment 39, wherein the engineered cyclic peptide is produced ribosomally in each host cell of the plurality of host cells.
41. The plurality of host cells of embodiment 39 or embodiment 40, wherein the engineered cyclic peptide is exogenously supplied to each host cell of the plurality of host cells.
42. The plurality of host cells of any one of embodiments 39-41, wherein the engineered cyclic peptide contains more than 3 and less than 80 amino acid residues.
43. The plurality of host cells of any one of embodiments 39-42, wherein each host cell of the plurality of host cells is a mammalian host cell.
44. The plurality of host cells of any one of embodiments 39-43, wherein each host cell of the plurality of host cells is an insect host cell.
45. The plurality of host cells of any one of embodiments 39-44, wherein the payload comprises a therapeutic gene.
46. The plurality of host cells of any one of embodiments 39-45, wherein the plurality of host cells comprises at least 10,000 host cells.
47. The plurality of host cells of any one of embodiments 39-46, wherein the engineered cyclic peptide comprises an amino acid sequence having at least 90% or more sequence identity to any one of the amino acid sequences selected from the group consisting of SEQ ID NO: 34—SEQ ID NO: 62, SEQ ID NO: 64—SEQ ID NO: 228, and SEQ ID NO: 230.
48. A method of producing an adeno-associated virus (AAV) vector composition of increased viral titer and/or transduction efficiency, the method comprising: (a) culturing a plurality of host cells permissive for AAV replication under conditions suitable for recombinant AAV production, wherein each host cell of the plurality of host cells comprises an engineered cyclic peptide and further comprises:
(i) at least one AAV replication gene;
(ii) at least one AAV capsid encoding gene;
(iii) at least one AAV helper gene; and
(iv) a nucleotide sequence operably linked between two functional AAV internal terminal repeats (ITRs), wherein the nucleotide sequence encodes a payload; and (b) producing the AAV vector composition of increased viral titer and/or transduction efficiency from the plurality of host cells, wherein the AAV vector composition has an increased viral titer and/or transduction efficiency which is at least a 20%, 40%, 60%, 80%, 100%, 200%, or 500% greater than an viral titer and/or transduction efficiency of a reference AAV vector composition produced from a plurality of reference host cells under essentially identical conditions, wherein each reference host cell of the plurality of reference host cells comprises the elements (i)-(iv) of the plurality of host cells and does not comprise the engineered cyclic peptide.
49. The method of embodiment 48, wherein the engineered cyclic peptide is produced ribosomally in each host cell of the plurality of host cells.
50. The method of embodiment 48, wherein the engineered cyclic peptide is exogenously supplied to each host cell of the plurality of host cells.
51. The method of any one of embodiments 48-50, wherein each host cell of the plurality of host cells is a mammalian host cell.
52. The method of any one of embodiments 48-51, wherein each host cell of the plurality of host cells is an insect host cell.
53. The method of any one of embodiments 48-52, wherein the plurality of host cells comprises at least 10,000 host cells.
54. The method of any one of embodiments 48-53, wherein the engineered cyclic peptide is not essentially present in the AAV vector composition of increased viral titer and/or transduction efficiency.
55. The method of any one of embodiments 48-54, wherein the engineered cyclic peptide comprises an amino acid sequence having at least 90% or more sequence identity to any one of the amino acid sequences selected from the group consisting of SEQ ID NO: 34—SEQ ID NO: 62, SEQ ID NO: 64—SEQ ID NO: 228, and SEQ ID NO: 230.
56. A method of obtaining an engineered cyclic peptide capable of increasing viral titer and/or transduction efficiency of lentivirus vector composition, the method comprising:
(a) culturing a first plurality of host cells permissive for lentivirus replication under conditions suitable for recombinant lentivirus production, wherein each host cell of the first plurality of host cells comprises:
(i) at least one lentiviral gag gene;
(ii) at least one lentiviral pol gene;
(iii) at least one lentiviral rev gene;
(iv) at least one env gene; and
(v) an engineered cyclic peptide produced from a polypeptide encoded by a first nucleotide sequence, wherein (vi) the first nucleotide sequence is operably linked to a Psi sequence or (vii) the first nucleotide sequence is associated with a second nucleotide sequence comprising a barcode that comprises identifying information regarding the engineered cyclic peptide produced in the host cell, and the second nucleotide sequence is operably linked to a Psi sequence, thereby obtaining a first plurality of lentivirus vectors comprising the first nucleotide sequence and/or the second nucleotide sequence from the first plurality of host cells;
(b) optionally, repeating the following steps one or more times in cycles:
(b1) allowing a plurality of lentivirus vectors of the previous cycle to infect a plurality of host cells of the present cycle permissive for lentivirus replication; and
(b2) culturing the plurality of host cells of the present cycle under conditions suitable for recombinant lentivirus production, wherein each host cell of the plurality of host cells of the present cycle comprises the elements (i)-(iv) of the first plurality of host cells, and further comprises the first nucleotide sequence operably linked to the Psi sequence producing the engineered cyclic peptide, thereby obtaining a plurality of lentivirus vectors of the present cycle comprising the first nucleotide sequence;
(c) allowing the first plurality of lentivirus vectors or the plurality of lentivirus vectors of the present cycle to infect a final plurality of host cells; and
(d) determining one or more engineered cyclic peptides capable of increasing viral titer and/or transduction efficiency of lentivirus vector composition by analyzing nucleotide sequences operably linked to the Psi sequence from (i) the final plurality of host cells and/or (ii) a final plurality of lentivirus vectors produced in the final plurality of host cells.
57. The method of embodiment 56, wherein in step (a), the first nucleotide sequence is operably linked to the Psi sequence, and the method comprises culturing the final plurality of host cells under conditions suitable for recombinant lentivirus production, wherein each host cell of the final plurality of host cells comprises the elements (i)-(iv) of the first plurality of host cells, and further comprises the first nucleotide sequence operably linked to the Psi sequence and producing the engineered cyclic peptide encoded by the first nucleotide sequence, thereby producing the final plurality of lentivirus vectors from the final plurality of host cells and determining the engineered cyclic peptide by analyzing nucleotide sequences operably linked to the Psi sequence from the final plurality of lentivirus vectors.
58. The method of embodiment 57 or embodiment 56, wherein each host cell of first plurality of host cells and each host cell of final plurality of host cells are mammalian host cells.
59. The method of any one of embodiments 56-58, wherein the first nucleotide sequence operably linked to the Psi sequence further encodes a reporter, a therapeutic payload or a selectable marker.
60. The method of any one of embodiments 56-59, wherein the first plurality of host cells at step (a) comprises at least 1,000 host cells each producing a unique engineered cyclic peptide.
61. The method of any one of embodiments 56-60, further comprising (e): generating new lentivirus vectors in the presence of an engineered cyclic peptide obtained in step (d), thereby producing the lentivirus vector composition of increased viral titer and/or transduction efficiency.
62. The method of embodiment 61, which produces the lentivirus vector composition having a characteristic, which is at least 2-fold higher than a corresponding characteristic of a reference lentivirus vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from the group consisting of viral titer and transduction efficiency.
63. The method of embodiment 61, wherein the engineered cyclic peptide is not essentially present in the lentivirus vector composition of increased viral titer and/or transduction efficiency.
64. A plurality of host cells permissive for lentivirus replication, wherein each host cell of the plurality of host cells comprises an engineered cyclic peptide and further comprises:
(i) at least one lentiviral gag gene;
(ii) at least one lentiviral pol gene;
(iii) at least one lentiviral rev gene;
(iv) at least one env gene; and
(v) a nucleotide sequence operably linked to a Psi sequence, wherein the nucleotide sequence encodes a payload (e.g., therapeutic gene);
wherein the engineered cyclic peptide increases a characteristic of lentivirus vectors produced by the plurality of host cells by at least 2-fold compared to a corresponding characteristic of lentivirus vectors produced by a plurality of reference host cells under essentially identical conditions, wherein each reference host cell of the plurality of reference host cells comprises the elements (i)-(iv) of the plurality of host cells and does not comprise the engineered cyclic peptide, and wherein the characteristic of lentivirus vectors is selected from the group consisting of viral titer and transduction efficiency.
65. The plurality of host cells of embodiment 64, wherein the engineered cyclic peptide is produced ribosomally in each host cell of the plurality of host cells.
66. The plurality of host cells of embodiment 64, wherein the engineered cyclic peptide is exogenously supplied to each host cell of the plurality of host cells.
67. The plurality of host cells of any one of embodiments 64-66, wherein each host cell of the plurality of host cells is a mammalian host cell.
68. The plurality of host cells of any one of embodiments 64-67, wherein the plurality of host cells comprises at least 10,000 host cells.
69. The plurality of host cells of any one of embodiments 64-68, wherein the payload comprises a therapeutic gene.
70. A method of producing a lentivirus vector composition of increased viral titer and/or transduction efficiency, the method comprising:
(a) culturing a plurality of host cells permissive for lentiviral replication under conditions suitable for recombinant lentiviral production, wherein each host cell of the plurality of host cells comprises an engineered cyclic peptide and further comprises:
(i) at least one lentiviral gag gene;
(ii) at least one lentiviral pol gene;
(iii) at least one lentiviral rev gene;
(iv) at least one env gene; and
(v) a nucleotide sequence operably linked to a Psi sequence, wherein the nucleotide sequence encodes a payload (e.g., therapeutic gene); and
(b) producing the lentivirus vector composition of increased viral titer and/or transduction efficiency from the plurality of host cells, wherein the lentivirus vector composition has an increased viral titer and/or transduction efficiency which is at least a 20%, 40%, 60%, 80%, 100%, 200%, or 500% greater than a viral titer and/or transduction efficiency of a reference lentivirus vector composition produced in a plurality of reference host cells under essentially identical conditions, wherein each reference host cell of the plurality of reference host cells comprises the elements (i)-(v) of the plurality of host cells and does not comprise the engineered cyclic peptide.
71. The method of embodiment 70, wherein the engineered cyclic peptide is produced ribosomally in each host cell of the plurality of host cells.
72. The method of embodiment 70, wherein the engineered cyclic peptide is exogenously supplied to each host cell of the plurality of host cells.
73. The method of any one of embodiments 70-72, wherein each host cell of the plurality of host cells is a mammalian host cell.
74. The method of any one of embodiments 70-73, wherein each host cell of the plurality of host cells is an insect host cell.
75. The method of any one of embodiments 70-74, wherein the plurality of host cells comprises at least 10,000 host cells.
76. The method of any one of embodiments 70-75, wherein the engineered cyclic peptide is not essentially present in the lentivirus vector composition of increased viral titer and/or transduction efficiency.
77. A kit for obtaining a viral vector composition of increased viral titer and/or transduction efficiency, comprising at least one engineered cyclic peptide and a non-peptide macromolecule selected from the group consisting of: a linear or branched polyethyleneimine (PEI), PEI dendrimer, a polypropyleneimine (PPI), Poly(amidoamine) (PAA) and dendrimers (PAMAM), cationic cyclodextrin, polyalkylamine, a polyhydroxyalkylamine, poly(butyleneimine) (PBI), spermine, a N-substituted polyallylamine, N-substituted chitosan, a N-substituted polyornithine, a N-substituted polylysine (PLL), a N-substituted polyvinylamine, poly(β-amino ester), hyperbranched poly(amino ester) (h-PAE), networked poly(amino ester) (n-PAE), poly(4-hydroxy-1-proline ester) (PHP-ester), a poly-β-aminoacid, 1,2-dioleoyl-3-trimethylammonium-propane (DOTAP), 1,2-dimyristoyl-3-trimethylammonium-propane (DMTAP), 3β-[N-(N′,N′-dimethylaminoethane)-carbamoyl] cholesterol (DC-Chol), N-[1-(2,3-dioleyloxy)propyl]-N,N,N-trimethylammonium chloride (DOTMA), 1,2-dipalmitoyl-3-trimethylammonium-propane (DPTAP), cationic liposome, and dendrimer.

In some particular embodiments of the methods and compositions disclosed above, the engineered cyclic peptide has between 4 and 20 amino acid residues and comprises one or more of the following peptide motifs selected from the group consisting of: ARO_HYD_POL-motif: [‘F’, ‘W’, ‘Y’] followed by [‘G’, ‘A’, ‘V’, ‘I’, ‘L’, ‘M’, ‘P’] followed by [‘S’, ‘T’, ‘Q’, ‘N’, ‘C’]; ARO_POL_ARO-motif: [‘F’, ‘W’, ‘Y’] followed by [‘S’, ‘T’, ‘Q’, ‘N’, ‘C’] followed by [‘F’, ‘W’, ‘Y’]; ARO_POL_POS-motif: [‘F’, ‘W’, ‘Y’] followed by [‘S’, ‘T’, ‘Q’, ‘N’, ‘C’] followed by [‘K’, ‘R’, ‘H’]; HYD_ARO_POL-motif: [‘G’, ‘A’, ‘V’, ‘I’, ‘L’, ‘M’, ‘P’] followed by [‘F’, ‘W’, ‘Y’] followed by [‘S’, ‘T’, ‘Q’, ‘N’, ‘C’]; HYD_HYD_POL-motif: [‘G’, ‘A’, ‘V’, ‘I’, ‘L’, ‘M’, ‘P’] followed by [‘G’, ‘A’, ‘V’, ‘I’, ‘L’, ‘M’, ‘P’] followed by [‘S’, ‘T’, ‘Q’, ‘N’, ‘C’]; HYD_NEG_POL-motif: [‘G’, ‘A’, ‘V’, ‘I’, ‘L’, ‘M’, ‘P’] followed by [‘D’, ‘E’] followed by [‘S’, ‘T’, ‘Q’, ‘N’, ‘C’]; HYD_POS_POL-motif: [‘G’, ‘A’, ‘V’, ‘I’, ‘L’, ‘M’, ‘P’] followed by [‘K’, ‘R’, ‘H’] followed by [‘S’, ‘T’, ‘Q’, ‘N’, ‘C’]; NEG_HYD_POL-motif: [‘D’, ‘E’] followed by [‘G’, ‘A’, ‘V’, ‘I’, ‘L’, ‘M’, ‘P’] followed by [‘S’, ‘T’, ‘Q’, ‘N’, ‘C’]; NEG_POL_ARO-motif: [‘D’, ‘E’] followed by [‘S’, ‘T’, ‘Q’, ‘N’, ‘C’] followed by [‘F’, ‘W’, ‘Y’]; NEG_POL_NEG-motif: [‘D’, ‘E’] followed by [‘S’, ‘T’, ‘Q’, ‘N’, ‘C’] followed by [‘D’, ‘E’]; NEG_POL_POS-motif: [‘D’, ‘E’] followed by [‘S’, ‘T’, ‘Q’, ‘N’, ‘C’] followed by [‘K’, ‘R’, ‘H’]; POL_HYD_ARO-motif: [‘S’, ‘T’, ‘Q’, ‘N’, ‘C’] followed by [‘G’, ‘A’, ‘V’, ‘I’, ‘L’, ‘M’, ‘P’] followed by [‘F’, ‘W’, ‘Y’]; POL_HYD_HYD-motif: [‘S’, ‘T’, ‘Q’, ‘N’, ‘C’] followed by [‘G’, ‘A’, ‘V’, ‘I’, ‘L’, ‘M’, ‘P’] followed by [‘G’, ‘A’, ‘V’, ‘I’, ‘L’, ‘M’, ‘P’]; POL_HYD_NEG-motif: [‘S’, ‘T’, ‘Q’, ‘N’, ‘C’] followed by [‘G’, ‘A’, ‘V’, ‘I’, ‘L’, ‘M’, ‘P’] followed by [‘D’, ‘E’]; POL_HYD_POL-motif: [‘S’, ‘T’, ‘Q’, ‘N’, ‘C’] followed by [‘G’, ‘A’, ‘V’, ‘I’, ‘L’, ‘M’, ‘P’] followed by [‘S’, ‘T’, ‘Q’, ‘N’, ‘C’]; POL_HYD_POS-motif: [‘S’, ‘T’, ‘Q’, ‘N’, ‘C’] followed by [‘G’, ‘A’, ‘V’, ‘I’, ‘L’, ‘M’, ‘P’] followed by [‘K’, ‘R’, ‘H’]; POL_NEG_ARO-motif: [‘S’, ‘T’, ‘Q’, ‘N’, ‘C’] followed by [‘D’, ‘E’] followed by [‘F’, ‘W’, ‘Y’]; POL_NEG_HYD-motif: [‘S’, ‘T’, ‘Q’, ‘N’, ‘C’] followed by [‘D’, ‘E’] followed by [‘G’, ‘A’, ‘V’, ‘I’, ‘L’, ‘M’, ‘P’]; POL_NEG_NEG-motif: [‘S’, ‘T’, ‘Q’, ‘N’, ‘C’] followed by [‘D’, ‘E’] followed by [‘D’, ‘E’]; POL_NEG_POS-motif: [‘S’, ‘T’, ‘Q’, ‘N’, ‘C’] followed by [‘D’, ‘E’] followed by [‘K’, ‘R’, ‘H’]; POL_POL_ARO-motif: [‘S’, ‘T’, ‘Q’, ‘N’, ‘C’] followed by [‘S’, ‘T’, ‘Q’, ‘N’, ‘C’] followed by [‘F’, ‘W’, ‘Y’]; POL_POL_HYD-motif: [‘S’, ‘T’, ‘Q’, ‘N’, ‘C’] followed by [‘S’, ‘T’, ‘Q’, ‘N’, ‘C’] followed by [‘G’, ‘A’, ‘V’, ‘I’, ‘L’, ‘M’, ‘P’]; POL_POL_NEG-motif: [‘S’, ‘T’, ‘Q’, ‘N’, ‘C’] followed by [‘S’, ‘T’, ‘Q’, ‘N’, ‘C’] followed by [‘D’, ‘E’]; POL_POL_POL-motif: [‘S’, ‘T’, ‘Q’, ‘N’, ‘C’] followed by [‘S’, ‘T’, ‘Q’, ‘N’, ‘C’] followed by [‘S’, ‘T’, ‘Q’, ‘N’, ‘C’]; POL_POL_POS-motif: [‘S’, ‘T’, ‘Q’, ‘N’, ‘C’] followed by [‘S’, ‘T’, ‘Q’, ‘N’, ‘C’] followed by [‘K’, ‘R’, ‘H’]; POS_HYD_POL-motif: [‘K’, ‘R’, ‘H’] followed by [‘G’, ‘A’, ‘V’, T, ‘L’, ‘M’, ‘P’] followed by [‘S’, ‘T’, ‘Q’, ‘N’, ‘C’]; POS_POL_ARO-motif: [‘K’, ‘R’, ‘H’] followed by [‘S’, ‘T’, ‘Q’, ‘N’, ‘C’] followed by [‘F’, ‘W’, ‘Y’]; and POS_POL_POS-motif: [‘K’, ‘R’, ‘H’] followed by [‘S’, ‘T’, ‘Q’, ‘N’, ‘C’] followed by [‘K’, ‘R’, ‘H’].

### EXAMPLES

Aspects of the present teachings may be further understood in light of the following examples, which should not be construed as limiting the scope of the present teachings in any way. Certain aspects of the present invention, including, but not limited to, general cell culture propagation methods, general methods and reagents of producing recombinant AAV vectors in host cells, AAV vector extraction from host cells, generating and screening intein-based libraries of cyclic peptides were disclosed in the earlier published applications: U.S. Pat. Nos. 5,173,414 A, 5,139,941 A, 6,924,128 B2, 6,642,051 B1, 6,632,670 B1, 20210145929 A1, 7,271,002 B2, 20220162642 A1, 8,802,417 B2, 20200248205 A1, 9,528,126 B2, 11/261,463 B2, 11/078,464 B2, 9,040,462 B2; 20070105140 A1, 20040091966 A1, 20030219723 A1, the contents of which are incorporated herein by reference in its entirety.

### Example 1. Improving AAV Vectors Manufacturability by Cyclic Peptides Discovered Through a High Throughput Screen of Intein-Based Peptide Library

Host cell lines used for manufacturing of AAV vectors are not optimized for AAV production. Cell environment can be efficiently modulated by intracellular cyclic peptides, which can affect AAV packaging and infectivity in a variety of ways. Endogenously generated cyclic peptides are advantageous in that they do not need to cross the cell membrane. This eliminates the usual restrictions related to small molecule discovery that requires compounds to be able to cross the cell membrane in order to modulate intracellular targets. Cyclic peptides have a variety of advantages over small molecules. Firstly, their modularity makes their construction simple. Second, they can optionally be genetically encoded and ribosomally expressed, ensuring high local concentration. Third, they can be chemically synthesized offering a wide array of derivatization options. Fourth, cyclic peptides typically have higher rigidity. This increases affinity and specificity toward their target. It has been speculated that this results from lower entropy in the Gibbs free energy equation. Fifth, cyclic peptides are resistant to exopeptidase degradation as they lack amino and carboxyl termini. Sixth, some cyclic peptides may have enhanced membrane permeability compared to linear counterparts. Seventh, because the cyclic peptide diversity scales exponentially, chemical diversity is practically limitless. Eighth, because cyclic peptides can be encoded on DNA and generated ribosomally it is easy to link biochemical function to chemical identity. Ninth, because cyclic peptides can be encoded on DNA and generated ribosomally, these biochemical libraries can be “selected” as opposed to screened (e.g. traditional drug discovery). Tenth, genetically encoded cyclic peptides serve as good starting points for synthetic chemistry derivatization. Eleventh, cyclic peptides containing natural amino acids, while more resistant to degradation compared to linear peptides, can still be degraded, allowing easy removal compared to small molecules.

Because mechanisms by which engineered cyclic peptides may increase AAV packaging and infectivity during viral particle assembly/packaging in the host cell are diverse (e.g. increasing/decreasing favorability of capsid stoichiometries, altering post translational modification, altering rAAV DNA payload methylation, altering DNA packaging, altering full:empty ratios, altering proteolytic process, altering intracellular trafficking, altering exposure history to different pH levels, altering allosteric signaling networks of capsid proteins/assemblies, altering rAAV viral escape, altering transcriptional profiles of specific genes or globally, inhibiting genes that interfere with viral synthesis, activating genes that enhance viral synthesis, and others) and have not been explored, a high throughput, competitive enrichment of an rAAV-vectored split intein-ligated cyclic peptide library was performed to select for cyclic peptides that enhanced the yield and/or functional properties of rAAV. Split-intein circular ligation of peptides and proteins (SICLOPPS) is a well-known biotechnology technique that permits the creation of cyclic peptides. These peptides are produced by ribosomal protein synthesis, followed by an intein mediated splicing event that ligates a specified peptide sequence into a loop. Details on methods of cyclic peptide library generation and use can be found in U.S. Pat. No. 9,040,462 B2, US 20070105140 A1, US 20040091966 A1, and US 20030219723 A1, incorporated herein.

To perform a selective enrichment of AAV manufactuability enhancing engineered cyclic peptides in host cells permissive for AAV replication, a genetically encoded cyclic peptide library was inserted into the AAV packaging cell line (e.g., a cell containing the components necessary for AAV biosynthesis) flanked by AAV ITRs, and peptides were transcribed, translated, and cyclized endogenously. The presence of a unique engineered cyclic peptide in each host cell resulted in different types and different degrees of modulation to various cellular components (e.g. inactivating a hydrolase, hyper stabilizing a cofactor regeneration enzyme, altering transcription factor activity, altering stress response, etc.). DNAs encoding/identifying specific engineered cyclic peptides expressed in the host cell were packaged in AAV capsids, where each capsid contained DNA encoding/identifying the specific engineered cyclic peptide. The host cell's ability to efficiently produce highly functional AAV vectors comprising DNA that encodes/identifies engineered cyclic peptides is influenced by the cell's molecular genetic state, which can be modulated by the engineered cyclic peptide. The cell's molecular genetic state may become more or less favorable to rAAV viral synthesis as a result of the presence of the engineered cyclic peptide. Produced AAV capsids were used to infect naive host cells permissive for AAV replication. Infection by these AAVs appeared to proceed with varying efficiency, where the efficiency of the infection was determined by infectivity-modulating properties of the specific peptides. Peptides that positively modulated AAV packaging and infectivity resulted in increased infection rates for corresponding AAV vectors, in which DNAs encoding these peptides were present. Thus, proportions of host cells having AAV manufacturability-enhancing engineered cyclic peptide gene sequences were increased after one round of infection (see FIG. 2). Similarly, proportions of host cells having AAV-manufacturability-reducing engineered cyclic peptide gene sequences were depleted after one round of infection (see FIG. 2). Proportions of host cells having engineered cyclic peptide gene sequences with little or no effect on rAAV manufacturability were reduced after one round of infection because engineered cyclic peptide gene sequences that enhanced rAAV manufacturability were able to out-compete neutral engineered cyclic peptides (see FIG. 2). After the first infection round, resulting AAV particles were collected and used for infection of new naive host cells permissive for AAV replication (the second infection round). Again, during the second infection round, AAV viral particles of higher infectivity levels (due to effect of the peptide on the packaging cell), and/or AAV viral particles that more efficiently packaged the engineered cyclic peptide DNA sequence, were able to infect more naive cells and/or more effectively deliver/transduce the DNA encoding/identifying the peptide; thus, populations of host cells having AAV-enhancing peptides were further enriched.

In contrast, cyclic peptide gene sequences that negatively affected AAV manufacturability were packaged less efficiently and/or were packaged in less infectious, lower quality capsids. These capsids were less capable of infecting naive cells. The DNA encoding these less efficacious peptides were produced, packaged, and/or transduced less effectively and were effectively eliminated from host cell culture after 2-3 re-infections (e.g., outcompeted).

The described process has been repeated 2-4 times using up to eighteen different engineered cyclic peptide library architectures, and different enrichment strategies. rAAVs harboring DNA encoding engineered cyclic peptides that increased AAV packaging and infectivity were successfully selected from a starting library of random engineered cyclic peptides. Thus, genetically encoded, virally vectored peptide libraries can be used to select for AAV performance enhancing peptides. Technical details of the described screen are provided below.

### Reagents and Materials Used in this Example

AAVpro 293T human embryonic kidney cell line (HEK-293T) was obtained from Takara Bio. Fetal Bovine Serum (FBS), Dulbecco's modified Eagle medium (DMEM) and the penicillin/streptomycin cocktail were obtained from Genesee Scientific. pAAV-CMV Vector, pAAV-ZsGreenl Vector, AAVpro® Packaging Rep-Cap Plasmid (AAV2), pHelper Vector were obtained from Takara Bio. AAV Extraction Solution A and AAV Extraction Solution B were obtained from Takara Bio. pTargeT™ Mammalian Expression Vector System was obtained from Promega. pMiniT Vector, NEB@ PCR Cloning Kit were obtained from New England Biolabs. EcoRI-HF, BamHI-HF, T4 DNA Ligase, NEB 10-Beta Electrocompetent Cells, and NEB Stable Competent Cells were obtained from New England Biolabs. JetOptimus transfection reagent was obtained from Polyplus.

Generation of SICLOPPS Peptide Library.

Production of cyclic peptide library was performed essentially as described in Townend J E, Tavassoli A. Traceless Production of Cyclic Peptide Libraries in E. coli. ACS Chem Biol. 2016 Jun. 17; 11(6):1624-30.

The coding sequence of SICLOPPS cyclic peptide generator inteins and eGFP reporter protein were synthesized by Integrated DNA Technologies with EcoRI and BamHI restriction sites flanking the coding sequence. These fragments were cloned into pMiniT Vectors as described in the NEB protocol (Ligation Protocol for NEB PCR Cloning Kit, NEB). To monitor enrichment of AAV-enhancing constructs in the population of host cells, a GFP reporter was incorporated into AAV capsids. A 2A peptide sequence (linker) followed by eGFP reporter coding sequence was inserted prior to the STOP codon of the cyclic peptide generator coding sequence using Golden Gate Assembly. Different lengths of cyclic peptide were generated by saturation mutagenesis of the cyclic peptide generator-2A-eGFP pMiniT Vector as described above, followed by transformation into NEB 10-beta Electrocompetent cells as described by the NEB protocol (Electroporation Protocol (C3020), NEB).

Plasmids were prepared from the resulting transformants and the various length cyclic peptide generator-2A-eGFP encoding DNA fragments were then digested out of the pMiniT vector using EcoRI-HF and BamHI-HF and ligated downstream of the ITR flanked constitutive CMV promoter of the pAAV-CMV vector using the EcoRI and BamHI restriction sites before transformation into NEB Stable Competent cells as described in the NEB protocol (High Efficiency Transformation for NEB© Stable Competent E. coli (C3040H), NEB). Plasmids prepared from the resulting transformants were used in AAV production.

Two different intein-based cyclic peptide libraries were created, one producing peptides of 5-20 aa in length, and another producing cyclic peptides of 20-50 aa in length. Amino acids located in the region of the peptide to be cyclized were randomized using saturation mutagenesis with NNK degenerate codons and the DNA constructed with oligos purchased from IDT. The resulting engineered cyclic peptide coding sequences comprised one constant amino acid “scar” (typically cysteine or serine), and 4 or more random NNK codons.

Individual cyclic peptide generators of interest were PCR amplified from viral AAV DNA, purified by gel electrophoresis and cloned into the pTargeT™ Mammalian Expression Vector System as described in the Promega protocol (pTARGET™ Mammalian Expression Vector System, product #A1410, Promega). Sanger sequencing was used to determine the identities of the individual cyclic peptides encoded on each vector.

AAV Production and Purification.

50-70% confluent HEK-293T cells (˜10\hat{ }6 total cells) grown in DMEM supplemented with 5% FBS were triple transfected with pHelper Vector, pAAV-CMV Vector containing cyclic peptide generator-2A-eGFP payload, and AAVpro® Packaging Rep-Cap Plasmid (AAV2) in a 1:1:1 molar ratio normalized to the plasmid size using JetOptimus Transfection Reagent in 100 mm TC treated petri dish.

24 hours after transfection, a medium change to DMEM with 2% FBS was performed. On day 3 post-transfection, cells were collected and processed. eGFP expression in packaging HEK-293T cells was quantified using a Tali Image Based Cytometer (Invitrogen). Cells were lysed in an acidic buffer (AAV Extraction Solution A), the homogenates were cleared from debris by centrifugation, and the pH was neutralized using HEPES buffer (AAV Extraction Solution B).

Subsequent rounds of cyclic peptide enrichment were carried out by transducing 50-70% confluent HEK-293T cells (˜10\hat{ }6 total cells) grown in DMEM supplemented with 5% FBS with a 1:10 to 1:10000 dilution of prepared AAV solution from above in DMEM with 5% FBS. Cells were double transfected with pHelper Vector and AAVpro® Packaging Rep-Cap Plasmid (AAV2) in a 1:1 molar ratio normalized to the plasmid size using JetOptimus Transfection Reagent in 100 mm TC treated petri dish. AAVs were harvested as described above.

AAV Titration.

Primers binding the eGFP reporter protein were used to measure the virus titer with quantitative polymerase chain reaction (qPCR). Before releasing the viral DNA from the particles, all extra-viral DNA was removed by digestion with DNase I. Then, the viral DNA was released by alkaline lysis. The qPCR was performed using the PowerUp™ SYBR™ Green Master Mix (Applied Biosystems), and primers against the zsGreen obtained from Integrated DNA Technologies. The extracted viral DNA and a serial dilution of a viral plasmid containing z as a standard were measured using the CFX96 Touch Real-Time PCR Detection System and the CFX Maestro Software (Bio-Rad, Hercules, CA).

Biological Viral Titer Measurement

HEK-293T were reseeded in a 24 well tissue culture treated plate at a density of 1-2*10\hat{ }4 cells/well in 0.5 mL of DMEM with 10% FBS. Cells were cultured overnight. 10-fold serial dilutions of prepared AAV2 particle solutions using DMEM with 10% FBS were used to infect the HEK-293T cells. Dilutions ranged from 1:10 to 1:10000. On Day 3 after infection, cells were detached using Trypsin/EDTA and analyzed for eGFP expression by image-based cytometry.

Sequencing

DNA is extracted from prepared AAV solutions of interest as described above. The coding sequence of the cyclic peptides were amplified by PCR. The resulting product is purified by gel electrophoresis and gel DNA extraction before being sequenced. (NovaSeq PE150).

Cyclic Peptide Validation

50-70% confluent HEK-293T cells (˜10\hat{ }6 total cells) grown in DMEM supplemented with 5% FBS were quadruple transfected with pHelper Vector, pAAV-ZsGreenl Vector, AAVpro® Packaging Rep-Cap Plasmid (AAV2), and pTargeT Mammalian Expression Vector with cloned individual cyclic peptide generator of interest in a 1:1:1:1 molar ratio normalized to the plasmid size using JetOptimus Transfection Reagent in 100 mm TC treated petri dish. AAV preparation is conducted as described above. Biological viral titer measurement is performed as described above.

### Example 2. AAV DNA Barcoding-Based Discovery of CPs

This example illustrates DNA barcoding-based discovery of CPs that improve AAV production and in vivo performance.

It may be desirable to implement an approach that completely removes the cyclic peptide sequence from the transgene plasmid during enrichment, allowing for discovery of cyclic peptides (CP) that enhance packaging of therapeutically relevant payloads. Such an approach provides a good way to perform in vivo discovery, where it would be undesirable to transduce a cyclic peptide generator into a living animal model. This can be accomplished through use of a DNA barcoding based approach. A barcoding based approach provides for a powerful screen, which allows for the rapid identification of viral production-enhancing cyclic peptides. This approach can be visualized in FIG. 6. FIG. 3 exemplifies the application in an in vivo discovery context. FIG. 13 exemplifies how CP-associated barcodes are quantified after transduction.

In this approach, a DNA barcode is placed into the mobilizable portion of the plasmid between the ITRs, while the cyclic peptide generator is on the vector backbone. In this arrangement, the cyclic peptide sequence will not be packaged into a cell, but the cyclic peptide's impact on viral activity can be determined by its associated, mobilizable DNA barcode. Two examples of how to implement DNA barcoded cyclic peptide AAV libraries are provided below.

A plasmid containing a DNA barcode is flanked by two ITRs, optionally with a GFP, luciferase, or therapeutic gene payload. Also on the plasmid, but outside of the ITRs is a cyclic peptide generator.

Packaging cells are transfected with the DNA library configured to produce a library of cyclic peptides. This CP library, along with 3 helper plasmids for AAV production (if components needed for packaging are not already supplied in the cell), are quadruply transfected into packaging cells.

The cyclic peptide coding sequence is configured not to be packaged by AAV. Instead, an ITR-flanked DNA barcode is associated with the cyclic peptide. This barcode, when separated from its associated cyclic peptide DNA sequence (e.g. packaged into a viral particle) can be used to identify its associated cyclic peptide (i.e. that was on the same plasmid). The cyclic peptide encoding DNA may also include additional DNA payload elements: fluorescent proteins (GFP), reporter enzymes (luciferase), therapeutic genes, and more. Elements required for viral production can be supplied via triple transfection of a rep-cap plasmid (pRC) and a helper plasmid (pHelp). Alternatively, these elements can also be expressed from stable genomic integrations of the genes, helper viruses, and so forth.

Viral packaging occurs. DNA encoding engineered cyclic peptides is associated with a DNA barcode. The DNA barcode is operably linked to packaging sequences (e.g., flanked by two ITRs) which will cause the DNA barcode to be packaged into an AAV. The cyclic peptide, being simultaneously expressed during packaging, is also present during packaging and exerts its biological effect. However, the cyclic peptide coding sequence is not flanked by ITRs and is not packaged into viral particles. Instead, the DNA barcode is used to identify the associated cyclic peptide.

In this way, cyclic peptides that enhance AAV production will enhance the packaging of their associated DNA barcodes into AAV particles (or increase TU:VG ratios of particles harboring the associated DNA barcodes) and the DNA barcode inside of the AAV particle can be used to identify the cyclic peptide that was present (e.g., associated with the DNA barcode) during the packaging process.

In contrast, cyclic peptides that reduce AAV production will reduce the efficiency of their associated DNA barcodes from being packaged into AAV particles (or reduce the TU:VG ratios of particles harboring the associated DNA barcodes). In this way, a cyclic peptide's impact on viral production can be determined by analysis of AAVs harboring the associated DNA barcode.

Then, the AAV-barcode library is harvested. Each barcode-DNA containing AAV contains a barcode that corresponds to a cyclic peptide that was present during viral packaging. This provides a way to link the quantity or infectiousness of AAVs containing a particular barcode with cyclic peptide that was present during viral packaging.

The viral titer of the library is preferentially determined at this point to determine the appropriate dosage for transduction in the next step. Too much viral material leads to toxicity. Too little viral material leads to under-sampling of DNA barcodes. We aim for about 1E4-1E5 AAV viral particles per cell with AAV2, but this will be different for different serotypes. 1E5 VG per cell typically results in 10-100 transducing units in AAV2 (this is AAV serotype and packaging methodology dependent).

These viral particles can then be transduced into cells, for example in vitro or in vivo (e.g., via tail vein injection of 1E13 VGs). It is important to note that the barcode approach is important for the in vivo discovery approach, where it is much less practical to configure the cells of a living animal for viral production. That is, because cells in an animal are not suitable as packaging cells, an approach that does not rely on direct amplification of cyclic peptide viral DNA is required. And for this problem, the exemplified cyclic peptide barcoding approach provides a solution because the barcodes can be easily analyzed by collecting the DNA of transduced animal cells. Transduction can optionally be monitored by observing the luminescence or fluorescence of a transduced payload if configured to allow it.

After a certain number of days, viral DNA is isolated from the cells and subjected to NGS. The naive DNA plasmid library and the remainder of the non-transduced viral library is also NGS-ed. The number of days depends on the particular experiment, but in general, it is at least 5 days. However, it can be preferable to wait for months, depending on the animal model, if desiring to identify highly stable transduction events.

The sequencing data can then be analyzed to determine which cyclic peptides (indicated by corresponding DNA barcodes) are desirable. The relative frequency of different barcodes (corresponding to different cyclic peptides) is used to determine which cyclic peptides enhanced viral titer and/or infectivity (e.g. by exerting their effect during packaging).

By analyzing the DNA barcodes associated with specific cyclic peptide sequences and determining enrichment, the functional impacts of cyclic peptide library members can be easily quantified. NGS-based analysis of the DNA barcodes provides the necessary information to identify cyclic peptides that improve in vivo performance, even in the absence of cyclic peptide DNA sequence.

Assuming that each unique barcode sequence has already been associated with a specific cyclic peptide sequence (e.g., through gene chip synthesis described above), barcode sequences can be mapped to cyclic peptide identity (e.g., using a python dictionary) using the following steps:
1) Perform standard NGS analysis and quality control to make a list of DNA reads from raw data files.
2) Count the number of occurrences or DNA barcode reads associated with each unique cyclic peptide in the naive, AAV packaged, and post-selection library data files. This information provides the initial quantitative measure of the representation of cyclic peptide sequences associated with each barcode.
3) Normalize the read counts within each library to allow for meaningful comparisons between the libraries. While there are many approaches, we typically use frequency which is the number of reads for a particular cyclic peptide divided by the total number of reads for all cyclic peptides.
4) Calculate the enrichment of each cyclic peptide sequence by comparing its frequency in the post-selection library to its frequency in the naive library and/or packaged library.
5) The fold change or enrichment ratio quantifies the extent to which a cyclic peptide sequence has become more or less represented in the library as a result of the selection process.
6) A fold change greater than 1 indicates an enrichment, while a fold change less than 1 suggests a depletion.
7) Comparing enrichments across all three conditions, naive, packaged, and transduced, provides insight not only into impacts of cyclic peptides on in vivo AAV performance, but also AAV production and titer. This provides a way to simultaneously optimize manufacturability and therapeutic aspects of the material.

Exemplary ways to implement DNA barcoded cyclic peptide AAV libraries are described below.

Barcoded cyclic peptide libraries can be built in a variety of ways:

One approach is to construct the cyclic peptide and barcode library with pre-mapped associations. This can be easily achieved using gene chip DNA synthesis. In this case an oligo DNA library is synthesized. The cyclic peptide coding sequence and barcode are synthesized in a pair on the same oligo. The cyclic peptide coding sequence is placed on the left/5′ of the oligo with arbitrary cloning sequences (e.g. for golden gate or Gibson assembly) to the 5′. It is important to note that the cyclic peptide DNA sequence only corresponds to the actual cyclic peptide encoding nucleotides and not the intein splicing machinery. In the middle of the oligo is a multiple cloning site into which the intein machinery and ITR DNA sequences will be inserted. Finally, on the right/3′ side of the oligo we have a pre-determined DNA barcode followed by a second cloning sequence. The specific sequence of the barcode is arbitrary, but the key point is that it can be assigned when the library is designed and synthesized, meaning that we will always know the mapping between cyclic peptide and barcode. This oligo sequence is then cloned into a destination plasmid, which provides half of the intein splicing machinery for the cyclic peptide sequence for the cyclic peptide sequence as well as the optional payload and the 3′ ITR for the DNA barcode. The resulting circular plasmid is halfway done and is only missing the other half of the intein machinery for the cyclic peptide generator and the 5′ ITR (we are skipping obvious accessory sequences for simplicity). The missing elements are provided by insertion of DNA containing the other half of the cyclic peptide generator intein machinery as well as the 5′ ITR into the multiple cloning site that separates the barcode and cyclic peptide. This cloning step generates a cyclic peptide sequence outside of the ITR that is associated with a pre-programmed DNA barcode sequence inside of the ITRs (and optional payload genes if desired). The approach has the advantage of allowing for exceptional control over library composition and enables machine-guided design of cyclic peptide diversity.

Another way to implement a barcoded cyclic peptide library is to use a random DNA barcode (for example a string or random “N” nucleotides long enough to ensure that the diversity exceeds the cyclic peptide library's diversity by a few orders of magnitude) inside of the ITRs along with a random cyclic peptide library. In this case, we start off not knowing which cyclic peptides and barcodes are associated. At this point, long-read DNA sequencing technology is used to associate the DNA barcodes with the cyclic peptide DNA library sequences. After this step, a short read sequencing technology can be used to provide the sequencing depth required. This approach is much simpler and cheaper to implement.

### Example 3. Lentivirus Production with CPs

This example illustrates methodologies for Cyclic peptide-enhanced lentiviral vector production.
1) Cells were seeded at 4-5×10\hat{ }6 cells per 10-cm plate, 24 hours prior to transfection, to achieve 80-90% confluence at the time of transfection.
2) 7.0 μg of lentiviral vector plasmid DNA was diluted in sterile water to a volume of 600 μl along with a cyclic peptide generator that expresses a cyclic peptide.
3) Titration of cyclic peptide plasmid DNA concentrations, from 0.5 ng to 50 ng, was used in this Example. However, the amount of required plasmid DNA depends on many factors, including the specific cyclic peptide being expressed, the cyclic peptide generator (e.g. more efficient cyclic peptide generators may need less DNA), and promoter strength (the pCMV promoter driving strong expression is used in this Example).
4) Between 4-8 control conditions (depending on number of experimental conditions) were also prepared that are identical to the above, except that no cyclic peptide plasmid was added.
5) This DNA was added to a tube of Lenti-X Packaging Single Shots and vortexed to mix.
6) The mixture was incubated for 10 minutes at room temperature to allow the formation of nanoparticle complexes, then briefly centrifuged.
7) The entire 600 μl of nanoparticle complex solution was added dropwise to the packaging cells, which were then rocked gently to mix.
8) Lentiviral packaging. Cells were incubated at 37° C. with 5% CO2, initially for 4 hours or overnight, then with an additional 6 ml of fresh complete growth medium for another 24-48 hours.
9) Lentiviral supernatants were harvested. They were centrifuged or filtered to remove cellular debris, using a filter of cellulose acetate or polysulfone, not nitrocellulose.
10) Virus production was verified and can be quantified using Lenti-X GoStix™ Plus or any other standard methodology. Then, the virus can be used to transduce target cells or stored at −80° C.
11) Next, for each viral production condition (e.g., different cyclic peptide packaging conditions, CP dosages, and controls), a three order of magnitude serial dilution at 10-fold dilution steps was performed.
12) All viral samples were then transduced, harboring a GFP transgene, into fresh HEK293 cells.
13) After 3 days, the transduced cells were collected, and analyzed them by flow cytometry (see FIG. 26).
14) Finally, the biological titer (flow cytometry) and physical titer (GoStix) were normalized to the no-CP control to determine the fold improvements in both viral titer and infectiousness. The results are reported in FIG. 25.
15) While in this Example cyclic peptides were added in a separate plasmid, the cyclic peptide generator can also be supplied from a genomically integrated construct in the packaging cells. Alternatively, cyclic peptides can also be exogenously added.

### Example 4. Discovery of Mechanism of Cyclic Peptides with Computational Structural Biology, Chemoinformatics, and Machine Learning

Mechanistic understanding streamlines implementation of the methods and compositions disclosed herein in two ways: 1. Understanding the cyclic peptide target relationship allows for a more rational approach to combining cyclic peptides possessing orthogonal mechanisms of action. 2. Understanding of mechanism allows for rational and library-based approaches to optimize cyclic peptide compounds using synthetic chemistry.

The first challenge was to build a matrix of cyclic peptide and target interactions. Cyclic peptides make one axis of the matrix and targets make the other axis. At this stage the matrix was empty as we had no information about the interactions.

To determine the interactions, we started off with classical molecular docking strategies. We used autodock GPU for accelerated docking. We started off by randomly selecting highly enriched cyclic peptide and potential target pairs. We saved all statistics from all docking runs, including binding energies, poses, cluster counts, RMSDs, etc . . .

This provided a first-pass approach to identify possible cyclic peptide target interactions. However, it was very slow and the number of hits we found was low. In order to speed things up, we built a machine learning model that was trained on the results of the docking. It predicted a binding energy as a function of cyclic peptide and target input. The model architecture was a multi-input funnel transformer model. A cyclic peptide sequence input enters one leg of a set of transformer blocks, while a protein sequence input enters a parallel set of transformer blocks. After several transformer blocks, the vector representations are concatenated and fed into a final set of transformer blocks. After several transformer block passes, a single neuron predicts the binding energy from the final layer of the transformer block. The number of transformer blocks and all their parameters are customizable (we found 3-4 blocks to be an ideal balance between speed and GPU performance). The model was designed to be exceptionally easy to customize and nearly any parameter is easily customizable.

This model, after training on the docking data was able to predict the results of molecular docking. While not as accurate as an actual docking experiment, it ran between 100,000-10,000,000 times faster than molecular docking, making for a much more efficient way of probing cyclic peptide mechanism.

Using this model, we then predicted the entire pairwise interaction matrix for typically between 100M to 2B pairwise interactions. Next, we sorted all of these interactions by binding energy, with the lowest energies being the best. Finally, these best binding energies, which are predicted to be the most likely cyclic peptide target interactions were selected for another round of docking. In this way, the machine learning model used the result of docking to triage subsequent rounds of docking.

A second round of more computationally expensive docking was performed. This time, a substantial increase in the number of very tight docking interactions was observed. This is expected because the machine learning model triaged the pairwise interactions expected to form tight interactions. In the cases where the machine learning model was wrong, the data was still useful because we then proceeded to retrain the machine learning algorithm from scratch with the larger dataset. In this way the machine learning model iteratively plugged the holes in its training dataset.

10,000 to 100,000 docking runs (depending on cycle number, average protein size, and average ligand size), using autodock-GPU, was observed to be a good size to sample the machine learning algorithm's accuracy.

In early iterations, the machine learning algorithm's accuracy on test and validation datasets trended in the right direction, but clearly had problems learning and generalizing the interaction space between cyclic peptides targets.

However, with each iteration, the machine learning algorithm's accuracy on test and validation datasets became more and more accurate. After about 15 iterations, the machine learning algorithm's accuracy was comparable to the classical docking algorithm. In fact, there is reason to believe that the machine learning algorithm is superior in that it can generalize better than the docking algorithm. This is because some crystal structures may have biologically implausible structures that distort docking energy calculations. However, because the machine learning algorithm evaluates many diverse interactions by looking only at sequence, potential outlier data points are less likely to be learned by a model that generalizes well due to a robust training protocol.

The inventors used machine learning models to predict binding energy of all interesting cyclic peptides found to be enriched in by NGS (implying an interaction between the cyclic peptide and some molecular species in the cell). Because all of the strongly predicted interactions were docked, it is possible to visualize and confirm potential interactions between cyclic peptides and putative targets using the computational tools. While there are many orders of magnitude more such structural images than could be included, FIG. 28A, FIG. 28B, FIG. 28C, FIG. 28D, FIG. 28E, and FIG. 28F provide some representative examples.

Increasing dataset diversity and size has been shown to dramatically improve model performance. We found this to be the case as well. Another interesting aspect of our cyclic peptide approach is that it allowed us to exploit the vast scientific body of knowledge from drug discovery and medicinal chemistry because cyclic peptides are small molecules. To leverage this advantage, we modified the above machine learning model's training dataset such that it now included non-cyclic peptide small molecules as well as a much more diverse set of proteins from all major compound databases that provide biophysical measurements of chemical-target interactions (e.g. kd, ki, ic50, ec50, etc. . . . ). In addition, we adjusted the machine learning model's prediction task. We added the 4 most common drug-target interaction measurements: Kd, Ki, IC50, EC50 (in addition to molecular docking binding energy). By including non-cyclic peptide molecules and non-human proteins, we were able to dramatically increase dataset size and diversity. We also started to dock many of these non-cyclic peptide compounds as well in order to build calibrate biophysical interaction measurements to computational energy calculations. The result of these changes was a substantial increase in prediction performance (as evaluated by mean square error of test and validation datasets) of each compound-target interaction metric compared to training models with single-metric prediction tasks. A major benefit of our computational approach that was enabled by our cyclic peptide viral packaging technology is that it provided estimates of cyclic peptide-target biophysical interactions. For example, FIG. 27 illustrates the cyclic peptide target IC50 matrix. This is only a small subset of the cyclic peptides and targets. Also, a similar chart exists for Kd, Ki, EC50, and binding energy. Many other parameters can be predicted as well, including enrichment score.

The computational strategy described herein can be used in a variety of ways to accelerate the discovery of valuable cyclic peptides. For example, this approach enables the rapid computational pre-screening of cyclic peptide libraries for those cyclic peptides that are most likely to exert desirable effects on viral production, with the resulting computational hits synthesized for in vitro characterization, enabling facile computational cyclic peptide library design. In another embodiment, these computational tools can be used to computationally evaluate synthetic cyclic peptides (e.g., containing non-canonical side chains or N-methylations) for their ability to improve viral production. In other embodiment, the same machine learning models can be used to predict and identify non-peptide small molecules that might enhance viral production (e.g., using enrichment factor as a proxy for how well the compound is expected to improve viral production). In other embodiments, computational tools are used to determine the mechanism of cyclic peptide toxicity. In other embodiments, the computational tools are used to identify gene targets that merit attention for investigation into their role in viral packaging. In other embodiments, the computational tools incorporate atomistic molecular dynamics simulations or coarse-grained simulations. In other embodiments, different machine learning architectures are used.

### Example 5. Exogenous Screening of Cyclic Peptides that Enhance Lentiviral Packaging

In some embodiments, it is desirable to identify cyclic peptides that are capable of being added to viral production exogenously. For example, this is required for use of any non-natural cyclic peptide chemistry in CPs. In this Example, a validation scheme for exogenously added CPs is disclosed.
1) Libraries of synthetic cyclic peptides are ordered from a reputable chemical synthesis company. Each peptide is dissolved in a solvent such as DMF or DMSO to create a stock solution. The specific solvent will depend on the properties of the cyclic peptide.
2) Prepare serial dilutions for each cyclic peptide at 1000× stocks (if solubility allows) for final concentrations of 100 μM, 20 μM, 5 μM, 1 μM, 200 nM, 50 nM, 10 nM.
3) One day before transfection, seed 3×10\hat{ }4 HEK293T cells per well in collagen-coated 96-well tissue culture plates with 120 μl of growth medium. Use tetracycline-free FBS in the growth medium. Aim for 60% confluence at the time of transfection. Additionally, add the appropriate amount of cyclic peptide dilution to each well. Our serial dilution is row-wise, so the synthetic cyclic peptide additives will be at the lowest dilution (highest concentration) at row A of the 96 well plate, with increasing dilutions for each subsequent row, with the final row a set of 12 controls (preferably half solvent and half no solvent).
4) Prepare the transfer vector plasmid DNA, encoding a GFP transgene. Dilute 40-80 ng of the plasmid DNA with sterile water to a final volume of 10 μl and mix thoroughly.
5) Open the foil seal of the Lenti-X Packaging Single Shots 96-well Plate (VSV-G). Add 10 μl of the diluted plasmid DNA. This will dissolve the lyophilized Xfect Transfection Reagent and Lenti-X packaging plasmids in each well.
6) Tap the plate gently to ensure the reagents are mixed and incubate for 10 minutes at room temperature to allow the nanoparticle complexes to form. The cyclic peptide formulations can impact nanoparticle complexation. If this is desired, cyclic peptides can optionally be added at this step.
7) Ensure each well's contents have dissolved fully, then mix the solution by gently pipetting up and down. Transfer the entire 10 μl of transfection complex solution dropwise to the corresponding well of HEK293T cells.
8) Incubate the transfected cells at 37° C., 5% CO2.
9) After 16 hours, add an additional 80 μl of fresh complete growth medium to each well and incubate for another 24-48 hours. If desired, additional cyclic peptide formulation can be added, but this is discouraged due to increased complexity and time.
10) After incubation, harvest the LVV-containing supernatants from each well. The lentiviral preparations may be optionally purified to reduce the potential impact of cyclic peptides to interfere with downstream analysis. However, this purification step can be skipped in the interest of prosecuting a high throughput screening protocol, and deferring a higher resolution characterization for a subsequent analysis.
11) Verify virus production using Lenti-X GoStix™ Plus or other standard methodologies and store the virus at −80° C. if not used immediately.
12) Perform a 3 order of magnitude serial dilution at 10-fold dilution steps for each viral production condition (different cyclic peptide packaging conditions, CP dosages, and controls).
13) Transduce the resulting lentiviruses into fresh HEK293 cells.
14) After 3 days, collect the transduced cells and analyze GFP expression by flow cytometry.
15) Calculate both the biological titer (using flow cytometry data) and physical titer (using GoStix data). Normalize the values from each cyclic peptide condition to the average of the controls (produced without cyclic peptides) to determine fold improvements in viral titer and infectiousness.
16) The results can be easily plotted to facilitate identification of cyclic peptide variants that, when added exogenously, can enhance lentiviral production.

In the exemplified high-throughput screening campaign, different synthetic cyclic peptides exert varying effects on the viral production process. The effects may be classified into several broad categories:
a) Toxic Effects: Some cyclic peptides may be toxic to the packaging cells, causing reduced cell viability or cell death. This will be evident by a decrease in cell density and poor cell health under microscopic examination. These peptides would not be suitable for further exploration, as maintaining cell viability is crucial in this system. This effect is typically dose-dependent.
b) Antiviral Effects: Some peptides might act as antivirals, reducing the replication or packaging of lentiviral vectors. This might be indicated by a reduction in physical titer without corresponding cell toxicity. If the peptide has antiviral properties, this may manifest as a reduction in both biological and physical titer, without an associated decrease in cell viability. These peptides would not be appropriate for improving lentivirus production, but they may have other interesting applications in antiviral research. We would plan to report such compounds for the benefit of humanity and have no plans to pursue commercial rights to them.
c) Enhancement of Viral Packaging: Ideally, some cyclic peptides will enhance viral packaging, possibly through interactions with the viral proteins or cellular machinery involved in the packaging process. This will be represented by an increase in the physical titer, and possibly an increased biological titer if the peptide enhances the infectivity of the virus. The optimal concentration of these peptides might vary, suggesting dose-responsive effects. The screening results should indicate which concentrations should be further evaluated in subsequent characterization studies.

By analyzing the biological and physical titer data normalized to the control condition (no cyclic peptide), one can gain insights into the effects of each cyclic peptide on the viral packaging process and cell health. One can differentiate between peptides that improve packaging efficiency, increase viral infectivity, or negatively impact these processes. From these results, one can select promising peptides for further investigation, such as dose-optimization studies, mechanism of action studies, and potential therapeutic applications.

This approach is preferably informed by a data-driven, computational approach (described in the computational example). This computational approach allows us to focus the comparatively expensive high throughput screening time only on compounds that are predicted to have a very high likelihood of enhancing viral production.

This high-throughput screening process thus enables to rapidly identify cyclic peptides that can be used to enhance lentiviral vector production, a crucial component of gene therapy and CAR-T cell therapy. The insights gained can improve the efficiency and scalability of these therapeutic technologies and may also shed light on novel biological interactions between cyclic peptides and the viral packaging process.

In conclusion, this screening process allows for rapidly characterizing the effects of a vast array of synthetic cyclic peptides on lentiviral vector production, leading to valuable insights for both therapeutic applications and basic scientific research.

### Example 6. Exogenous Screening of Cyclic Peptides that Enhance AAV Packaging

The same basic approach exogenous screening approach can be applied to AAV with adjustments that are known to a person skilled in the art. The basic steps of this screening approach remain the same as those outlined for lentiviral vector production, with modifications suitable for AAV, as outlined below.
1) HEK293T cells are seeded in a 96-well plate one day prior to transfection, aiming for 60% confluence at the time of transfection.
2) Each cyclic peptide is dissolved in DMSO (or appropriate solvent) to create a 1000×stock solution. Serial dilutions are prepared at final concentrations of 100 μM, 20 μM, 5 μM, 1 μM, 200 nM, 50 nM, 10 nM, 2 nM.
3) The cyclic peptides are added to the cells as described for lentivirus example.
4) AAV packaging plasmids, helper plasmids (providing necessary Adenovirus helper functions), and a transfer vector plasmid encoding a GFP transgene are prepared. The ratio of these plasmids in the transfection mixture follow standard AAV production protocols, with the exception that they are optimized for small scale packaging.
5) The diluted plasmid DNA are added to the cells. Transfection can be performed using a suitable method such as calcium phosphate, PEI, or lipofection.
6) Following transfection, the cells are incubated for 48-72 hours to allow AAV production.
7) AAV is harvested from cells and supernatant. AAV purification might be necessary if the potential impact of cyclic peptides on downstream analysis is a concern. High throughput purification systems for AAV are commercially available but are not cost effective in high throughput screening methods.
8) The viral titer is quantified. Physical titer is quantified by qPCR (or ddPCR), while biological titer is determined by transduction of target cells and analysis of GFP expression by flow cytometry.
9) The titer data is analyzed to assess the effect of each cyclic peptide on AAV production relative to titer data from AAVs packaged in the absence of exogenously added cyclic peptide. Cyclic peptide hits are further characterized in subsequent experiments.
10) This screening process thus enables the identification of cyclic peptides that can enhance AAV production. As in the lentiviral system, peptides might exert different effects on AAV production, including toxicity, antiviral effects, or enhancement of viral packaging.

### Example 7. Cell Line Growth Optimization

Scaling up stable cell lines is a major challenge of viral vector performance. In the context of AAV, the Rep proteins are known to be particularly cytotoxic, while at the same time indispensable for viral production. Rep proteins, particularly Rep78 and Rep68, have been found to be cytotoxic, causing cell cycle arrest and apoptosis. This can make challenging to generate stable cell lines for producing AAV vectors. As such, finding a way to address the cytotoxicity while also maintaining the protein's function would dramatically improve the scalability of viral production with stable cell lines. This example illustrates how the cyclic peptide viral production technology can be used to optimize the properties of a stable producer cell line for viral production.

A modified selection system that incorporates elements of the AAV selection system and the lentiviral packaging system is established.

A lentiviral transgene comprising two components is constructed: a Rep gene encoding a Rep protein, preferably under control of an inducible promoter system (to adjust selection stringency) and a multiple cloning site, and preferably a selectable marker (e.g., G418/Geneticin to select for stable genome integration effects).

A DNA expressed cyclic peptide library operably linked to two or more AAV ITRs, optionally including a reporter protein, is generated by standard molecular cloning techniques known to those in the art and exemplified in FIG. 16.

This ITR-cyclic peptide library is then inserted into the lentiviral multiple cloning site. The resulting lentiviral transfer vector plasmid DNA now contains a cyclic peptide library capable of being packaged into AAV particles, a Rep protein expression construct (preferably under inducible expression control), which will create the selective pressure, and preferably a selectable marker, which can be used to select for successful lentiviral transductions. This plasmid, being generated in E. co/i, is not under any selective pressure from the Rep protein at this point because E. co/i cell is not a mammalian cell (expression is not toxic).

Next, lentiviral packaging commences using methods described previously. Because the effector compound for Rep expression is withheld, the impact of Rep, while not eliminated, is mitigated. This is enough to permit lentiviral packaging. The resulting lentiviral particles are harvested.

Next, fresh cells (e.g. HEK293) are prepared and transduced by the lentiviral vectors. The effector compound to induce expression of the Rep protein is added. Additionally, the antibiotic compounds are added to select for cells that were successfully transduced by lentivirus, while killing off those that were not. The result is a cell library in which each cell expresses the cytotoxic Rep proteins at an amount determined by the effector compound concentration as well as expression of a cyclic peptide that may serve to detoxify one or more of the Rep proteins.

This cell library is then subcultured into a large volume of fresh media and a selection experiment is allowed to proceed. Cells that succumb to Rep mediated cytotoxicity will die or grow slowly. On the other hand, cells that manage to successfully detoxify the Rep protein (e.g., via the cyclic peptide) rapidly multiply and are enriched in the large volume of cell culture. It is also possible that cells mutate the Rep and “cheat.”

After a certain amount of time, fractions of the cells are collected. These cells are then doubly transfected with plasmids that configure them to start to produce AAV particles. It is critical to note that the Rep protein is not supplied at this step, because it must be supplied on the chromosomal insertion. This selects for a functional Rep protein. The ITR-flanked cyclic peptide sequence on the genome of the cell is then mobilized as the cell's AAV production environment mobilizes the AAV genome and starts packaging it into viral particles. The viral particles are then harvested and can be optionally sequenced via NGS.

Finally, the DNA is harvested, and the cyclic peptide library can be reinserted into fresh lentiviral Rep vector for another round of selection. This enrichment scheme can repeat multiple times, preferably being tracked by NGS. Over multiple rounds of enrichment, cyclic peptides that detoxify Rep are selected for. These cyclic peptide generators can then be permanently integrated into the genome, along with Rep and other AAV packaging genes to make a highly productive and scalable stable cell line.

### Example 8. Producing the AAV Vector Composition of Increased Viral Titer and/or Transduction Efficiency

1. A 100-mm cell culture dish was reseeded with 4.0×10\hat{ }6 Hek293T cells in high glucose DMEM culture medium supplemented with 10% FBS.
2. One day after plating, the cells (70-80% confluence) were co-transfected with 3.2ug pRC2 which encodes Rep and Cap genes (SEQ ID NO: 1-7), 4.6ug pHelper which encodes Adenovirus helper genes (SEQ ID NO: 16-19, 22, 23), 2.2ug payload vector, and 1-100ng engineered cyclic peptide expression vector (if using). A jetOPTIMUS DNA transfection reagent i used during the transfection protocol (from Polyplus).
3. 12-16 hours after transfection, media was replaced with low glucose DMEM culture medium supplemented with 1% FBS, 1×Glutamax, 10 mM HEPES, and 0.075% sodium bicarbonate.
4. Cells and media were harvested 4 days after transfection and frozen in 1 mL aliquots.
5. Frozen aliquots were thawed and lysed using AAVMax Lysis Reagent (Life Technologies, USA, A50520). 100 uL of AAVMax Lysis buffer was added to 900 uL of cell and media. Mixture was incubated for 2 hours with rotating. Debris was pelleted at 5,000xg for 15 minutes. AAV containing supernatant was collected.
6. The physical and biological titers of the harvested AAV samples were determined by qPCR and biological viral titer measurement as described in Example 1.

The physical titer fold improvement was calculated by dividing the physical titer of AAVs packaged in cells expressing a CP by the physical titer of cells that are not expressing a CP. FIG. 20 (Part A) depicts the fold improvements in physical titer of AAVs packaged in cells expressing individual relative to AAVs packaged in cells under essentially identical conditions but with no CP expression.

The fold improvement in biological titer was calculated by dividing the percentage of GFP expressing (transduced) cells after transduction with AAVs packaged in cells expressing a CP by the percentage of GFP expressing (transduced) cells after transduction with AAVs packaged in cells that are not expressing a CP. FIG. 20 (Part B) and FIG. 21 depict the fold improvements in biological titer of AAVs packaged in cells expressing individual relative to AAVs packaged in cells under essentially identical conditions but with no CP expression.

Full, but non-functional viral particles represent a major manufacturing challenge for AAV gene therapies. At present, there is no process capable of purifying the functional viral particles from the full, but non-functional fraction. FIG. 23 depicts the CP-mediated depletion of full, but non-functional virions. A greater percentage of full AAV capsids packaged in cells expressing a cyclic peptide were capable of viral transduction relative to the full AAV capsids packaged in cells expressing no cyclic peptide. Therefore, the cyclic peptides depicted in FIG. 23 improve the transduction efficiency by reducing full, but non-functional viral capsids during AAV production.

A standard triple transfection protocol can also be used (Life Technologies, USA, AAV-MAX Helper-Free AAV Production System, VPC 2.0) in this Example, and may be used in other methods disclosed herein.

Required Materials: Viral Production Cells 2.0, specific plasmids, an AAV-MAX Transfection Kit are needed, in addition to other specified reagents and equipment.

Cell Preparation: Expand cells until they reach a density of about 4 to 6 million cells per mL.

Transfection Procedure (Day 0): Determine viable cell density and percent viability. Cells should have reached a density of approximately 4.5 to 6 million viable cells/mL with viability≥95%. Dilute the cells to a final density of 3 million viable cells/mL with fresh Viral Production Medium supplemented with 4 mM GlutaMAX™ Supplement. Immediately add AAV MAX Enhancer and incubate the cells in a 37° C. incubator with a humidified atmosphere of 8% CO2 on an orbital shaker.

Preparation of Transfection Complexes: Prepare transfer plasmid DNA, Rep/Cap plasmid DNA, and helper plasmid DNA at the recommended ratio. Then prepare transfection complexes involving the AAV MAX Transfection Reagent, Viral Plex™ Complexation Buffer, and AAV MAX Transfection Booster.

Incubation: Incubate the transfection complexes at room temperature for 20 to 30 minutes, then transfer the solution to the shaker flask(s). Incubate the cells in a 37° C. incubator with a humidified atmosphere of 8% CO2 on an orbital shaker for approximately 72 hours.

Harvesting AAV Particles: Approximately 70 to 72 hours post-transfection, add AAV-MAX Lysis Buffer directly to the culture flask at a 1:10 dilution and swirl to distribute the lysis buffer. Then, incubate at 37° C. for at least 2 hours on an orbital shaker. After incubation, transfer the cell lysate to a container and centrifuge. Transfer the supernatant containing crude AAV particles to an appropriate storage container.

Storage: Harvested crude AAV particles can be stored at −80° C. for long-term storage. [00713] pRC2, which encodes the Rep and Cap genes for AAV2, was selected for this experiment because AAV2 is the best characterized AAV serotype at present. However, alternative serotypes may be selected based on several desired characteristics: the cell or tissue types being targeted, the safety profile of the serotype or desired payload, and/or the current manufacturing process, Rep/Cap plasmid sets for additional serotypes are commercially available from numerous sources (e.g., Takara Bio USA Inc and Life Technologies, USA Inc). These plasmids commonly encode the Rep gene from AAV2 and substitute the Cap gene for a custom serotype. The helper plasmid for AAV packaging is compatible with all serotypes.

AAV replication genes, AAV capsid encoding genes, and AAV helper genes suitable for use in the methods disclosed herein (e.g., for production of adeno-associated virus (AAV) vector composition in host cells) are commercially available, and a skilled person would be able to identify a combination of at least one AAV replication gene, at least one AAV capsid encoding gene and at least one AAV helper gene to be expressed in particular host cells to produce a functional AAV vector composition. For example, Takara Bio USA offers AAVpro plasmid sets for AAV2 (Cat. #6230), AAV1 (Cat. #6672), AAV5 (Cat. #6650), and AAV6 (Cat. #6651). These sets contain the relevant Rep/Cap plasmid that expresses the Rep gene of AAV2 and the Cap gene of each serotype (e.g., pRC2), the helper plasmid that expresses adenovirus E2A, E4, and VA (e.g., pHelper), and a payload vector (e.g., pCMV). In another example, Life Technologies, USA offers the AAV-MAX Control Plasmids Kit (Cat. #A47672) which consists of an optimized mix of three plasmids needed to produce adeno-associated virus serotype 2 (AAV2)-expressing GFP: pAAV-CMV-emGFP, pAAV-Rep2Cap2, and pHelper. These three plasmids must be used with a HEK293 cell line that stably expresses the adenovirus E1 gene. The kit contains an optimized mix of the three plasmids, as well as a separate tube of each plasmid, providing users with flexibility to change plasmid ratios if desired. There are a variety of other commercially available AAV plasmid kits.

In another example, methods for generating a plurality of host cells comprising proteins necessary for production of AAV vector compositions are disclosed in U.S. patent Ser. No. 10/415,020 B2 and U.S. patent Ser. No. 10/072,250 B2, incorporated by reference. Examples of host cell compositions comprising elements essential for AAV vector formation include host cell having (a) a nucleic acid encoding the Rep protein, (b) a nucleic acid encoding the Cap protein, and (c) a nucleic acid encoding adenovirus-derived elements. These nucleic acids can be inserted into a plasmid or a viral vector as one or more nucleic acid constructs capable of providing the elements, and then the plasmid or the viral vector can be introduced into the host cells. Examples of a nucleic acid constructs include a pRC2-mi342 vector and pHelper vector (manufactured by TAKARA BIO Inc.), which are commercially available plasmids. In embodiments where host cells are insect cells, and a baculovirus vector is used for introduction of nucleic acid constructs for (a)-(c), vectors such as Bac-Rep and Bac-Cap may be used. In some embodiments, the nucleic acid encoding the Cap protein encodes not only the Cap protein but also an assembly-activating protein (AAP) necessary for formation of an AAV particle in an open reading frame different from that of the encoded Cap protein (Proc. Natl. Acad. Sci. USA, 2010, Vol. 107, pp. 10220-10225). Examples of host cells for preparing AAV vector composition-producing host cells include mammalian cells such as human, monkey, and rodent cells, and non-limiting examples thereof include cells having high transformation efficiency, such as a 293 cell (ATCC CRL-1573), a 293T/17 cell (ATCC CRL-11268), a 293F cell, a 293FT cell (all manufactured by Life Technologies, USA), a G3T-hi cell (WO2006/035829), an Sf9 cell (ATCC CRL-1711), and an AAV293 cell (manufactured by Stratagene Corp.) which are all commercially available cell lines for viral production.

### Example 9. Producing the Lentivirus Vector Compositions of Increased Viral Titer and/or Transduction Efficiency

In the methods disclosed herein, a protocol may be used for transfection and lentivirus production with Lenti-X Packaging Single Shots, using pre-aliquoted, lyophilized, single tubes of Xfect™ Transfection Reagent premixed with an optimized formulation of Lenti-X lentiviral packaging plasmids (Takara Bio USA, Inc).
1. A 100-mm cell culture dish was seeded with 4.0×10\hat{ }6 Hek293T cells in high glucose DMEM culture medium supplemented with 10% FBS is used.
2. One day after plating, the cells reached 70-80% confluence.
a. 7ug of lentivirus payload vector and 1-100ng engineered cyclic peptide expression vector (if using) were prepared in 600 uL of pure water.
b. 600 uL of the above mixture was transferred into LentiX Packaging Single Shot tube and the entire mixture was mixed by vortexing before incubating for 10 minutes at room temperature.
c. Entire mixture was transferred into 100-mm cell culture dish.
3. Media were harvested 3 days after transfection.
4. Physical titer is measured using LentiX Go-Stix (Takara Bio USA, Inc).
5. Biological titer is measured as described in Example 1.
6. Fold improvements in viral titer are calculated as described in Example 1.

FIG. 25 depicts exemplary improvements in both physical and biological titer of lentivirus packaged in cells expressing the cyclic peptides as described in this Example.

The Takara Bio USA Lenti-X Packaging Single Shots (Integrase Deficient) (Cat. #631278) was used in this Example because it provides a one-step production method to generate high-titer lentivirus. Each tube contains premeasured, lyophilized Xfect Transfection Reagent and an optimized formulation of lentiviral packaging plasmids. The integration deficient pseudotype was chosen for safety. The choice of pseudotype may be based on a variety of priorities including tissue tropism, cytotoxicity concerns, safety, and/or manufacturing efficiency.

Lentivirus essential genes (e.g., at least one lentiviral gag gene, at least one lentiviral pol gene, at least one lentiviral rev gene, and at least one lentiviral-compatible env gene) suitable for use in the methods disclosed herein (e.g., for production of lentivirus vector composition in host cells) are commercially available, and a skilled person would be able to identify a combination of at least one lentiviral gag gene, at least one lentiviral pol gene, at least one lentiviral rev gene, and at least one lentiviral-compatible env gene to be expressed in particular host cells to produce a functional lentivirus vector composition. For example, there are a variety of commercially available lentivirus plasmid kits. Takara Bio USA offers VSV-G Lenti-X Packaging Single Shots (Cat. #631276) as well as an envelope free Lenti-X Packaging Single Shots (Cat. #631294) where the user can use a custom envelope plasmid. In another example, complete description of a lentiviral packaging protocol with links to specific sequences on Addgene and references to nucleic acid sequences can be found in Wickersham I R, et al., Lentiviral vectors for retrograde delivery of recombinases and transactivators. Cold Spring Harb Protoc. 2015 Apr. 1; 2015(4):368-74. In yet another example, production of lentivirus vector composition in host cells are described in U.S. Pat. No. 6,207,455 B1, U.S. Pat. No. 9,057,056 B2 and U.S. Pat. No. 9,102,943 B2, incorporated herein by reference. Examples of host cell compositions comprising elements essential for production of lentivirus vector composition include packaging cell lines such as PG13 (ATCC CRL-10686), PA317 (ATCC CRL-9078), GP+E-86 or GP+ envAM-12 (U.S. Pat. No. 5,278,056), or Psi-Crip (Proc. Natl. Acad. Sci. USA, 85:6460-6464 (1988)). In some embodiments, the at least one lentiviral-compatible env gene is an envelope gene encoding a glycoprotein from an enveloped virus. In one particular embodiment, at least one env gene encodes the protein comprising amino acid sequence set forth in SEQ ID NO:27.

In some embodiments, for preventing appearance of a replication competent lentivirus particle, a gag-pol gene and an env gene are not located in proximity in a host cell used in the disclosed methods. For example, it may be preferable to use a host cell having a gal-pol gene and an env gene integrated at different positions on a chromosome, or a host cell having a plasmid containing a gag-pol gene and another plasmid containing an env gene.

The env gene is not limited to one encoding an envelope protein derived from the same virus as the lentivirus vector composition to be produced. In some embodiments, a host cell for pseudotyped packaging which has an env gene derived from a heterologous virus is used in the disclosed methods. For example, an env gene derived from Moloney murine leukemia virus (MoMLV), vesicular stomatitis virus (VSV), gibbon ape leukemia virus (GaLV), or a gene encoding a protein that can function as env can be used as the env gene.

In some embodiments, a lentivirus vector composition comprises human immunodeficiency virus (HIV)-derived vector, feline immunodeficiency virus (FIV)-derived vector, or simian immunodeficiency virus (SIV)-derived vector. In some embodiments, lentiviruse vectors (including HIV, SIV, feline immunodeficiency virus (FIV) and equine infectious anemia virus (EIAV)) depend on several viral regulatory genes in addition to the structural gag-pol-env genes for efficient intracellular replication. Lentiviruses use more complex strategies than classical retroviruses for gene regulation and viral replication, with the packaging signals apparently spreading across the entire viral genome. These additional genes display a web of regulatory functions during the lentiviral life cycle. For example, upon HIV-1 infection, transcription is up-regulated by the expression of Tat through interaction with an RNA target (TAR) in the LTR. Expression of the full-length and spliced mRNAs is then regulated by the function of Rev which interacts with RNA elements present in the gag region and in the env region (RRE) (S. Schwartz et al., J. Virol., 66:150-159 [1992]). Nuclear export of gag-pol and env mRNAs is dependent on the Rev function. In addition to these two essential regulatory genes, a list of accessory genes, including vif vpr, vpx, vpu, and nef are also present in the viral genome and their effects on efficient virus production and infectivity have been demonstrated, although they are not absolutely required for virus replication (K. and F. Wong-Staal, Microbiol. Rev., 55:193-205 [1991]; R. A. Subbramanian and E. A. Cohen, J. Virol. 68:6831-6835 [1994]; and D. Trono, Cell 82:189-192 [1995]).

In some embodiments, for the construction of replication-defective lentiviral vectors, the attenuated HIV-1 constructs are used. In one embodiment, the expression vector can synthesize all viral structural proteins but lacks the packaging signal function (“pHP”), includes a strong promoter (yet preferably not a native HIV-1 LTR), the gag-pol gene, the RRE element and the rev gene. In some embodiments, an expression construct (pHP-1) which contained a modified 5′ HIV-1 LTR, a novel major splice donor site based on RSV splice sequences, the entire gag-pol-env, vif, vpr, vpu, tat, and rev genes, a selectable gpt marker gene, and an SV40 polyadenylation signal are used as disclosed in U.S. Pat. No. 6,207,455 BL.

### Example 10. Producing a Lentivirus Vector Composition of Increased Viral Titer and/or Transduction Efficiency by Using an Engineered Cyclic Peptide

Producing highly infectious lentiviral particles at scale, similar to AAV, presents significant challenges, particularly when using recombinant systems with numerous modifications and differences from wild-type (WT) systems. These differences can lead to deoptimizations that are not entirely understood. For example, vectors that retain certain elements, such as VSV-G envelope glycoprotein or accessory genes like Vpr and Vif, can be toxic to cells. Inactivation or deletion of these genes can reduce cellular toxicity but may negatively impact transgene expression. Achieving an optimal balance between viral replication, transgene expression, and cellular toxicity remains a crucial challenge in the development of safe and effective lentivirus-based vectors for clinical use. Consequently, there is a need to improve the performance and productivity of lentiviral packaging systems while maintaining an exceptional safety profile.

The same basic approach described for AAV can be applied to lentivirus despite differences in their distinct lifecycles. Lentivirus is a member of the Retroviridae family, a group of enveloped RNA viruses with a unique replication cycle that involves reverse transcription of their RNA genome into DNA, followed by integration of the DNA into the host cell's genome. Example members include human immunodeficiency virus (HIV), simian immunodeficiency virus (SIV), and feline immunodeficiency virus (FIV). These integrating RNA viruses can infect a broad range of human and animal cells.

The lentiviral genome consists of several genes, including gag, pol, and env, which encode structural proteins, enzymes, and regulatory proteins. Key coding genes include gag (encoding matrix, capsid, and nucleocapsid proteins), pol (encoding reverse transcriptase, integrase, and protease), and env (encoding envelope glycoproteins gp120 and gp41). These genes play essential roles in viral replication, assembly, gene expression, immune evasion, and interaction with the host cell.

The viral RNA contains various elements, including promoters, terminators, and other regulatory sequences, that control viral gene expression and replication. The genome also contains regions that are transcribed into non-coding RNAs, which may have regulatory functions. The viral RNA is packaged into nucleocapsids, which are protected by the capsid and envelope proteins.

Lentiviral replication involves reverse transcription of the viral RNA into DNA, which then integrates into the host cell genome. This integrated provirus serves as a template for viral gene expression and production of new viral particles. To create replication-defective lentiviral vectors, essential genes required for viral replication are removed from the transfer vector and provided in trans by the packaging cell line or plasmid DNA. This prevents the emergence of replication-competent viral particles.

The capsid and envelope are essential for the replication and spread of lentiviruses. The capsid protects the viral RNA from the host's immune system and ensures the virus can enter and infect cells. The envelope glycoproteins, gp120 and gp41, play crucial roles in various functions necessary for the virus to replicate and spread, including attachment to the host cell, entry into the host cell, uncoating of the capsid, reverse transcription of the viral RNA, integration into the host genome, assembly of new virions, and regulation of the host immune response.

Recombinant lentiviral systems have been developed to enable the packaging of genes into lentiviral particles, which can then be transferred to cells through infection with these recombinant lentiviral particles. A variety of recombinant lentiviral packaging systems, including 2nd, 3rd, and 4^{th} generation systems, have been designed to balance safety and productivity. Each generation aims to minimize the possibility of generating replication-competent lentiviruses while maintaining efficient gene delivery.

Although there are differences among these systems, the general concept involves dividing the single genome of the wild-type (WT) lentivirus into multiple plasmids to prevent the emergence of replication-competent viral particles. Recombinant systems typically comprise a transfer vector, also known as an expression plasmid (containing the desired genes to be packaged into the recombinant viral vector, such as GFP), in conjunction with one or more packaging plasmids that incorporate envelope genes and other essential genes required for viral packaging, including gag, pro, pol, vpr, tat, rev, env, and others.

Design and production of lentiviral vectors is known in the art: (Designing Lentiviral Vectors for Gene Therapy of Genetic Diseases, 2021; Clinical use of lentiviral vectors, 2017; New developments in lentiviral vector design, production and purification, 2013; Lentiviral Vector Bioprocessing, 2021; Retroviral Vectors for Cancer Gene Therapy, 2016; Concise review on optimized methods in production and transduction of lentiviral vectors in order to facilitate immunotherapy and gene therapy, 2020).

Examples of elements that may be required in certain embodiments for lentiviral vector production include:
1) Structural Proteins—Core Structure Formation
a) Gag (Encoded by gag gene): The Gag protein is a precursor polyprotein that plays a central role in the formation of the viral core structure. Gag undergoes proteolytic processing to generate matrix protein (MA), capsid protein (CA), and nucleocapsid protein (NC). MA is responsible for membrane binding and targeting Gag to the plasma membrane, CA forms the conical capsid structure that encloses the viral RNA and proteins, and NC binds tightly to the viral RNA, protecting it from digestion by nucleases.
b) MA (Matrix Protein): The matrix protein lies beneath the viral envelope and associates with the inner leaflet of the host cell membrane. It provides structural support to the virion and plays a role in the assembly and release of new virions.
c) Nucleocapsid (NC): Binds tightly to the viral RNA, protecting it from digestion by nucleases.
2) Structural Proteins—Capsid Assembly and Stability
a) Capsid protein (CA): Forms the conical capsid structure that encloses the viral RNA and proteins. It plays a crucial role in capsid assembly and stability, ensuring the integrity of the viral core. CA interacts with both viral and host factors to orchestrate proper capsid formation during viral assembly.
3) Structural Proteins—Envelope
a) Env (Encoded by env gene): Encodes envelope glycoproteins (gp120 and gp41) responsible for viral entry into target cells. gp120 is involved in binding to the host receptor (CD4) and co-receptor (CCR5 or CXCR4), initiating the viral entry process. gp41 mediates fusion of the viral and host cell membranes, allowing entry of the viral core into the target cell.
4) Replication Proteins—Pol (Encoded by pol gene): Encodes enzymes essential for viral replication.
a) Reverse Transcriptase (RT): RT is a multifunctional enzyme responsible for the conversion of the viral RNA genome into DNA through reverse transcription. It possesses both RNA-dependent DNA polymerase activity and RNase H activity, which degrades the RNA template within the RNA-DNA hybrid during reverse transcription.
b) RNase H: RNase H is an endonuclease that specifically degrades the RNA strand of the RNA-DNA hybrid during reverse transcription. This activity is crucial for the synthesis of the viral DNA.
c) Integrase (IN): IN catalyzes the integration of the viral DNA into the host cell genome. It cleaves the terminal dinucleotides from the viral DNA ends and mediates the strand transfer reaction, inserting the viral DNA into the host chromosomal DNA.
d) Protease (PR): PR is responsible for the proteolytic processing of the precursor polyproteins into individual functional proteins during viral maturation. It cleaves Gag and Gag-Pol polyproteins at specific sites, leading to the formation of mature viral proteins required for assembly and infectivity.
5) Regulatory Proteins:
a) Tat (Encoded by tat gene): Transcriptional activator that regulates viral gene expression.
b) Rev (Encoded by rev gene): Facilitates the nuclear export of viral mRNAs, ensuring efficient viral protein synthesis.
6) Exemplary Accessory Proteins:
a) Nef (Encoded by nef gene): Modulates immune response, enhances viral replication, and promotes infectivity.
b) Vpr (Encoded by vpr gene): Induces cell cycle arrest and facilitates nuclear import of the preintegration complex.
c) Vif (Encoded by vif gene): Counteracts host antiviral protein APOBEC3G, allowing efficient viral replication.
d) Vpu (Encoded by vpu gene): Enhances virion release and facilitates CD4 degradation.
e) Vpx (Encoded by vpx gene): Encoded by HIV-2 and SIV, functions in association with Vpr.
6) Nucleic Acid Elements:
a) RNA genome: The viral genome is composed of single-stranded RNA that carries the genetic information of the virus.
b) Inverted repeats: Flanking the unique segments of the viral genome, these repeats are essential for circularization and replication of the viral genome.
c) Open reading frames (ORFs): Segments of the viral genome that encode proteins.
d) Promoters and enhancers: Regulatory elements involved in the control of gene expression.
e) Transcriptional regulatory elements: Regions of the viral genome involved in the regulation of viral gene expression.
f) Termination signals: Sequences that signal the end of transcription.
g) Long terminal repeats (LTRs): Regions at the ends of the viral genome that contain important regulatory elements.
h) Polyadenylation signal: Sequence that directs the addition of a poly(A) tail to the viral mRNA.

### Exemplary Recombinant Lentivirus Vector Systems

A variety of lentiviral systems have been developed, including replication competent, attenuated, and replication incompetent. Recombinant lentiviral systems allow genes to be packaged into lentiviral particles and subsequently transferred to cells through infection with these recombinant lentiviral vectors. Recombinant lentiviral packaging systems come in a variety of forms, attempting to balance safety and productivity. Each system has been designed to reduce the possibility of generating replication-competent lentivirus while maintaining efficient gene delivery. Such recombinant systems comprise a transfer vector, also known as an expression plasmid (which contains the desired genes to be packaged into the recombinant viral vector, such as GFP), along with one or more packaging plasmids that provide the essential genes required for viral replication and packaging.

In one embodiment, the invention introduces engineered CPs that enhance various aspects of lentiviral vector production, including viral replication, yield, scalability, infectiousness, and decreased cytotoxic effects on host cells. These advancements significantly improve the suitability of lentivirus vectors for applications such as human gene therapy, gene transfer, and other applications in biotechnology and medicine.

Cyclic peptide disclosed herein can enhance production of lentivirus vectors.

In one embodiment, CPs are used to enhance production of lentivirus by an increase infectious titer, defined as the number of infectious units per mL, physical titer, defined as the number of viral particles per mL, or TU:VG ratio, defined as the ratio of transducing units to viral genomes. The embodiment can be described as follows:
1) Cell Component: The cell component of this embodiment involves the use of a cell line configured to express the essential lentiviral genes Gag, Pol, and Env. These genes provide the necessary factors for lentiviral replication and packaging.
2) Viral Component: The viral component of this embodiment includes a transfer vector carrying a therapeutic gene payload. The transfer vector contains the necessary lentiviral elements, such as the capsid protein (e.g., CA), reverse transcriptase (RT), integrase (IN), and protease (PR), as well as essential regulatory elements (e.g., LTR, packaging signal). The therapeutic gene payload is positioned within the lentiviral genome for efficient packaging and delivery to target cells. The lentivirus is a member of the Retroviridae family, including lentiviral derivatives, hybrids of more than one line, or chimeras of more than one type of virus (e.g., lentivirus-AAV, lentivirus-AdV).
3) CP embodiment variations:
a) A CP generator gene sequence (e.g., SICLOPPS) is stably integrated into the genome of the packaging cell.
b) A CP produced by synthetic chemistry is added exogenously to the growth media.
c) A CP generator gene sequence (e.g., SICLOPPS) is added to the transfer vector in such a way that it is not packaged, for example by placing the CP generator outside of the terminal repeats (e.g. on a shuttle plasmid)

In another embodiment, CPs are discovered using an enrichment-based approach.

A large CP generator gene sequence library is inserted into the transfer vector as a recombinant payload gene. The lentivirus transfer vector bearing the CP library is then transfected, along with other packaging plasmids, into the packaging cell. Upon transfection, the plasmids begin to express.
1) The packaging cell line generates the necessary materials for recombinant viral production and packaging of the CP library variant(s) within the cell.
2) Simultaneously, the CP generator is expressed from the expression plasmid or transfer vector resulting in the intracellular biosynthesis of cyclic peptide(s) encoded by the CP generator variant(s).

The CPs then bind their targets, causing a perturbation in the cell's physiological state.
1) If this CP-induced cellular perturbation improves the cell's ability to produce viral particles by any metric (e.g., yield, quality/infectiousness), then the CP gene sequence will be more efficiently packaged and will increase its relative representation of the population.
2) In contrast, if this CP-induced cellular perturbation reduces the cell's ability to generate viral particles, then the CP gene sequence will be less efficiently packaged and will be depleted or eliminated from the population.

CP-induced cellular perturbations may alter several host cell or lentivirus-specific processes, including viral replication, genome synthesis, viral assembly, and egress. The efficiency of these processes can be dramatically altered by the cellular context or perturbations like mutations or changes in gene expression. The resulting library of viral particles will contain a subset of the original CP diversity:
1) Lentiviral particles harboring CP gene sequences that reduced viral packaging are reduced or eliminated from the library (these CPs might be antivirals, and indeed might find utility in such applications)
2) Viral particles harboring CP gene sequences that improved viral packaging are increased or enriched in the library
3) The CP gene sequences that had no impact on the viral packaging are also reduced due to competition from Viral particles harboring CPs gene sequences that improved viral packaging and outcompeted the neutral variants.

Resulting lentivirus vectors, each harboring one or more cyclic peptide generator sequences can then be used to infect fresh packaging cells configured to generate additional lentivirus vectors.
1) For viral particles to be generated, the transfer vector/expression plasmid be present inside of the packaging cell. This requires that viral particles be functional and able to successfully deliver their genetic payload into the cell.
2) CP gene sequences in non-functional viral particles (e.g. they interfere with viral production) are eliminated.
3) In contrast, viral particles that harbor CP gene sequences that previously altered the packaging cell in a way that resulted in enhanced infectivity, quality, payload delivery, or simply a higher population of viral particles with the same infectiousness, will result in the delivery of more of these performance-enhancing CP payloads into packaging cells.
4) Viral particles that are better able to deliver their CP gene sequence payload into the cell (e.g. because there are more of them or because the CP enhances viral production) gain a selective advantage and can increase their representation in the population.

This increases the number of cells that will generate viral particles harboring CP gene sequences that enhance viral production. In other words: Engineered cyclic peptides that enhance viral production enhance the packaging of their own coding DNA into more or better viral particles.

In summary, CP gene sequences that increase infectiousness or viral yield gain a selective advantage and become enriched in the population. CP gene sequences that decrease lentivirus production are rapidly depleted. CP gene sequences can be isolated and analyzed for their impact on lentivirus production.

At each step, the CP library can be subjected to NGS to understand the composition of the entire library. This provides information about which variants are most likely to be useful for increasing physical and biological titer. CP gene sequences that show very high-performance enhancements can be synthesized and tested individually for their ability to increase viral production.

In addition, after enrichment, the library can be screened directly to identify viral production enhancing CPs.

### Example 11. Producing a Herpes Simplex Virus (HSV) Vector Composition of Increased Viral Titer and/or Transduction Efficiency by Using an Engineered Cyclic Peptide

In some embodiments, engineered cyclic peptides are used to obtain recombinant HSV vector composition of increasing viral titer and/or transduction efficiency. Essential genes and components for recombinant HSV packaging are known in the art. Similar to AAV, producing highly infectious HSV particles at scale presents significant challenges, particularly when using recombinant systems that have numerous modifications and differences from the wild-type (WT) systems. These differences can lead to deoptimizations that are not entirely understood. For example, vectors that retain the ICP0 and ICP22 IE genes are toxic to cells. Inactivation or deletion of these genes, particularly the ICP0 gene, can reduce cellular toxicity but may negatively impact transgene expression. Achieving an optimal balance between viral replication, transgene expression, and cellular toxicity remains a crucial challenge in the development of safe and effective HSV-based vectors for clinical use. Consequently, there is a need to improve the performance and productivity of HSV packaging systems while maintaining an exceptional safety profile.

The same basic approach described for AAV can be applied to HSV despite differences in HSV's distinct lifecycle. Herpes simplex virus (HSV) is a member of the Herpesviridae family, a group of large enveloped DNA viruses with a lytic-latent replication cycle. Example members include simplex virus-1 (HSV-1), herpes simplex virus-2 (HSV-2), human cytomegalovirus (HCMV), varicella-zoster virus (VZV), Epstein-Barr virus (EBV), Kaposi's sarcoma-associated herpesvirus (KSHV), human herpesvirus 6 and/or human herpesvirus 7.

These non-integrating DNA viruses can infect a broad range of human and animal cells. The viral genome consists of over 80 genes and is composed of two unique segments, UL (unique long) and US (unique short), each flanked by inverted repeats that encode critical diploid genes, as well as inverted repeat regions. These inverted repeats are essential for circularization and replication of the viral genome.

The genome contains a variety of genes encoding viral proteins, including structural proteins, enzymes, and regulatory proteins. Key coding genes include UL19 (VP5), UL38 (VP19C), UL18 (VP23), UL26.5 (VP21), UL35 (VP26), UL19 (VP28), UL26 (VP30 and VP35), UL48 (VP16), UL37 (VP19A), UL49 (VP22), UL54 (VP27), UL31 (UL31), and more. These genes play essential roles in viral replication, assembly, gene expression, immune evasion, and interaction with the host cell.

The viral DNA contains various elements, including promoters, terminators, and other regulatory sequences, that control viral gene expression and replication. The genome also contains regions that are transcribed into non-coding RNAs, which may have regulatory functions. The viral DNA is packaged into nucleocapsids, which are protected by the capsid and tegument proteins.

HSV replication involves temporally regulated expression of its genes in waves, referred to as cascade regulation. This regulation involves the sequential expression of immediate-early (IE) genes, early (E) genes, and late (L) genes. The IE genes are the first to be expressed and function to regulate the subsequent expression of E and L genes. Removal of the essential IE genes ICP27 and ICP4 renders the virus completely defective and incapable of expressing E genes involved in viral genome replication and L genes functioning in progeny virion assembly. These replication-defective viruses can be grown on complementing cells that express (complement) the missing ICP4 and ICP27 gene products. After propagation, they can then be used to infect non-complementing cells, where the viral genome resides as a stable nuclear episome, facilitating long-term transgene expression.

Design of HSV vectors is known for someone skilled in the art: (HSV Recombinant Vectors for Gene Therapy, 2010). While there are differences between the systems, the general concept involves using a replication-defective HSV genome as the transfer vector. The essential genes required for viral replication are provided in trans by the packaging cell line, a helper virus, plasmid DNA, etc. . . . . This prevents the emergence of replication-competent viral particles.

The capsid and tegument are essential for the replication and spread of HSV. The capsid protects the viral genome from the host's immune system and helps ensure the virus can enter and infect cells. The tegument plays a crucial role in various functions necessary for the virus to replicate and spread, including attachment to the host cell, entry into the host cell, uncoating of the capsid, transport of the viral DNA to the nucleus, replication of the viral DNA, assembly of new virions, and regulation of the host immune response. Examples of elements that may be required in certain embodiments for HSV vector production include:

Capsid (mostly early genes): VP5 (encoded by UL19 gene): Main component of the capsid, involved in the formation and stability of the viral capsid; VP19C (encoded by UL38 gene): Found in the capsid, but its specific function is not well defined; VP23 (encoded by UL18 gene): Present in the capsid and important for virus replication and formation of infectious viral particles; VP21 (encoded by UL26.5 gene): Functions in capsid assembly and DNA packaging.

Other exemplary capsid proteins may include: VP26 (encoded by UL35 gene): Plays a role in capsid assembly and stability; VP28 (encoded by UL19 gene): Involved in the assembly and stability of the capsid; VP30 (encoded by UL26 gene): Participates in capsid assembly and packaging of viral DNA; VP35 (encoded by UL26 gene): Contributes to capsid stability and assembly.

### Exemplary Tegument Proteins May Include

1) Early genes: VP16 (encoded by UL48 gene): Involved in the regulation of viral gene expression and host immune response; VP19A (encoded by UL37 gene): Functions in tegument assembly and stability; VP22 (encoded by UL49 gene): Participates in tegument assembly and is important for efficient viral replication.
2) Late genes: VP24 (encoded by UL35 gene): Plays a role in tegument assembly and virion egress; VP27 (encoded by UL54 gene): Involved in the regulation of host immune response and viral gene expression; VP39 (encoded by UL26 gene): Required for efficient tegument assembly and stability; UL31 (encoded by UL31 gene): Participates in tegument assembly and virion maturation.

In certain embodiments, additional tegument components include:
1) Lipids: Lipid molecules are present in the tegument and contribute to its structure and stability.
2) Carbohydrates: Carbohydrate molecules are found in the tegument and may be involved in interactions with host cells.
3) Nucleic acids: Tegument can contain viral and host nucleic acids, which may play roles in viral replication and modulation of host responses.

In certain embodiments, additional essential regulatory proteins include: ICP0 (Infected Cell Protein 0, encoded by the RL2 gene): Functions as a key regulator of viral gene expression, contributing to viral replication and establishment of infection; ICP4 (Infected Cell Protein 4, encoded by the IE175 gene): Essential for viral gene expression, serving as a transcriptional activator of both early and late genes; ICP22 (Infected Cell Protein 22, encoded by the US1 gene): Plays a role in regulating viral gene expression, particularly in the control of viral transcription and splicing; ICP27 (Infected Cell Protein 27, encoded by the UL54 gene): Essential for viral gene expression and post-transcriptional processing of viral RNA.

In certain embodiments, essential Nucleic Acid Elements include:
1) Terminal Repeat Long (TRL) and Inverted Repeat Long (IRL): Flanking sequences at the ends of the long unique segment (UL) of the HSV-1 genome.
2) Internal Repeat Short (IRS) and Terminal Repeat Short (TRS): Flanking sequences at the ends of the short unique segment (Us) of the HSV-1 genome.
3) Pac (Packaging Sequence): A specific sequence located within the unique long (UL) region that is recognized by the viral packaging machinery during the encapsidation of viral DNA.

In certain embodiments, additional regulatory elements include regulatory elements involved in gene expression, replication, and genome organization.

A variety of HSV systems have been developed, including replication competent, attenuated, replication incompetent. Recombinant HSV systems allow genes to be packaged into HSV particles and subsequently transferred to cells through infection with these recombinant HSV vectors. Recombinant HSV packaging systems come in a variety of forms, attempting to balance safety and productivity. Each system has been designed to reduce the possibility of generating replication-competent HSV while maintaining efficient gene delivery. Such recombinant systems comprise a transfer vector, also known as an expression plasmid (which contains the desired genes to be packaged into the recombinant viral vector, such as GFP), along with a packaging cell line that provides the essential genes required for viral replication and packaging.

CPs can be used to improve HSV production. In one embodiment, CPs are used to enhance production of HSV by an increase infectious titer, defined as the number of infectious units per mL, physical titer, defined as the number of viral particles per mL, or TU:VG ratio, defined as the ratio of transducing units to viral genomes. The embodiment is described as follows:
1) Cell Component: The cell component of this embodiment involves the use of a HEK293 cell line that has been modified to express the essential viral genes ICP4, ICP22, ICP27, and ICP47. These genes provide the necessary factors for viral replication and packaging. The modified HEK293 cell line serves as a stable cell line to produce HSV viral vector.
2) Viral Component: The viral component of this embodiment includes a transfer vector carrying a therapeutic gene payload. The transfer vector contains the necessary viral elements, such as the capsid proteins (e.g., VP5, VP19C, VP23, VP21) and essential regulatory elements (e.g. TRL/IRL, IRS/TRS, pac sequence). The therapeutic gene payload is positioned within the viral genome for efficient packaging and delivery to target cells. The virus is a member of the Herpesviridae, their derivatives, hybrids of more than one line, or chimeras of more than one type of virus (e.g. HSV-lenti, HSV-AAV, HSV-AdV).
3) CP embodiment variations:
a) A CP generator gene sequence (e.g. SICLOPPS) is stably integrated into the genome of the packaging cell.
b) A CP produced by synthetic chemistry is added exogenously to the growth media.
c) A CP generator gene sequence (e.g. SICLOPPS) is added to the transfer vector in such a way that it is not packaged, for example by placing the CP generator outside of the terminal repeats (e.g. on a shuttle plasmid)

In another embodiment, CPs are discovered using an enrichment-based approach.

A large CP gene sequence library is inserted into the transfer plasmid as a recombinant payload gene. The HSV expression plasmid bearing the CP gene sequence library is then transfected, along with other packaging plasmids, into the packaging cell. Upon transfection, the plasmids begin to express the cyclic peptide(s) encoded by the gene sequence variant(s).The packaging cell line generates the necessary materials for recombinant viral production and packaging of the CP gene sequence library variant(s) within the cell. The CPs then bind their targets, causing a perturbation in the cell's physiological state. If this CP-induced cellular perturbation improves the cell's ability to produce high-quality viral particles by any metric (e.g., yield, quality/infectiousness), then the CP gene sequence will be more efficiently packaged and will increase its relative representation of the population. In contrast, if this CP-induced cellular perturbation reduces the cell's ability to generate viral particles, then the CP gene sequence will be less efficiently packaged and will be depleted or eliminated from the population. CP-induced cellular perturbations may alter several host cell or HSV-specific processes, including viral replication, DNA synthesis, viral assembly, and egress. The efficiency of these processes can be dramatically altered by the cellular context or perturbations like mutations or changes in gene expression. The resulting library of viral particles will contain a subset of the original CP diversity:
1) Viral particles harboring CP gene sequences that reduced viral packaging are reduced or eliminated from the library (these CPs might be antivirals, and indeed might find utility in such applications).
2) Viral particles harboring CP gene sequences that improved viral packaging are increased or enriched in the library.
3) The CP gene sequences that had no impact on the viral packaging are also reduced due to competition from Viral particles harboring CP gene sequences that improved viral packaging and outcompeted the neutral variants.

Resulting HSV vectors, each harboring one or more cyclic peptide generator sequences can then be used to infect fresh packaging cells configured to generate additional HSV vectors. For viral particles to be generated, the transfer vector/expression plasmid is present inside of the packaging cell. This requires that viral particles be functional and able to successfully deliver their genetic payload into the cell. CP gene sequences in non-functional viral particles (e.g. they interfere with viral production) are eliminated. In contrast, viral particles that harbor CP gene sequences that previously altered the packaging cell in a way that resulted in enhanced infectivity, quality, payload delivery, or simply a higher population of viral particles with the same infectiousness, will result in the delivery of more of these performance-enhancing CP gene sequence payloads into packaging cells. Viral particles that are better able to deliver their CP gene sequence payload into the cell (e.g. because there are more of them or because the encoded CPs enhance viral production) gain a selective advantage and can increase their representation in the population. This increases the number of cells that will generate viral particles harboring CP gene sequences that enhance viral production. In other words: Engineered cyclic peptides that enhance viral production enhance the packaging of their own coding DNA into more or better viral particles.

### Example 12. Producing an Adenovirus (AdV) Vector Composition of Increased Viral Titer and/or Transduction Efficiency by Using an Engineered Cyclic Peptide

In some embodiments, engineered cyclic peptides are used to obtain recombinant adenovirus (AdV) vector composition of increasing viral titer and/or transduction efficiency. Essential genes and components for recombinant adenovirus (AdV) packaging are known in the art. Exemplary genes and components are indicated below. Inverted Terminal Repeats (ITRs) are necessary for the initiation of viral DNA replication and packaging of the AdV genome into viral capsids. AdV packaging signal (ψ) is a cis-acting element required for the encapsidation of the viral genome into the viral capsid. AdV early (E) genes are crucial for viral replication and gene expression. AdV E1A and E1B are involved in the activation of other viral genes and cellular factors necessary for viral replication. AdV E2A and E2B encode essential components of the viral DNA replication machinery, including the DNA polymerase and the preterminal protein. AdV E4 contributes to efficient viral DNA replication, late gene expression, and host cell shutoff. AdV late (L) genes encode structural proteins, including capsid proteins (e.g., hexon, penton base, and fiber), core proteins, and other components required for viral assembly and maturation. For recombinant AdV packaging, the transfer vector should include the gene of interest, ITRs, and the packaging signal to facilitate replication and packaging. The packaging cell line should provide the essential AdV genes, such as the E1, E2, and E4 genes, in trans to support viral replication, gene expression, and assembly of viral particles. By supplying these genes in trans and using a packaging cell line with a deleted E1 region, the production of replication-defective recombinant AdV particles can be achieved, improving the safety profile of the viral vector for gene therapy applications.

Producing highly infectious adenoviral particles at scale, similar to AAV and lentivirus, presents significant challenges, particularly when using recombinant systems with numerous modifications and differences from wild-type (WT) systems. These differences can lead to deoptimizations that are not entirely understood. For example, vectors that retain the E1A and E1B early genes in adenoviral systems can be toxic to cells. Inactivation or deletion of these genes can reduce cellular toxicity but may negatively impact viral production. Achieving an optimal balance between viral replication, transgene expression, and safety remains a crucial challenge in the development of safe and effective adenovirus-based vectors for clinical use. Consequently, there is a need to improve the performance and productivity of adenoviral packaging systems while maintaining an acceptable safety profile.

The same basic approach described for AAV and lentivirus can be applied to adenovirus despite differences in their distinct lifecycles. Adenovirus is a non-enveloped DNA virus with a linear, double-stranded DNA genome. The Adenoviridae family includes various human and animal adenoviruses with diverse tropisms and pathogenicity.

The adenoviral genome consists of several genes, including early genes (E1A, E1B, E2A, E2B, E3, and E4), late genes (L1-L5), and non-coding elements, which encode structural proteins, enzymes, and regulatory proteins. Key coding genes include hexon (L3 gene), penton base (L2 gene), and fiber (L5 gene) for the viral capsid, and protein VII (L1 gene), protein V (L3 gene), and terminal protein (L1 gene) for the viral core. These genes play essential roles in viral replication, assembly, gene expression, immune evasion, and interaction with the host cell.

The viral DNA contains various elements, including promoters, enhancers, inverted terminal repeats (ITRs), and packaging sequences, that control viral gene expression and replication. The viral DNA is packaged into the viral core, which is protected by the icosahedral capsid.

Adenoviral replication involves the replication of the viral DNA, transcription of viral genes, and assembly of new viral particles. Adenoviral replication involves the replication of viral DNA, transcription of viral genes, and assembly of new viral particles. The early genes (E1A, E1B, E2A, E2B, E3, and E4) are involved in viral and cellular gene expression regulation, viral DNA replication, and host immune response modulation, while the late genes (L1-L5) encode structural proteins for the capsid and core assembly. The capsid proteins, including hexon, penton base, and fiber, are essential for the replication and spread of adenoviruses. The capsid protects the viral DNA from the host's immune system and ensures the virus can enter and infect cells. The fiber protein plays a crucial role in binding to the host cell receptor and initiating viral entry.

Recombinant adenoviral systems have been developed to enable the packaging of genes into adenoviral particles, which can then be transferred to cells through infection with these recombinant adenoviral particles. A variety of recombinant adenoviral packaging systems, including replication competent, attenuated, and replication-deficient, have been designed to balance safety and productivity. Each system aims to minimize the possibility of generating replication-competent adenoviruses while maintaining efficient gene delivery.

To create replication-defective adenoviral vectors, essential genes required for viral replication are removed from the transfer vector and provided in trans by the packaging cell line or plasmid DNA. This prevents the emergence of replication-competent viral particles.

Although there are differences among these systems, the general concept involves dividing the single genome of the wild-type (WT) adenovirus into multiple plasmids to prevent the emergence of replication-competent viral particles. Recombinant systems typically comprise a transfer vector, also known as an expression plasmid (containing the desired genes to be packaged into the recombinant viral vector, such as GFP), in conjunction with a packaging cell line that provides the essential genes required for viral replication and packaging.

Design and production of lentiviral vectors is known in the art: Adenovirus Vectors for Gene Therapy, Vaccination and Cancer Gene Therapy, 2013; Adenovirus Biology, Recombinant Adenovirus, and Adenovirus Usage in Gene Therapy, 2021; High-Capacity Adenoviral Vectors: Expanding the Scope of Gene Therapy, 2020; Adenoviral gene therapy, 2002; US20040161848A1 (incorporated herein by reference), Adenoviral vectors and related systems, and methods of manufacture and use).

Examples of elements that may be required in certain embodiments for adenoviral vector production include:
1) Capsid Proteins: Hexon (encoded by L3 gene): Major capsid protein that forms the outer surface of the viral particle and is involved in viral entry; Penton base (encoded by L2 gene): Forms the vertex of the icosahedral capsid and anchors the fiber protein; Fiber (encoded by L5 gene): Projects from the penton base and is responsible for binding to the host cell receptor, initiating viral entry.
2) Core Proteins: Protein VII (encoded by L1 gene): Binds and condenses the viral DNA within the viral core; Protein V (encoded by L3 gene): Contributes to the stability of the viral core; Terminal protein (encoded by L1 gene): Covalently attached to the 5′ ends of the viral DNA and plays a role in viral DNA replication.
3) Early Genes: E1A (encoded by E1A gene): Involved in the regulation of viral and cellular gene expression, promoting cell cycle progression and viral replication; E1B (encoded by E1B gene): Functions in the inhibition of apoptosis and regulation of cellular gene expression; E2A (encoded by E2A gene): Encodes the DNA-binding protein, which is involved in viral DNA replication and transcription regulation; E2B (encoded by E2B gene): Encodes the viral DNA polymerase, which is essential for viral DNA replication.
4) Late Genes: L1-L5 (encoded by L1-L5 genes): Encode structural proteins involved in capsid and core assembly, as well as the stability of the viral particle; E3 (encoded by E3 gene): Encodes proteins that modulate host immune response and facilitate viral egress; E4 (encoded by E4 gene): Encodes proteins involved in viral DNA replication, transcription regulation, and host cell shutoff.
5) Exemplary Non-coding Elements: Inverted Terminal Repeats (ITRs): Present at both ends of the viral genome and play a role in viral DNA replication and packaging; Packaging Sequence (W): Located near the left end of the viral genome, it is recognized by the viral packaging machinery during the encapsidation of viral DNA; Promoters and enhancers: Regulatory elements involved in the control of viral gene expression.

A variety of adenoviral systems have been developed, including replication competent, attenuated, and replication-deficient. Recombinant adenoviral systems allow genes to be packaged into adenoviral particles and subsequently transferred to cells through infection with these recombinant adenoviral vectors. Recombinant adenoviral packaging systems come in a variety of forms, attempting to balance safety and productivity. Each system has been designed to reduce the possibility of generating replication-competent adenovirus while maintaining efficient gene delivery. Such recombinant systems comprise a transfer vector, also known as an expression plasmid (which contains the desired genes to be packaged into the recombinant viral vector, such as GFP), along with a packaging cell line that provides the essential genes required for viral replication and packaging.

In one embodiment, the present disclosure teaches engineered CPs that enhance various aspects of adenoviral vector production, including viral replication, yield, scalability, infectiousness, and decreased cytotoxic effects on host cells. These advancements significantly improve the suitability of adenoviral vectors for applications such as human gene therapy, gene transfer, and other applications in biotechnology and medicine.

CPs can be used to improve adenoviral production. In one embodiment, CPs are used to enhance production of adenovirus by an increase infectious titer, defined as the number of infectious units per mL, physical titer, defined as the number of viral particles per mL, or TU:VG ratio, defined as the ratio of transducing units to viral genomes. The embodiment is described as follows:
1) Cell Component: The cell component of this embodiment involves the use of a cell configured to express the essential adenoviral genes E1, E2, and E4. These genes provide the necessary factors for adenoviral replication and packaging.
2) Viral Component: The viral component of this embodiment includes a transfer vector carrying a therapeutic gene payload. The transfer vector contains the necessary adenoviral elements, such as early genes (E1A, E1B, E2A, E2B, E3, and E4), late genes (L1-L5), and non-coding elements (e.g., inverted terminal repeats (ITRs), packaging sequences). The therapeutic gene payload is positioned within the adenoviral genome for efficient packaging and delivery to target cells. Adenovirus is a member of the Adenoviridae family, and recombinant systems may include adenoviral derivatives, hybrids of more than one serotype, or chimeras of more than one type of virus (e.g., adenovirus-AAV, adenovirus-lentivirus).
3) CP embodiment variations:
a) A CP generator gene sequence (e.g., SICLOPPS) is stably integrated into the genome of the packaging cell.
b) A CP produced by synthetic chemistry is added exogenously to the growth media.
c) A CP generator gene sequence (e.g. SICLOPPS) is added to the transfer vector in such a way that it is not packaged, for example by placing the CP generator outside of the terminal repeats (e.g. on a shuttle plasmid)

In another embodiment, CPs are discovered using an enrichment-based approach. A large CP gene sequence library is inserted into the transfer vector as a recombinant payload gene. The adenoviral transfer vector bearing the CP gene sequence library is then transfected, along with other packaging plasmids, into the packaging cell. Upon transfection, the plasmids begin to express the encoded cyclic peptide. The packaging cell line generates the necessary materials for recombinant viral production and packaging of the CP gene sequence library variant(s) within the cell. The CPs then bind their targets, causing a perturbation in the cell's physiological state. If this CP-induced cellular perturbation improves the cell's ability to produce viral particles by any metric (e.g., yield, quality/infectiousness), then the CP gene sequence will be more efficiently packaged and will increase its relative representation of the population. In contrast, if this CP-induced cellular perturbation reduces the cell's ability to generate viral particles, then the CP expression cassette will be less efficiently packaged and will be depleted or eliminated from the population. CP-induced cellular perturbations may alter several host cell or adenoviral-specific processes, including viral replication, genome synthesis, viral assembly, and egress. The efficiency of these processes can be dramatically altered by the cellular context or perturbations like mutations or changes in gene expression. The resulting library of viral particles will contain a subset of the original CP diversity:
1) Adenoviral particles harboring CPs gene sequences that reduced viral packaging are reduced or eliminated from the library (these CPs might be antivirals, and indeed might find utility in such applications)
2) Viral particles harboring CPs gene sequences that improved viral packaging are increased or enriched in the library
3) The CPs that had no impact on the viral packaging are also reduced due to competition from viral particles harboring CPs gene sequences that improved viral packaging and that outcompeted the neutral variants.

Resulting adenoviral vectors, each harboring one or more cyclic peptide generator sequences can then be used to infect fresh packaging cells configured to generate additional adenoviral vectors. For viral particles to be generated, the transfer vector/expression plasmid is present inside of the packaging cell. This requires that viral particles be functional and able to successfully deliver their genetic payload into the cell. CP gene sequences in non-functional viral particles (e.g. they interfere with viral production) are eliminated. In contrast, viral particles that harbor CP gene sequences that previously altered the packaging cell in a way that resulted in enhanced infectivity, quality, payload delivery, or simply a higher population of viral particles with the same infectiousness, will result in the delivery of more of these performance-enhancing CP gene sequence payloads into packaging cells. Viral particles that are better able to deliver their CP gene sequence payload into the cell (e.g. because there are more of them or because the CP enhances viral production) gain a selective advantage and can increase their representation in the population. This increases the number of cells that will generate viral particles harboring CP gene sequences that enhance viral production. In other words: Engineered cyclic peptides that enhance viral production enhance the packaging of their own coding DNA into more or better viral particles.

### Example 13. Cell-Free Non-Natural Amino Acid-Containing CP Discovery

In one embodiment, cyclic peptides comprising non-natural amino acids can be identified using cell free technology approaches. One exemplary approach for AAV includes the following steps:

Generate a cyclic peptide expression library on a circularized, ITR-flanked DNA sequence.

The DNA encoding the portion of the peptide sequence to be cyclized has codons that can be translated to synthetic amino acids, typically stop codons or quadruplet codons.

The DNA sequence is encapsulated into microfluidic droplets (i.e. using a droplet generator) such that an average of one DNA per droplet is achieved

A rolling circle amplification step occurs in order to amplify the ITR-flanked cyclic peptide generator sequence.

After the rolling circle amplification step, a cell-free expression mixture is added to the droplet; the cell free transcription translation mixture comprises both tRNAs charged with their natural amino acid as well as tRNAs charged with non-natural amino acids allowing non-natural amino acids to be added to the polypeptide change at specific positions, typically at stop codons and/or quadruplet codons, allowing for site-specific incorporation of multiple non-natural amino acids within the region of interest (i.e. the portion of the polypeptide to be cyclized).

The cell free expression reaction generates SICLOPPS constructs that contain non-natural amino acids in the peptide sequence to be cyclized, so that when intein-mediated peptide splicing occurs, a ribosomally produced cyclic peptide containing non-natural amino acids is produced.

This droplet, now containing engineered cyclic peptides with non-natural amino acids as well as the ITR-flanked cyclic peptide generator coding sequence concatemers, is then merged with a droplet containing packaging cells that are essentially configured to produce AAV vectors except that they are missing the required ITR-flanked AAV transgene, which is supplied by the merging of the two droplets, though the AAV transgene concatemers remain extracellular.

The merged droplet contains ribosomally generated cyclic peptides with non-natural amino acids, ITR-flanked cyclic peptide generator sequence concatemers generated by rolling circle amplification which served as cell free expression constructs, and packaging cells that only require the addition of a viral transgene for packaging. The droplets are then subjected toelectroporation by passing the droplet through a microfluidic electric field (i.e. the DNA is electroporated into the packaging cells) allowing the ITR-flanked cyclic peptide generator sequences enter the packaging cells, creating a complete packaging cell construct (i.e. with the addition of the AAV transgene). Depending on the parameters of the electroporation, cyclic peptides may enter the cell as well. Alternatively, cyclic peptides may not enter the cell via electroporation. In this case, cyclic peptides would be required to cross the cell membrane to exert influence over the packaging process or exert their effects on extracellular targets.

Packaging may then occur under the influence of the cyclic peptides with non-natural amino acids.

After viral packaging, the droplets are broken, releasing the produced viruses.

The viruses can then be sequenced directly as well as transduced into new host cells, where the transduced AAV genomes can be subsequently purified and sequenced.

By comparing DNA sequence frequencies of cyclic peptide generator library members before viral packaging, after packaging, and after transduction, it is possible to identify ribosomally expressed cyclic peptides with non-natural amino acids that enhance viral production.

In addition, this also may select for cyclic peptides that are able to cross the cell membrane, providing a high throughput approach for studying and perfecting the properties of cyclic peptides that optimize viral production.

The resulting non-natural, engineered cyclic peptides may then be incorporated into viral production processes.

It is known in the art that many non-natural amino acids can be ribosomally incorporated into polypeptides and cyclic peptides (Nonproteinogenic deep mutational scanning of linear and cyclic peptides; Joseph M. Rogers, Toby Passioura, and Hiroaki Suga; 2018). N-methylated amino acids may provide enhanced membrane permeability and have been demonstrated to be ribosomally incorporated into polypeptides (Ribosomal Synthesis of N-Methyl Peptides; Alexander O. Subtelny, Matthew C. T. Hartman, and Jack W. Szostak; 2008). Beta amino acids increase protease resistance of cyclic peptides and have similarly been demonstrated to be ribosomally incorporated into polypeptides (Protein Synthesis with Ribosomes Selected for the Incorporation of 3-Amino Acids; Rumit Maini, et. al.; 2015). D amino acids have been shown to have increased protease resistance, are extensively used in peptide therapeutics, and have been demonstrated to be ribosomally incorporated into polypeptides (Initiating translation with D-amino acids; Yuki Goto, Hiroshi Murakami, Hiroaki Suga; 2008). Incorporation of gamma amino acids can introduce more flexibility into the peptide chain, which can be useful in binding to larger or more diverse targets. In fact, multiple non-natural amino acids can be ribosomally incorporated into cyclic peptides (Ribosomal Elongation of Cyclic T-Amino Acids using a Reprogrammed Genetic Code; Takayuki Katoh, Hiroaki Suga; 2020). These examples are non-limiting because it has been shown that there are many non-natural amino acids that can be ribosomally incorporated into cyclic peptides.

In some embodiments it may be preferable or more economical to chemically synthesize cyclic peptides instead relying on ribosomal expression. Ribosomally produced cyclic peptides excel as a means of navigating chemical space given the ease with which high diversity compound libraries can be generated and the sequence-function linkage enabling large scale functional analysis. This high degree of engineerability is due in part to recent advances in DNA sequencing and synthesis. However, these valuable attributes do not always translate to situations in which manufacturing scale and economics are required. This is especially important in the case of non-natural amino acids which are challenging to express in mammalian cells. Therefore, it is obvious to one skilled in the art that engineered cyclic peptides, especially those comprising non-natural amino acids, are preferentially synthesized via synthetic chemistry and added exogenously. The ability to easily exploit engineered cyclic peptides that are produced either ribosomally or via synthetic chemistry provides an exceptional benefit. Example 14. In another embodiment, genetically encoded and ribosomally expressed cyclic peptides that are found to improve production of one virus, such as lentivirus, may be subsequently screened for their ability to inhibit viral replication or production of a second virus, like HSV. In one embodiment, a nested virus construct, for example a lentivirus genome inside of an HSV genome (i.e. the lentivirus genome is a payload of the HSV viral vector), is packaged inside of an HSV capsid.

Such a nested viral construct (e.g. lentivirus nested in HSV host virus) can be produced using a first plurality of HSV packaging cells configured to produce HSV viral vectors, but not configured to produce lentiviral vectors. This cell line contains the necessary components to package a replication deficient HSV host vector but not all of the essential components needed to package a lentiviral guest vector. This cell line produces a first plurality of HSV host viral particles comprising a nested guest virus (e.g. lentivirus).

Next, the host HSV viral particles harboring a nested guest lentiviral genome (resembling a provirus residing in the genome of a host virus instead of a host cell) are used to infect a second plurality of lentiviral packaging cells, which are configured to produce the guest lentivirus of the nested lentivirus, but not the HSV host virus.

The second plurality of packaging cells also express one or preferably multiple cyclic peptides that collectively enhance packaging of the nested virus (e.g. lentivirus in this example), while simultaneously possessing potent anti-HSV properties. In this way the host virus packaging can exploit a highly efficient and large payload size of viral vector system like HSV as a helper host virus that can efficiently deliver the majority of gene functions for efficient production of the nested host virus (e.g. lentivirus), with the remainder being supplied by the second plurality of packaging cells.

The very properties that make host viruses suitable for use as a helper virus (i.e. high replication efficiency), increase the probability of viral contamination. Thus, cyclic peptides can be used to enhance the production of a desired viral product (e.g. lentivirus) in a second plurality of packaging cells by ensuring that the helper virus (e.g. HSV) does not compete with the desired viral vector packaging process and that the helper virus primarily provides helper.

Thus, cyclic peptides provide an approach for both enhancing viral production of one virus directly (as described through this specification) and indirectly, by improving purity of the lentiviral product through antiviral activity against a helper host virus (e.g. HSV).

In one embodiment, such cyclic peptides are easily found by taking a library of cyclic peptides that was enriched for activities that improve viral production as described in this specification and performing an enrichment in the context of a second virus that we wish to repress.

While the enrichment will favor amplification of cyclic peptides that improve viral production of both viruses, next gen sequencing can be used to identify cyclic peptides that were strongly enriched for one virus but depleted for another virus. These cyclic peptides would provide a means of ensuring that helper virus contamination would be strongly reduced, while simultaneously supporting the production of the desired viral vector product.

In other non-limiting embodiments, the nested guest virus could be AAV, lentivirus, adenovirus, baculovirus, Herpesviruses, Poxviruses, Parvoviruses, Reoviruses, Picornaviruses, Coronaviruses, Togaviruses, Flaviviruses, Rhabdoviruses, Paramyxoviruses, Orthomyxoviruses, Bunyaviruses, Arenaviruses, Filoviruses, Retroviruses, SARS, Hepadnaviruses.

In other non-limiting embodiments, the vector/helper virus (the virus inside which the nested provirus resides) could be lentivirus, adenovirus, baculovirus, Herpesviruses, Poxviruses, Parvoviruses, Reoviruses, Picornaviruses, Coronaviruses, Togaviruses, Flaviviruses, Rhabdoviruses, Paramyxoviruses, Orthomyxoviruses, Bunyaviruses, Arenaviruses, Filoviruses, Retroviruses, SARS, Hepadnaviruses.

### Example 15. Generation of Cyclic Peptides Using SICLOPPS Variations

In some embodiments, the cyclic peptide generator is a SICLOPPS construct.

In some embodiments, the SICLOPPS cyclic peptide generator is a single polypeptide construct that splices, releasing a cyclic peptide and the spent intein. One piece SICLOPPS constructs have been described extensively in the literature.

In other embodiments, the SICLOPPS cyclic peptide generator exploits a 2 piece intein splicing architecture inspired from three piece split inteins. (Synthetic Two-piece and Three-piece Split Inteins for Protein trans-Splicing; Wenchang Sun, Jing Yang, Xiang-Qin Liu; 2004). This approach comprises 2 pieces: a substrate element and a splicing enzyme element.

The Substrate Element consists of a C-terminal portion of the intein followed by the peptide to be cyclized (which would typically be synonymous with the N and C exteins, as it integrates both the N and C-exteins), and finally an N-terminal portion of the intein. The orientation is as follows: [Intein C-terminus-Peptide to be cyclized (C-extein/N-extein)—Intein N-terminus]. This construct acts as the substrate for the splicing reaction, with the intein N and C terminal peptide sequences acting as recognition elements for the splicing enzyme described below.

The Splicing Enzyme Element is composed of the non-overlapping Intein N-terminus (which would typically be the C-terminus in a standard SICLOPPS architecture) fused, preferably with a linker, to the Intein C-terminus (normally the N-terminus in a standard SICLOPPS architecture). The orientation is: [Intein N-terminus—(optional linker)—Intein C-terminus]. This piece acts as a catalytically active enzyme that recognizes the substrate element (via the substrate's intein N and C terminal peptide sequences) and catalyzes the splicing reaction on the Peptide to be cyclized (which would typically be considered the C-extein/N-extein in standard splicing reactions).

In practical applications, when these two elements are expressed in cells, the splicing enzyme element recognizes the N and C intein arms on the substrate element and catalyzes the splicing reaction. This results in the release of a cyclic peptide and the linear N and C intein as waste products. The splicing enzyme element remains catalytically active and can perform multiple rounds of splicing reactions on new substrate elements. Cyclic peptide libraries can be generated on the substrate element, allowing for efficient expression of high diversity cyclic peptide libraries.

This adaptation of the three-piece intein strategy for SICLOPPS allows for a more efficient cyclic peptide generation process with fewer waste products, making it suitable for high-throughput production of cyclic peptides for various applications, such as drug discovery and protein engineering. In addition, this approach has a substantial advantage in that it enables a large savings in sequence space. This is a very important consideration when using SICLOPPS to discover cyclic peptides in viruses that have limited packaging capacity like AAV.

In some embodiments, the linker sequences for the splicing enzyme element would be the result of a library screen. This is known to people in the art (Tuning the Flexibility of Glycine-Serine Linkers To Allow Rational Design of Multidomain Proteins; Martijn van Rosmalen, Mike Krom, and Maarten Merkx; 2017). A simple selection can be applied to identify a functional construct: an ITR-flanked splicing enzyme element comprising a linker library is generated and transformed into cells configured to produce AAV along with a cyclic peptide Substrate Element. The cyclic peptide is chosen to be one that has been previously shown to substantially increase viral titer. After allowing the cells to package viral particles for a while (the preferred time depends on the specific virus), the viral library (comprising encapsidated variants of the splicing element linker library) is collected, and sequenced. By analyzing the sequencing data, it can be determined which splicing enzyme elements were functional and yielded cyclic peptides by observing which splicing enzyme elements were enriched. These splicing enzyme elements can then be further evolved, evaluated, and used as cyclic peptide generators.

In some embodiments a one-piece SICLOPPS cyclic peptide generator may have a degradation tag on it. This has been shown to increase efficiency of recycling of intein splicing waste products (Traceless Production of Cyclic Peptide Libraries in E. coli; Jaime E. Townend and Ali Tavassoli; 2016). In further embodiments, a degradation tag may be applied to a two-piece SICLOPPS architecture described above. In this case, the degradation tag is fused to the substrate element (comprising the peptide to be spliced and the N and C intein recognition sequences), so that after cyclic peptide splicing, the linear waste product (comprising the N and C terminal intein recognition sequences) can be degraded. This allows the cyclic peptide to be tracelessly produced while the intein splicing enzyme is free to catalyze additional cyclization reactions.

### Example 16. In Some Embodiments, the Cyclic Peptide Generator is Encoded on the Genome of an RNA Virus

In some embodiments, the cyclic peptide that is expressed by the virus may target RNA structures of the virus or the host. It has been shown and we have previously identified cyclic peptides that can engage and modulate RNA structure.

We successfully demonstrated that cyclic peptides (CPs) have a wide range of modulatory capabilities within cellular systems, which include interacting with and altering the functionality of riboswitches. In a model system involving E. coli, we engineered an essential gene to be regulated by a theophylline-dependent riboswitch. In the absence of theophylline, the mRNA for the gene of interest adopted a structure that inhibited translation, resulting in a lethal phenotype. We then introduced a CP to the system and showed that it could interact with the mRNA, changing its conformation to one that allowed translation of the essential gene, effectively rescuing the cells from the lethal state. This interaction between the CP and the riboswitch-regulated mRNA resulted in increased cell viability. Furthermore, we demonstrated that CPs can be provided exogenously, confirming their ability to penetrate cell membranes. Our work illustrates the potential of CPs as powerful tools for modulating not only protein functions but also nucleic acid-based regulatory systems.

Thus, cyclic peptides provide a powerful tool for regulating nucleic acid regulation, particularly in the context of RNA viruses or viruses with complex mRNA regulatory networks; for example, Adenovirus has been shown to have over 10,000 alternatively spliced transcripts. Cyclic peptides represent an ideal tool for manipulating this highly complex RNA splicing biology.

In some embodiments, the cyclic peptide may disrupt protein-nucleic acid interactions. While cyclic peptides have been shown to be an excellent tool for targeting protein-protein interactions (Design and Application of a DNA-Encoded Macrocyclic Peptide Library; Zhengrong Zhu et. al.; 2018, cyclic peptide inhibitors of RNA virus N protein using DNA-tagged affinity panning), the application of cyclic peptides to modulating protein-nucleic acid interactions have only recently begun to be studied (Recent Trends in Cyclic Peptides as Therapeutic Agents and Biochemical Tools; Joon-Seok Choi, Sang Hoon Joo; 2020). However, such interactions are of critical importance to RNA viruses. This insight has led some to search for antiviral agents that disrupt essential RNA-protein interactions (Inhibition of Both HIV-1 Reverse Transcription and Gene Expression by a Cyclic Peptide that Binds the Tat-Transactivating Response Element (TAR) RNA; Matthew S. Lalonde, et. al.; 2011; A cyclic peptide mimic of an RNA recognition motif of human La protein is a potent inhibitor of hepatitis C virus; Asit Kumar Manna, et. al.; 2013). Others have shown However, as we show for multiple viruses, including both DNA viruses (AAV) and RNA viruses (lentivirus), cyclic peptides can substantially increase viral production. And because of what is known in the art with respect to cyclic peptides being able to target RNA-protein interactions in viruses, the application of cyclic peptides to RNA viruses presents a particularly unique and enabling aspect. In other words: Given the known ability of cyclic peptides to target RNA-protein interactions, their application to RNA viruses represents a particularly unique and promising avenue for exploration.

In some embodiments, the cyclic peptide generator may be translationally fused to one or more other proteins. In further embodiments the cyclic peptide generator fusion may be released, by protease cleavage, splicing reactions, 2A self-cleaving peptides, etc . . .

In such embodiments, regulation of cyclic peptide expression requires different strategies due to the lack of DNA regulatory elements like promoters and transcription factors.

In these cases, the cyclic peptide generator may be part of a polyprotein.

Alternatively, the cyclic peptide generator may use a dedicated Internal Ribosome Entry Site (IRES) upstream of the cyclic peptide-encoding sequence. This RNA element can directly recruit ribosomes and initiate translation in the middle of the viral RNA, allowing control over cyclic peptide expression without reliance on canonical promoters.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method of obtaining an engineered cyclic peptide capable of increasing viral titer and/or transduction efficiency of a viral vector composition, the method comprising:
(a) culturing a first plurality of host cells permissive for replication of a virus under conditions suitable for recombinant viral production, wherein each host cell of the first plurality of host cells comprises:
(i) at least one viral replication gene essential for the replication of the virus;
(ii) at least one viral structural gene essential for formation of viral capsids;
(iii) at least one additional viral gene necessary to produce the virus in the host cells; and
(iv) an engineered cyclic peptide produced from a polypeptide encoded by a first nucleotide sequence, wherein (v) the first nucleotide sequence is operably linked to one or more viral-specific packaging sequences necessary for encapsulation of the first nucleotide sequence within the viral capsids or (vi) the first nucleotide sequence is associated with a second nucleotide sequence comprising a barcode that comprises identifying information regarding the engineered cyclic peptide produced in the host cell, and the second nucleotide sequence is operably linked to the one or more viral-specific packaging sequences necessary for encapsulation of the second nucleotide sequence within the viral capsids,
thereby obtaining a first plurality of viral vectors comprising the first nucleotide sequence and/or the second nucleotide sequence from the first plurality of host cells;
(b) optionally, repeating the following steps one or more times in cycles:
(b1) allowing a plurality of viral vectors of the previous cycle to infect a plurality of host cells of the present cycle permissive for replication of the virus; and
(b2) culturing the plurality of host cells of the present cycle under conditions suitable for recombinant viral production, wherein each host cell of the plurality of host cells of the present cycle comprises the elements (i)-(iii) of the first plurality of host cells, and further comprises the first nucleotide sequence operably linked to the one or more viral-specific packaging sequences producing the engineered cyclic peptide, thereby obtaining a plurality of viral vectors of the present cycle comprising the first nucleotide sequence;
(c) allowing the first plurality of viral vectors or the plurality of viral vectors of the present cycle to infect a final plurality of host cells; and
(d) determining one or more engineered cyclic peptides capable of increasing viral titer and/or transduction efficiency of the viral vector composition by analyzing nucleotide sequences operably linked to the one or more viral-specific packaging sequences from (i) the final plurality of host cells and/or (ii) a final plurality of viral vectors produced in the final plurality of host cells.

2. The method of claim 1, wherein in step (a), the first nucleotide sequence is operably linked to the one or more viral-specific packaging sequences, and the method comprises culturing the final plurality of host cells under conditions suitable for recombinant viral production, wherein each host cell of the final plurality of host cells comprises the elements (i)-(iii) of the first plurality of host cells, and further comprises the first nucleotide sequence operably linked to one or more viral-specific packaging sequences and producing the engineered cyclic peptide encoded by the first nucleotide sequence, thereby producing the final plurality of viral vectors from the final plurality of host cells and determining the engineered cyclic peptide by analyzing nucleotide sequences operably linked to the one or more viral-specific packaging sequences from the final plurality of viral vectors.

3. The method of claim 1, wherein
(i) the viral vector composition is an adeno-associated virus (AAV) vector composition;
(ii) the at least one viral replication gene comprises at least one AAV replication gene;
(iii) the at least one viral structural gene comprises at least one AAV capsid encoding gene;
(iv) the at least one additional viral gene comprises at least one AAV helper gene; and
(v) the one or more viral-specific packaging sequences comprise at least two functional AAV inverted terminal repeats (ITRs).

4. The method of claim 1, wherein
(i) the viral vector composition is a lentivirus vector composition;
(ii) the at least one viral replication gene comprises at least one lentiviral pol gene;
(iii) the at least one viral structural gene comprises at least one lentiviral gag gene and at least one env gene;
(iv) the at least one additional viral gene comprises at least one lentiviral rev gene; and
(v) the one or more viral-specific packaging sequences comprise a Psi sequence.

5. The method of claim 1, wherein each host cell of first plurality of host cells and each host cell of final plurality of host cells are mammalian host cells.

6. The method of claim 1, wherein the first nucleotide sequence operably linked to the one or more viral-specific packaging sequences further encodes a reporter, a therapeutic payload or a selectable marker.

7. The method of claim 1, wherein the first plurality of host cells at step (a) comprises at least 1,000 host cells each producing a unique engineered cyclic peptide.

8. The method of claim 1, further comprising (e): generating new viral vectors in the presence of an engineered cyclic peptide obtained in step (d), thereby producing the viral vector composition of increased viral titer and/or transduction efficiency.

9. The method of claim 8, which produces the viral vector composition having a characteristic, which is at least 2-fold higher than a corresponding characteristic of a reference viral vector composition produced in a plurality of reference host cells under essentially identical conditions, wherein each reference host cell of the plurality of reference host cells comprises the elements (i)-(iii) of the first plurality of host cells and does not comprise the first nucleotide sequence and the engineered cyclic peptide, and wherein the characteristic is selected from the group consisting of viral titer and transduction efficiency.

10. The method of claim 8, wherein the engineered cyclic peptide is not essentially present in the viral vector composition of increased viral titer and/or transduction efficiency.

11. A plurality of host cells permissive for replication of a virus, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary to produce the virus in the host cells; and
(iv) a nucleotide sequence operably linked to one or more viral-specific packaging sequences necessary for encapsulation of the nucleotide sequence within the viral capsids, wherein the nucleotide sequence encodes a payload;
wherein the engineered cyclic peptide increases a characteristic of viral vectors produced by the plurality of host cells by at least 2-fold compared to a corresponding characteristic of viral vectors produced by a plurality of reference host cells under essentially identical conditions, wherein each reference host cell of the plurality of reference host cells comprises the elements (i)-(iii) of the plurality of host cells and does not comprise the engineered cyclic peptide, and wherein the characteristic of viral vectors is selected from the group consisting of viral titer and transduction efficiency.

12. The plurality of host cells of claim 11, wherein
(i) the virus is an adeno-associated virus (AAV);
(ii) the at least one viral replication gene comprises at least one AAV replication gene;
(iii) the at least one viral structural gene comprises at least one AAV capsid encoding gene;
(iv) the at least one additional viral gene comprises at least one AAV helper gene; and
(v) the one or more viral-specific packaging sequences comprise at least two functional AAV inverted terminal repeats (ITRs).

13. The plurality of host cells of claim 11, wherein
(i) the virus is a lentivirus;
(ii) the at least one viral replication gene comprises at least one lentiviral pol gene;
(iii) the at least one viral structural gene comprises at least one lentiviral gag gene and at least one env gene;
(iv) the at least one additional viral gene comprises at least one lentiviral rev gene; and
(v) the one or more viral-specific packaging sequences comprise a Psi sequence.

14. The plurality of host cells of claim 11, wherein the engineered cyclic peptide is produced ribosomally in each host cell of the plurality of host cells.

15. The plurality of host cells of claim 11, wherein the engineered cyclic peptide is exogenously supplied to each host cell of the plurality of host cells.

16. The plurality of host cells of claim 11, wherein each host cell of the plurality of host cells is a mammalian host cell.

17. The plurality of host cells of claim 11, wherein each host cell of the plurality of host cells is an insect host cell.

18. The plurality of host cells of claim 11, wherein the payload comprises a therapeutic gene.

19. The plurality of host cells of claim 11, wherein the plurality of host cells comprises at least 10,000 host cells.

20. A method of producing a viral vector composition of increased viral titer and/or transduction efficiency, the method comprising:
(a) culturing a plurality of host cells permissive for replication of a virus under conditions suitable for recombinant viral production, wherein each host cell of the plurality of host cells comprises an engineered cyclic peptide and further comprises:
(i) at least one viral replication gene essential for the replication of the virus;
(ii) at least one viral structural gene essential for formation of viral capsids;
(iii) at least one additional viral gene necessary to produce the virus in the host cells; and
(iv) a nucleotide sequence operably linked to one or more viral-specific packaging sequences necessary for encapsulation of the nucleotide sequence within the viral capsids, wherein the nucleotide sequence encodes a payload; and
(b) producing the viral vector composition of increased viral titer and/or transduction efficiency from the plurality of host cells, wherein the viral vector composition has an increased viral titer and/or transduction efficiency which is at least a 20%, 40%, 60%, 80%, 100%, 200%, or 500% greater than a viral titer and/or transduction efficiency of a reference viral vector composition produced in a plurality of reference host cells under essentially identical conditions, wherein each reference host cell of the plurality of reference host cells comprises the elements (i)-(iv) of the plurality of host cells and does not comprise the engineered cyclic peptide.

21. The method of claim 20, wherein
(i) the viral vector composition is an adeno-associated virus (AAV) vector composition;
(ii) the at least one viral replication gene comprises at least one AAV replication gene;
(iii) the at least one viral structural gene comprises at least one AAV capsid encoding gene;
(iv) the at least one additional viral gene comprises at least one AAV helper gene; and
(v) the one or more viral-specific packaging sequences comprise at least two functional AAV inverted terminal repeats (ITRs).

22. The method of claim 20, wherein
(i) the viral vector composition is a lentivirus vector composition;
(ii) the at least one viral replication gene comprises at least one lentiviral pol gene;
(iii) the at least one viral structural gene comprises at least one lentiviral gag gene and at least one env gene;
(iv) the at least one additional viral gene comprises at least one lentiviral rev gene; and
(v) the one or more viral-specific packaging sequences comprise a Psi sequence.

23. The method of claim 20, wherein the engineered cyclic peptide is produced ribosomally in each host cell of the plurality of host cells.

24. The method of claim 20, wherein the engineered cyclic peptide is exogenously supplied to each host cell of the plurality of host cells.

25. The method of claim 20, wherein each host cell of the plurality of host cells is a mammalian host cell.

26. The method of claim 20, wherein each host cell of the plurality of host cells is an insect host cell.

27. The method of claim 20, wherein the plurality of host cells comprises at least 10,000 host cells.

28. The method of claim 20, wherein the engineered cyclic peptide is not essentially present in the viral vector composition of increased viral titer and/or transduction efficiency.


SEQUENCE LISTING (230 sequences)

SEQ ID NO: 1 - protein, 312 aa, Adeno-associated virus 2
  source 1..312: note = rep40 protein, exemplary of rep
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 2 - protein, 397 aa, Adeno-associated virus 2
  source 1..397: note = rep52 protein, exemplary of rep
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 3 - protein, 536 aa, Adeno-associated virus 2
  source 1..536: note = rep68 protein, exemplary of rep
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 4 - protein, 621 aa, Adeno-associated virus 2
  source 1..621: note = rep78 protein, exemplary of rep
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 5 - protein, 735 aa, Adeno-associated virus 2 [sequence omitted]

SEQ ID NO: 6 - protein, 598 aa, Adeno-associated virus 2
  source 1..598: note = VP2 capsid protein, exemplary of cap
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 533 aa, Adeno-associated virus 2
  source 1..533: note = VP3 capsid protein, exemplary of cap
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - DNA, 145 bp, Adeno-associated virus 2
  source 1..145: mol_type = genomic DNA; note = 5' ITR (Inverted Terminal Repeat)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 9 - DNA, 145 bp, Adeno-associated virus 2
  source 1..145: mol_type = genomic DNA; note = 3' ITR (Inverted Terminal Repeat)
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 10 - DNA, 1872 bp, Adeno-associated virus 1
  source 1..1872: mol_type = genomic DNA; note = rep
atgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataa

SEQ ID NO: 11 - DNA, 2211 bp, Adeno-associated virus 1
  source 1..2211: mol_type = genomic DNA; note = cap
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 12 - protein, 610 aa, Adeno-associated virus 5
  source 1..610: note = rep22 protein, exemplary of rep
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 13 - protein, 724 aa, Adeno-associated virus 5
  source 1..724: note = VP1 capsid protein, exemplary of cap
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - protein, 623 aa, Adeno-associated virus 6
  source 1..623: note = rep78 protein, exemplary of rep
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 15 - protein, 736 aa, Adeno-associated virus 6
  source 1..736: note = VP1 capsid protein, exemplary of cap
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - protein, 289 aa, Human adenovirus 5
  source 1..289: note = E1A protein, exemplary of AAV Helper gene
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 17 - protein, 496 aa, Human adenovirus 5
  source 1..496: note = E1B55K, exemplary of AAV Helper gene
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 18 - protein, 529 aa, Human adenovirus 5
  source 1..529: note = E2A, exemplary of AAV Helper gene
MASREEEQRETTPERGRGAARRPPTMEDVSSPSPSPPPPRAPPKKRMRRRIESEDEEDSSQDALVPRTPSPRPSTSAADLAIAPKKKKKRPSPKPERPPSPEVIVDSEEEREDVALQMVGFSNPPVLIKHGKGGKRTVRRLNEDDPVARGMRTQEEEEEPSEAESEITVMNPLSVPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 19 - protein, 294 aa, Human adenovirus 5
  source 1..294: note = E4off6, exemplary of AAV Helper gene
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 20 - DNA, 70 bp, Human adenovirus 1
  source 1..70: mol_type = genomic DNA; note = VA-I viral associated RNA, exemplary of AAV Helper gene
gccggccggactgcggcgaacgggggtttgcctccccgtcatgcaagaccccgcttgcaaattcctccgg

SEQ ID NO: 21 - DNA, 70 bp, Human adenovirus 1; identical to SEQ ID NO: 20
  source 1..70: mol_type = genomic DNA; note = VA-II viral associated RNA, exemplary of AAV Helper gene

SEQ ID NO: 22 - DNA, 163 bp, Human adenovirus 5
  source 1..163: mol_type = genomic DNA; note = VA-I viral associated RNA, exemplary of AAV Helper gene
agcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctcctttt

SEQ ID NO: 23 - DNA, 157 bp, Human adenovirus 5
  source 1..157: mol_type = genomic DNA; note = VA-II viral associated RNA, exemplary of AAV Helper gene
ggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagcccc

SEQ ID NO: 24 - protein, 116 aa, Human immunodeficiency virus 1
  source 1..116: note = rev, exemplary of Lentivirus Helper gene
MAGRSGDSDEDLLKAVRLIKFLYQSNPPPNPEGTRQARRNRRRRWRERQRQIHSISERILSTYLGRSAEPVPLQLPPLERLTLDCNEDCGTSGTQGVGSPQILVESPTILESGAKE

SEQ ID NO: 25 - protein, 1003 aa, Human immunodeficiency virus 1
  source 1..1003: note = pol, exemplary of Lentivirus Helper gene
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 26 - protein, 500 aa, Human immunodeficiency virus 1
  source 1..500: note = gag, exemplary of Lentivirus Helper gene
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 27 - protein, 511 aa, Vesicular stomatitis virus G
  source 1..511: note = VSV-G, exemplary of Lentivirus envelope gene
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 28 - DNA, 234 bp, Human immunodeficiency virus 1
  source 1..234: mol_type = genomic DNA; note = 3' LTR (Delta-U3)
tgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttcca

SEQ ID NO: 29 - DNA, 181 bp, Human immunodeficiency virus 1
  source 1..181: mol_type = genomic DNA; note = 5' LTR (truncated)
tgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagaccc

SEQ ID NO: 30 - protein, 882 aa, Herpes simplex virus 1
  source 1..882: note = UL5, exemplary of AAV Helper gene
MAAAGGERQLDGQKPGPPHLQQPGDRPAVPGRAEAFLNFTSMHGVQPILKRIRELSQQQLDGAQVPHLQWFRDVAALESPAGLPLREFPFAVYLITGNAGSGKSTCVQTINEVLDCVVTGATRIAAQNMYAKLSGAFLSRPINTIFHEFGFRGNHVQAQLGQYPYTLTSNPASLEDLQRRDLTYYWEVILDLTKRALAASGGEELRNEFRALAALERTLGLAEGALTRLAPATHGALPAFTRSNVIVIDEAGLLGRHLLTAVVYCWWMINALYHTPQYAARLRPVLVCVGSPTQTASLESTFEHQKLRCSVRQSENVLTYLICNRTLREYARLSYSWAIFINNKRCVEHEFGNLMKVLEYGLPITEEHMQFVDRFVVPENYITNPANLPGWTRLFSSHKEVSAYMAKLHAYLKVTREGEFVVFTLPVLTFVSVKEFDEYRRLTHQPGLTIEKWLTANASRITNYSQSQDQDAGHMRCEVHSKQQLVVARNDVTYVLNSQIAVTARLRKLVFGFSGTFRAFEAVLRDDSFVKTQGETSVEFAYRFLSRLIFSGLISFYNFLQRPGLDATQRTLAYARMGELTAEILSLRPKSSGVPTQASVMADAGAPGERAFDFKQLGPRDGGPDDFPDDDLDVIFAGLDEQQLDVFYCHYTPGEPETTAAVHTQFALLKRAFLGRFRILQELFGEAFEVAPFSTYVDNVIFRGCEMLTGSPRGGLMSVALQTDNYTLMGYTYARVFAFADELRRRHATANVAELLEEAPLPYVVLRDQHGFMSVVNTNISEFVESIDSTELAMAINADYGISSKLAMTITRSQGLSLDKVAICFTPGNLRLNSAYVAMSRTTSSEFLRMNLNPLRERHERDDVISEHILSALRDPNVVIVY

SEQ ID NO: 31 - protein, 750 aa, Herpes simplex virus 1
  source 1..750: note = UL8, exemplary of AAV Helper gene
MDTADIVWVEESVSAITLYAVWLPPRAREYFHALVYFVCRNAAGEGRARFAEVSVTATELRDFYGSADVSVQAVVAAARAATTPAASPLEPLENPTLWRALYACVLAALERQTGPVALFAPLRIGSDPRTGLVVKVERASWGPPAAPRAALLVAEANIDIDPMALAARVAEHPDARLAWARLAAIRDTPQCASAASLTVNITTGTALFAREYQTLAFPPIKKEGAFGDLVEVCEVGLRPRGHPQRVTARVLLPRDYDYFVSAGEKFSAPALVALFRQWHTTVHAAPGALAPVFAFLGPEFEVRGGPVPYFAVLGFPGWPTFTVPATAESARDLVRGAAAAYAALLGAWPAVGARVVLPPRAWPGVASAAAGCLLPAVREAVARWHPATKIIQLLDPPAAVGPVWTARFCFPGLRAQLLAALADLGGSGLADPHGRTGLARLDALVVAAPSEPWAGAVLERLVPDTCNACPALRQLLGGVMAAVCLQIEETASSVKFAVCGGDGGAFWGVFNVDPQDADAASGVIEDARRAIETAVGAVLRANGLRLRHPLCLALEGVYTHAVAWSQAGVWFWNSRDNTDHLGGFPLRGPAYTTAAGVVRDTLRRVLGLTTACVPEEDALTARGLMEDACDRLILDAFNKRLDAEYWSVRVSPFEASDPLPPTAFRGGALLDAEHYWRRVVRVCPGGGESVGVPVDLYPRPLVLPPVDCAHHLREILREIELVFTGVLAGVWGEGGKFVYPFDDKMSFLFA

SEQ ID NO: 32 - protein, 1058 aa, Herpes simplex virus 1
  source 1..1058: note = UL52, exemplary of AAV Helper gene
MGQEDGNRGERRAAGTPVEVTALYATDGCVITSSIALLTNSLLGAEPVYIFSYDAYTHDGRADGPTEQDRFEESRALYQASGGLNGDSFRVTFCLLGTEVGGTHQARGRTRPMFVCRFERADDVAALQDALAHGTPLQPDHIAATLDAEATFALHANMILALTVAINNASPRTGRDAAAAQYDQGASLRSLVGRTSLGQRGLTTLYVHHEVRVLAAYRRAYYGSAQSPFWFLSKFGPDEKSLVLTTRYYLLQAQRLGGAGATYDLQAIKDICATYAIPHAPRPDTVSAASLTSFAAITRFCCTSQYARGAAAAGFPLYVERRIAADVRETSALEKFITHDRSCLRVSDREFITYIYLAHFECFSPPRLATHLRAVTTHDPNPAASTEQPSPLGREAVEQFFCHVRAQLNIGEYVKHNVTPRETVLDGDTAKAYLRARTYAPGALTPAPAYCGAVDSATKMMGRLADAEKLLVPRGWPAFAPASPGEDTAGGTPPPQTCGIVKRLLRLAATEQQGPTPPAIAALIRNAAVQTPLPVYRISMVPTGQAFAALAWDDWARITRDARLAEAVVSAEAAAHPDHGALGRRLTDRIRAQGPVMPPGGLDAGGQMYVNRNEIFNGALAITNIILDLDIALKEPVPFRRLHEALGHFRRGALAAVQLLFPAARVDPDAYPCYFFKSACRPGPASVGSGSGLGNDDDGDWFPCYDDAGDEEWAEDPGAMDTSHDPPDDEVAYFDLCHEVGPTAEPRETDSPVCSCTDKIGLRVCMPVPAPYVVHGSLTMRGVARVIQQAVLLDRDFVEAIGSYVKNFLLIDTGVYAHGHSLRLPYFAKIAPDGPACGRLLPVFVIPPACKDVPAFVAAHADPRRFHFHAPPTYLASPREIRVLHSLGGDYVSFFERKASRNALEHFGRRETLTEVLGRYNVQPDAGGTVEGFASELLGRIVACIETHFPEHAGEYQAVSVRRAVSKDDWVLLQLVPVRGTLQQSLSCLRFKHGRASRATARTFVALSVGANNRLCVSLCQQCFAAKCDSNRLHTLFTIDAGTPCSPSVPCSTSQPSS

SEQ ID NO: 33 - protein, 1196 aa, Herpes simplex virus 1
  source 1..1196: note = UL29, exemplary of AAV Helper gene
METKPKTATTIKVPPGPLGYVYARACPSEGIELLALLSARSGDSDVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGENFTYPLPFFNRPLNRLLFEAVVGPAAVALRCRNVDAVARAAAHLAFDENHEGAALPADITFTAFEASQGKTPRGGRDGGGKGPAGGFEQRLASVMAGDAALALESIVSMAVFDEPPTDISAWPLFEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGGVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASSLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 34 - protein, 6 aa, synthetic construct
  source 1..6: note = Cyclic peptide that was found to increase AAV viral titer.
CKDYFS

SEQ ID NO: 35 - protein, 6 aa, synthetic construct
  source 1..6: note = Cyclic peptide that was found to increase or decrease AAV viral titer in a dose-responsive manner.
CYNHAR

SEQ ID NO: 36 - protein, 6 aa, synthetic construct
  source 1..6: note = Cyclic peptide that was found to enhance AAV viral titer. We observed log10 enrichment score of 3.737, mean 77 experimental replicates. We observed excellent mean log10 enrichment scores for this cyclic peptide across multiple AAV serotypes: AAV1_4.0; AAV2_3.5; AAV5_3.9; AAV6_3.9. This CP possesses the following motifs: HYD_POS_POL-motif; POL_HYD_POL-motif.
CPQFGR

SEQ ID NO: 37 - protein, 5 aa, synthetic construct
  source 1..5: note = Cyclic peptide that was found to enhance AAV TU:VG ratio and lentiviral titer.
CSSLT

SEQ ID NO: 38 - protein, 6 aa, synthetic construct
  source 1..6: note = Cyclic peptide that was found to enhance AAV TU:VG ratio.
CTHKVS

SEQ ID NO: 39 - protein, 5 aa, synthetic construct
  source 1..5: note = Cyclic peptide that was found to enhance AAV TU:VG ratio.
CCRPH

SEQ ID NO: 40 - protein, 5 aa, synthetic construct
  source 1..5: note = Cyclic peptide that was found to enhance AAV TU:VG ratio and lentiviral titer.
CKYEE

SEQ ID NO: 41 - protein, 5 aa, synthetic construct
  source 1..5: note = Cyclic peptide that was found to enhance AAV TU:VG ratio.
CRVSY

SEQ ID NO: 42 - protein, 6 aa, synthetic construct
  source 1..6: note = Cyclic peptide that was found to enhance AAV TU:VG ratio.
CQVFQL

SEQ ID NO: 43 - protein, 6 aa, synthetic construct
  source 1..6: note = Cyclic peptide that was found to enhance lentiviral titer.
CGSTKS

SEQ ID NO: 44 - protein, 5 aa, synthetic construct
  source 1..5: note = Cyclic peptide that was found to enhance lentiviral titer.
CPYTD

SEQ ID NO: 45 - protein, 6 aa, synthetic construct
  source 1..6: note = Cyclic peptide that was found to enhance lentiviral titer.
CPLQVP

SEQ ID NO: 46 - protein, 5 aa, synthetic construct
  source 1..5: note = Cyclic peptide that was found to enhance AAV viral titer.
CSARR

SEQ ID NO: 47 - protein, 6 aa, synthetic construct
  source 1..6: note = Cyclic peptide that was found to enhance AAV viral titer.
CPLRSP

SEQ ID NO: 48 - protein, 5 aa, synthetic construct
  source 1..5: note = Cyclic peptide that was found to enhance AAV viral titer
CAAAS

SEQ ID NO: 49 - protein, 5 aa, synthetic construct
  source 1..5: note = Cyclic peptide that was found to enhance AAV viral titer
CADTV

SEQ ID NO: 50 - protein, 6 aa, synthetic construct
  source 1..6: note = Cyclic peptide that was found to enhance AAV viral titer; We observed log10 enrichment score of 2.959, mean of 79 experimental replicates. We observed excellent mean log10 enrichment scores for this cyclic peptide across multiple AAV serotypes: AAV1_3.0; AAV2_3.1; AAV5_2.8; AAV6_2.7. This CP possesses the following motifs: POL_HYD_POL-motif; HYD_ARO_POL-motif; POL_HYD_HYD-motif.
CANMPY

SEQ ID NO: 51 - protein, 5 aa, synthetic construct
  source 1..5: note = Cyclic peptide that was found to enhance AAV viral titer
CARFR

SEQ ID NO: 52 - protein, 5 aa, synthetic construct
  source 1..5: note = Cyclic peptide that was found to enhance AAV viral titer
CETDV

SEQ ID NO: 53 - protein, 5 aa, synthetic construct
  source 1..5: note = Cyclic peptide that was found to enhance AAV viral titer
CPKNP

SEQ ID NO: 54 - protein, 5 aa, synthetic construct
  source 1..5: note = Cyclic peptide that was found to enhance AAV viral titer
CQSRS

SEQ ID NO: 55 - protein, 6 aa, synthetic construct
  source 1..6: note = Cyclic peptide that was found to enhance AAV viral titer; We observed log10 enrichment score of 3.258, mean of 74 experimental replicates. We observed excellent mean log10 enrichment scores for this cyclic peptide across multiple AAV serotypes: AAV1_3.4; AAV2_3.3; AAV5_3.2; AAV6_3.1. Cyclo-CQVTRM possesses the following motifs: POL_HYD_POL-motif; POL_POL_HYD-motif; POS_HYD_POL-motif.
CQVTRM

SEQ ID NO: 56 - protein, 6 aa, synthetic construct
  source 1..6: note = Cyclic peptide that was found to enhance AAV viral titer; We observed log10 enrichment score of 3.653, mean of 76 experimental replicates. We observed excellent mean log10 enrichment scores for this cyclic peptide across multiple AAV serotypes: AAV1_3.5; AAV2_4.0; AAV5_3.0; AAV6_3.4. Cyclo-CSDPSA possesses the following motifs: POL_HYD_POL-motif; NEG_HYD_POL-motif; POL_NEG_HYD-motif; POL_POL_NEG-motif.
CSDPSA

SEQ ID NO: 57 - protein, 6 aa, synthetic construct
  source 1..6: note = Cyclic peptide that was found to enhance AAV viral titer
CSESCP

SEQ ID NO: 58 - protein, 5 aa, synthetic construct
  source 1..5: note = Cyclic peptide that was found to enhance AAV viral titer
CSVPY

SEQ ID NO: 59 - protein, 5 aa, synthetic construct
  source 1..5: note = Cyclic peptide that was found to enhance AAV viral titer
CTCKK

SEQ ID NO: 60 - protein, 5 aa, synthetic construct
  source 1..5: note = Cyclic peptide that was found to enhance AAV viral titer
CYPLH

SEQ ID NO: 61 - protein, 5 aa, synthetic construct
  source 1..5: note = Cyclic peptide that was found to enhance AAV viral titer
CYSPF

SEQ ID NO: 62 - protein, 6 aa, synthetic construct
  source 1..6: note = Cyclic peptide that was found to enhance AAV viral titer; We observed log10 enrichment score of 3.986, mean of 70 experimental replicates. We observed excellent mean log10 enrichment scores for this cyclic peptide across multiple AAV serotypes: AAV1_4.2; AAV2_4.1; AAV5_3.6; AAV6_3.9. Cyclo-CYVTTD possesses the following motifs: ARO_HYD_POL-motif; POL_POL_NEG-motif; NEG_POL_ARO-motif.
CYVTTD

SEQ ID NO: 63 - DNA, 126 bp, Human immunodeficiency virus 1
  source 1..126: mol_type = genomic DNA; note = Psi, exemplary of Lentivirus packaging signal
ctctctcgacgcaggactcggcttgctgaagcgcgcacggcaagaggcgaggggcggcgactggtgagtacgccaaaaattttgactagcggaggctagaaggagagagatgggtgcgagagcgtc

SEQ ID NO: 64 - protein, 6 aa, synthetic construct
  source 1..6: note = Exemplary cyclic peptide, motif HYD_HYD_POL-motif, log10 enrichment score 3.875
CGVWAA

SEQ ID NO: 65 - protein, 6 aa, synthetic construct
  source 1..6: note = Exemplary cyclic peptide, motif POL_HYD_POL-motif, log10 enrichment score 3.166
CHTVNL

SEQ ID NO: 66 - protein, 6 aa, synthetic construct
  source 1..6: note = Exemplary cyclic peptide, motif ARO_POL_ARO-motif, log10 enrichment score 3.585
CFCRRY

SEQ ID NO: 67 - protein, 6 aa, synthetic construct
  source 1..6: note = Exemplary cyclic peptide, motif POL_POL_HYD-motif, log10 enrichment score 3.001
CVMLSS

SEQ ID NO: 68 - protein, 6 aa, synthetic construct
  source 1..6: note = Exemplary cyclic peptide, motif HYD_ARO_POL-motif, log10 enrichment score 2.979
CYHLLW

SEQ ID NO: 69 - protein, 6 aa, synthetic construct
  source 1..6: note = Exemplary cyclic peptide, motif HYD_POS_POL-motif, log10 enrichment score 3.833
CPQKAR

SEQ ID NO: 70 - protein, 6 aa, synthetic construct
  source 1..6: note = Exemplary cyclic peptide, motif POL_HYD_ARO-motif, log10 enrichment score 4.237
CMFTLT

SEQ ID NO: 71 - protein, 6 aa, synthetic construct
  source 1..6: note = Exemplary cyclic peptide, motif POS_POL_POS-motif, log10 enrichment score 3.533
CRNRML

SEQ ID NO: 72 - protein, 6 aa, synthetic construct
  source 1..6: note = Exemplary cyclic peptide, motif POL_POL_POS-motif, log10 enrichment score 3.305
CRTLWT

SEQ ID NO: 73 - protein, 6 aa, synthetic construct
  source 1..6: note = Exemplary cyclic peptide, motif POL_HYD_ARO-motif, log10 enrichment score 3.676
CQVFQP

SEQ ID NO: 74 - protein, 6 aa, synthetic construct
  source 1..6: note = Exemplary cyclic peptide, motif POL_HYD_POS-motif, log10 enrichment score 3.231
CLHPYT

SEQ ID NO: 75 - protein, 6 aa, synthetic construct
  source 1..6: note = Exemplary cyclic peptide, motif POS_HYD_POL-motif, log10 enrichment score 3.987
CRICLP

SEQ ID NO: 76 - protein, 6 aa, synthetic construct
  source 1..6: note = Exemplary cyclic peptide, motif POS_POL_POS-motif, log10 enrichment score 3.873
CTRSHF

SEQ ID NO: 77 - protein, 6 aa, synthetic construct
  source 1..6: note = Exemplary cyclic peptide, motif POL_POL_ARO-motif, log10 enrichment score 3.305
CSWNNR

SEQ ID NO: 78 - protein, 6 aa, synthetic construct
  source 1..6: note = Exemplary cyclic peptide, motif POL_NEG_HYD-motif, log10 enrichment score 2.895
CNDLPR

SEQ ID NO: 79 - protein, 6 aa, synthetic construct
  source 1..6: note = Exemplary cyclic peptide, motif ARO_HYD_POL-motif, log10 enrichment score 3.098
CHSTYL

SEQ ID NO: 80 - protein, 6 aa, synthetic construct
  source 1..6: note = Exemplary cyclic peptide, motif HYD_HYD_POL-motif, log10 enrichment score 2.806
CRYELL

SEQ ID NO: 81 - protein, 6 aa, synthetic construct
  source 1..6: note = Exemplary cyclic peptide, motif POL_HYD_HYD-motif, log10 enrichment score 4.185
CPVGCV

SEQ ID NO: 82 - protein, 6 aa, synthetic construct
  source 1..6: note = Exemplary cyclic peptide, motif POL_POL_POL-motif, log10 enrichment score 2.583
CTYYRN

SEQ ID NO: 83 - protein, 6 aa, synthetic construct
  source 1..6: note = Exemplary cyclic peptide, motif POL_HYD_POL-motif, log10 enrichment score 2.891
CRLPQP

SEQ ID NO: 84 - protein, 6 aa, synthetic construct
  source 1..6: note = Exemplary cyclic peptide, motif POL_HYD_HYD-motif, log10 enrichment score 3.312
CQVLQL

SEQ ID NO: 85 - protein, 6 aa, synthetic construct
  source 1..6: note = Exemplary cyclic peptide, motif POL_POL_ARO-motif, log10 enrichment score 3.692
CTFTPT

SEQ ID NO: 86 - protein, 6 aa, synthetic construct
  source 1..6: note = Exemplary cyclic peptide, motif ARO_POL_POS-motif, log10 enrichment score 4.346
CHDLKF

SEQ ID NO: 87 - protein, 6 aa, synthetic construct
  source 1..6: note = Exemplary cyclic peptide, motif POL_HYD_POS-motif, log10 enrichment score 2.758
CLRRRY

SEQ ID NO: 88 - protein, 5 aa, synthetic construct
  source 1..5: note = Exemplary cyclic peptide, motif ARO_POL_ARO-motif, log10 enrichment score 3.715
CLWCF

SEQ ID NO: 89 - protein, 6 aa, synthetic construct
  source 1..6: note = Exemplary cyclic peptide, motif POL_POL_POS-motif, log10 enrichment score 2.818
CRTTQT

SEQ ID NO: 90 - protein, 6 aa, synthetic construct
  source 1..6: note = Exemplary cyclic peptide, motif POL_POL_POL-motif, log10 enrichment score 3.149
CSPNLS

SEQ ID NO: 91 - protein, 6 aa, synthetic construct
  source 1..6: note = Exemplary cyclic peptide, motif HYD_NEG_POL-motif, log10 enrichment score 3.42
CMRLEN

SEQ ID NO: 92 - protein, 6 aa, synthetic construct
  source 1..6: note = Exemplary cyclic peptide, motif POL_POL_ARO-motif, log10 enrichment score 3.189
CYRPLS

SEQ ID NO: 93 - protein, 5 aa, synthetic construct
  source 1..5: note = Exemplary cyclic peptide, motif NEG_POL_POS-motif, log10 enrichment score 3.534
SKYEE

SEQ ID NO: 94 - protein, 6 aa, synthetic construct
  source 1..6: note = Exemplary cyclic peptide, motif POL_HYD_HYD-motif, log10 enrichment score 4.132
SGVGAA

SEQ ID NO: 95 - protein, 6 aa, synthetic construct
  source 1..6: note = Exemplary cyclic peptide, motif POL_HYD_HYD-motif, log10 enrichment score 2.662
CPLLVP

SEQ ID NO: 96 - protein, 6 aa, synthetic construct
  source 1..6: note = Exemplary cyclic peptide, motif HYD_ARO_POL-motif, log10 enrichment score 3.241
CLVPPY

SEQ ID NO: 97 - protein, 6 aa, synthetic construct
  source 1..6: note = Exemplary cyclic peptide, motif POL_HYD_HYD-motif, log10 enrichment score 3.875
CCVVYR

SEQ ID NO: 98 - protein, 6 aa, synthetic construct
  source 1..6: note = Exemplary cyclic peptide, motif POL_HYD_NEG-motif, log10 enrichment score 4.25
CRTADD

SEQ ID NO: 99 - protein, 6 aa, synthetic construct
  source 1..6: note = Exemplary cyclic peptide, motif HYD_NEG_POL-motif, log10 enrichment score 3.365
CPKNLD

SEQ ID NO: 100 - protein, 6 aa, synthetic construct
  source 1..6: note = Exemplary cyclic peptide, motif POL_HYD_HYD-motif, log10 enrichment score 4.105
CPPQVP

SEQ ID NO: 101 - protein, 6 aa, synthetic construct
  source 1..6: note = Exemplary cyclic peptide, motif POL_POL_NEG-motif, log10 enrichment score 3.484
CELLRN

SEQ ID NO: 102 - protein, 6 aa, synthetic construct
  source 1..6: note = Exemplary cyclic peptide, motif HYD_POS_POL-motif, log10 enrichment score 2.885
CLLPRN

SEQ ID NO: 103 - protein, 6 aa, synthetic construct
  source 1..6: note = Exemplary cyclic peptide, motif POL_POL_POL-motif, log10 enrichment score 4.693
CQQQTQ

SEQ ID NO: 104 - protein, 6 aa, synthetic construct
  source 1..6: note = Exemplary cyclic peptide, motif POS_HYD_POL-motif, log10 enrichment score 2.84
CYLFRL

SEQ ID NO: 105 - protein, 6 aa, synthetic construct
  source 1..6: note = Exemplary cyclic peptide, motif HYD_NEG_POL-motif, log10 enrichment score 3.588
CSVECV

SEQ ID NO: 106 - protein, 6 aa, synthetic construct
  source 1..6: note = Exemplary cyclic peptide, motif POL_NEG_HYD-motif, log10 enrichment score 3.283
CDLILR

SEQ ID NO: 107 - protein, 6 aa, synthetic construct
  source 1..6: note = Exemplary cyclic peptide, motif POL_POL_NEG-motif, log10 enrichment score 3.069
CGSTES

SEQ ID NO: 108 - protein, 6 aa, synthetic construct
  source 1..6: note = Exemplary cyclic peptide, motif POS_HYD_POL-motif, log10 enrichment score 3.693
CTTHMS

SEQ ID NO: 109 - protein, 6 aa, synthetic construct
  source 1..6: note = Exemplary cyclic peptide, motif HYD_ARO_POL-motif, log10 enrichment score 3.868
CALWPY

SEQ ID NO: 110 - protein, 6 aa, synthetic construct
  source 1..6: note = Exemplary cyclic peptide, motif NEG_HYD_POL-motif, log10 enrichment score 3.257
CHLDMN

SEQ ID NO: 111 - protein, 6 aa, synthetic construct
  source 1..6: note = Exemplary cyclic peptide, motif POL_POL_ARO-motif, log10 enrichment score 3.921
CTWYRN

SEQ ID NO: 112 - protein, 6 aa, synthetic construct
  source 1..6: note = Exemplary cyclic peptide, motif HYD_HYD_POL-motif, log10 enrichment score 2.71
CWKLLP

SEQ ID NO: 113 - protein, 6 aa, synthetic construct
  source 1..6: note = Exemplary cyclic peptide, motif POL_POL_POS-motif, log10 enrichment score 3.155
CSTTTR

SEQ ID NO: 114 - protein, 6 aa, synthetic construct
  source 1..6: note = Exemplary cyclic peptide, motif POS_HYD_POL-motif, log10 enrichment score 3.134
CTMHVS

SEQ ID NO: 115 - protein, 6 aa, synthetic construct
  source 1..6: note = Exemplary cyclic peptide, motif HYD_HYD_POL-motif, log10 enrichment score 3.313
CRHWVL

SEQ ID NO: 116 - protein, 6 aa, synthetic construct
  source 1..6: note = Exemplary cyclic peptide, motif ARO_POL_ARO-motif, log10 enrichment score 3.006
CYTWTL

SEQ ID NO: 117 - protein, 6 aa, synthetic construct
  source 1..6: note = Exemplary cyclic peptide, motif HYD_POS_POL-motif, log10 enrichment score 3.8
CAHSPL

SEQ ID NO: 118 - protein, 6 aa, synthetic construct
  source 1..6: note = Exemplary cyclic peptide, motif POS_HYD_POL-motif, log10 enrichment score 2.701
CPFKPS

SEQ ID NO: 119 - protein, 6 aa, synthetic construct
  source 1..6: note = Exemplary cyclic peptide, motif POL_POL_POS-motif, log10 enrichment score 3.087
CTKYRN

SEQ ID NO: 120 - protein, 6 aa, synthetic construct
  source 1..6: note = Exemplary cyclic peptide, motif POL_POL_ARO-motif, log10 enrichment score 3.292
CWRLGS

SEQ ID NO: 121 - protein, 6 aa, synthetic construct
  source 1..6: note = Exemplary cyclic peptide, motif POS_POL_POS-motif, log10 enrichment score 3.076
CHVTPR

SEQ ID NO: 122 - protein, 6 aa, synthetic construct
  source 1..6: note = Exemplary cyclic peptide, motif ARO_HYD_POL-motif, log10 enrichment score 3.535
CPVEYV

SEQ ID NO: 123 - protein, 6 aa, synthetic construct
  source 1..6: note = Exemplary cyclic peptide, motif HYD_NEG_POL-motif, log10 enrichment score 3.117
CSKNPD

SEQ ID NO: 124 - protein, 6 aa, synthetic construct
  source 1..6: note = Exemplary cyclic peptide, motif POL_HYD_ARO-motif, log10 enrichment score 3.447
CSGSMF

SEQ ID NO: 125 - protein, 6 aa, synthetic construct
  source 1..6: note = Exemplary cyclic peptide, motif ARO_HYD_POL-motif, log10 enrichment score 3.381
CPTWVT

SEQ ID NO: 126 - protein, 6 aa, synthetic construct
  source 1..6: note = Exemplary cyclic peptide, motif POL_HYD_HYD-motif, log10 enrichment score 4.172
CGVRAA

SEQ ID NO: 127 - protein, 7 aa, synthetic construct
  source 1..7: note = Exemplary cyclic peptide, motif POL_HYD_HYD-motif, log10 enrichment score 3.623
CLLLLPA

SEQ ID NO: 128 - protein, 6 aa, synthetic construct
  source 1..6: note = Exemplary cyclic peptide, motif POL_HYD_HYD-motif, log10 enrichment score 3.636
CPLQVL

SEQ ID NO: 129 - protein, 6 aa, synthetic construct
  source 1..6: note = Exemplary cyclic peptide, motif ARO_POL_POS-motif, log10 enrichment score 3.028
CPLYNR

SEQ ID NO: 130 - protein, 6 aa, synthetic construct
  source 1..6: note = Exemplary cyclic peptide, motif POL_HYD_POL-motif, log10 enrichment score 2.729
CRKHNL

SEQ ID NO: 131 - protein, 6 aa, synthetic construct
  source 1..6: note = Exemplary cyclic peptide, motif HYD_HYD_POL-motif, log10 enrichment score 2.888
CRVPVP

SEQ ID NO: 132 - protein, 6 aa, synthetic construct
  source 1..6: note = Exemplary cyclic peptide, motif POS_POL_POS-motif, log10 enrichment score 3.048
CRNSPR

SEQ ID NO: 133 - protein, 6 aa, synthetic construct
  source 1..6: note = Exemplary cyclic peptide, motif POS_HYD_POL-motif, log10 enrichment score 3.256
CRHRGT

SEQ ID NO: 134 - protein, 5 aa, synthetic construct
  source 1..5: note = Exemplary cyclic peptide, motif ARO_POL_ARO-motif, log10 enrichment score 2.967
CFFRW

SEQ ID NO: 135 - protein, 6 aa, synthetic construct
  source 1..6: note = Exemplary cyclic peptide, motif POS_HYD_POL-motif, log10 enrichment score 3.062
CGSTKP

SEQ ID NO: 136 - protein, 7 aa, synthetic construct
  source 1..7: note = Exemplary cyclic peptide, motif POL_HYD_POS-motif, log10 enrichment score 3.505
CLRHHTT

SEQ ID NO: 137 - protein, 6 aa, synthetic construct
  source 1..6: note = Exemplary cyclic peptide, motif POL_POL_ARO-motif, log10 enrichment score 2.778
CWPLST

SEQ ID NO: 138 - protein, 6 aa, synthetic construct
  source 1..6: note = Exemplary cyclic peptide, motif POL_HYD_ARO-motif, log10 enrichment score 4.011
CGWHSP

SEQ ID NO: 139 - protein, 6 aa, synthetic construct
  source 1..6: note = Exemplary cyclic peptide, motif POL_HYD_POS-motif, log10 enrichment score 2.897
CPRHSV

SEQ ID NO: 140 - protein, 6 aa, synthetic construct
  source 1..6: note = Exemplary cyclic peptide, motif POL_POL_ARO-motif, log10 enrichment score 2.804
CQYGPY

SEQ ID NO: 141 - protein, 6 aa, synthetic construct
  source 1..6: note = Exemplary cyclic peptide, motif POL_HYD_POL-motif, log10 enrichment score 4.035
CKKCTA

SEQ ID NO: 142 - protein, 6 aa, synthetic construct
  source 1..6: note = Exemplary cyclic peptide, motif POL_HYD_HYD-motif, log10 enrichment score 3.213
CPLQIP

SEQ ID NO: 143 - protein, 5 aa, synthetic construct
  source 1..5: note = Exemplary cyclic peptide, motif POS_POL_POS-motif, log10 enrichment score 3.01
CQRQR

SEQ ID NO: 144 - protein, 6 aa, synthetic construct
  source 1..6: note = Exemplary cyclic peptide, motif HYD_ARO_POL-motif, log10 enrichment score 3.729
CPPLWT

SEQ ID NO: 145 - protein, 6 aa, synthetic construct
  source 1..6: note = Exemplary cyclic peptide, motif HYD_ARO_POL-motif, log10 enrichment score 3.394
CPKLYQ

SEQ ID NO: 146 - protein, 6 aa, synthetic construct
  source 1..6: note = Exemplary cyclic peptide, motif POL_POL_POS-motif, log10 enrichment score 2.651
CCHGGT

SEQ ID NO: 147 - protein, 6 aa, synthetic construct
  source 1..6: note = Exemplary cyclic peptide, motif POL_HYD_HYD-motif, log10 enrichment score 3.312
CSANAV

SEQ ID NO: 148 - protein, 6 aa, synthetic construct
  source 1..6: note = Exemplary cyclic peptide, motif ARO_POL_ARO-motif, log10 enrichment score 3.032
CWYGPY

SEQ ID NO: 149 - protein, 6 aa, synthetic construct
  source 1..6: note = Exemplary cyclic peptide, motif POL_HYD_HYD-motif, log10 enrichment score 3.199
CHQMPP

SEQ ID NO: 150 - protein, 6 aa, synthetic construct
  source 1..6: note = Exemplary cyclic peptide, motif POL_HYD_ARO-motif, log10 enrichment score 2.923
CSAFSS

SEQ ID NO: 151 - protein, 5 aa, synthetic construct
  source 1..5: note = Exemplary cyclic peptide, motif POL_HYD_HYD-motif, log10 enrichment score 4.026
CGVKE

SEQ ID NO: 152 - protein, 6 aa, synthetic construct
  source 1..6: note = Exemplary cyclic peptide, motif POL_HYD_ARO-motif, log10 enrichment score 3.556
CQAFQL

SEQ ID NO: 153 - protein, 6 aa, synthetic construct
  source 1..6: note = Exemplary cyclic peptide, motif POL_HYD_ARO-motif, log10 enrichment score 3.101
CLFFPP

SEQ ID NO: 154 - protein, 5 aa, synthetic construct
  source 1..5: note = Exemplary cyclic peptide, motif POL_POL_POL-motif, log10 enrichment score 3.059
SSSLT

SEQ ID NO: 155 - protein, 6 aa, synthetic construct
  source 1..6: note = Exemplary cyclic peptide, motif POL_HYD_HYD-motif, log10 enrichment score 3.395
CGMDYT

SEQ ID NO: 156 - protein, 6 aa, synthetic construct
  source 1..6: note = Exemplary cyclic peptide, motif POL_HYD_HYD-motif, log10 enrichment score 2.985
CVTNPP

SEQ ID NO: 157 - protein, 5 aa, synthetic construct
  source 1..5: note = Exemplary cyclic peptide, motif NEG_POL_NEG-motif, log10 enrichment score 5.215
CDCND

SEQ ID NO: 158 - protein, 6 aa, synthetic construct
  source 1..6: note = Exemplary cyclic peptide, motif POL_NEG_NEG-motif, log10 enrichment score 5.22
CDDVLG

SEQ ID NO: 159 - protein, 6 aa, synthetic construct
  source 1..6: note = Exemplary cyclic peptide, motif POL_NEG_ARO-motif, log10 enrichment score 5.392
CDFLTH

SEQ ID NO: 160 - protein, 5 aa, synthetic construct
  source 1..5: note = Exemplary cyclic peptide, motif POL_NEG_POS-motif, log10 enrichment score 4.976
CDKEY

SEQ ID NO: 161 - protein, 6 aa, synthetic construct
  source 1..6: note = Exemplary cyclic peptide, motif POL_NEG_POS-motif, log10 enrichment score 5.94
CDKVAT

SEQ ID NO: 162 - protein, 5 aa, synthetic construct
  source 1..5: note = Exemplary cyclic peptide, motif POL_NEG_ARO-motif, log10 enrichment score 5.255
CDWKK

SEQ ID NO: 163 - protein, 6 aa, synthetic construct
  source 1..6: note = Exemplary cyclic peptide, motif NEG_POL_NEG-motif, log10 enrichment score 4.896
CERADE

SEQ ID NO: 164 - protein, 6 aa, synthetic construct
  source 1..6: note = Exemplary cyclic peptide, motif POL_NEG_NEG-motif, log10 enrichment score 5.073
CERQED

SEQ ID NO: 165 - protein, 6 aa, synthetic construct
  source 1..6: note = Exemplary cyclic peptide, motif HYD_NEG_POL-motif, log10 enrichment score 5.17
CESDAE

SEQ ID NO: 166 - protein, 6 aa, synthetic construct
  source 1..6: note = Exemplary cyclic peptide, motif POS_POL_ARO-motif, log10 enrichment score 5.321
CFHTYQ

SEQ ID NO: 167 - protein, 6 aa, synthetic construct
  source 1..6: note = Exemplary cyclic peptide, motif POS_POL_ARO-motif, log10 enrichment score 4.949
CFMSPK

SEQ ID NO: 168 - protein, 6 aa, synthetic construct
  source 1..6: note = Exemplary cyclic peptide, motif ARO_POL_POS-motif, log10 enrichment score 4.768
CHLKSW

SEQ ID NO: 169 - protein, 6 aa, synthetic construct
  source 1..6: note = Exemplary cyclic peptide, motif NEG_POL_NEG-motif, log10 enrichment score 4.917
CLDDSE

SEQ ID NO: 170 - protein, 6 aa, synthetic construct
  source 1..6: note = Exemplary cyclic peptide, motif POS_POL_ARO-motif, log10 enrichment score 4.889
CPHQFR

SEQ ID NO: 171 - protein, 6 aa, synthetic construct
  source 1..6: note = Exemplary cyclic peptide, motif POL_NEG_NEG-motif, log10 enrichment score 4.949
CSDEEK

SEQ ID NO: 172 - protein, 6 aa, synthetic construct
  source 1..6: note = Exemplary cyclic peptide, motif POL_NEG_NEG-motif, log10 enrichment score 4.94
CSDETI

SEQ ID NO: 173 - protein, 6 aa, synthetic construct
  source 1..6: note = Exemplary cyclic peptide, motif POL_NEG_ARO-motif, log10 enrichment score 5.022
CSEWGM

SEQ ID NO: 174 - protein, 6 aa, synthetic construct
  source 1..6: note = Exemplary cyclic peptide, motif POL_NEG_ARO-motif, log10 enrichment score 4.703
CVTEWS

SEQ ID NO: 175 - protein, 6 aa, synthetic construct
  source 1..6: note = Exemplary cyclic peptide, motif POL_NEG_ARO-motif, log10 enrichment score 4.959
CVYQDY

SEQ ID NO: 176 - protein, 6 aa, synthetic construct
  source 1..6: note = Exemplary cyclic peptide, motif NEG_POL_ARO-motif, log10 enrichment score 5.202
CYRQED

SEQ ID NO: 177 - protein, 7 aa, synthetic construct
  source 1..7: note = Exemplary cyclic peptide, motif ARO_HYD_POL-motif, log10 enrichment score 2.336
CCAWPSA

SEQ ID NO: 178 - protein, 7 aa, synthetic construct
  source 1..7: note = Exemplary cyclic peptide, motif POS_HYD_POL-motif, log10 enrichment score 2.307
CCTMHMS

SEQ ID NO: 179 - protein, 7 aa, synthetic construct
  source 1..7: note = Exemplary cyclic peptide, motif POL_HYD_ARO-motif, log10 enrichment score 2.938
CTLYHRT

SEQ ID NO: 180 - protein, 7 aa, synthetic construct
  source 1..7: note = Exemplary cyclic peptide, motif POL_HYD_HYD-motif, log10 enrichment score 2.866
CLASSLT

SEQ ID NO: 181 - protein, 7 aa, synthetic construct
  source 1..7: note = Exemplary cyclic peptide, motif HYD_ARO_POL-motif, log10 enrichment score 3.251
CVTLLLY

SEQ ID NO: 182 - protein, 7 aa, synthetic construct
  source 1..7: note = Exemplary cyclic peptide, motif POS_HYD_POL-motif, log10 enrichment score 3.188
CRPSSSS

SEQ ID NO: 183 - protein, 7 aa, synthetic construct
  source 1..7: note = Exemplary cyclic peptide, motif POL_HYD_POS-motif, log10 enrichment score 2.617
CSGRLPS

SEQ ID NO: 184 - protein, 7 aa, synthetic construct
  source 1..7: note = Exemplary cyclic peptide, motif POL_POL_POL-motif, log10 enrichment score 2.6
CPTLLSS

SEQ ID NO: 185 - protein, 7 aa, synthetic construct
  source 1..7: note = Exemplary cyclic peptide, motif POL_HYD_ARO-motif, log10 enrichment score 3.462
CLFTLSY

SEQ ID NO: 186 - protein, 7 aa, synthetic construct
  source 1..7: note = Exemplary cyclic peptide, motif POS_HYD_POL-motif, log10 enrichment score 2.794
CLRLSSL

SEQ ID NO: 187 - protein, 7 aa, synthetic construct
  source 1..7: note = Exemplary cyclic peptide, motif POL_HYD_HYD-motif, log10 enrichment score 3.195
CLPSSPP

SEQ ID NO: 188 - protein, 7 aa, synthetic construct
  source 1..7: note = Exemplary cyclic peptide, motif POL_HYD_HYD-motif, log10 enrichment score 2.662
CGTLPSY

SEQ ID NO: 189 - protein, 7 aa, synthetic construct
  source 1..7: note = Exemplary cyclic peptide, motif POL_POL_POL-motif, log10 enrichment score 2.752
CSGSGLT

SEQ ID NO: 190 - protein, 7 aa, synthetic construct
  source 1..7: note = Exemplary cyclic peptide, motif HYD_POS_POL-motif, log10 enrichment score 2.945
CSTLRST

SEQ ID NO: 191 - protein, 7 aa, synthetic construct
  source 1..7: note = Exemplary cyclic peptide, motif POL_HYD_HYD-motif, log10 enrichment score 2.847
CPLSPIS

SEQ ID NO: 192 - protein, 7 aa, synthetic construct
  source 1..7: note = Exemplary cyclic peptide, motif POS_HYD_POL-motif, log10 enrichment score 3.316
CTPQNHP

SEQ ID NO: 193 - protein, 7 aa, synthetic construct
  source 1..7: note = Exemplary cyclic peptide, motif ARO_HYD_POL-motif, log10 enrichment score 3.297
CFLSRSG

SEQ ID NO: 194 - protein, 7 aa, synthetic construct
  source 1..7: note = Exemplary cyclic peptide, motif POL_HYD_HYD-motif, log10 enrichment score 3.05
CPPPPLP

SEQ ID NO: 195 - protein, 7 aa, synthetic construct
  source 1..7: note = Exemplary cyclic peptide, motif POL_POL_POS-motif, log10 enrichment score 2.792
CSTRTLP

SEQ ID NO: 196 - protein, 7 aa, synthetic construct
  source 1..7: note = Exemplary cyclic peptide, motif POL_POL_POL-motif, log10 enrichment score 2.866
CSILLLT

SEQ ID NO: 197 - protein, 7 aa, synthetic construct
  source 1..7: note = Exemplary cyclic peptide, motif POS_HYD_POL-motif, log10 enrichment score 2.662
CRVTLSN

SEQ ID NO: 198 - protein, 7 aa, synthetic construct
  source 1..7: note = Exemplary cyclic peptide, motif POL_POL_POS-motif, log10 enrichment score 3.759
CLSYTSK

SEQ ID NO: 199 - protein, 7 aa, synthetic construct
  source 1..7: note = Exemplary cyclic peptide, motif POL_POL_ARO-motif, log10 enrichment score 3.586
CPLSTYP

SEQ ID NO: 200 - protein, 7 aa, synthetic construct
  source 1..7: note = Exemplary cyclic peptide, motif HYD_POS_POL-motif, log10 enrichment score 2.989
CLVLPHS

SEQ ID NO: 201 - protein, 7 aa, synthetic construct
  source 1..7: note = Exemplary cyclic peptide, motif POL_HYD_HYD-motif, log10 enrichment score 2.964
CLLLHYS

SEQ ID NO: 202 - protein, 7 aa, synthetic construct
  source 1..7: note = Exemplary cyclic peptide, motif POL_POL_ARO-motif, log10 enrichment score 2.569
CPSSFTS

SEQ ID NO: 203 - protein, 7 aa, synthetic construct
  source 1..7: note = Exemplary cyclic peptide, motif ARO_HYD_POL-motif, log10 enrichment score 2.583
CFATPTL

SEQ ID NO: 204 - protein, 7 aa, synthetic construct
  source 1..7: note = Exemplary cyclic peptide, motif POL_HYD_ARO-motif, log10 enrichment score 3.138
CPRHNLF

SEQ ID NO: 205 - protein, 7 aa, synthetic construct
  source 1..7: note = Exemplary cyclic peptide, motif POL_HYD_POL-motif, log10 enrichment score 3.358
CPQHRHY

SEQ ID NO: 206 - protein, 8 aa, synthetic construct
  source 1..8: note = Exemplary cyclic peptide, motif HYD_POS_POL-motif, log10 enrichment score 2.5
CLSPHNCR

SEQ ID NO: 207 - protein, 8 aa, synthetic construct
  source 1..8: note = Exemplary cyclic peptide, motif NEG_HYD_POL-motif, log10 enrichment score 4.025
CVQRIFDI

SEQ ID NO: 208 - protein, 8 aa, synthetic construct
  source 1..8: note = Exemplary cyclic peptide, motif POS_HYD_POL-motif, log10 enrichment score 2.595
CPSYAHHA

SEQ ID NO: 209 - protein, 8 aa, synthetic construct
  source 1..8: note = Exemplary cyclic peptide, motif POS_POL_POS-motif, log10 enrichment score 2.948
CHNRTVCR

SEQ ID NO: 210 - protein, 8 aa, synthetic construct
  source 1..8: note = Exemplary cyclic peptide, motif POL_POL_POL-motif, log10 enrichment score 3.562
CGPSTSQA

SEQ ID NO: 211 - protein, 8 aa, synthetic construct
  source 1..8: note = Exemplary cyclic peptide, motif POL_HYD_POS-motif, log10 enrichment score 2.906
CVKRIARW

SEQ ID NO: 212 - protein, 8 aa, synthetic construct
  source 1..8: note = Exemplary cyclic peptide, motif POL_HYD_HYD-motif, log10 enrichment score 3.391
CLASWLLA

SEQ ID NO: 213 - protein, 8 aa, synthetic construct
  source 1..8: note = Exemplary cyclic peptide, motif POL_POL_ARO-motif, log10 enrichment score 4.246
CYLLQLSS

SEQ ID NO: 214 - protein, 8 aa, synthetic construct
  source 1..8: note = Exemplary cyclic peptide, motif HYD_HYD_POL-motif, log10 enrichment score 3.19
CWPRPLLL

SEQ ID NO: 215 - protein, 8 aa, synthetic construct
  source 1..8: note = Exemplary cyclic peptide, motif POL_HYD_HYD-motif, log10 enrichment score 2.796
CLNPSQVV

SEQ ID NO: 216 - protein, 8 aa, synthetic construct
  source 1..8: note = Exemplary cyclic peptide, motif POS_POL_POS-motif, log10 enrichment score 2.449
CRMISPCR

SEQ ID NO: 217 - protein, 8 aa, synthetic construct
  source 1..8: note = Exemplary cyclic peptide, motif POL_HYD_ARO-motif, log10 enrichment score 3.506
CPLQVFQL

SEQ ID NO: 218 - protein, 8 aa, synthetic construct
  source 1..8: note = Exemplary cyclic peptide, motif POS_POL_POS-motif, log10 enrichment score 2.771
CRTQLTIR

SEQ ID NO: 219 - protein, 8 aa, synthetic construct
  source 1..8: note = Exemplary cyclic peptide, motif ARO_HYD_POL-motif, log10 enrichment score 2.321
CSMFITVF

SEQ ID NO: 220 - protein, 8 aa, synthetic construct
  source 1..8: note = Exemplary cyclic peptide, motif POS_HYD_POL-motif, log10 enrichment score 2.238
CAIHHACS

SEQ ID NO: 221 - protein, 8 aa, synthetic construct
  source 1..8: note = Exemplary cyclic peptide, motif POL_HYD_HYD-motif, log10 enrichment score 3.846
CMPSSLLL

SEQ ID NO: 222 - protein, 8 aa, synthetic construct
  source 1..8: note = Exemplary cyclic peptide, motif HYD_POS_POL-motif, log10 enrichment score 3.575
CPIYAHAK

SEQ ID NO: 223 - protein, 8 aa, synthetic construct
  source 1..8: note = Exemplary cyclic peptide, motif POL_POL_NEG-motif, log10 enrichment score 3.192
CETTAGGS

SEQ ID NO: 224 - protein, 8 aa, synthetic construct
  source 1..8: note = Exemplary cyclic peptide, motif ARO_POL_POS-motif, log10 enrichment score 3.736
CSNSCYCR

SEQ ID NO: 225 - protein, 8 aa, synthetic construct
  source 1..8: note = Exemplary cyclic peptide, motif HYD_POS_POL-motif, log10 enrichment score 3.075
CQNATVRC

SEQ ID NO: 226 - protein, 8 aa, synthetic construct
  source 1..8: note = Exemplary cyclic peptide, motif POL_HYD_HYD-motif, log10 enrichment score 2.413
CVMGHHYS

SEQ ID NO: 227 - protein, 8 aa, synthetic construct
  source 1..8: note = Exemplary cyclic peptide, motif POS_POL_ARO-motif, log10 enrichment score 3.229
CRQFYMPR

SEQ ID NO: 228 - protein, 9 aa, synthetic construct
  source 1..9: note = Exemplary cyclic peptide, motif ARO_POL_POS-motif, log10 enrichment score 2.301
CVYSRSTCR

SEQ ID NO: 229 - protein, 224 aa, synthetic construct
  source 1..224: note = Exemplary of cyclic peptide generator
  VARIANT 38: note = Any amino acid or no amino acid
  VARIANT 39: note = Any amino acid or no amino acid
  VARIANT 40: note = Any amino acid or no amino acid
  VARIANT 41: note = Any amino acid or no amino acid
  VARIANT 42: note = Any amino acid or no amino acid
  VARIANT 43: note = Any amino acid or no amino acid
  VARIANT 44: note = Any amino acid or no amino acid
  VARIANT 45: note = Any amino acid or no amino acid
  VARIANT 46: note = Any amino acid or no amino acid
  VARIANT 47: note = Any amino acid or no amino acid
  VARIANT 48: note = Any amino acid or no amino acid
  VARIANT 49: note = Any amino acid or no amino acid
  VARIANT 38..49: note = This region may encompass 3-12 residues
MVKVIGRRSLGVQRIFDIGLPQDHNFLLANGAIAHNCXXXXXXXXXXXXCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIKTTNPGVSAWQVNTAYTAGQLVTYNGKTYKCLQPHTSLAGWEPSNVPALWQLQ

SEQ ID NO: 230 - protein, 6 aa, synthetic construct
  source 1..6: note = Cyclic peptide that was found to enhance AAV viral titer
CRRYHA